(19) United States
(12) Patent Application Publication
(10) Pub. No.: US 2020/0009266 A1
(43) Pub. Date: Jan. 9, 2020

(54) IN VIVO GENE THERAPY FOR IMMUNE DEFICIENCIES

(71) Applicants: Fred Hutchinson Cancer Research Center, Seattle, WA (US); Seattle Children's Hospital d/b/a Seattle Children's Research Institute, Seattle, WA (US)

(72) Inventors: Frieda Chan, Bellevue, WA (US); Olivier Humbert, Seattle, WA (US); Hans-Peter Kiem, Seattle, WA (US); Jennifer E. Adair, Seattle, WA (US); David Rawlings, Seattle, WA (US); Andrew Scharenberg, Seattle, WA (US); Troy Torgerson, Seattle, WA (US)

(73) Assignees: Fred Hutchinson Cancer Research Center, Seattle, WA (US); Seattle Children's Hospital d/b/a Seattle Children's Research Institute, Seattle, WA (US)

(21) Appl. No.: 16/486,442

(22) PCT Filed: Feb. 15, 2018
(86) PCT No.: PCT/US18/18439

§ 371 Date: Aug. 15, 2019

Related U.S. Application Data
(60) Provisional application No. 62/459,450, filed on Feb. 15, 2017.

(51) Int. Cl. A61K 48/00; A61K 38/19; A61K 31/395; A61P 37/00

(52) U.S. Cl. CPC A61K 48/005; A61P 37/00; A61K 31/395; A61K 38/193

(57) ABSTRACT

In vivo gene therapies for immune deficiencies are described. The in vivo gene therapies utilize a foamy viral vector including a PGK promoter with a therapeutic gene. The foamy viral vector can be beneficially administered with cell mobilization into the peripheral blood.

### CROSS-REFERENCE TO RELATED APPLICATIONS

This application claims priority to U.S. Provisional Application No. 62/459,450 filed on Feb. 15, 2017, which is incorporated herein by reference in its entirety as if fully set forth herein.

### STATEMENT REGARDING FEDERALLY SPONSORED RESEARCH OR DEVELOPMENT

This invention was made with government support under grant AI097100 awarded by the National Institutes of Health. The government has certain rights in the invention.

### REFERENCE TO SEQUENCE LISTING

A computer readable text file, entitled “17-065-WO-PCT Sequence Listing_ST25.txt” created on or about Feb. 15, 2018, with a file size of 183 KB, contains the sequence listing for this application and is hereby incorporated by reference in its entirety.

### FIELD OF THE DISCLOSURE

In vivo gene therapies for immune deficiencies are described. The in vivo gene therapies utilize a foamy viral vector including a PGK promoter associated with a therapeutic gene. The foamy viral vector can be beneficially administered with cell mobilization into the peripheral blood.

### BACKGROUND OF THE DISCLOSURE

More than 80 primary immune deficiency diseases are recognized by the World Health Organization. These diseases are characterized by an intrinsic defect in the immune system in which, in some cases, the body is unable to produce any or enough antibodies against infection. In other cases, cellular defenses to fight infection fail to work properly. Typically, primary immune deficiencies are inherited disorders.

Secondary, or acquired, immune deficiencies are not the result of inherited genetic abnormalities, but rather occur in individuals in which the immune system is compromised by factors outside the immune system. Examples include trauma, viruses, chemotherapy, toxins, and pollution. Acquired immunodeficiency syndrome (AIDS) is an example of a secondary immune deficiency disorder caused by a virus, the human immunodeficiency virus (HIV), in which a depletion of T lymphocytes renders the body unable to fight infection.

X-linked severe combined immunodeficiency (SCID-X1) is both a cellular and humoral immune depletion caused by mutations in the common gamma chain gene (γC), which result in the absence of T and natural killer (NK) lymphocytes and the presence of nonfunctional B lymphocytes. SCID-X1 is fatal in the first two years of life unless the immune system is reconstituted, for example, through bone marrow transplant (BMT) or gene therapy.

Because most individuals lack a matched donor for BMT or non-autologous gene therapy, haploidentical parental bone marrow depleted of mature T cells is often used; however, complications include graft versus host disease (GVHD), failure to make adequate antibodies hence requiring long-term immunoglobulin replacement, late loss of T cells due to failure to engraft hematopoietic stem and progenitor cells (HSPCs), chronic warts, and lymphocyte dysregulation.

Fanconi anemia (FA) is an inherited blood disorder that leads to bone marrow failure. It is characterized, in part, by a deficient DNA-repair mechanism. At least 20% of patients with FA develop cancers such as acute myeloid leukemias, and cancers of the skin, liver, gastrointestinal tract, and gynecological systems. The skin and gastrointestinal tumors are usually squamous cell carcinomas. The average age of patients who develop cancer is 15 years for leukemia, 16 years for liver tumors, and 23 years for other tumors.

Cells from FA patients display a characteristic hypersensitivity to agents that produce interstrand DNA crosslinks such as mitomycin C or diepoxybutane. FA genes define a multicomponent pathway involved in cellular responses to DNA cross-links. Five of the FA genes (FANCA, FANCC, FANCE, FANCF and FANCG) have been cloned and the FANCA, FANCC and FANCG proteins have been shown to form a molecular complex with primarily nuclear localization. A number of mutations in the FANCC gene have been identified which are correlated with FA of differing degrees of severity.

An alternative therapeutic approach to BMT and non-autologous gene therapy in immune and blood disorder failures is ex vivo HSPC gene therapy, where blood or bone marrow derived HSPCs are enriched from patients, transduced with viral vectors to deliver a functional therapeutic gene (e.g., a γC gene for SCID-X1 or a FancA gene for FA), and transplanted back to the patient. The first generation ex vivo gene therapy for SCID-X1 used murine leukemia virus-based gammaretroviral (RV) delivery and showed significant long-term clinical improvement in treated patients. However, 5/20 patients unexpectedly developed T cell leukemia, resulting in the death of one patient. These findings precipitated intense interest in utilization of self-inactivating (SIN) viral vectors and SIN-lentiviral vectors (LV) as alternative vector platforms. While SIN-RV and SIN-LV are currently used in the clinical setting with considerable success, ex vivo gene therapy still faces multiple challenges that include the: 1) extensive ex vivo manipulation of HSPCs required to prepare them for therapeutic use that results in loss of multipotency potential and/or reduced fitness for engraftment following transplantation, 2) various conditioning regimens used to enhance engraftment of gene modified HSPCs add considerable genotoxic risks to patients, and 3) requirement of advanced infrastructures for the collection, culture, transduction, validation, and re-infusion of HSPCs, consequently restricting this form of treatment to a select few institutions worldwide.

With these limitations in mind, treatment using in vivo gene therapy, which includes the direct delivery of a viral vector to a patient, was explored. In vivo gene therapy is a simple and attractive approach because it may not require any genotoxic conditioning (or could require less genotoxic conditioning) nor ex vivo cell processing and thus could be adopted at many institutions worldwide, including those in developing countries, as the therapy could be administered through an injection, similar to what is already done worldwide for the delivery of vaccines.

Foamy virus (FV) vectors are non-pathogenic integrating retroviruses, which are highly effective for HSPC gene therapy and potentially safer than SIN-RV and LV. For example, foamy vector proviruses integrate less frequently in genes than LV vectors, and have a reduced propensity to transactivate nearby genes than LV or RV vectors. These properties likely contribute to their safety as established in the canine model and in the murine xenotransplantation model. Unlike VSV-G pseudotyped LV vectors, FV vectors are resistant to human serum inactivation, which gives them a specific advantage during in vivo delivery and would allow for multiple infusions of the same FV vector if multiple dosages were required.

The feasibility of in vivo gene therapy in canine SCID-X1 with intravenous injection of FV vector expressing human codon optimized γC driven by the short elongation factor-1 alpha promoter (EF1α; EF1α.γC.FV) was previously demonstrated. Successful lymphocyte expansion was reported in these animals but clonal diversity and the T-cell receptor (TCR) repertoire were low. Ultimately, all animals were euthanized due to infections, highlighting the acute need for novel protocols and reagents to achieve clinically meaningful outcomes in patients.

### SUMMARY OF THE DISCLOSURE

The current disclosure provides systems and methods that improve the kinetics of T cell correction and expansion in immune deficient subjects beyond that achieved by the prior art. The systems and methods utilize a foamy viral vector including a human phosphoglycerate kinase (PGK) promoter (instead of an EF1α promoter) to drive expression of therapeutic genes, such as γC for SCID or FancA for FA. Use of the PGK promoter can be beneficially combined with mobilization, for example with the combination of granulocyte-colony-stimulating factor (G-CSF) and AMD3100 prior to FV vector administration. This addition enhances FV vector transduction of HSPCs, which normally reside in the bone marrow stoma. These conditions markedly increased both kinetics and clonal diversity of lymphocyte reconstitution, and also correlated with more robust thymopoiesis. These significant enhancements further ongoing efforts to bring genetic therapies to patients in dire need of immune system reconstitution due to primary or secondary immune deficiencies.

### BRIEF DESCRIPTION OF THE FIGURES

Many of the figures submitted herein are better understood in color. Applicants consider the color versions of the drawings as part of the original submission and reserve the right to present color images of the drawings in later proceedings.

FIG. 1. Increased transgene expression in CD34+ cells transduced with PGK.GFP.2A.γC.FV. Human and canine CD34+ cells enriched from mobilized peripheral blood and steady state bone marrow, respectively, were transduced with PGK.GFP.2A.γC.FV or EF1α.GFP.2A.γC.FV at an MOI of 10. GFP expression was measured by flow cytometry at 6 days post transduction and the fraction of GFP+ cells as well as mean fluorescence intensity (MFI) of the GFP+ fraction are shown for each vector. Filled gray histograms are from untransduced cells.

FIGS. 2A-2C. Competitive injection of SCID-X1 dogs with EF1α and PGK FV vectors. FIG. 2A) Dogs R2258 and R2260 were injected with a combination of FV vectors PGK.γC.FV and EF1α.γC.FV containing the fluorophores eGFP or mCherry. T2A: Thosea asigna virus 2A self-cleaving peptide. FIG. 2B) Kinetics of lymphocyte reconstitution (lymphocytes per μL peripheral blood) in R2258 and R2260. Range of lymphocyte counts in healthy dogs is shown by horizontal dashed lines. FIG. 2C) Long-term analysis of gene marking in peripheral blood lymphocytes from R2258 and R2260 for the PGK and EF1α FV vectors based on fluorophore expression. Lymphocyte population was defined based on forward and side scatter.

FIG. 3. Animals treated with in vivo FV vector gene therapy.

FIGS. 4A-4E. Enhanced T-lymphocyte reconstitution with G-CSF/AMD3100 treatment prior to FV vector injection. FIG. 4A) Schematic of experiment with G-CSF/AMD3100 treatment prior to FV vector injection. FIG. 4B) Flow cytometry plot of peripheral blood CD34+ cells in non-mobilized (H866) or mobilized (H867) newborn canines at 6 hours post treatment. FIG. 4C) Kinetics of gene marking based on fluorophore expression in circulating lymphocytes from dogs treated with different FV vectors with or without G-CSF/AMD3100 mobilization. Lymphocyte population was defined based on forward and side scatter. FIG. 4D) Kinetics of lymphocyte reconstitution (lymphocytes per μL peripheral blood) in the same animals described in FIG. 4C. FIG. 4E) Kinetics of CD3+ cells reconstitution (cells per μL peripheral blood) in the same animals described in FIG. 4C during the first 7 months post-treatment. In FIG. 4D and FIG. 4E, normal range of lymphocyte/CD3+ cell counts is shown by horizontal dashed lines. Animal R2203 only survived for 119 days post treatment.

FIGS. 5A, 5B. Representative phenotypic panel for different blood cell lineages from treated canines. FIG. 5A) Peripheral blood gene marking as determined by mCherry or GFP in different lymphocyte subsets from animal R2258 at 620 days post treatment. Fraction of CD4 and CD8 subsets within CD3+ cells is shown on the right. FIG. 5B) Gene marking in different cell lineages from the same animal defined by forward and side scatter. For monocytes and granulocytes, CD3+ cells were first gated out to exclude contaminating lymphocytes.

FIGS. 6A-6C. Levels of gene marking detected in myeloid and B cells obtained from treated canines. Gene marking was determined based on phenotypic panel described in FIGS. 5A-5B in granulocytes (FIG. 6A), monocytes (FIG. 6B) and B lymphocytes (FIG. 6C) from peripheral blood of treated animals.

FIGS. 7A-7C. Clonal diversity as determined by retroviral integration site (RIS) analysis in non-mobilized and G-CSF/AMD3100 mobilized dogs prior to FV vector injection. FIG. 7A) Clonal diversity in non-mobilized canines R2258 (left) and R2260 (right) at the indicated time points post treatment. FIG. 7B) Clonal diversity in mobilized canines H864 (left) and H867 (right) at the indicated time points post treatment with G-CSF/AMD3100 and vector PGK.γC.FV. In all graphs, unique RISs are plotted based on the number of times the RIS was sequenced and normalized to the percentage of total RISs captured at each time point for each animal. Total number of unique RISs is shown on top of each bar. Captured RISs appearing at a frequency greater than 1% in each sample are represented by boxes in each graph. Boxes are colored in white if they were identified at a single time point, or in matching colors if they were identified in more than one time point at a frequency higher than 1%. The gray portion of the graph depicts all RISs with a frequency lower than 1% at each time point. FIG. 7C) Location of RIS events analyzed in peripheral blood of FV-vector treated canines. Each color shows the location of a unique integration event that corresponds to the color of the bar graphs depicted in FIGS. 7A, 7B. Locations are defined as: chromosome (chr) number_bp location_sense (+) or antisense (−) orientation of insertion event.

FIGS. 8A-8D. Thymic output in FV vector treated animals with and without G-CSF/AMD3100 mobilization. FIG. 8A) Fraction of CD45RA+ cells within the CD3+ population in peripheral blood of non-mobilized animals R2258 and R2260. FIG. 8B) Fraction of CD3+CD45RA+ cells in animals H864 and H867 treated with G-CSF/AMD3100 mobilization and vector PGK.γC.FV. FIG. 8C) T cell receptor excision circles (TRECs) levels in peripheral blood of the same animals described in FIG. 8A. FIG. 8D) TRECs levels in peripheral blood of the same animals described in FIG. 8B. In FIG. 8A and FIG. 8B, dashed line shows average % CD45RA+ from normal dog; in FIG. 8C and FIG. 8D, dashed line shows TRECs levels from normal littermate control.

FIGS. 9A-9C. CD4/CD8 and TCR alpha/beta (ab) lymphocyte subtypes in FV vector-treated canines. FIG. 9A) Bar graph depicting CD4+, CD8+, CD4/CD8 double positive and CD4/CD8 double negative cells within the CD3+ population as determined by flow cytometry staining in non-mobilized/FV vector treated animals R2258 and R2260. FIG. 9B) Same analysis as in FIG. 9A in mobilized/ FV vector treated animals H864 and H867. FIG. 9C) Percentage TCR alpha/beta (ab) lymphocytes within the CD3+ population in animals H864 and H867, and in normal dog.

FIGS. 10A, 10B. T cell receptor (TCR) diversity as determined by TCR Vbeta spectratyping in mobilized FV vector treated dogs. Rearrangement of the TCR beta chain was assessed by PCR amplification of complementary DNA using 17 different primer pairs (annotated on top) at various time points post treatment in a normal littermate control H866 (FIG. 10A) and in treated SCID-X1 dogs H864 and H867 (FIG. 10B).

FIG. 11. TCR Vbeta spectratyping analysis in non-mobilized, FV vector treated dogs. Rearrangement of the TCR beta chain was assessed by PCR amplification of complementary DNA using 17 different primer pairs (annotated on top) at different time points post treatment (Month, Mo) in non-mobilized, FV treated dogs R2258 and R2260.

FIGS. 12A-12C. Validation of T-lymphocyte function in cells obtained from FV vector treated SCID-X1 canines. FIG. 12A) STAT3 phosphorylation (pSTAT3) was measured in peripheral blood mononuclear cells (PBMCs) isolated from animals R2258 and R2260 (non-mobilized, 485 days post treatment), or from a normal littermate control, and cultured in vitro with no, low, or high levels of IL-21. pSTAT3 signal is gated from CD3+ cells. FIG. 12B) pSTAT3 phosphorylation was assessed in mobilized animals H864 and H867 at 1-year post treatment and compared to a littermate control (normal) as described in FIG. 12A. Results are expressed as fold increase in cells expressing pSTAT3 when exposed to IL-21 as compared to untreated cells. Error bars show standard deviation for duplicate reactions and no statistical difference was noted between normal and H864 or H867 datasets using a Welch's t test (p=0.32 and p=0.11, respectively). FIG. 12C) Proliferative response to phytohemagglutinin (PHA) of PBMCs isolated from the same animals and same time point as in FIG. 12A. Cell proliferation was determined by dilution of CellTracker™ dye (Thermo Fisher Scientific, Waltham, Mass.).

FIGS. 13A, 13B. Immunoglobulin responses in treated SCID-X1 canines. FIG. 13A) Bacteriophage immunoglobulin response in serum of FV treated SCID-X1 dogs (mobilized=square lines, non-mobilized=diamond lines), and in a normal control (“x” line) plus or minus two standard deviations (dashed lines). Animals were injected with a first dose of bacteriophage φX174 at 8 to 12 months post-FV vector treatment, and with a second dose 6 weeks later. Primary and secondary immune responses to injection were assessed at 1, 2, and 4 weeks, compared to pre-injection levels, and expressed as the rate of phage inactivation or K value (Kv) (Material and Methods). FIG. 13B) Quantitative measurement of the 3 main classes of immunoglobulin in serum of mobilized animals at 15 months post treatment as compared to a normal control. Standard range is based on normal 1 year old dog.

FIGS. 14A-14E. Complete blood count analysis in all treated dogs. Graphs depict absolute numbers (counts per μL blood) of FIG. 14A) total white blood cells, FIG. 14B) neutrophils, FIG. 14C) monocytes, FIG. 14D) lymphocytes, and FIG. 14E) platelets in peripheral blood. Dotted line denotes average range of blood cell counts of normal dogs.

FIG. 15. Retroviral integration site (RIS) analysis of tissues obtained from R2258 and R2260 at time of necropsy. Clonal diversity in the different tissues annotated on x-axis was compared to peripheral blood (PB), with total number of RIS events identified in the sample listed on top of the corresponding bar. In all graphs, unique RISs are plotted based on the number of times the RIS was sequenced and normalized to the percentage of total RISs captured in each sample for each animal. Captured RISs appearing at a frequency greater than 1% in each sample are represented by boxes in each bar. Boxes are colored white if they were identified in a single sample, or in matching colors if they were identified in more than one sample at a frequency higher than 1%. Colored numbers in gray portion depict RISs identified in more than one sample with a frequency lower than 1%. BM=bone marrow.

FIG. 16. Frequency of shared and unique RIS events in tissues obtained from animals R2258 and R2260. RISs from each tissue described in FIG. 15 were compared with peripheral blood (PB) and with each other to determine overlap in multiple tissues (black), but absent from PB (dark gray), or that were unique to a specific tissue and absent from PB (light gray).

FIGS. 17A, 17B. Frequency of unique RIS events in ovary and testis (FIG. 17A) and in semen (FIG. 17B).

FIG. 18. Frequency of shared and unique RIS events observed in semen collected from mobilized canine H867. Semen was collected and gDNA was isolated and subjected to RIS analysis. A total of 16 RISs were identified in this sample and compared with all known PB RISs (total of 4,105 RISs; FIG. 7B) to determine overlap (black). 12 integration sites were unique to the semen sample (white) but were only detected as single hit (FIG. 17B), representing 2.28% of all hits. Because the animal remains alive and well, full biodistribution analysis is not available.

FIGS. 19A, 19B. FIG. 19A) Comparison of lymphocyte reconstitution of canines indicated in FIG. 19B with human ex vivo clinical trial (filled and open circle lines) from Hacein-Bey-Abina S et al. (2014) NEJM 371: 1407-1417. FIG. 19B) same canine data as in FIG. 19A with details of specific in vivo FV vectors used.

FIG. 20. Gene marking and donor chimerism detected in H084 following transplantation and chemo-selection. Dark squares with solid line represent percentage of gene marking following transplantation of RSCPSMPG′ISCEW2 lentiviral vector-transduced donor (H089) cells in recipient (H084) peripheral blood. Open squares with dotted line represent percentage of donor chimerism in recipient (H084) peripheral blood. Dark triangles denote the two rounds of combination chemotherapy (O6BG and BCNU) administered to recipient.

FIG. 21. Vector map of 506 PGK.GFP.2A.γC.

FIG. 22. Vector map of 18 pRRLsinCPPT-Pgk-FancA-wpre.

FIG. 23. Vector map of 506 PGK.FancA.

FIGS. 24A-24C. FIG. 24A) Schematics of the different FANCA viral vectors. FIG. 24B) MMC survival assay of FancA−/− GM06914 cells transduced with MOIs of 1 or 5 LV-FANCA or FV-FANCA or left untransduced. Survival was assessed by crystal violet staining after exposure to MMC for 7 days. VVT denotes FancA−/− cells that have been complemented with a functional FancA gene. FIG. 24C) Fraction of GFP+ cells measured by flow cytometry of FancA−/− GM06914 cells transduced with FV-FANCA-GFP or FV-GFP and grown in the presence of increasing MMC concentrations for 7 days.

FIG. 25. Exemplary sequences supporting the disclosure.

### DETAILED DESCRIPTION

More than 80 primary immune deficiency diseases are recognized by the World Health Organization. These diseases are characterized by an intrinsic defect in the immune system in which, in some cases, the body is unable to produce any enough, or effective antibodies against infection. In other cases, cellular defenses to fight infection fail to work properly. Typically, primary immune deficiencies are inherited disorders. Patients with inherited immune deficiencies such as adenosine deaminase deficient (ADA)-severe combined immunodeficiency (SCID), X-linked SCID, chronic granulomatous disease (CGD), Wiskott-Aldrich Syndrome, and Fanconi anemia (FA) can benefit from gene therapy, which provides a functioning gene to an affected patient to compensate for the defective one.

Secondary, or acquired, immune deficiencies are not the result of inherited genetic abnormalities, but rather occur in individuals in which the immune system is compromised by factors outside the immune system. Examples include trauma, viruses, chemotherapy, toxins, and pollution. Acquired immunodeficiency syndrome (AIDS) is an example of a secondary immune deficiency disorder caused by a virus, the human immunodeficiency virus (HIV), in which a depletion of T lymphocytes renders the body unable to fight infection. Patients with secondary immune deficiencies can also benefit from genetic therapies.

X-linked SCID (SCID-X1) is both a cellular and humoral immune depletion caused by mutations in the common gamma chain gene (γC; e.g., SEQ ID NOs: 1-3), which results in the absence of T and natural killer (NK) lymphocytes and the presence of nonfunctional B lymphocytes. While mutations on other loci, such as jak3, pnp, ada, and rag (e.g., SEQ ID NOs: 6-10) can lead to non-X-linked severe combined immunodeficiency, half of all SCID cases are X-linked. SCID-X1 is fatal in the first two years of life unless the immune system is reconstituted, for example, through bone marrow transplant (BMT) or gene therapy. Since most individuals lack a matched donor for BMT or gene therapy, haploidentical parental bone marrow depleted of mature T cells is often used (Buckley R H et al. (1999) NEJM 340(7): 508-516; Pai S Y et al. (2014) NEJM 371(5): 434-446); however, complications include graft versus host disease (GVHD), failure to make adequate antibodies hence requiring long-term immunoglobulin replacement, late loss of T cells due to failure to engraft HSPCs, chronic warts, and lymphocyte dysregulation.

FA is an inherited blood disorder that leads to bone marrow failure. It is characterized, in part, by a deficient DNA-repair mechanism that increases a person's risk for a variety of cancers. For example, at least 20% of patients with FA develop cancers including acute myeloid leukemias and cancers of the skin, liver, gastrointestinal tract, and gynecological systems. The skin and gastrointestinal tumors are usually squamous cell carcinomas. The average age of patients who develop cancer is 15 years for leukemia, 16 years for liver tumors, and 23 years for other tumors (D'Andrea A D et al. (1997) Blood 90(5): 1725-1736; Garcia-Higuera I et al. (1999) Curr. Opin. Hematol. 2: 83-88; Hejna J A et al. (2000) Am. J. Hum. Genet. 66(5): 1540-1551).

Cells from FA patients display a characteristic hypersensitivity to agents that produce interstrand DNA crosslinks such as mitomycin C or diepoxybutane. FA genes define a multicomponent pathway involved in cellular responses to DNA cross-links. Five of the FA genes (FANCA, FANCC, FANCE, FANCF and FANCG; e.g., SEQ ID NOs: 16-20) have been cloned and the FANCA, FANCC and FANCG proteins have been shown to form a molecular complex with primarily nuclear localization. FANCC also localizes in the cytoplasm. Different FA proteins have few or no known sequence motifs with no strong homologs of the FANCA, FANCC, FANCE, FANCF, and FANCG proteins in non-vertebrate species. FANCF has weak homology of unknown significance to an E. coli RNA binding protein. The two most frequent complementation groups are FA-A and FA-C which together account for 75%-80% of FA patients. Multiple mutations have been recognized in the FANCA gene that span 80 kb and include at least 43 exons. FANCC has been found to have 14 exons and spans 80 kb. A number of mutations in the FANCC gene have been identified which are correlated with FA of differing degrees of severity.

An alternative therapeutic approach to BMT and non-autologous gene therapy in immune and blood disorder failures is ex vivo HSPC gene therapy, where blood or bone marrow derived HSPCs are enriched from patients, transduced with viral vectors to deliver a functional therapeutic gene (e.g., a γC gene for SCID-X1 or a FancA gene for FA), and transplanted back to the patient. The first generation ex vivo gene therapy for SCID-X1 used murine leukemia virus-based gammaretroviral (RV) delivery (Cavazzana-Calvo M et al. (2000) Science 288: 669-672; Gaspar H B et al. (2004) Lancet 364: 2181-2187) and showed significant long-term clinical improvement in treated patients. However, 5/20 patients unexpectedly developed T cell leukemia, resulting in the death of one patient. These findings precipitated intense interest in utilization of self-inactivating (SIN) viral vectors and SIN-lentiviral vectors (LV) as alternative vector platforms (Cartier N et al. (2009) Science 326: 818-823; Cavazzana-Calvo M et al. (2010) Nature 467: 318-322). Ex vivo gene therapy for FA has posed challenges also, as FA stem cells have increased sensitivity to free radical-induced DNA damage during ex vivo culture and manipulation. Transduction of cells from FA patients with a safety modified LV vector carrying a FANCA gene under conditions that reduced oxidative stress improved the survival of these cells in ex vivo culture (Becker P S et al. (2010) Gene Therapy 17: 1244-1252). While SIN-RV and SIN-LV are currently used in some clinical settings with considerable success (Hacein-Bey-Abina S et al. (2014) NEJM 371(15): 1407-1417; De Ravin S S et al. (2016) Sci Transl Med. 8(335): 335ra357), the vector is but one consideration for an immunodeficiency like FA, where two decades of clinical research has only underscored the need to improve many aspects of ex vivo gene therapy in FA, including the number and quality of gene-corrected FA HSPCs, the therapeutic vector, the transduction protocol to be used for the correction of FA HSPCs, and the potential conditioning of the patients (Adair J E et al. (2016) Current gene therapy 16(5): 338-348). Thus, ex vivo gene therapy still faces multiple challenges that include the: 1) extensive ex vivo manipulation of HSPCs required to prepare them for therapeutic use that results in loss of multipotency potential and/or reduced fitness for engraftment following transplantation, 2) various conditioning regimens used to enhance engraftment of gene modified HSPCs add considerable genotoxic risks to the patients, and 3) requirement of advanced infrastructures for the collection, culture, transduction, validation, and re-infusion of HSPCs, consequently restricting this form of treatment to a select few institutions worldwide.

With these limitations in mind, treatment using in vivo gene therapy, which includes the direct delivery of the viral vector to the patient, has been explored. In vivo gene therapy may have a number of advantages including no requirement for HSPC harvesting, in vitro culture, and reinfusion; and no, or less requirement, for genotoxic conditioning. The absence of ex vivo cell processing may promote better HSPC engraftment and result in production of cells of all lineages. Moreover, in vivo gene therapy could be adopted at many institutions worldwide, including those in developing countries, as the therapy could be administered through an injection, similar to what is already done worldwide for the delivery of vaccines.

Animal models have been used to study in vivo gene therapy. Neonatal intravenous injection of an RV vector into mice resulted in transduction of HSCs in the mice, and intravenous injection of an RV vector into dogs led to stable transduction of blood cells for over 3 years (Xu L et al. (2004) Molecular Therapy 10(1): 37-44; T. O'Malley and M. Haskins, unpublished data). These results suggest that this in vivo approach could be used for BM-directed gene therapy.

In vivo gene therapy has also been explored in a canine model of SCID-X1 (Humbert O et al. (2015) Blood 126: 262; Kennedy D R et al. (2011) Vet Immunol Immunopathol 142: 36-48; Burtner C R et al. (2014) Blood 123: 3578-3584). Canine SCID-X1 is caused by naturally occurring mutations in the γC gene and provides an excellent preclinical model because of nearly identical phenotypic characteristics as compared to human SCID-X1 (Noguchi M et al. (1993) Science 262: 1877-1880; Henthorn P S et al. (1994) Genomics 23, 69-74; Leonard W J (1996) Investig Med 44: 304-311). Both human and canine SCID-X1 are characterized by absent thymic T-cell development and dysregulated B cell germinal center responses leading to low immunoglobulin levels (IgA and IgG), failure to thrive, and early mortality due to viral and/or bacterial infection (Conley M E et al. (1990) J Clin Invest 85: 1548-1554; Gougeon M L et al. (1990) J Immunol 145: 2873-2879; Gendelman H E et al. (1991) J Virol 65: 3853-3863; Buckley R H et al. (1993) Seminars in Hematology 30: 92-104; Matthews D J et al. (1995) Blood 85: 38-42; Rosen A et al. (1995) Int Immunol 7: 625-633). The utility of the canine SCID-X1 model was previously validated by studies employing ex vivo HSPC gene therapy (reviewed in Felsburg P J et al. (2015) Human Gene Therapy 26: 50-56) and more recently by direct intravenous administration of the viral vector (Ting-De Ravin S S et al. (2006) Blood 107(8): 3091-3097).

The feasibility of in vivo gene therapy in canine SCID-X1 with intravenous injection of FV vector expressing human codon optimized γC driven by the short elongation factor-1 alpha promoter (EF1α; EF1α.γC.FV) was previously demonstrated (Burtner C R et al. (2014) Blood 123: 3578-3584). Successful lymphocyte expansion was reported in these animals but clonal diversity and the T-cell receptor (TCR) repertoire were low. Ultimately, all animals were euthanized due to infections.

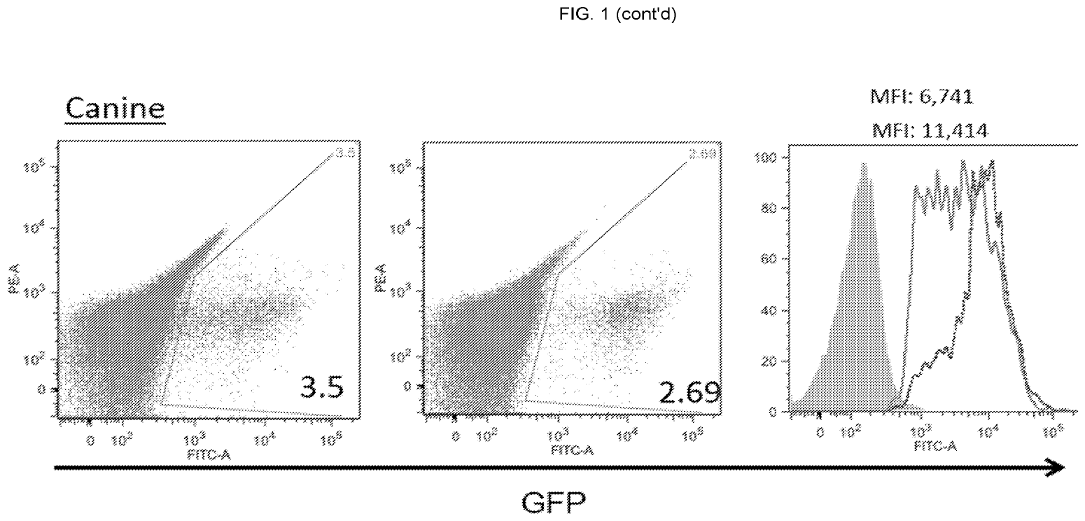

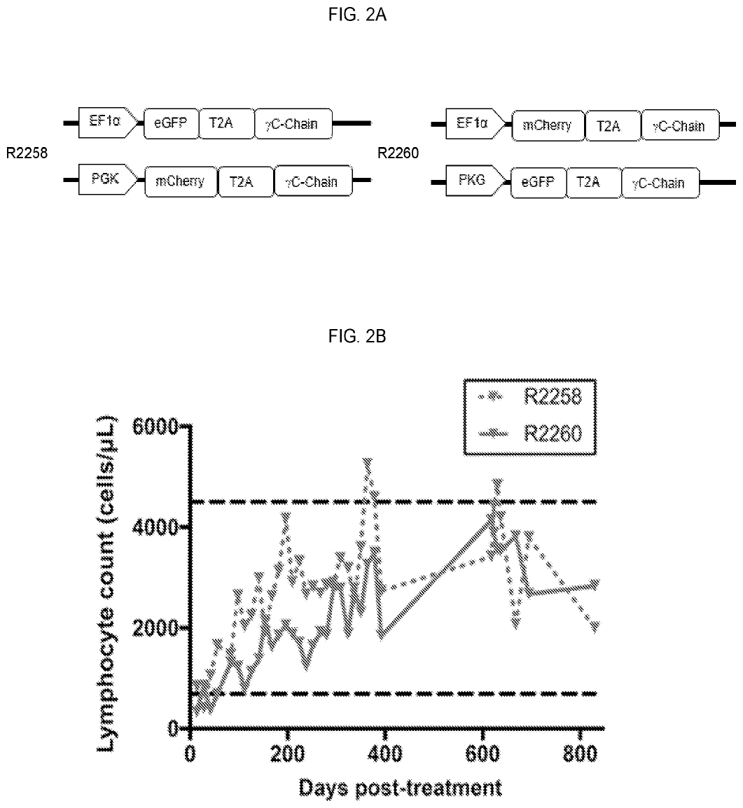

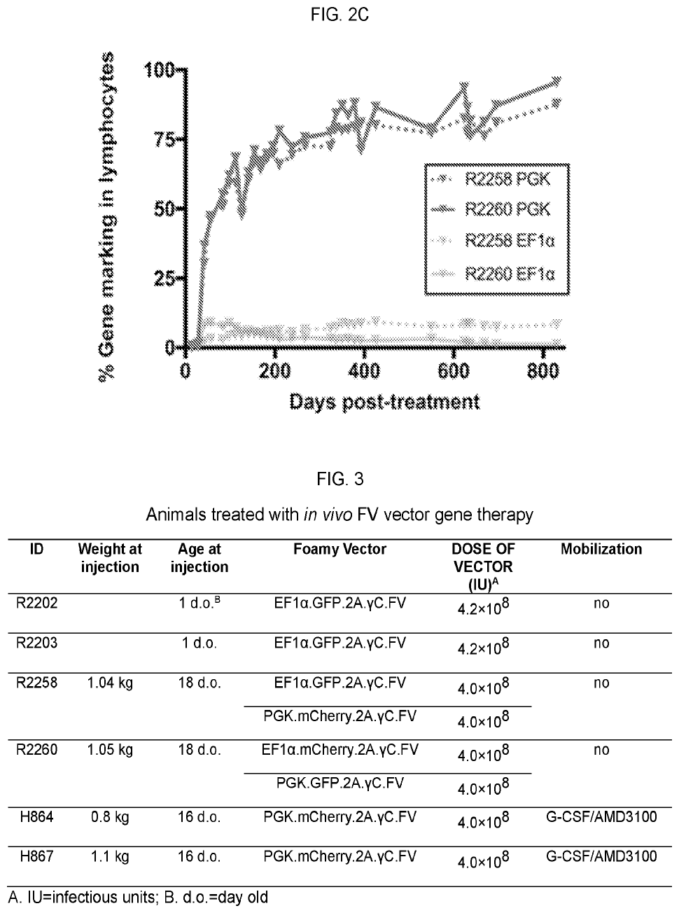

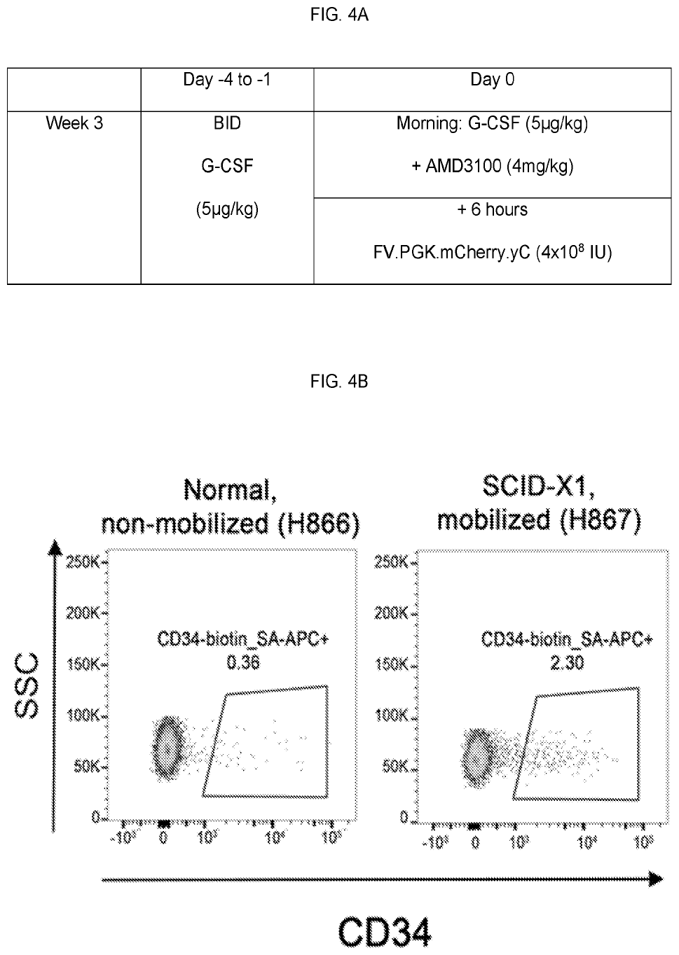

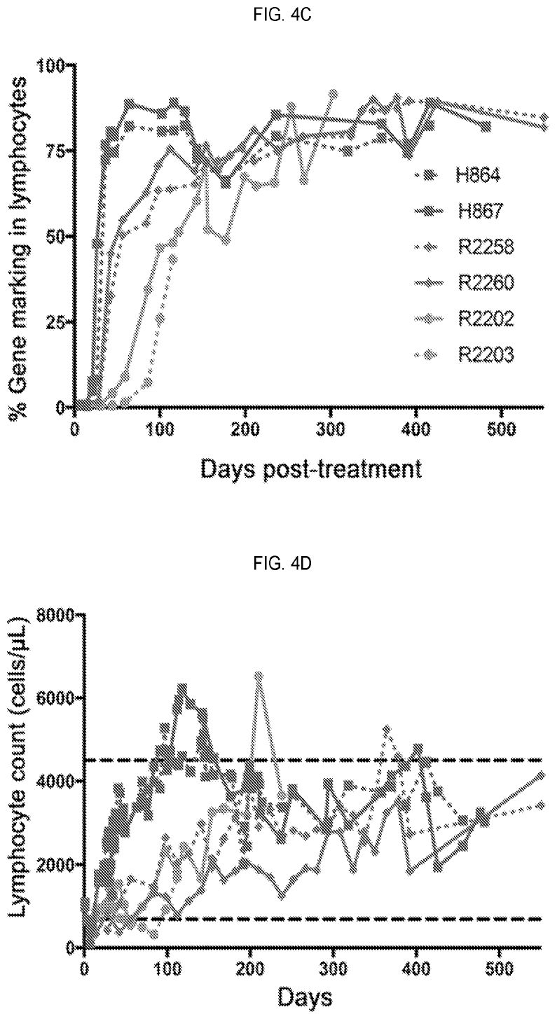

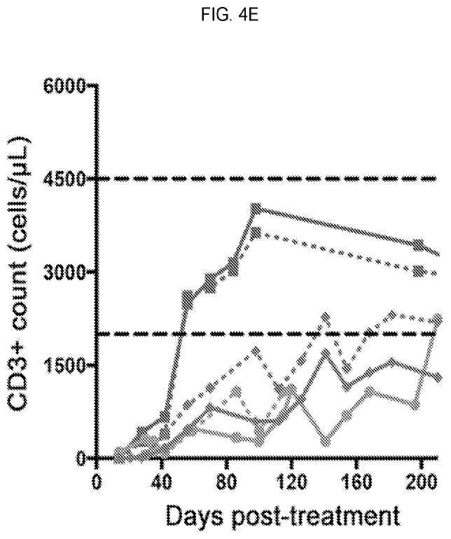

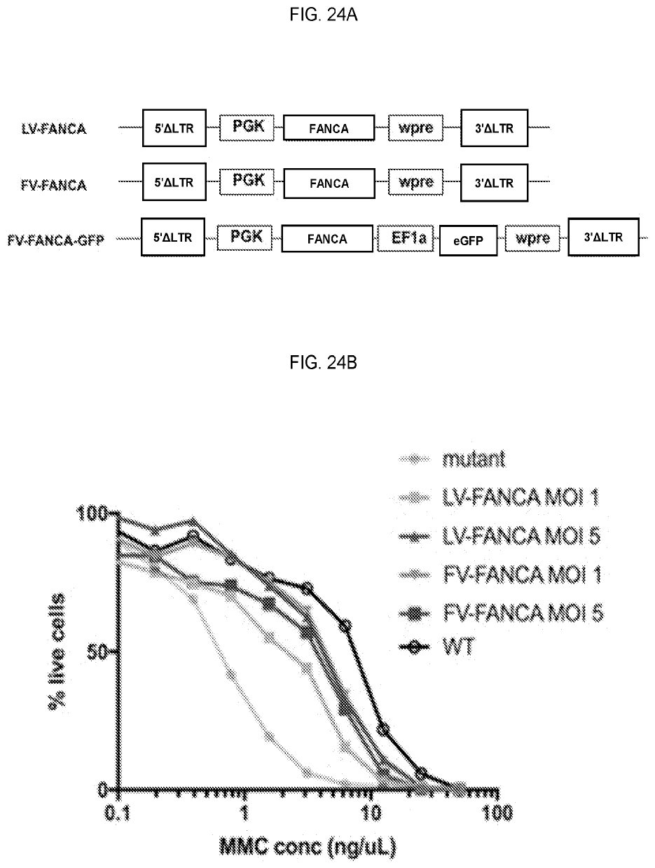

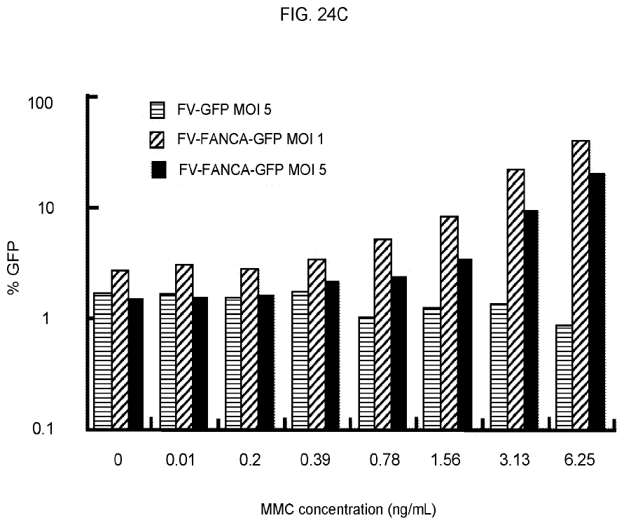

The current disclosure provides systems and methods that improve the kinetics of T cell correction and expansion beyond that achieved by the prior art. The systems and methods utilize a foamy viral vector including a human phosphoglycerate kinase (PGK) promoter (instead of an EF1α promoter) associated with a therapeutic gene. Regarding SCID particularly, intravenous delivery of an FV vector including a PGK promoter associated with γC (PGK.γC.FV) resulted in significantly improved T cell recovery compared to EF1α promoter in SCID-X1 canines (e.g., FIGS. 2B, 4D, and 4E). Regarding FA, delivery of an FV vector including a PGK promoter associated with a FancA gene in fanca −/− cells conferred resistance of these cells to the DNA crosslinking agent mitomycin C, suggesting that deficient DNA repair characteristics of FA can be overcome with delivery of a functional FancA gene as described herein (FIGS. 24B, 24C).

Ex vivo HSPC gene modification with retroviral vectors is generally performed with isolated CD34+ HSPCs. To obtain a larger number of CD34+ HSPCs for isolation, these cells can be mobilized. HSPC mobilization is a process whereby HSPCs move from the bone marrow into peripheral blood. This process has been invaluable in creating a source of HSPCs in blood that can be harvested to use in transplantation therapies for numerous diseases and disorders including inherited immunodeficiencies, bone marrow failure, myelodysplasia and many relapsed hematopoietic malignancies.

The bone marrow niche is a highly organized microenvironment which anchors HSPCs and regulates their self-renewal, proliferation and trafficking. The binding of stromal derived factor-1 (SDF-1, also known as CXCL-12) to its receptor (CXCR4) on HSPC plays a key role in HSPC retention within the bone marrow. Molecules with roles in cell adhesion such as vascular cell adhesion molecule-1 (VCAM-1), very late antigen 4 (VLA-4, α4β1 integrin), and stem cell factor (SCF) are also key in HSPC retention in the bone marrow. Agents that affect factors that tether HSPCs to the bone marrow niche can thus promote HSPC mobilization from bone marrow into blood.

While the benefits of cell mobilization for isolation and ex vivo manipulation are well-documented, the potential effects of mobilization in the context of in vivo gene therapies are less clear. For example, in vivo gene therapy relies on retroviral vectors successfully targeting and integrating into targeted cells after introduction into a subject. Mobilization can bring a heterogeneous population of cells out of the bone marrow, and therefore, could dilute the ability of gene therapy vectors to effectively target cells for treatment.

Importantly, the present disclosure unexpectedly found that cell mobilization performed in concert with in vivo FV vector injection improved immune reconstitution. Thus, in particular embodiments, use of a PGK promoter for in vivo gene therapy can be beneficially combined with cell mobilization prior to FV vector administration. The addition of cell mobilization can enhance FV vector transduction of relevant cells, which normally reside in the bone marrow stoma.

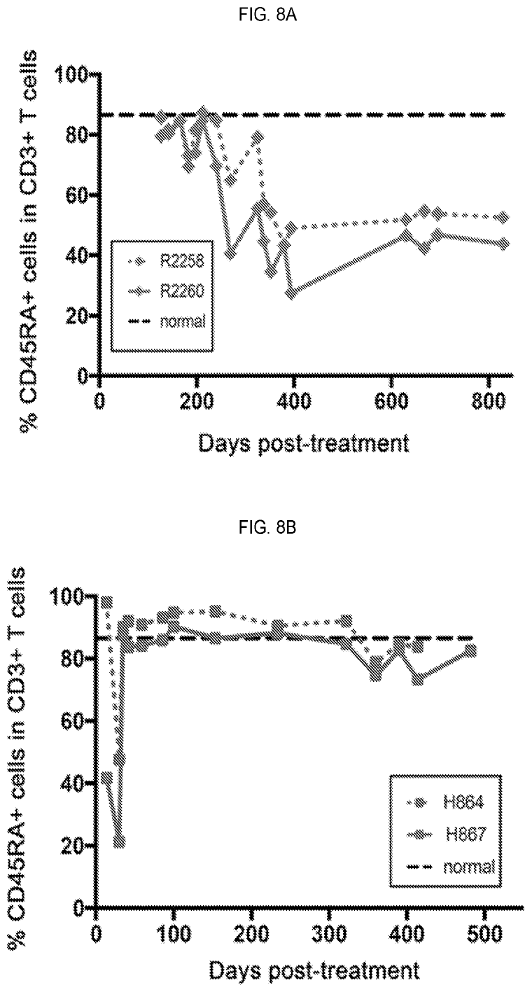

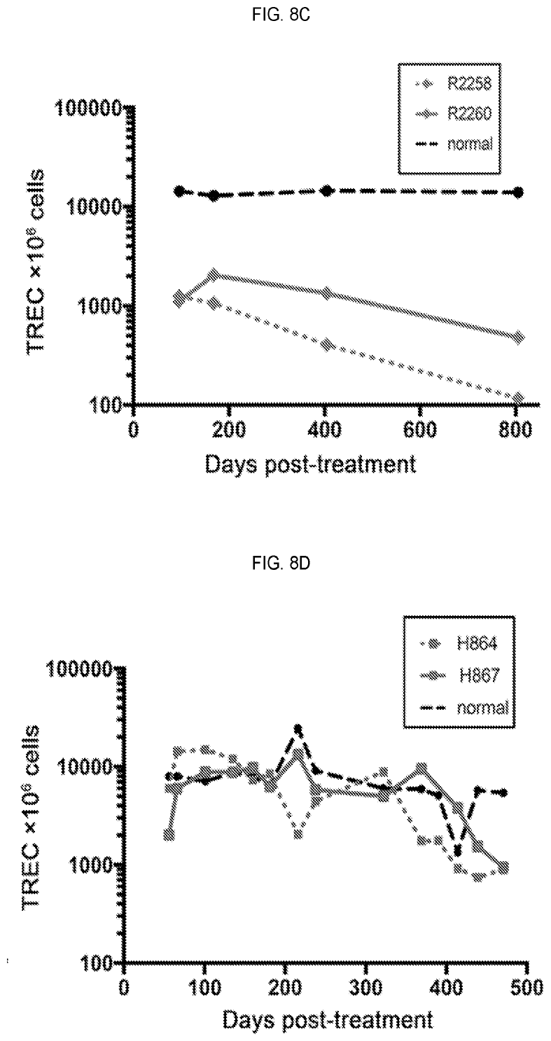

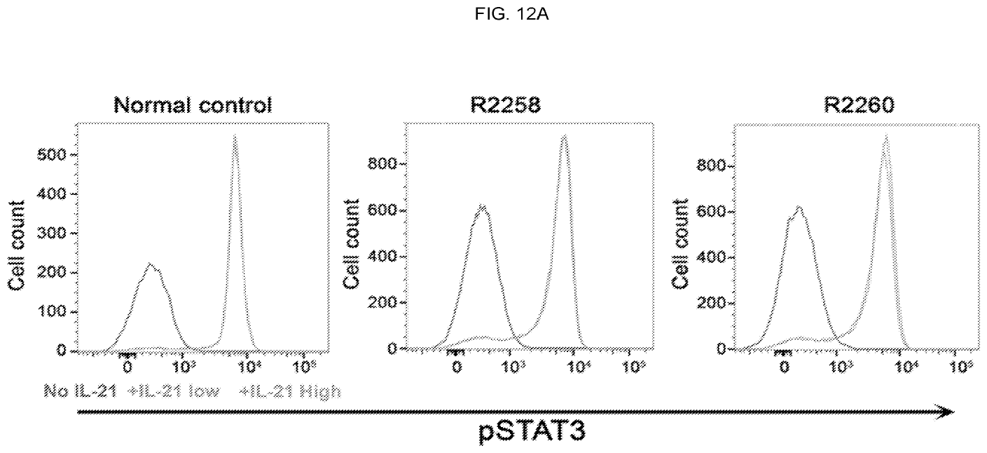

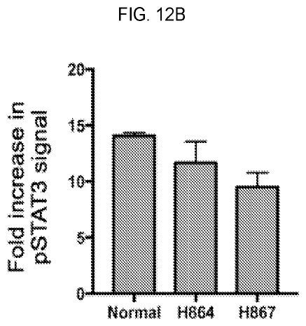

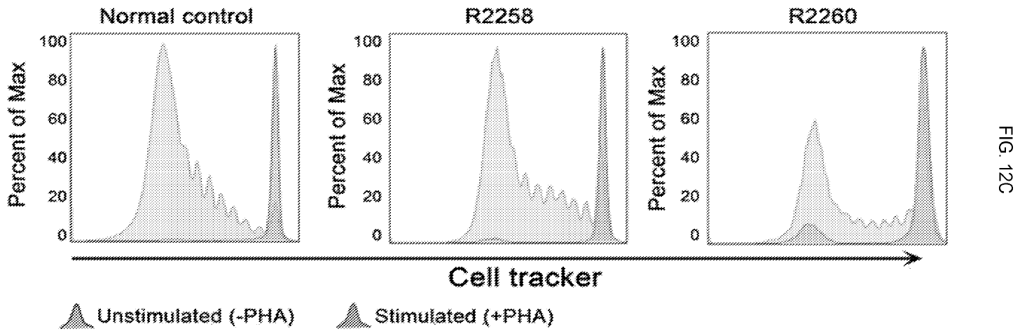

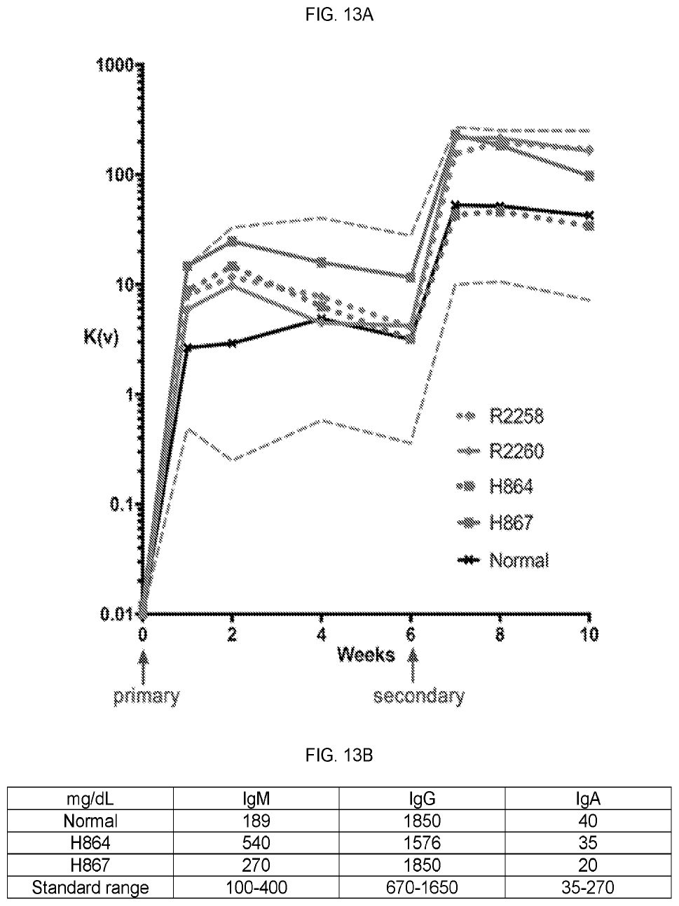

The present disclosure shows that cell mobilization with G-CSF/AMD3100 resulted in a 7-fold increase in circulating CD34+ cells (FIG. 4B). A PGK promoter associated with a γC gene in an FV vector combined with mobilization markedly increased both kinetics and clonal diversity of lymphocyte reconstitution, and also correlated with more robust thymopoiesis in SCID-X1 canines. The kinetics of CD3+ lymphocyte reconstitution was substantially increased, with greater CD3+ lymphocyte counts in mobilized animals injected with PGK.γC.FV, as compared to non-mobilized animals injected with EF1α.γC.FV, or competitively with PGK.γC.FV and EF1α.γC.FV, at a given time post treatment (2,500 cells/μL vs. 100 and 400 cells/μL 40 days post treatment, respectively, FIG. 4E). Greater clonal diversity and thymopoiesis was demonstrated in mobilized animals as compared to unmobilized controls. T-lymphocyte maturation was verified in these animals by rearrangement of T-cell receptor and expression of the co-receptors CD4 and CD8 (FIGS. 8A-8D, 9A-9C, 10A, 10B, 11). In addition, both T- and B-lymphocyte signaling programs were restored as evidenced by response to IL-21/mitogen activation, and by normal primary and secondary antibody response to immunization with neoantigen bacteriophage ΦX174 (FIGS. 12A-12C, 13A, 13B). Altogether, the results suggest remarkable potential for an accessible, portable and clinically translatable therapy for immune deficiencies, such as SCID-X1 and FA, using a combination of cell mobilization and in vivo FV vector delivery utilizing a PGK promoter associated with a therapeutic gene.

Aspects of the disclosure are now described in more detail as follows: (i) Foamy Viral Vectors; (ii) Optional Transposable Elements; (iii) PGK Promoter and Therapeutic Genes; (iv) Mobilization Factors; (v) Formulations; (vi) Methods of Use; (vii) Reference Levels Derived from Control Populations; and (viii) Kits.

(i) Foamy Viral Vectors. Foamy viruses (FVs) are the largest retroviruses known today and are widespread among different mammals, including all non-human primate species, however are absent in humans. This complete apathogenicity qualifies FV vectors as ideal gene transfer vehicles for genetic therapies in humans and clearly distinguishes FV vectors as gene delivery system from HIV-derived and also gammaretrovirus-derived vectors.

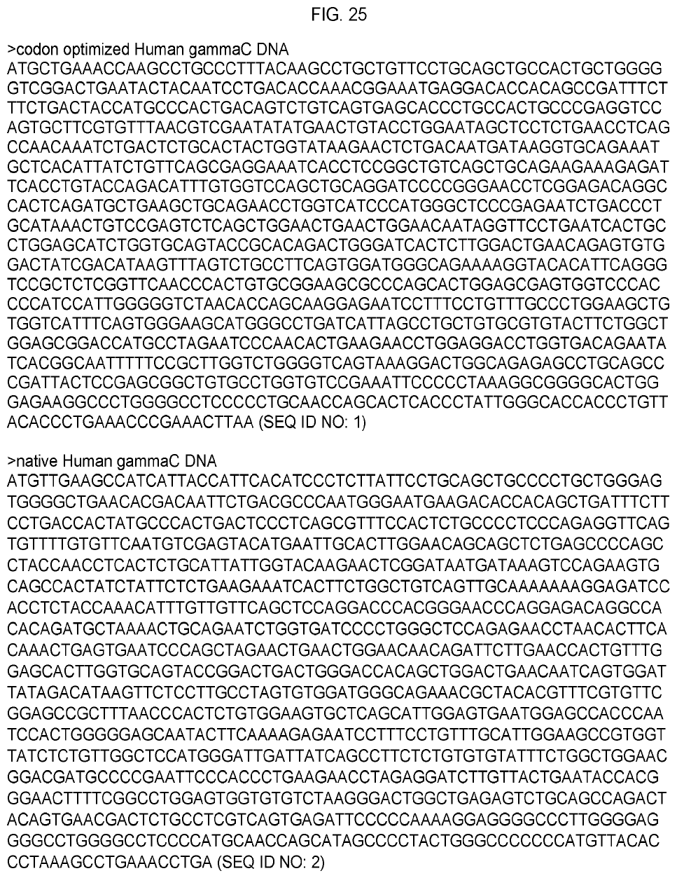

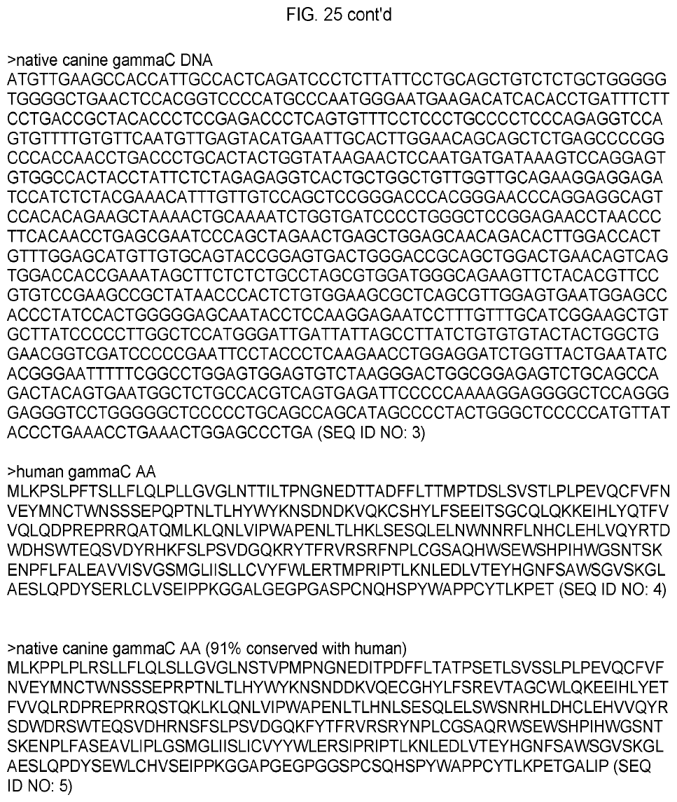

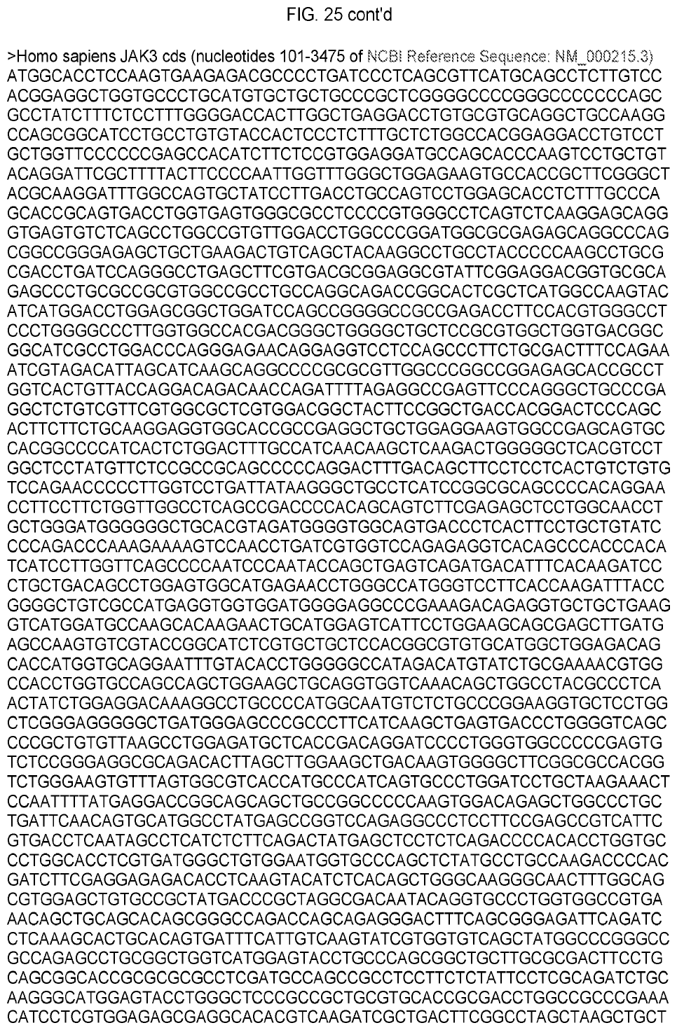

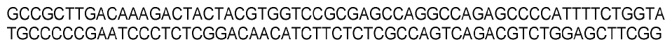

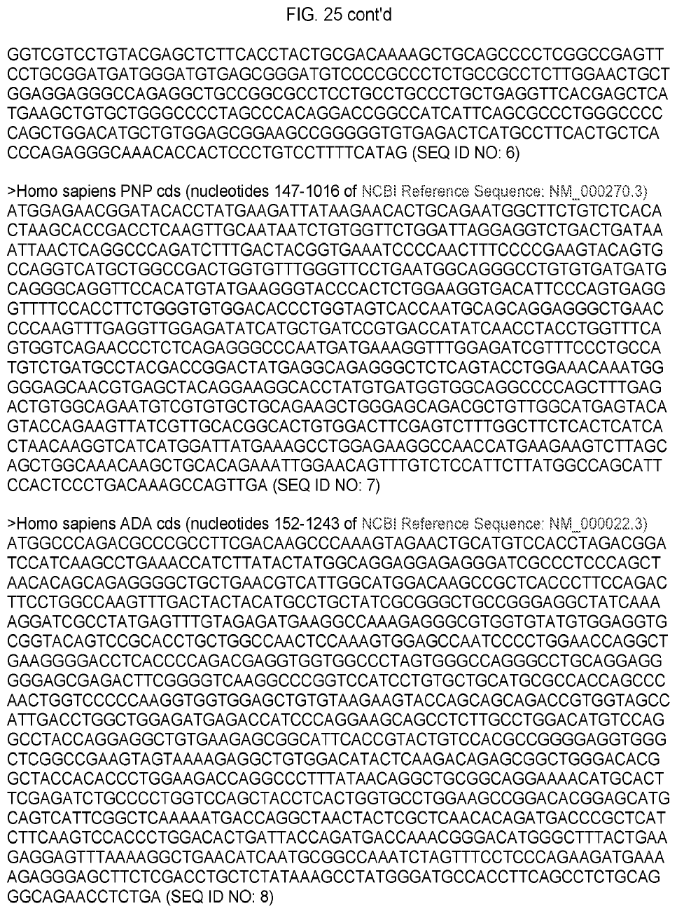

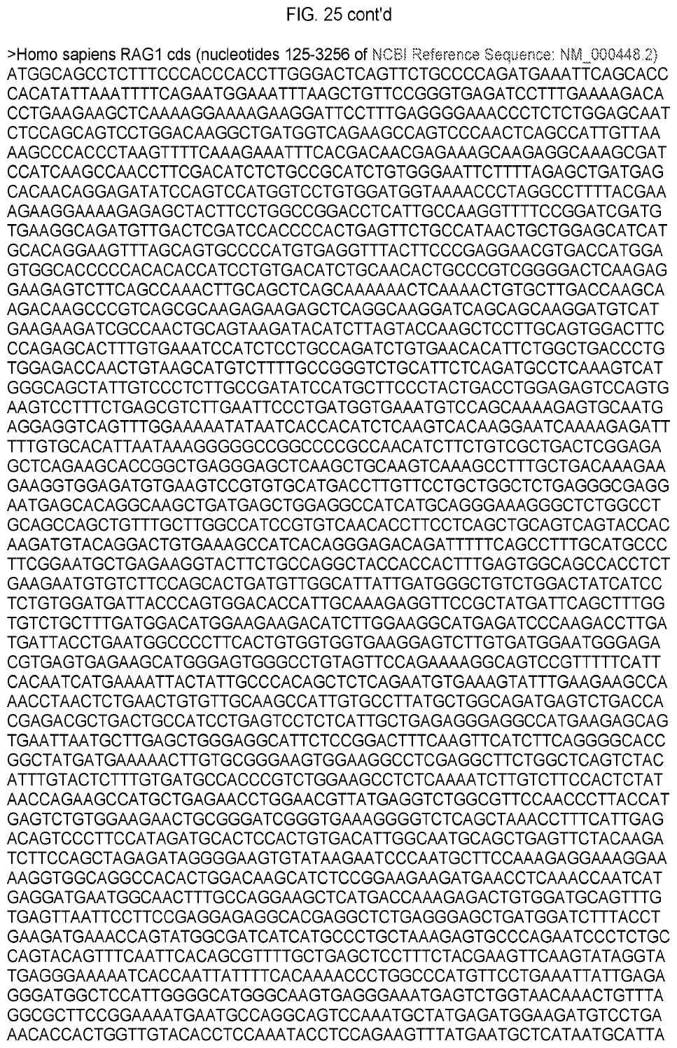

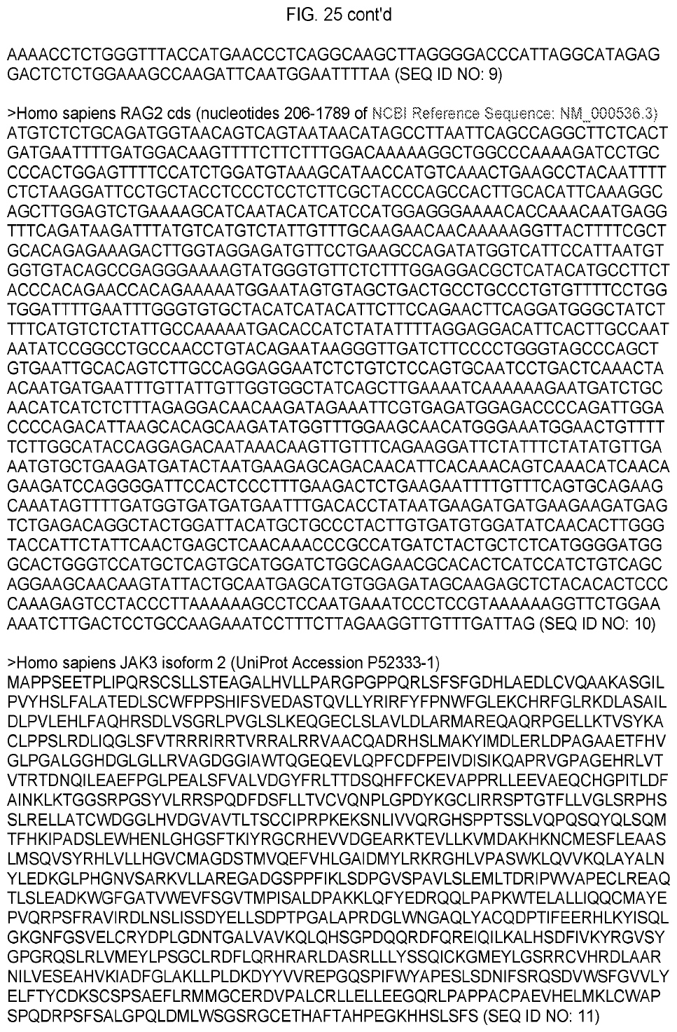

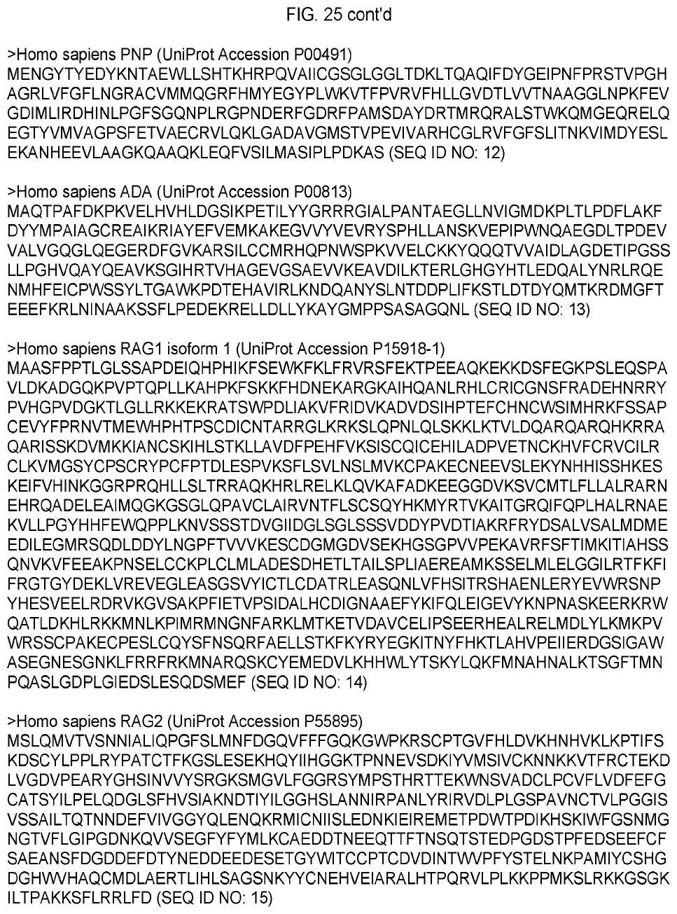

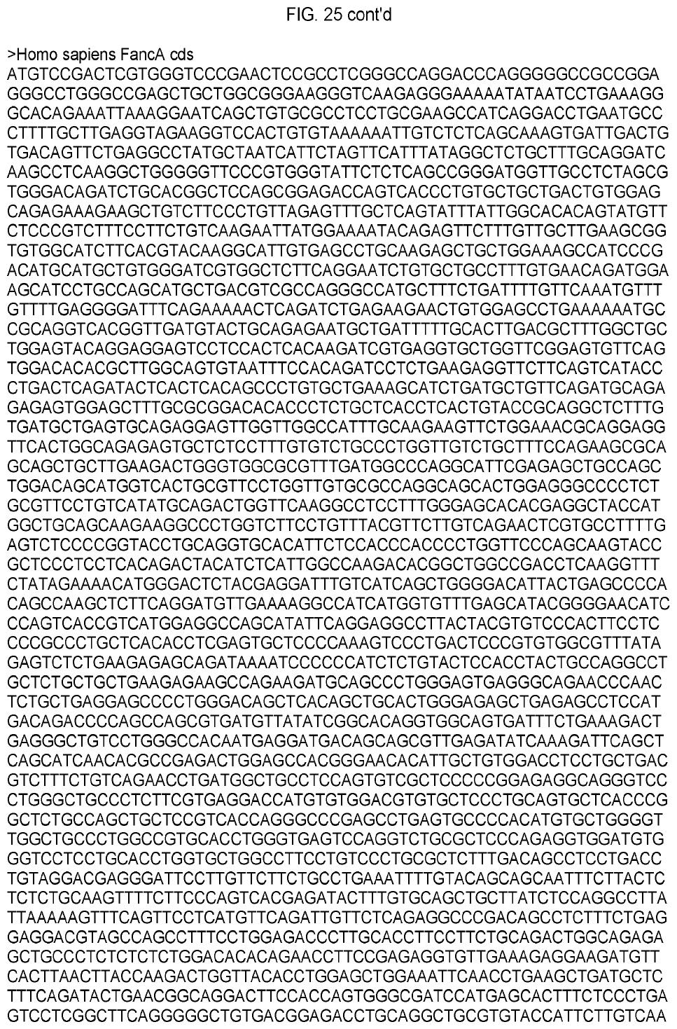

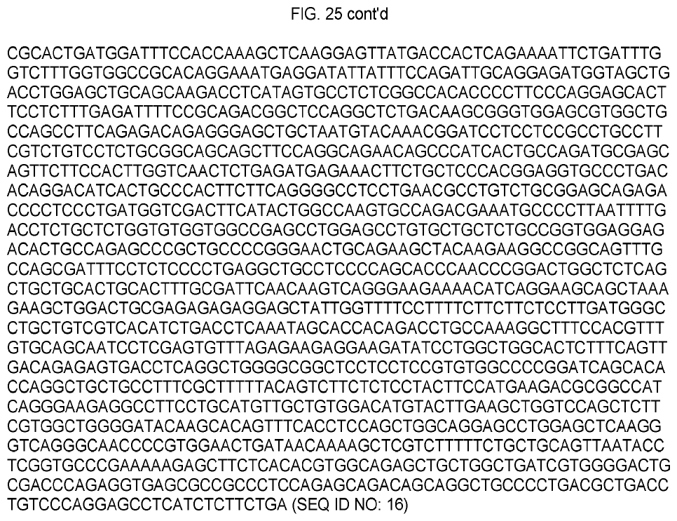

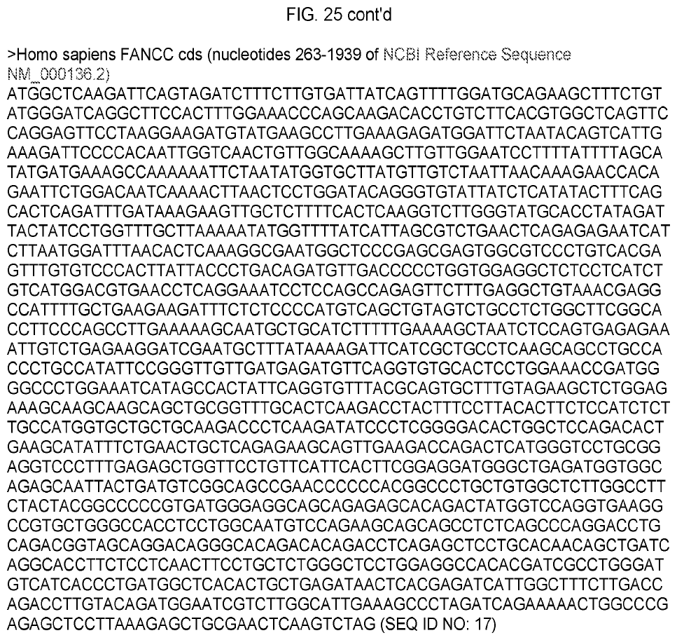

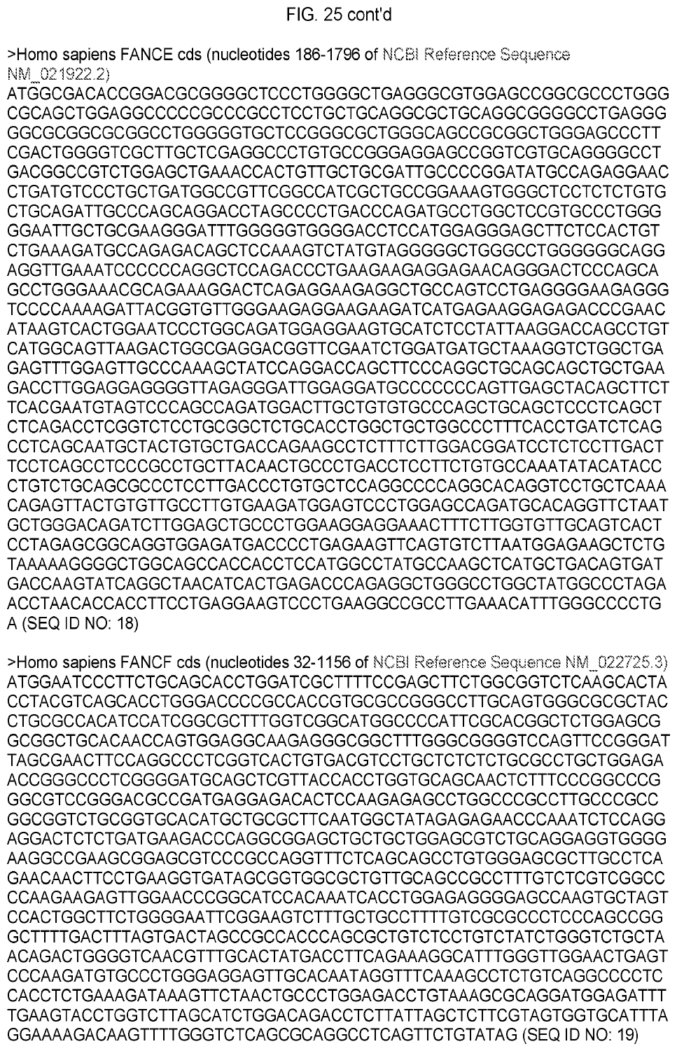

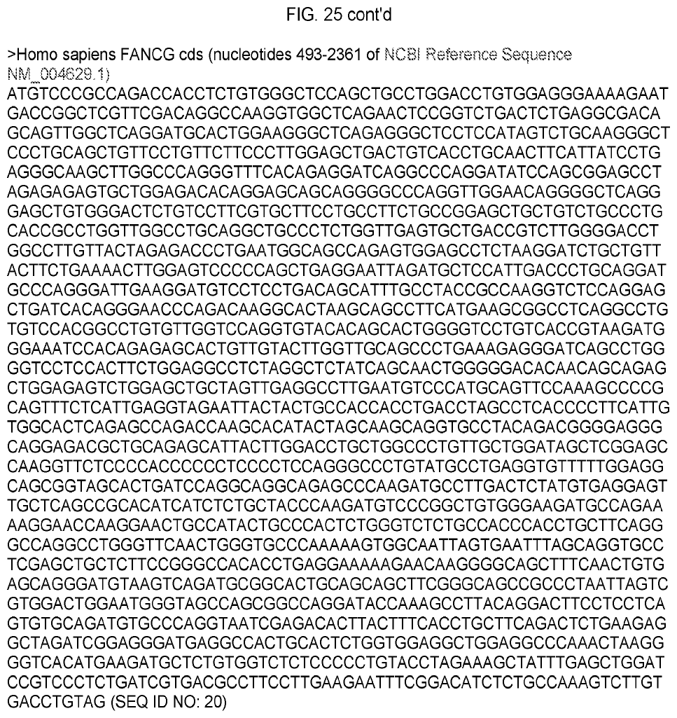

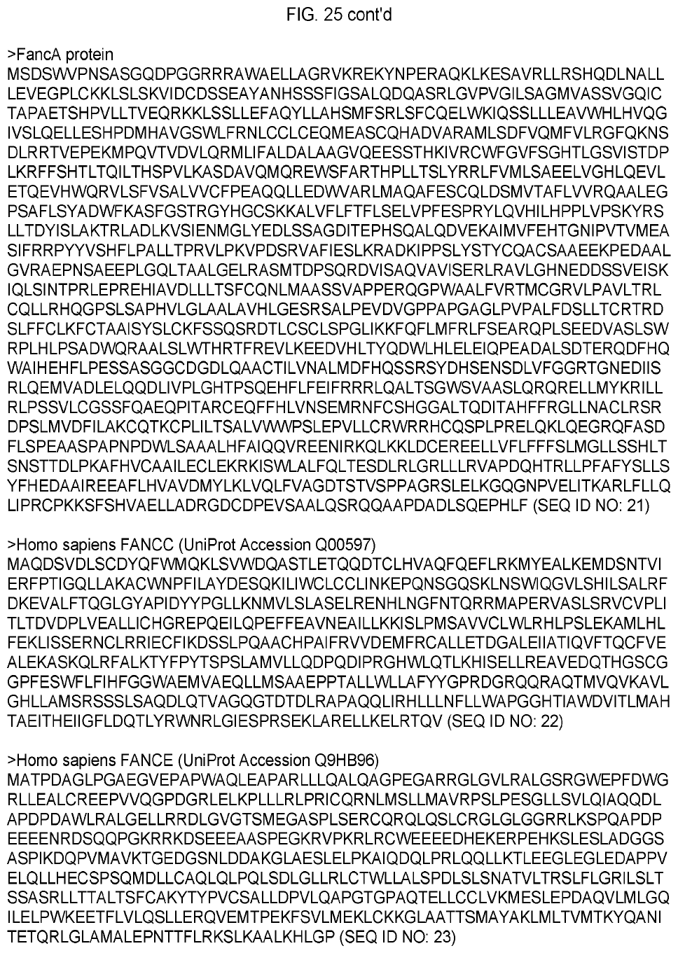

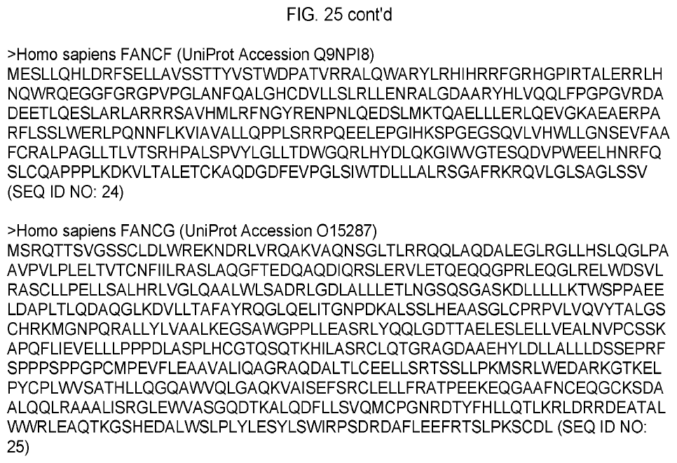

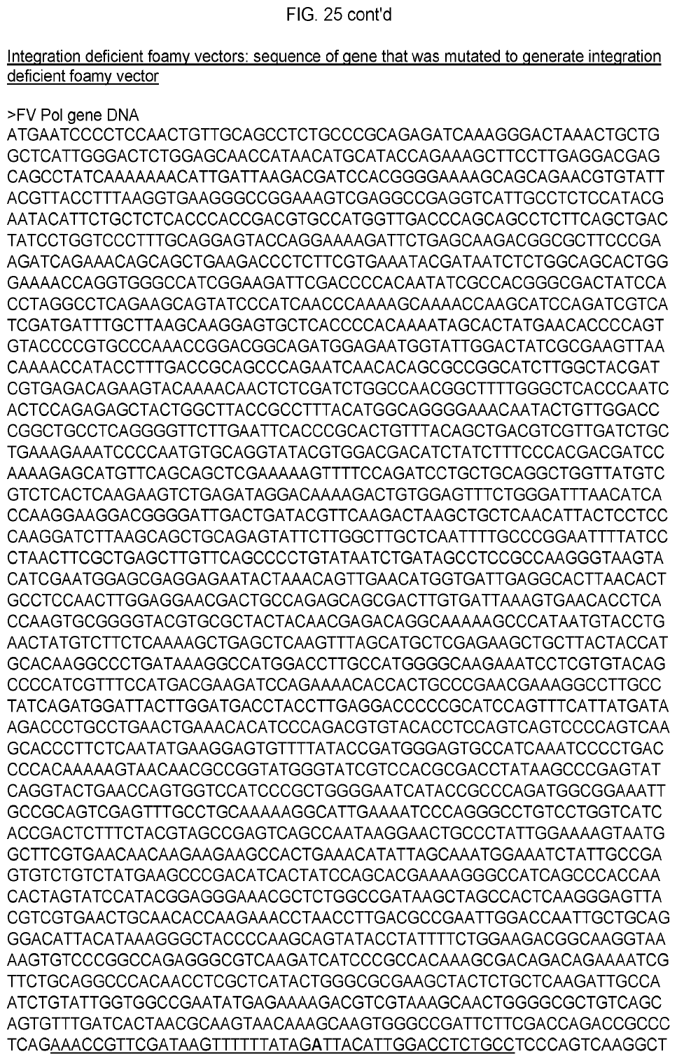

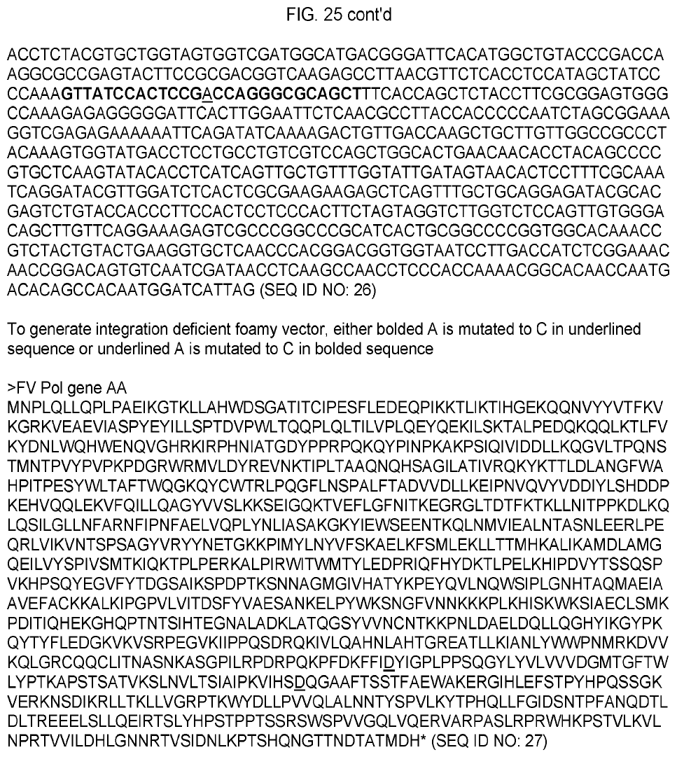

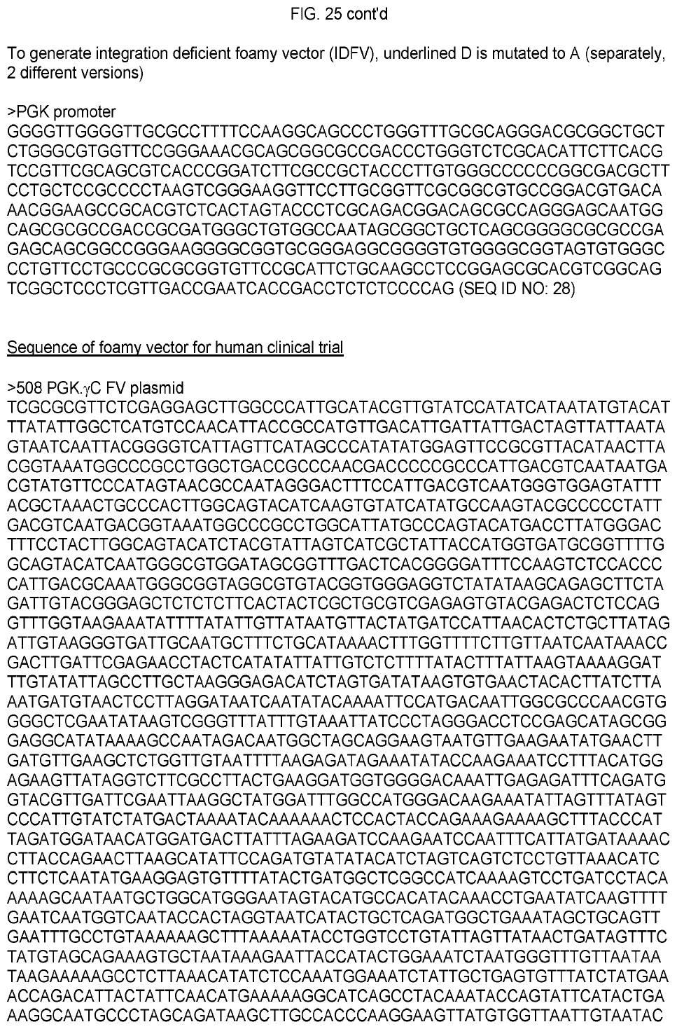

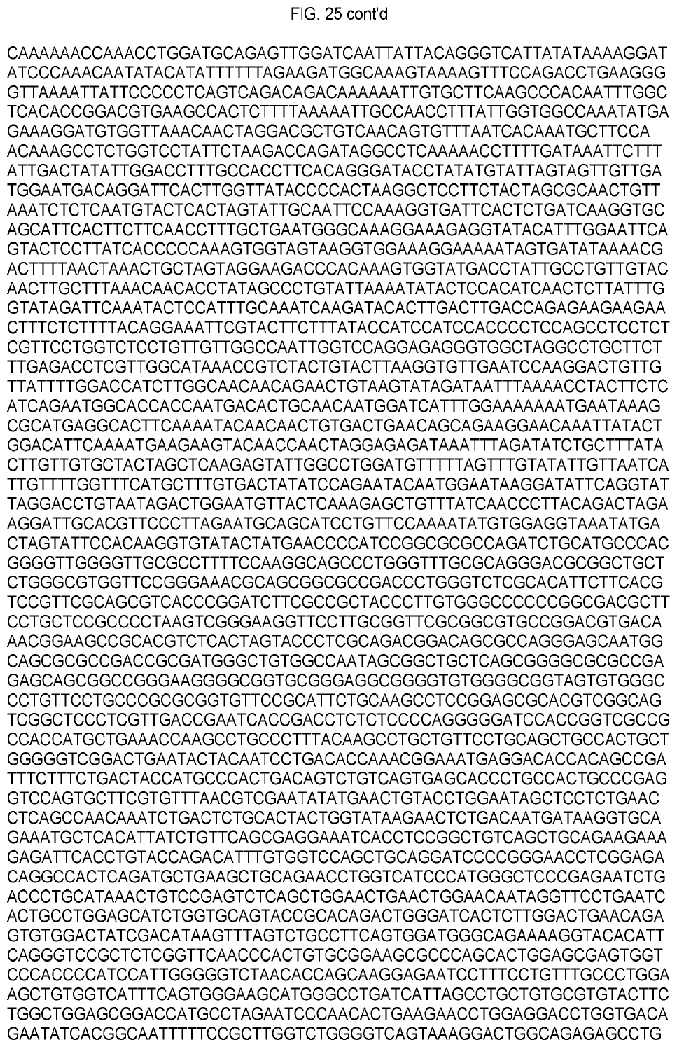

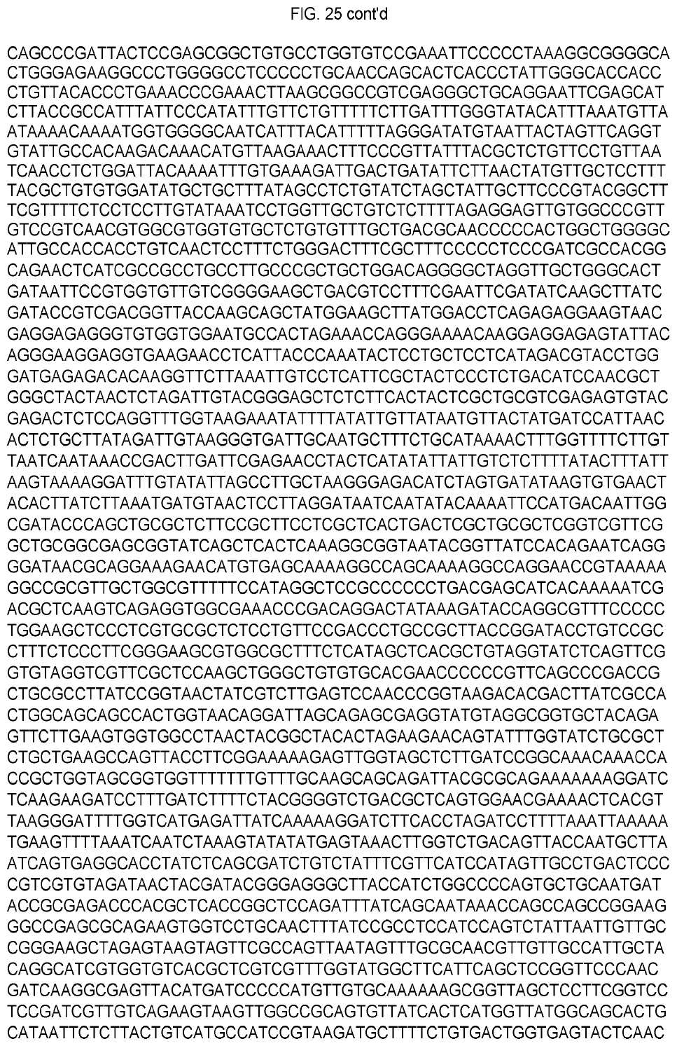

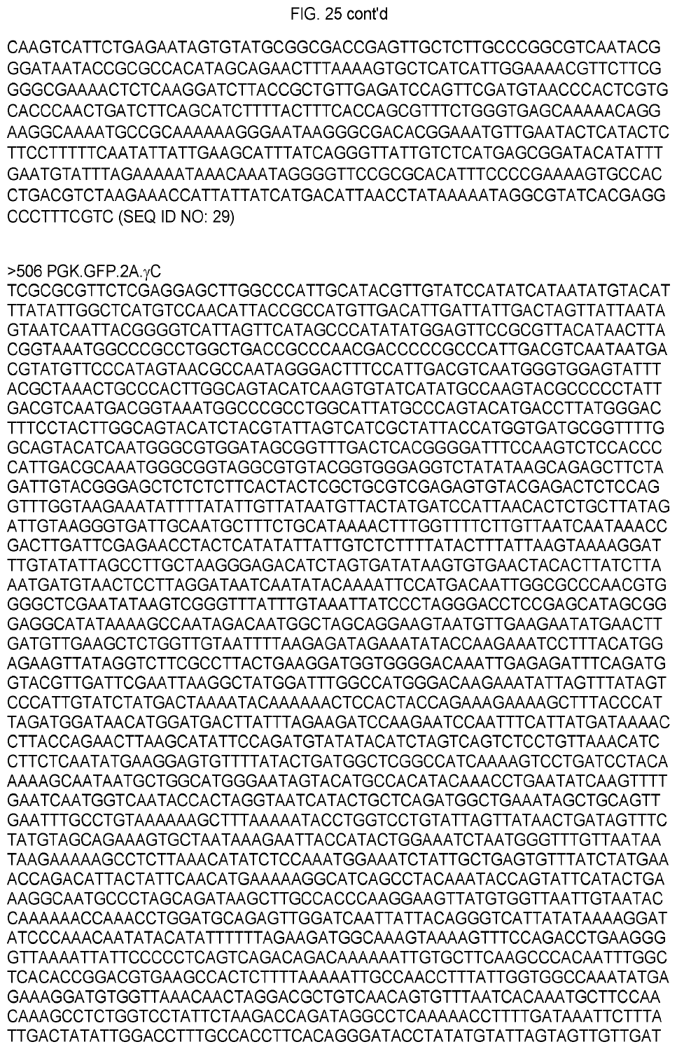

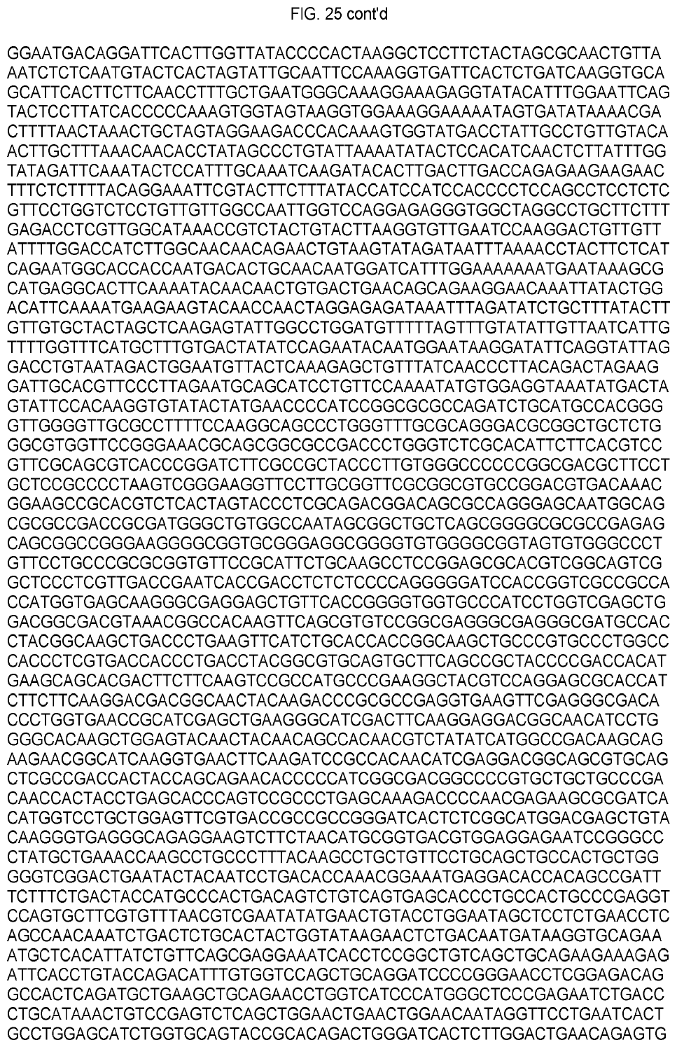

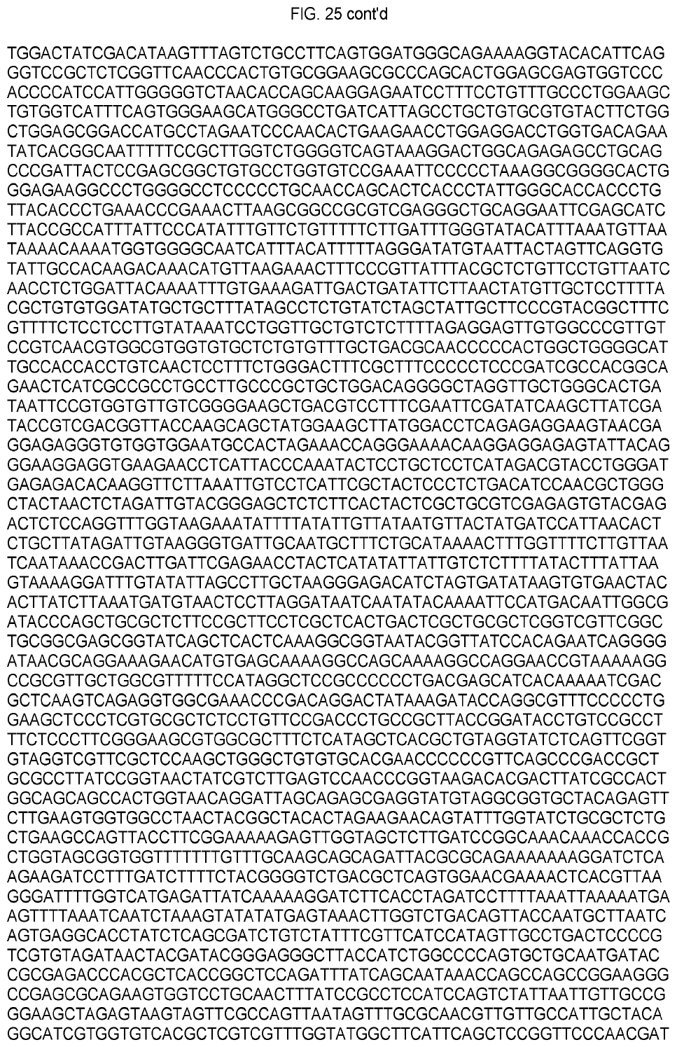

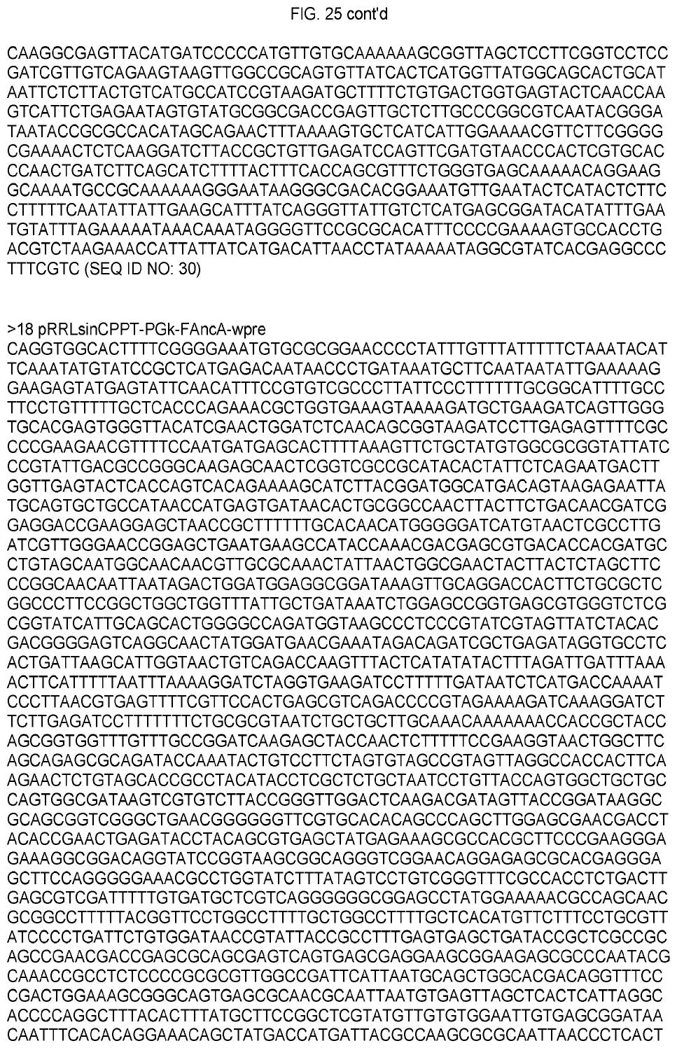

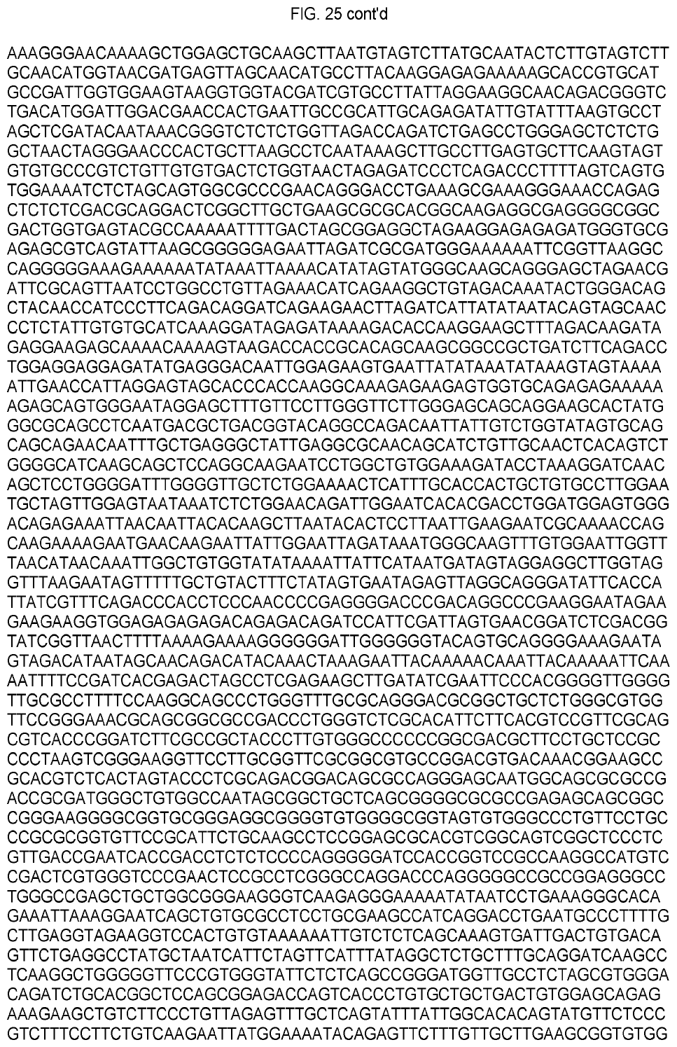

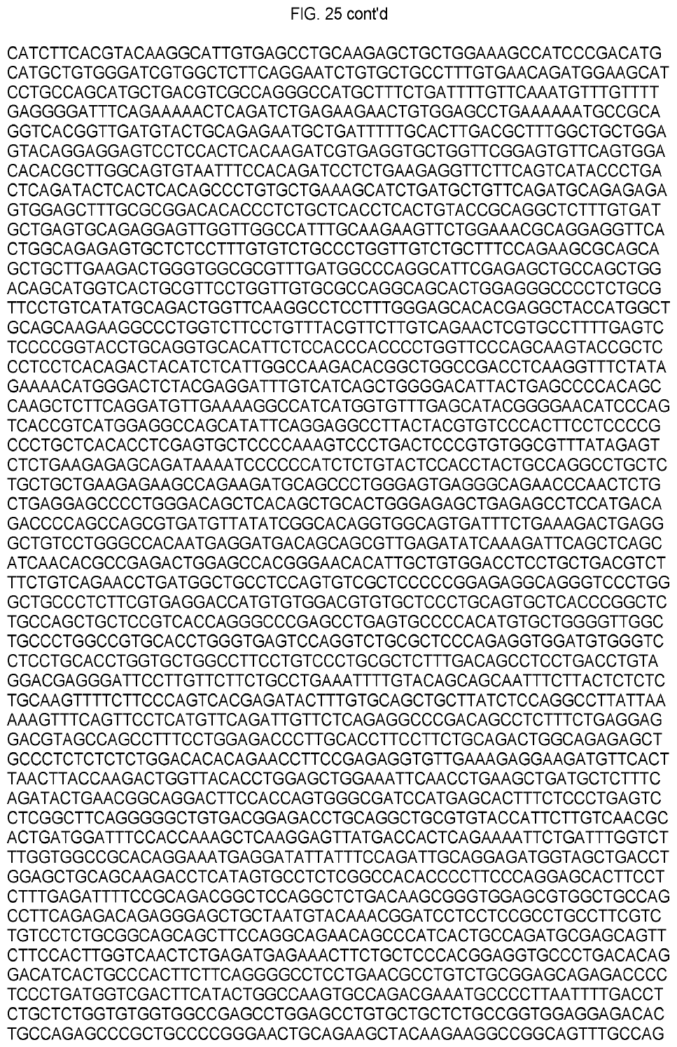

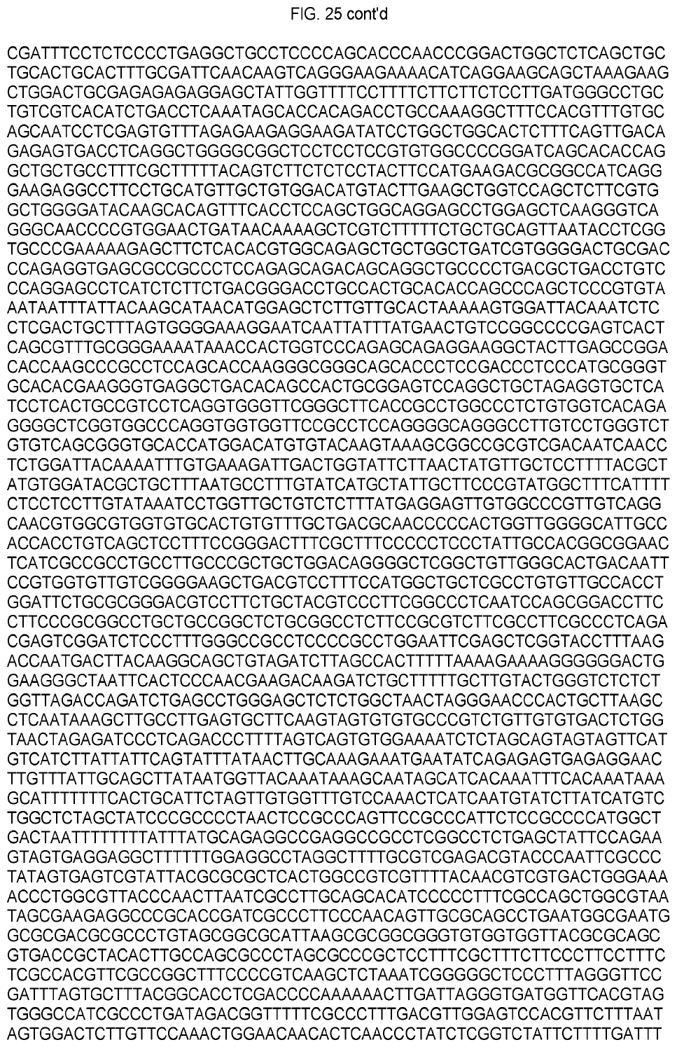

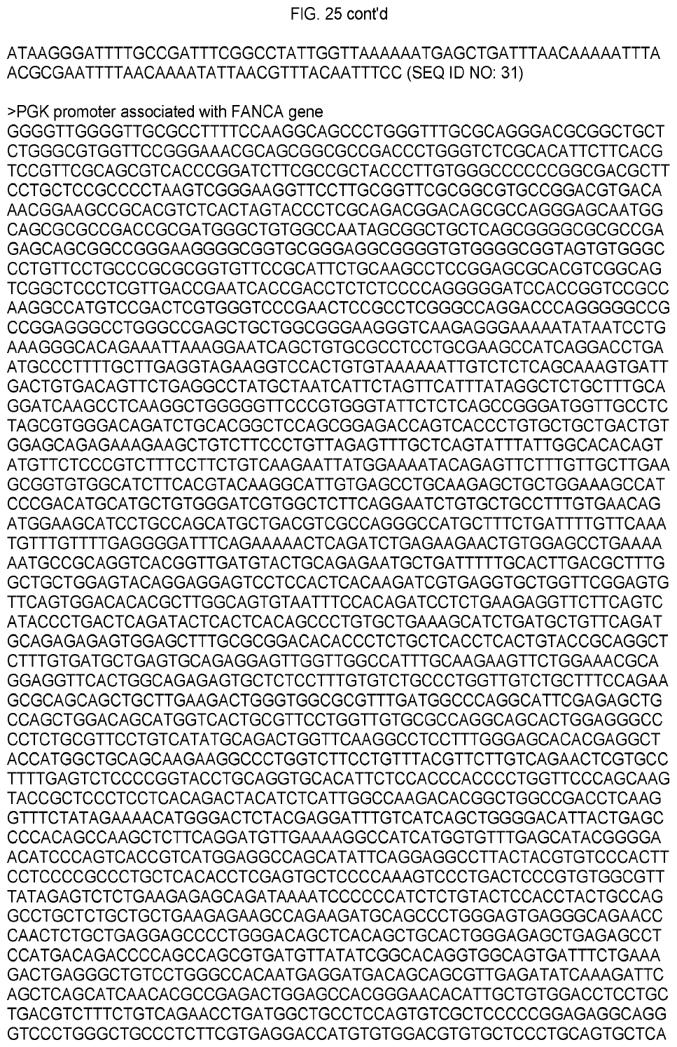

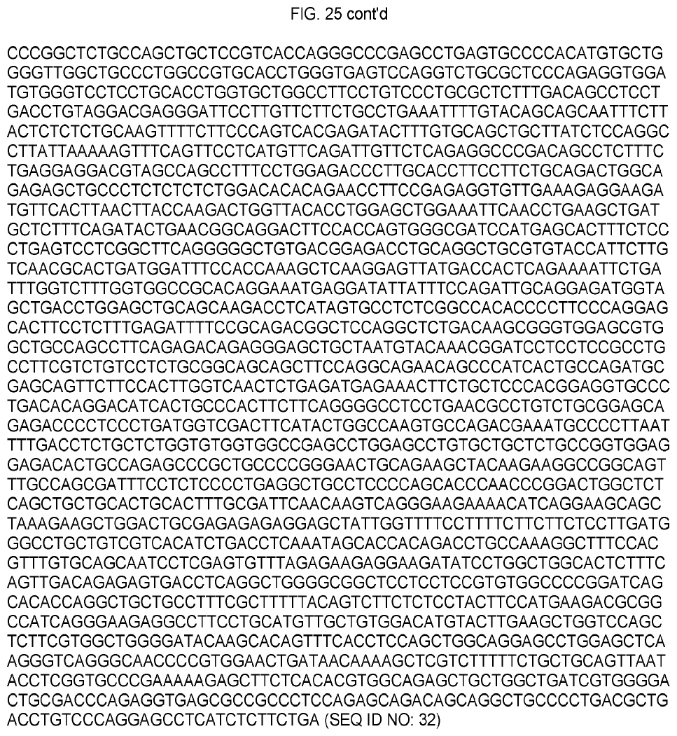

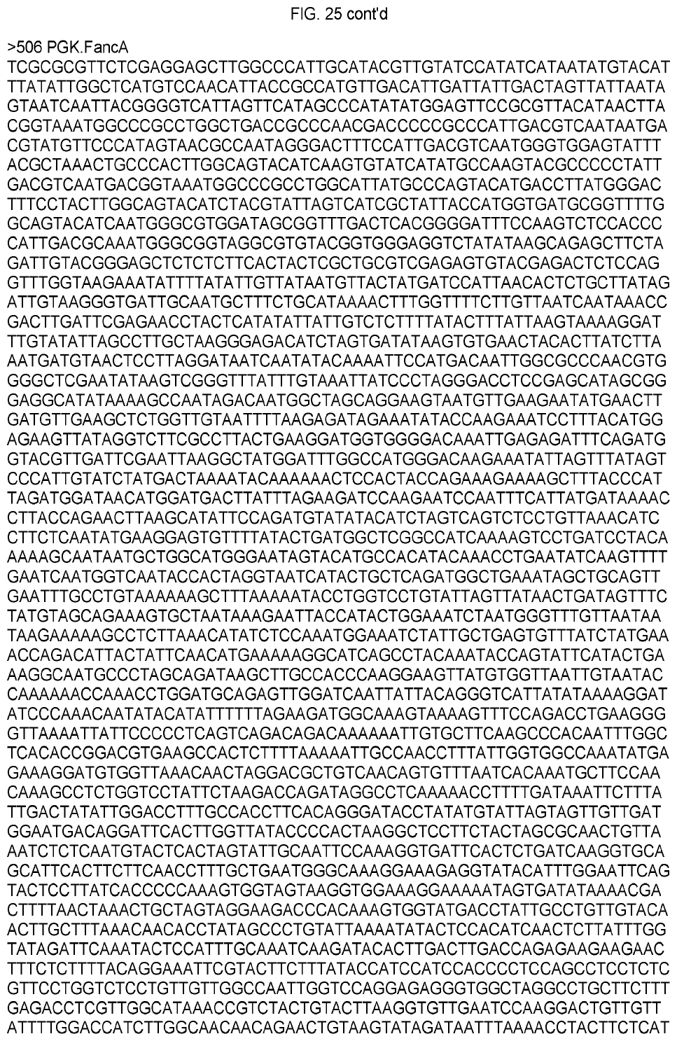

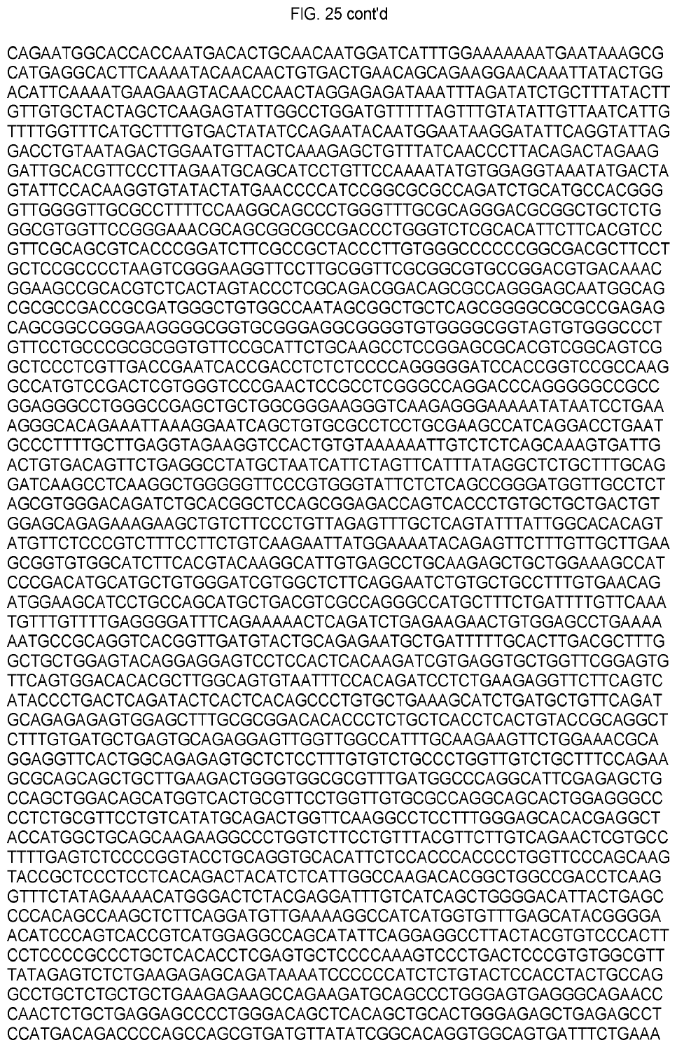

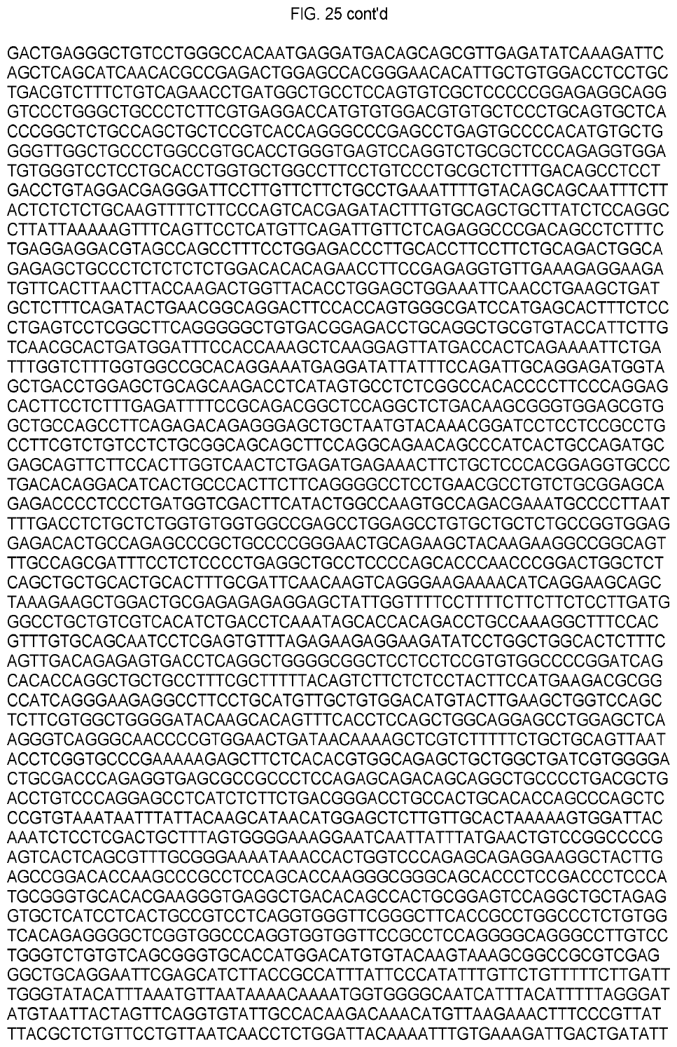

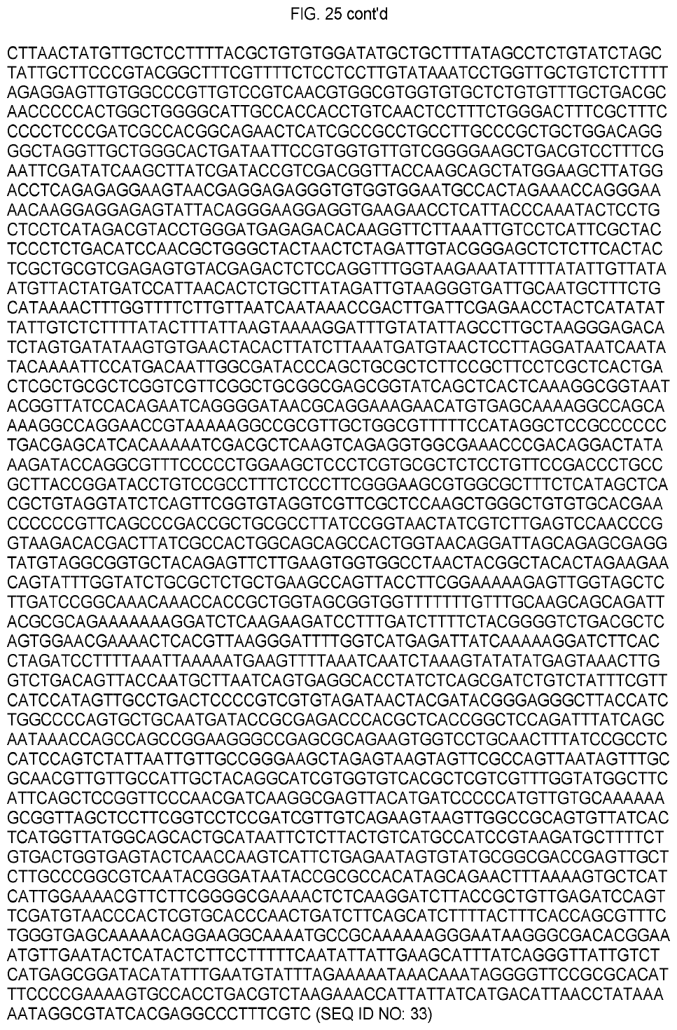

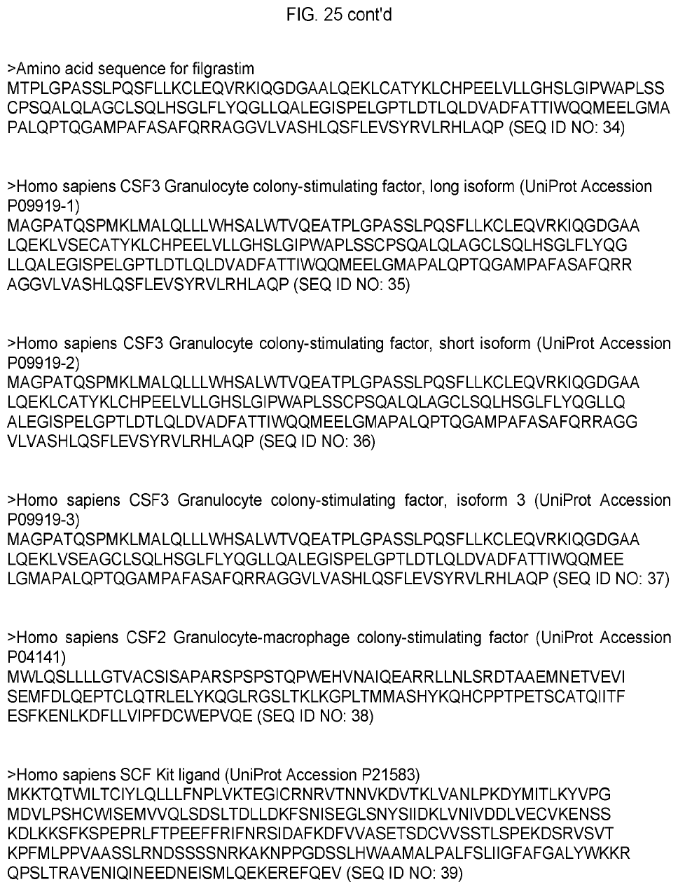

FV vectors are suitable for gene therapy applications because they can (1) accommodate large transgenes (>9kb), (2) transduce slowly dividing cells efficiently, and (3) integrate as a provirus into the genome of target cells, thus enabling stable long term expression of the transgene(s). FV vectors do need cell division for the pre-integration complex to enter the nucleus, however the complex is stable for at least 30 days and still infective. The intracellular half-life of the FV pre-integration complex is comparable to the one of lentiviruses and significantly higher than for gammaretroviruses, therefore FV are also—similar to LV vectors—able to transduce rarely dividing cells. FV vectors are natural self-inactivating vectors and characterized by the fact that they seem to have hardly any potential to activate neighboring genes. In addition, FV vectors can enter any cells known (although the receptor is not identified yet) and infectious vector particles can be concentrated 100-fold without loss of infectivity due to a stable envelope protein. FV vectors achieve high transduction efficiency in pluripotent HSPCs and have been used in animal models to correct monogenetic diseases such as leukocyte adhesion deficiency (LAD) in dogs and FA in mice. FV vectors are also used in preclinical studies of β-thalassemia. Point mutations can be made in Foamy Viruses to render them integration incompetent. For example, foamy viruses can be rendered integration incompetent by introducing point mutations into the highly conserved DD35E catalytic core motif of the foamy virus integrase sequence. See, for example, Deyle D R et al. (2010) J. Virol. 84(18): 9341-9349. As another example, an FV vector can be rendered integration deficient by introducing point mutations into the Pol gene of the FV vector. FIG. 25 shows FV Pol coding sequence (SEQ ID NO: 26) and FV Pol amino acid sequence (SEQ ID NO: 27) with indicated nucleotides or amino acid residues, respectively, that can be mutated to render the FV vector integration deficient.

(ii) Optional Transposable Elements. In particular embodiments, the efficiency of integration, the size of the DNA sequence that can be integrated, and the number of copies of a DNA sequence that can be integrated into a genome can be improved by using transposons. Transposons or transposable elements include a short nucleic acid sequence with terminal repeat sequences upstream and downstream. Active transposons can encode enzymes that facilitate the excision and insertion of nucleic acid into a target DNA sequence.

A number of transposable elements have been described in the art that facilitate insertion of nucleic acids into the genome of vertebrates, including humans. Examples include sleeping beauty (e.g., derived from the genome of salmonid fish); piggyback (e.g., derived from lepidopteran cells and/or the Myotis lucifugus); mariner (e.g., derived from Drosophila); frog prince (e.g., derived from Rana pipiens); Tol2 (e.g., derived from medaka fish); TcBuster (e.g., derived from the red flour beetle Tribolium castaneum) and spinON.

(iii) PGK Promoter and Therapeutic Genes. In particular embodiments, the PGK promoter is derived from the human gene encoding phosphoglycerate kinase (PGK). In particular embodiments, the PGK promoter includes binding sites for the Rap1p, Abflp, and/or Gcrlp transcription factors. In particular embodiments, the PGK promoter includes 500 base pairs: Start (0); Styl (21); Nspl—Sphl (40); Bpml—Eco57Ml (52); BaeGl—Bme1580I (63); Agel (111); BsmBl—Spel (246); BssS α l (252); Blpl (274); BsrDl (285); Stul (295); Bgll (301); Eael (308); AlwNl (350); EcoO109l—PpuMl (415); BspEl (420); Bsml (432); Earl (482); End (500). In particular embodiments, a PGK promoter includes SEQ ID NO: 28.

The PGK promoter will drive expression of a therapeutic gene. A PGK promoter associated with a therapeutic gene includes an orientation of a PGK promoter and therapeutic gene in such a way that results in expression of the therapeutic gene driven by the PGK promoter. The term “gene” refers to a nucleic acid sequence (used interchangeably with polynucleotide or nucleotide sequence) that encodes one or more therapeutic proteins as described herein. This definition includes various sequence polymorphisms, mutations, and/or sequence variants wherein such alterations do not substantially affect the function of the encoded one or more therapeutic proteins. The term “gene” may include not only coding sequences but also regulatory regions such as promoters, enhancers, and termination regions. The term further can include all introns and other DNA sequences spliced from the mRNA transcript, along with variants resulting from alternative splice sites. Gene sequences encoding the molecule can be DNA or RNA that directs the expression of the one or more therapeutic proteins. These nucleic acid sequences may be a DNA strand sequence that is transcribed into RNA or an RNA sequence that is translated into protein. The nucleic acid sequences include both the full-length nucleic acid sequences as well as non-full-length sequences derived from the full-length protein. The sequences can also include degenerate codons of the native sequence or sequences that may be introduced to provide codon preference in a specific cell type.

A gene sequence encoding one or more therapeutic proteins can be readily prepared by synthetic or recombinant methods from the relevant amino acid sequence. In particular embodiments, the gene sequence encoding any of these sequences can also have one or more restriction enzyme sites at the 5′ and/or 3′ ends of the coding sequence in order to provide for easy excision and replacement of the gene sequence encoding the sequence with another gene sequence encoding a different sequence. In particular embodiments, the gene sequence encoding the sequences can be codon optimized for expression in mammalian cells.

Particular examples of therapeutic genes and/or gene products to treat immune deficiencies can include: genes associated with SCID including γC, JAK3, IL7RA, RAG1, RAG2, DCLRE1C, PRKDC, LIG4, NHEJ1, CD3D, CD3E, CD3Z, CD3G, PTPRC, ZAP70, LCK, AK2, ADA, PNP, WHN, CHD7, ORAI1, STIM1, CORO1A, CIITA, RFXANK, RFXS, RFXAP, RMRP, DKC1, TERT, TINF2, DCLRE1B, and SLC46A1; FANC family genes including FancA, FancB, FancC, FancD1 (BRCA2), FancD2, FancE, FancF, FancG, FancI, FancJ (BRIP1), FancL, FancM, FancN (PALB2), FancO (RAD51C), FancP (SLX4), FancQ (ERCC4), FancR (RAD51), FancS (BRCA1), FancT (UBE2T), FancU (XRCC2), FancV (MAD2L2), and FancW (RFWD3); soluble CD40; CTLA; Fas L; antibodies to CD4, CD5, CD7, CD52, etc.; antibodies to IL1, IL2, IL6; an antibody to TCR specifically present on autoreactive T cells; IL4; IL10; IL12; IL13; IL1Ra, sIL1RI, sIL1RII; sTNFRI; sTNFRII; antibodies to TNF; P53, PTPN22, and DRB1*1501/DQB1*0602; globin family genes; WAS; phox; dystrophin; pyruvate kinase; CLN3; ABCD1; arylsulfatase A; SFTPB; SFTPC; NLX2.1; ABCA3; GATA1; ribosomal protein genes; TERT; TERC; DKC1; TINF2; CFTR; LRRK2; PARK2; PARK7; PINK1; SNCA; PSEN1; PSEN2; APP; SOD1; TDP43; FUS; ubiquilin 2; and/or C9ORF72.

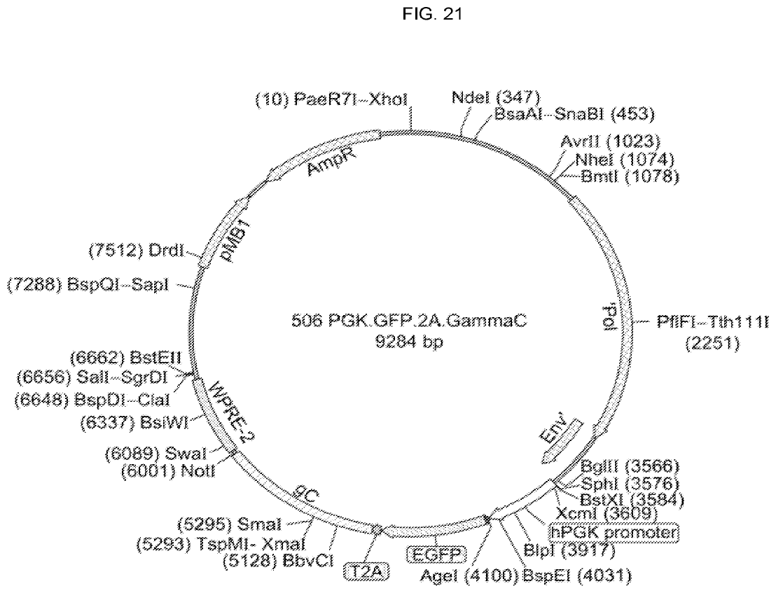

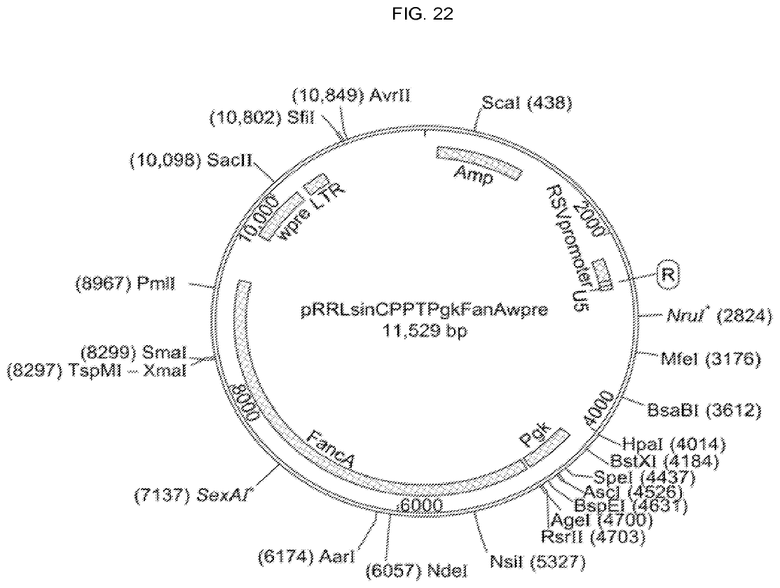

FIG. 25 provides sequences according to the current disclosure as follows: exemplary codon optimized Human γC DNA (SEQ ID NO: 1); exemplary native Human γC DNA (SEQ ID NO: 2); exemplary native canine γC DNA (SEQ ID NO: 3); exemplary human γC AA (SEQ ID NO: 4); and exemplary native canine γC AA (91% conserved with human) (SEQ ID NO: 5). Exemplary genes and proteins associated with SCID include: Homo sapiens JAK3 coding sequence (SEQ ID NO: 6); Homo sapiens PNP coding sequence (SEQ ID NO: 7); Homo sapiens ADA coding sequence (SEQ ID NO: 8); Homo sapiens RAG1 coding sequence (SEQ ID NO: 9); Homo sapiens RAG2 coding sequence (SEQ ID NO: 10); Homo sapiens JAK3 AA (SEQ ID NO: 11); Homo sapiens PNP AA (SEQ ID NO: 12); Homo sapiens ADA AA (SEQ ID NO: 13); Homo sapiens RAG1 AA (SEQ ID NO: 14); and Homo sapiens RAG2 AA (SEQ ID NO: 15). Exemplary genes and proteins associated with FA include: Homo sapiens FANCA coding sequence (SEQ ID NO: 16); Homo sapiens FANCC coding sequence (SEQ ID NO: 17); Homo sapiens FANCE coding sequence (SEQ ID NO: 18); Homo sapiens FANCF coding sequence (SEQ ID NO: 19); Homo sapiens FANCG coding sequence (SEQ ID NO: 20); Homo sapiens FANCA AA (SEQ ID NO: 21); Homo sapiens FANCC AA (SEQ ID NO: 22); Homo sapiens FANCE AA (SEQ ID NO: 23); Homo sapiens FANCF AA (SEQ ID NO: 24); and Homo sapiens FANCG AA (SEQ ID NO: 25). Further, exemplary integration deficient foamy vectors include, for example, FV Pol gene DNA (SEQ ID NO: 26; to generate integration deficient foamy vector (IDFV), either bolded A is mutated to C in underlined sequence or underlined A is mutated to C in bolded sequence); and FV Pol gene AA (SEQ ID NO: 27; to generate IDFV, underlined D is mutated to A (separately, 2 different versions). An exemplary sequence of a PGK promoter includes SEQ ID NO: 28. An exemplary sequence of foamy vector for human clinical trials includes 508 PGK.γC FV plasmid (SEQ ID NO: 29). An exemplary sequence of foamy virus vector containing a γC gene includes 506 PGK.GFP.2A.γC FV (SEQ ID NO: 30 and FIG. 21). An exemplary sequence of a lentiviral vector containing a FancA gene includes 18 pRRLsinCPPT-Pgk-FancA-wpre (SEQ ID NO: 31 and FIG. 22). An exemplary sequence of PGK promoter associated with FANCA gene includes SEQ ID NO: 32. An exemplary sequence of foamy virus vector containing a PGK promoter associated with a FANCA gene includes 506 PGK.FancA (SEQ ID NO: 33).

(iv) Mobilization Factors. Approved agents for HSPC mobilization include G-CSF, granulocyte macrophage colony stimulating factor (GM-CSF), AMD3100 and SCF.

G-CSF is a cytokine whose functions in HSPC mobilization can include the promotion of granulocyte expansion and both protease-dependent and independent attenuation of adhesion molecules and disruption of the SDF-1/CXCR4 axis. In particular embodiments, any commercially available form of G-CSF known to one of ordinary skill in the art can be used in the methods and formulations as disclosed herein, for example, Filgrastim (Neupogen®, Amgen Inc., Thousand Oaks, Calif.) and PEGylated Filgrastim (Pegfilgrastim, Neulasta®, Amgen Inc., Thousand Oaks, Calif.). In particular embodiments, G-CSF can include any of SEQ ID NOs: 34-37.

GM-CSF is a monomeric glycoprotein also known as colony-stimulating factor 2 (CSF2) that functions as a cytokine and is naturally secreted by macrophages, T cells, mast cells, natural killer cells, endothelial cells, and fibroblasts. In particular embodiments, any commercially available form of GM-CSF known to one of ordinary skill in the art can be used in the methods and formulations as disclosed herein, for example, Sargramostim (Leukine, Bayer Healthcare Pharmaceuticals, Seattle, Wash.) and molgramostim (Schering-Plough, Kenilworth, N.J.). In particular embodiments, GM-CSF can include SEQ ID NO: 38.

AMD3100 (Mozobil™, Plerixafor™; Sanofi-Aventis, Paris, France), a synthetic organic molecule of the bicyclam class, is a chemokine receptor antagonist and reversibly inhibits SDF-1 binding to CXCR4, promoting HSPC mobilization. AMD3100 is approved to be used in combination with G-CSF for HSPC mobilization in patients with myeloma and lymphoma. The structure of AMD3100 is:

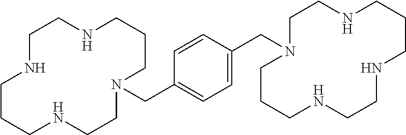 [SMILES: c1cc(CN2CCCNCCNCCCNCC2)ccc1CN1CCCNCCNCCCNCC1]

SCF, also known as KIT ligand, KL, or steel factor, is a cytokine that binds to the c-kit receptor (CD117). SCF can exist both as a transmembrane protein and a soluble protein. This cytokine plays an important role in hematopoiesis, spermatogenesis, and melanogenesis. In particular embodiments, any commercially available form of SCF known to one of ordinary skill in the art can be used in the methods and formulations as disclosed herein, for example, recombinant human SCF (Ancestim, Stemgen®, Amgen Inc., Thousand Oaks, Calif.). In particular embodiments, SCF can include SEQ ID NO: 39.

Chemotherapy used in intensive myelosuppressive treatments also mobilizes HSPCs to the peripheral blood as a result of compensatory neutrophil production following chemotherapy-induced aplasia. In particular embodiments, chemotherapeutic agents that can be used for mobilization of HSPCs include cyclophosphamide, etoposide, ifosfamide, cisplatin, and cytarabine.

Additional agents that can be used for cell mobilization include: CXCL12/CXCR4 modulators (e.g., CXCR4 antagonists: POL6326 (Polyphor, Allschwil, Switzerland), a synthetic cyclic peptide which reversibly inhibits CXCR4; BKT-140 (4F-benzoyl-TN14003; Biokine Therapeutics, Rehovit, Israel); TG-0054 (Taigen Biotechnology, Taipei, Taiwan); CXCL12 neutralizer NOX-A12 (NOXXON Pharma, Berlin, Germany) which binds to SDF-1, inhibiting its binding to CXCR4); Sphingosine-1-phosphate (S1P) agonists (e.g., SEW2871, Juarez J G et al. (2012) Blood 119: 707-716); vascular cell adhesion molecule-1 (VCAM) or very late antigen 4 (VLA-4) inhibitors (e.g., Natalizumab, a recombinant humanized monoclonal antibody against α4 subunit of VLA-4 (Zohren F et al. (2008) Blood 111: 3893-3895); B105192, a small molecule inhibitor of VLA-4 (Ramirez P et al. (2009) Blood 114: 1340-1343)); parathyroid hormone (Brunner S et al. (2008) Exp Hematol. 36: 1157-1166); proteasome inhibitors (e.g., Bortezomib, Ghobadi A et al. (2012) ASH Annual Meeting Abstracts. p. 583); Groβ, a member of CXC chemokine family which stimulates chemotaxis and activation of neutrophils by binding to the CXCR2 receptor (e.g., SB-251353, King A G et al. (2001) Blood 97: 1534-1542); stabilization of hypoxia inducible factor (HIF) (e.g., FG-4497, Forristal C E et al. (2012) ASH Annual Meeting Abstracts. p. 216); Firategrast, an α4β1 and α4β7 integrin inhibitor (α4β1/7) (Kim A G et al. (2016) Blood 128: 2457-2461); Vedolizumab, a humanized monoclonal antibody against the α4β7 integrin (Rosario Metal. (2016) Clin Drug Investig 36: 913-923); and BOP (N-(benzenesulfonyl)-L -prolyl-L-O-(1-pyrrolidinylcarbonyl) tyrosine) which targets integrins α9β1/α4β1 (Cao B et al. (2016) Nat Commun 7: 11007). Additional agents that can be used for HSPC mobilization are described in, for example, Richter R et al. (2017) Transfus Med Hemother 44:151-164, Bendall J L & Bradstock K F (2014) Cytokine & Growth Factor Reviews 25: 355-367, WO 2003043651, WO 2005017160, WO 2011069336, U.S. Pat. Nos. 5,637,323, 7,288,521, 9,782,429, US 2002/0142462, and US 2010/02268.

(v) Formulations. The FV vectors described herein can be formulated for administration to a subject. Formulations include an FV vector including a PGK promoter associated with a therapeutic gene (“active ingredient”) and one or more pharmaceutically acceptable carriers.

In particular embodiments, the formulations include active ingredients of at least 0.1% w/v or w/w of the formulation; at least 1% w/v or w/w of formulation; at least 10% w/v or w/w of formulation; at least 20% w/v or w/w of formulation; at least 30% w/v or w/w of formulation; at least 40% w/v or w/w of formulation; at least 50% w/v or w/w of formulation; at least 60% w/v or w/w of formulation; at least 70% w/v or w/w of formulation; at least 80% w/v or w/w of formulation; at least 90% w/v or w/w of formulations; at least 95% w/v or w/w of formulation; or at least 99% w/v or w/w of formulation.

Exemplary generally used pharmaceutically acceptable carriers include any and all absorption delaying agents, antioxidants, binders, buffering agents, bulking agents or fillers, chelating agents, coatings, disintegration agents, dispersion media, gels, isotonic agents, lubricants, preservatives, salts, solvents or co-solvents, stabilizers, surfactants, and/or delivery vehicles.

Exemplary antioxidants include ascorbic acid, methionine, and vitamin E.

Exemplary buffering agents include citrate buffers, succinate buffers, tartrate buffers, fumarate buffers, gluconate buffers, oxalate buffers, lactate buffers, acetate buffers, phosphate buffers, histidine buffers, and/or trimethylamine salts.

An exemplary chelating agent is EDTA.

Exemplary isotonic agents include polyhydric sugar alcohols including trihydric or higher sugar alcohols, such as glycerin, erythritol, arabitol, xylitol, sorbitol, or mannitol.

Exemplary preservatives include phenol, benzyl alcohol, meta-cresol, methyl paraben, propyl paraben, octadecyldimethylbenzyl ammonium chloride, benzalkonium halides, hexamethonium chloride, alkyl parabens such as methyl or propyl paraben, catechol, resorcinol, cyclohexanol, and 3-pentanol.

Stabilizers refer to a broad category of excipients which can range in function from a bulking agent to an additive which solubilizes the active ingredients or helps to prevent denaturation or adherence to the container wall. Typical stabilizers can include polyhydric sugar alcohols; amino acids, such as arginine, lysine, glycine, glutamine, asparagine, histidine, alanine, ornithine, L-leucine, 2-phenylalanin, glutamic acid, and threonine; organic sugars or sugar alcohols, such as lactose, trehalose, stachyose, mannitol, sorbitol, xylitol, ribitol, myoinisitol, galactitol, glycerol, and cyclitols, such as inositol; PEG; amino acid polymers; sulfur-containing reducing agents, such as urea, glutathione, thioctic acid, sodium thioglycolate, thioglycerol, alpha-monothioglycerol, and sodium thiosulfate; low molecular weight polypeptides (i.e., <10 residues); proteins such as human serum albumin, bovine serum albumin, gelatin or immunoglobulins; hydrophilic polymers such as polyvinylpyrrolidone; monosaccharides such as xylose, mannose, fructose and glucose; disaccharides such as lactose, maltose and sucrose; trisaccharides such as raffinose, and polysaccharides such as dextran. Stabilizers are typically present in the range of from 0.1 to 10,000 parts by weight based on therapeutic weight.

The formulations disclosed herein can be formulated for administration by, for example, injection. For injection, formulation can be formulated as aqueous solutions, such as in buffers including Hanks' solution, Ringer's solution, or physiological saline, or in culture media, such as Iscove's Modified Dulbecco's Medium (IMDM). The aqueous solutions can include formulatory agents such as suspending, stabilizing, and/or dispersing agents. Alternatively, the formulation can be in lyophilized and/or powder form for constitution with a suitable vehicle, e.g., sterile pyrogen-free water, before use.

Any formulation disclosed herein can advantageously include any other pharmaceutically acceptable carriers which include those that do not produce significantly adverse, allergic, or other untoward reactions that outweigh the benefit of administration. Exemplary pharmaceutically acceptable carriers and formulations are disclosed in Remington's Pharmaceutical Sciences, 18th Ed. Mack Printing Company, 1990. Moreover, formulations can be prepared to meet sterility, pyrogenicity, general safety, and purity standards as required by US FDA Office of Biological Standards and/or other relevant foreign regulatory agencies.

Formulations disclosed herein can include one or more mobilization factors. The one or more mobilization factors can include G-CSF/Filgrastim (Amgen), GM-CSF, AMD3100 (Sigma), SCF, and/or a chemotherapeutic agent. In particular embodiments, formulations disclosed herein can include: an FV vector including a PGK promoter associated with a therapeutic gene; and G-CSF/Filgrastim (Amgen). In particular embodiments, formulations disclosed herein can include: an FV vector including a PGK promoter associated with a therapeutic gene; G-CSF/Filgrastim (Amgen); and AMD3100. In particular embodiments, formulations disclosed herein can include: an FV vector including a PGK promoter associated with a therapeutic gene; and GM-CSF/Sargramostim (Amgen). In particular embodiments, formulations disclosed herein can include: an FV vector including a PGK promoter associated with a therapeutic gene; GM-CSF/Sargramostim (Amgen); and AMD3100. In particular embodiments, formulations disclosed herein can include: an FV vector including a PGK promoter associated with a therapeutic gene; and SCF/Ancestim (Amgen). In particular embodiments, formulations disclosed herein can include: an FV vector including a PGK promoter associated with a therapeutic gene; SCF/Ancestim (Amgen); and AMD3100.

(vi) Methods of Use. The formulations disclosed herein can be used for treating subjects (humans, veterinary animals (dogs, cats, reptiles, birds, etc.), livestock (horses, cattle, goats, pigs, chickens, etc.), and research animals (monkeys, rats, mice, fish, etc.). Treating subjects includes delivering therapeutically effective amounts. Therapeutically effective amounts include those that provide effective amounts, prophylactic treatments, and/or therapeutic treatments.

An “effective amount” is the amount of a formulation necessary to result in a desired physiological change in a subject. Effective amounts are often administered for research purposes.

A “prophylactic treatment” includes a treatment administered to a subject who does not display signs or symptoms of a condition to be treated or displays only early signs or symptoms of the condition to be treated such that treatment is administered for the purpose of diminishing, preventing, or decreasing the risk of developing the condition. Thus, a prophylactic treatment functions as a preventative treatment against a condition.

A “therapeutic treatment” includes a treatment administered to a subject who displays symptoms or signs of a condition and is administered to the subject for the purpose of reducing the severity or progression of the condition.

FV vectors can be administered in concert with HSPC mobilization. In particular embodiments, administration of an FV vector occurs concurrently with administration of one or more mobilization factors. In particular embodiments, administration of an FV vector follows administration of one or more mobilization factors. In particular embodiments, administration of an FV vector follows administration of a first one or more mobilization factors and occurs concurrently with administration of a second one or more mobilization factors.

The actual dose and amount of FV vectors and, in particular embodiments, of FV vectors and mobilization factors, administered to a particular subject and concordant mobilization procedure and schedule can be determined by a physician, veterinarian, or researcher taking into account parameters such as physical and physiological factors including target; body weight; type of condition; severity of condition; upcoming relevant events, when known; previous or concurrent therapeutic interventions; idiopathy of the subject; and route of administration, for example. In addition, in vitro and in vivo assays can optionally be employed to help identify optimal dosage ranges.

Therapeutically effective amounts of FV vector including a PGK promoter associated with a therapeutic gene can include doses ranging from, for example, 1×10⁷ to 50×10⁸ infection units (IU) or from 5×10⁷ to 20×10⁸ IU. In other examples, a dose can include 5×10⁷ IU, 6×10⁷ IU, 7×10⁷ IU, 8×10⁷ IU, 9×10⁷ IU, 1×10⁸ IU, 2×10⁸ IU, 3×10⁸ IU, 4×10⁸ IU, 5×10⁸ IU, 6×10⁸ IU, 7×10⁸ IU, 8×10⁸ IU, 9×10⁸ IU, 10×10⁸ IU, or more. In particular embodiments, a therapeutically effective amount of an FV vector including a PGK promoter associated with a therapeutic gene includes 4×10⁸ IU. In particular embodiments, a therapeutically effective amount of an FV vector including a PGK promoter associated with a therapeutic gene can be administered subcutaneously or intravenously. In particular embodiments, a therapeutically effective amount of an FV vector including a PGK promoter associated with a therapeutic gene can be administered following administration with one or more mobilization factors.

In particular embodiments, a therapeutically effective amount of G-CSF includes 0.1 μg/kg to 100 μg/kg. In particular embodiments, a therapeutically effective amount of G-CSF includes 0.5 μg/kg to 50 μg/kg. In particular embodiments, a therapeutically effective amount of G-CSF includes 0.5 μg/kg, 1 μg/kg, 2 μg/kg, 3 μg/kg, 4 μg/kg, 5 μg/kg, 6 μg/kg, 7 μg/kg, 8 μg/kg, 9 μg/kg, 10 μg/kg, 11 μg/kg, 12 μg/kg, 13 μg/kg, 14 μg/kg, 15 μg/kg, 16 μg/kg, 17 μg/kg, 18 μg/kg, 19 μg/kg, 20 μg/kg, or more. In particular embodiments, a therapeutically effective amount of G-CSF includes 5 μg/kg. In particular embodiments, G-CSF can be administered subcutaneously or intravenously. In particular embodiments, G-CSF can be administered for 1 day, 2 consecutive days, 3 consecutive days, 4 consecutive days, 5 consecutive days, or more. In particular embodiments, G-CSF can be administered for 4 consecutive days. In particular embodiments, G-CSF can be administered for 5 consecutive days. In particular embodiments, as a single agent, G-CSF can be used at a dose of 10 μg/kg subcutaneously daily, initiated 3, 4, 5, 6, 7, or 8 days before FV delivery. In particular embodiments, G-CSF can be administered as a single agent followed by concurrent administration with another mobilization factor. In particular embodiments, G-CSF can be administered as a single agent followed by concurrent administration with AMD3100. In particular embodiments, a treatment protocol includes a 5 day treatment where G-CSF can be administered on day 1, day 2, day 3, and day 4 and on day 5, G-CSF and AMD3100 are administered 6 to 8 hours prior to FV administration.

Therapeutically effective amounts of GM-CSF to administer can include doses ranging from, for example, 0.1 to 50 μg/kg or from 0.5 to 30 μg/kg. In particular embodiments, a dose at which GM-CSF can be administered includes 0.5 μg/kg, 1 μg/kg, 2 μg/kg, 3 μg/kg, 4 μg/kg, 5 μg/kg, 6 μg/kg, 7 μg/kg, 8 μg/kg, 9 μg/kg, 10 μg/kg, 11 μg/kg, 12 μg/kg, 13 μg/kg, 14 μg/kg, 15 μg/kg, 16 μg/kg, 17 μg/kg, 18 μg/kg, 19 μg/kg, 20 μg/kg, or more. In particular embodiments, GM-CSF can be administered subcutaneously for 1 day, 2 consecutive days, 3 consecutive days, 4 consecutive days, 5 consecutive days, or more. In particular embodiments, GM-CSF can be administered subcutaneously or intravenously. In particular embodiments, GM-CSF can be administered at a dose of 10 μg/kg subcutaneously daily initiated 3, 4, 5, 6, 7, or 8 days before FV delivery. In particular embodiments, GM-CSF can be administered as a single agent followed by concurrent administration with another mobilization factor. In particular embodiments, GM-CSF can be administered as a single agent followed by concurrent administration with AMD3100. In particular embodiments, a treatment protocol includes a 5 day treatment where GM-CSF can be administered on day 1, day 2, day 3, and day 4 and on day 5, GM-CSF and AMD3100 are administered 6 to 8 hours prior to FV administration. A dosing regimen for Sargramostim can include 200 μg/m², 210 μg/m², 220 μg/m², 230 μg/m², 240 μg/m², 250 μg/m², 260 μg/m², 270 μg/m², 280 μg/m², 290 μg/m², 300 μg/m², or more. In particular embodiments, Sargramostim can be administered for 1 day, 2 consecutive days, 3 consecutive days, 4 consecutive days, 5 consecutive days, or more. In particular embodiments, Sargramostim can be administered subcutaneously or intravenously. In particular embodiments, a dosing regimen for Sargramostim can include 250 μg/m²/day intravenous or subcutaneous and can be continued until a targeted cell amount is reached in the peripheral blood or can be continued for 5 days. In particular embodiments, Sargramostim can be administered as a single agent followed by concurrent administration with another mobilization factor. In particular embodiments, Sargramostim can be administered as a single agent followed by concurrent administration with AMD3100. In particular embodiments, a treatment protocol includes a 5 day treatment where Sargramostim can be administered on day 1, day 2, day 3, and day 4 and on day 5, Sargramostim and AMD3100 are administered 6 to 8 hours prior to FV administration.

In particular embodiments, a therapeutically effective amount of AMD3100 includes 0.1 mg/kg to 100 mg/kg. In particular embodiments, a therapeutically effective amount of AMD3100 includes 0.5 mg/kg to 50 mg/kg. In particular embodiments, a therapeutically effective amount of AMD3100 includes 0.5 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, or more. In particular embodiments, a therapeutically effective amount of AMD3100 includes 4 mg/kg. In particular embodiments, a therapeutically effective amount of AMD3100 includes 5 mg/kg. In particular embodiments, a therapeutically effective amount of AMD3100 includes 10 μg/kg to 500 μg/kg or from 50 μg/kg to 400 μg/kg. In particular embodiments, a therapeutically effective amount of AMD3100 includes 100 μg/kg, 150 μg/kg, 200 μg/kg, 250 μg/kg, 300 μg/kg, 350 μg/kg, or more. In particular embodiments, AMD3100 can be administered subcutaneously or intravenously. In particular embodiments, AMD3100 can be administered subcutaneously at 160-240 μg/kg 6 to 11 hours prior to FV delivery. In particular embodiments, a therapeutically effective amount of AMD3100 can be administered concurrently with administration of another mobilization factor. In particular embodiments, a therapeutically effective amount of AMD3100 can be administered following administration of another mobilization factor. In particular embodiments, a therapeutically effective amount of AMD3100 can be administered following administration of G-CSF. In particular embodiments, a treatment protocol includes a 5 day treatment where G-CSF is administered on day 1, day 2, day 3, and day 4 and on day 5, G-CSF and AMD3100 are administered 6 to 8 hours prior to FV injection.

Therapeutically effective amounts of SCF to administer can include doses ranging from, for example, 0.1 to 100 μg/kg/day or from 0.5 to 50 μg/kg/day. In particular embodiments, a dose at which SCF can be administered includes 0.5 μg/kg/day, 1 μg/kg/day, 2 μg/kg/day, 3 μg/kg/day, 4 μg/kg/day, 5 μg/kg/day, 6 μg/kg/day, 7 μg/kg/day, 8 μg/kg/day, 9 μg/kg/day, 10 μg/kg/day, 11 μg/kg/day, 12 μg/kg/day, 13 μg/kg/day, 14 μg/kg/day, 15 μg/kg/day, 16 μg/kg/day, 17 μg/kg/day, 18 μg/kg/day, 19 μg/kg/day, 20 μg/kg/day, 21 μg/kg/day, 22 μg/kg/day, 23 μg/kg/day, 24 μg/kg/day, 25 μg/kg/day, 26 μg/kg/day, 27 μg/kg/day, 28 μg/kg/day, 29 μg/kg/day, 30 μg/kg/day, or more. In particular embodiments, SCF can be administered for 1 day, 2 consecutive days, 3 consecutive days, 4 consecutive days, 5 consecutive days, or more. In particular embodiments, SCF can be administered subcutaneously or intravenously. In particular embodiments, SCF can be injected subcutaneously at 20 μg/kg/day. In particular embodiments, SCF can be administered as a single agent followed by concurrent administration with another mobilization factor. In particular embodiments, SCF can be administered as a single agent followed by concurrent administration with AMD3100. In particular embodiments, a treatment protocol includes a 5 day treatment where SCF can be administered on day 1, day 2, day 3, and day 4 and on day 5, SCF and AMD3100 are administered 6 to 8 hours prior to FV administration.

In particular embodiments, growth factors GM-CSF and G-CSF can be administered to mobilize HSPC in the bone marrow niches to the peripheral circulating blood to increase the fraction of HSPCs circulating in the blood. In particular embodiments, mobilization can be achieved with administration of G-CSF/Filgrastim (Amgen) and/or AMD3100 (Sigma). In particular embodiments, mobilization can be achieved with administration of GM-CSF/Sargramostim (Amgen) and/or AMD3100 (Sigma). In particular embodiments, mobilization can be achieved with administration of SCF/Ancestim (Amgen) and/or AMD3100 (Sigma). In particular embodiments, administration of G-CSF/Filgrastim precedes administration of AMD3100. In particular embodiments, administration of G-CSF/Filgrastim occurs concurrently with administration of AMD3100. In particular embodiments, administration of G-CSF/Filgrastim precedes administration of AMD3100, followed by concurrent administration of G-CSF/Filgrastim and AMD3100. US 20140193376 describes mobilization protocols utilizing a CXCR4 antagonist with a S1P receptor 1 (S1PR1) modulator agent. US 20110044997 describes mobilization protocols utilizing a CXCR4 antagonist with a vascular endothelial growth factor receptor (VEGFR) agonist.

Therapeutically effective amounts can be administered through any appropriate administration route such as by, injection, infusion, perfusion, and more particularly by administration by one or more of bone marrow, intravenous, intradermal, intraarterial, intranodal, intralymphatic, intraperitoneal injection, infusion, or perfusion).

In particular embodiments, methods of the present disclosure can restore T-cell mediated immune responses in a subject in need thereof. Restoration of T-cell mediated immune responses can include restoring thymic output and/or restoring normal T lymphocyte development.

In particular embodiments, restoring thymic output can include restoring the frequency of CD3+ T cells expressing CD45RA in peripheral blood to a level comparable to that of a reference level derived from a control population. In particular embodiments, restoring thymic output can include restoring the number of T cell receptor excision circles (TRECs) per 10⁶ maturing T cells to a level comparable to that of a reference level derived from a control population. The number of TRECs per 10⁶ maturing T cells can be determined as described in Example 1 and in Kennedy D R et al. (2011) Vet Immunol Immunopathol 142: 36-48.

In particular embodiments, restoring normal T lymphocyte development includes restoring the ratio of CD4+ cells: CD8+ cells to 2. In particular embodiments, restoring normal T lymphocyte development includes detecting the presence of αβ TCR in circulating T-lymphocytes. The presence of αβ TCR in circulating T-lymphocytes can be detected, for example, by flow cytometry using antibodies that bind an α and/or β chain of a TCR. In particular embodiments, restoring normal T lymphocyte development includes detecting the presence of a diverse TCR repertoire comparable to that of a reference level derived from a control population. TCR diversity can be assessed by TCRVβ spectratyping, which analyzes genetic rearrangement of the variable region of the TCRβ gene. Robust, normal spectratype profiles can be characterized by a Gaussian distribution of fragments sized across 17 families of TCRVβ segments. In particular embodiments, restoring normal T lymphocyte development includes restoring T-cell specific signaling pathways. Restoration of T-cell specific signaling pathways can be assessed by lymphocyte proliferation following exposure to the T cell mitogen phytohemagglutinin (PHA). In particular embodiments, restoring normal T lymphocyte development includes restoring white blood cell count, neutrophil cell count, monocyte cell count, lymphocyte cell count, and/or platelet cell count to a level comparable to a reference level derived from a control population.

In particular embodiments, methods of the present disclosure can improve the kinetics and/or clonal diversity of lymphocyte reconstitution in a subject in need thereof. In particular embodiments, improving the kinetics of lymphocyte reconstitution can include increasing the number of circulating T lymphocytes to within a range of a reference level derived from a control population. In particular embodiments, improving the kinetics of lymphocyte reconstitution can include increasing the absolute CD3+ lymphocyte count to within a range of a reference level derived from a control population. A range of can be a range of values observed in or exhibited by normal (i.e., non-immuno-compromised) subjects for a given parameter. In particular embodiments, improving the kinetics of lymphocyte reconstitution can include reducing the time required to reach normal lymphocyte counts as compared to a subject in need thereof not administered a therapy described herein. In particular embodiments, improving the kinetics of lymphocyte reconstitution can include increasing the frequency of gene corrected lymphocytes as compared to a subject in need thereof not administered a therapy described herein. In particular embodiments, improving the kinetics of lymphocyte reconstitution can include increasing diversity of clonal repertoire of gene corrected lymphocytes in the subject as compared to a subject in need thereof not administered a gene therapy described herein. Increasing diversity of clonal repertoire of gene corrected lymphocytes can include increasing the number of unique retroviral integration site (RIS) clones as measured by a RIS analysis. RIS analysis can be performed as described in Example 1.

In particular embodiments, methods of the present disclosure can restore bone marrow function in a subject in need thereof. In particular embodiments, restoring bone marrow function can include improving bone marrow repopulation with gene corrected cells as compared to a subject in need thereof not administered a therapy described herein. Improving bone marrow repopulation with gene corrected cells can include increasing the percentage of cells that are gene corrected. In particular embodiments, the cells are selected from white blood cells and bone marrow derived cells. In particular embodiments, the percentage of cells that are gene corrected can be measured using an assay selected from quantitative real time PCR and flow cytometry.

In particular embodiments, methods of the present disclosure can normalize primary and secondary antibody responses to immunization in a subject in need thereof. Normalizing primary and secondary antibody responses to immunization can include restoring B-cell and/or T-cell cytokine signaling programs functioning in class switching and memory response to an antigen. Normalizing primary and secondary antibody responses to immunization can be measured by a bacteriophage immunization assay. In particular embodiments, restoration of B-cell and/or T-cell cytokine signaling programs can be assayed after immunization with the T-cell dependent neoantigen bacteriophage ϕX174 as described in Example 1. In particular embodiments, normalizing primary and secondary antibody responses to immunization can include increasing the level of IgA, IgM, and/or IgG in a subject in need thereof to a level comparable to a reference level derived from a control population. In particular embodiments, normalizing primary and secondary antibody responses to immunization can include increasing the level of IgA, IgM, and/or IgG in a subject in need thereof to a level greater than that of a subject in need thereof not administered a gene therapy described herein. The level of IgA, IgM, and/or IgG can be measured by, for example, an immunoglobulin test. In particular embodiments, the immunoglobulin test includes antibodies binding IgG, IgA, IgM, kappa light chain, lambda light chain, and/or heavy chain. In particular embodiments, the immunoglobulin test includes serum protein electrophoresis, immunoelectrophoresis, radial immunodiffusion, nephelometry and turbidimetry. Commercially available immunoglobulin test kits include MININEPH™ (Binding site, Birmingham, UK), and immunoglobulin test systems from Dako (Denmark) and Dade Behring (Marburg, Germany). In particular embodiments, a sample that can be used to measure immunoglobulin levels includes a blood sample, a plasma sample, a cerebrospinal fluid sample, and a urine sample.

In particular embodiments, methods of the present disclosure can be used to treat SCID-X1. In particular embodiments, methods of the present disclosure can be used to treat SCID (e.g., JAK 3 kinase deficiency SCID, purine nucleoside phosphorylase (PNP) deficiency SCID, adenosine deaminase (ADA) deficiency SCID, MHC class II deficiency or recombinase activating gene (RAG) deficiency SCID). In particular embodiments, therapeutic efficacy can be observed through lymphocyte reconstitution, improved clonal diversity and thymopoiesis, reduced infections, and/or improved patient outcome. Therapeutic efficacy can also be observed through one or more of weight gain and growth, improved gastrointestinal function (e.g., reduced diarrhea), reduced upper respiratory symptoms, reduced fungal infections of the mouth (thrush), reduced incidences and severity of pneumonia, reduced meningitis and blood stream infections, and reduced ear infections. In particular embodiments, treating SCIDX-1 with methods of the present disclosure include restoring functionality to the γC-dependent signaling pathway. The functionality of the γC-dependent signaling pathway can be assayed by measuring tyrosine phosphorylation of effector molecules STAT3 and/or STAT5 following in vitro stimulation with IL-21 and/or IL-2, respectively. Tyrosine phosphorylation of STAT3 and/or STAT5 can be measured by intracellular antibody staining.

In particular embodiments, methods of the present disclosure can be used to treat FA. In particular embodiments, therapeutic efficacy can be observed through lymphocyte reconstitution, improved clonal diversity and thymopoiesis, reduced infections, and/or improved patient outcome. Therapeutic efficacy can also be observed through one or more of weight gain and growth, improved gastrointestinal function (e.g., reduced diarrhea), reduced upper respiratory symptoms, reduced fungal infections of the mouth (thrush), reduced incidences and severity of pneumonia, reduced meningitis and blood stream infections, and reduced ear infections. In particular embodiments, treating FA with methods of the present disclosure include increasing resistance of bone marrow derived cells to mitomycin C (MMC). In particular embodiments, the resistance of bone marrow derived cells to MMC can be measured by a cell survival assay in methylcellulose and MMC.

In particular embodiments, methods of the present disclosure can be used to treat hypogammaglobulinemia. Hypogammaglobulinemia is caused by a lack of B-lymphocytes and is characterized by low levels of antibodies in the blood. Hypogammaglobulinemia can occur in patients with chronic lymphocytic leukemia (CLL), multiple myeloma (MM), non-Hodgkin's lymphoma (NHL) and other relevant malignancies as a result of both leukemia-related immune dysfunction and therapy-related immunosuppression. Patients with acquired hypogammaglobulinemia secondary to such hematological malignancies, and those patients receiving post-HSPC transplantation are susceptible to bacterial infections. The deficiency in humoral immunity is largely responsible for the increased risk of infection-related morbidity and mortality in these patients, especially by encapsulated microorganisms. For example, Streptococcus pneumoniae, Haemophilus influenzae, and Staphylococcus aureus, as well as Legionella and Nocardia spp. are frequent bacterial pathogens that cause pneumonia in patients with CLL. Opportunistic infections such as Pneumocystis carinii, fungi, viruses, and mycobacteria also have been observed. The number and severity of infections in these patients can be significantly reduced by administration of immune globulin (Griffiths H et al. (1989) Blood 73: 366-368; Chapel H M et al. (1994) Lancet 343: 1059-1063).

In particular embodiments, formulations are administered to subjects to treat acute lymphoblastic leukemia (ALL), acute myelogenous leukemia (AML), adrenoleukodystrophy, agnogenic myeloid metaplasia, amegakaryocytosis/congenital thrombocytopenia, ataxia telangiectasia, β-thalassemia major, chronic granulomatous disease, chronic lymphocytic leukemia (CLL), chronic myelogenous leukemia (CML), chronic myelomonocytic leukemia, common variable immune deficiency (CVID), complement disorders, congenital agammaglobulinemia, Diamond Blackfan syndrome, familial erythrophagocytic lymphohistiocytosis, Hodgkin's lymphoma, Hurler's syndrome, hyper IgM, IgG subclass deficiency, juvenile myelomonocytic leukemia, metachromatic leukodystrophy, mucopolysaccharidoses, multiple myeloma, myelodysplasia, non-Hodgkin's lymphoma, paroxysmal nocturnal hemoglobinuria (PNH), primary immunodeficiency diseases with antibody deficiency, pure red cell aplasia, refractory anemia, Shwachmann-Diamond-Blackfan anemia, selective IgA deficiency, severe aplastic anemia, sickle cell disease, specific antibody deficiency, Wiskott-Aldridge syndrome, and/or X-linked agammaglobulinemia (XLA).

Particular embodiments include treatment of secondary, or acquired, immune deficiencies such as immune deficiencies caused by trauma, viruses, chemotherapy, toxins, and pollution. As previously indicated, acquired immunodeficiency syndrome (AIDS) is an example of a secondary immune deficiency disorder caused by a virus, the human immunodeficiency virus (HIV), in which a depletion of T lymphocytes renders the body unable to fight infection. Thus, as another example, a gene can be selected to provide a therapeutically effective response against an infectious disease. In particular embodiments, the infectious disease is human immunodeficiency virus (HIV). The therapeutic gene may be, for example, a gene rendering immune cells resistant to HIV infection, or which enables immune cells to effectively neutralize the virus via immune reconstruction, polymorphisms of genes encoding proteins expressed by immune cells, genes advantageous for fighting infection that are not expressed in the patient, genes encoding an infectious agent, receptor or coreceptor; a gene encoding ligands for receptors or coreceptors; viral and cellular genes essential for viral replication including; a gene encoding ribozymes, antisense RNA, small interfering RNA (siRNA) or decoy RNA to block the actions of certain transcription factors; a gene encoding dominant negative viral proteins, intracellular antibodies, intrakines and suicide genes. Exemplary therapeutic genes and gene products include α2β1; αvβ3; αvβ5; αvβ63; BOB/GPR15; Bonzo/STRL-33/TYMSTR; CCR2; CCR3; CCR5; CCR8; CD4; CD46; CD55; CXCR4; aminopeptidase-N; HHV-7; ICAM; ICAM-1; PRR2/HveB; HveA; α-dystroglycan; LDLR/α2MR/LRP; PVR; PRR1/HveC; and laminin receptor. A therapeutically effective amount for the treatment of HIV, for example, may increase the immunity of a subject against HIV, ameliorate a symptom associated with AIDS or HIV, or induce an innate or adaptive immune response in a subject against HIV. An immune response against HIV may include antibody production and result in the prevention of AIDS and/or ameliorate a symptom of AIDS or HIV infection of the subject, or decrease or eliminate HIV infectivity and/or virulence.

In particular embodiments, therapeutically effective amounts may provide function to immune and other blood cells, reduce or eliminate an immune-mediated condition; and/or reduce or eliminate a symptom of the immune-mediated condition.

In particular embodiments, particular methods of use include in the treatment of conditions where corrected cells have a selective advantage over non-corrected cells.

In particular embodiments, in vivo foamy gene delivery (with or without mobilization) can be combined with an in vivo selection marker. In particular embodiments, the in vivo selection marker can include MGMT P140K as described in Olszko M E et al. (2015) Gene Therapy 22: 591-595.

The drug resistant gene MGMT encoding human alkyl guanine transferase (hAGT) is a DNA repair protein that confers resistance to the cytotoxic effects of alkylating agents, such as nitrosoureas and temozolomide (TMZ). 6-benzylguanine (6-BG) is an inhibitor of AGT that potentiates nitrosourea toxicity and is co-administered with TMZ to potentiate the cytotoxic effects of this agent. Several mutant forms of MGMT that encode variants of AGT are highly resistant to inactivation by 6-BG, but retain their ability to repair DNA damage (Maze R et al. (1999) J. Pharmacol. Exp. Ther. 290: 1467-1474). P140KMGMT-based drug resistant gene therapy has been shown to confer chemoprotection to mouse, canine, rhesus macaques, and human cells, specifically hematopoetic cells (Zielske S P et al. (2003) J. Clin. Invest. 112: 1561-1570; Pollok K E et al. (2003) Hum. Gene Ther. 14: 1703-1714; Gerull S et al. (2007) Hum. Gene Ther. 18: 451-456; Neff T et al. (2005) Blood 105: 997-1002; Larochelle A et al. (2009) J. Clin. Invest. 119: 1952-1963; Sawai N et al. (2001) Mol. Ther. 3: 78-87).

In particular embodiments, combination with an in vivo selection marker will be a critical component for diseases without a selective advantage of gene-corrected cells. In SCID and some other immunodeficiencies and FA, corrected cells have an advantage and only transducing the therapeutic gene into a “few” HSPCs is sufficient for therapeutic efficacy. For other diseases like hemoglobinopathies (i.e., sickle cell disease and thalassemia) in which cells do not demonstrate a competitive advantage, in vivo selection of the gene corrected cells, such as in combination with an in vivo selection marker such as MGMT P140K, will select for the few transduced HSPCs, allowing an increase in the gene corrected cells and in order to achieve therapeutic efficacy. This approach can also be applied to HIV by making HSPCs resistant to HIV in vivo rather than ex vivo genetic modification.

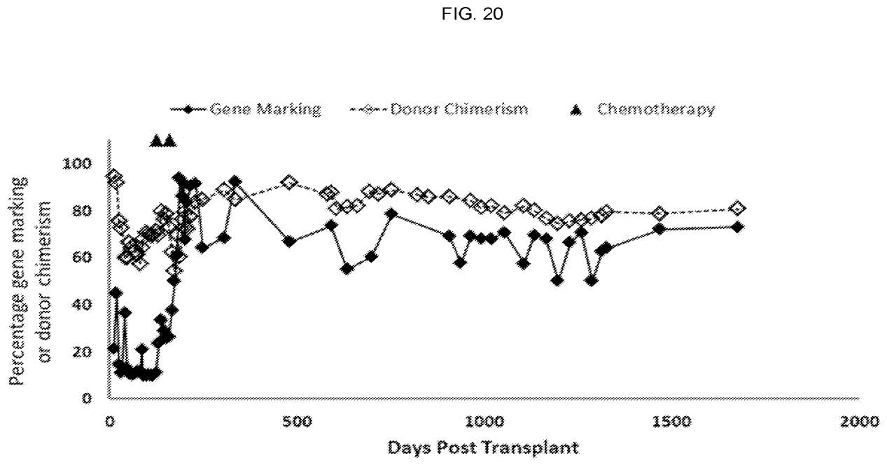

Supporting the discussion of the preceding paragraph, FIG. 20 shows selection of gene modified cells (as indicated by gene marking or chimerism) following 2 rounds of MGMT P140K based selection (indicated with triangles). This experiment was carried out in the canine model described elsewhere herein.

In the vectors, mobilization factors, formulations, and methods of use described herein, variants of protein and/or nucleic acid sequences can also be used. Variants include sequences with at least 70% sequence identity, 80% sequence identity, 85% sequence, 90% sequence identity, 95% sequence identity, 96% sequence identity, 97% sequence identity, 98% sequence identity, or 99% sequence identity to the protein and nucleic acid sequences described or disclosed herein wherein the variant exhibits substantially similar or improved biological function.

“% sequence identity” refers to a relationship between two or more sequences, as determined by comparing the sequences. In the art, “identity” also means the degree of sequence relatedness between protein and nucleic acid sequences as determined by the match between strings of such sequences. “Identity” (often referred to as “similarity”) can be readily calculated by known methods, including those described in: Computational Molecular Biology (Lesk, A. M., ed.) Oxford University Press, NY (1988); Biocomputing: Informatics and Genome Projects (Smith, D. W., ed.) Academic Press, NY (1994); Computer Analysis of Sequence Data, Part I (Griffin, A. M., and Griffin, H. G., eds.) Humana Press, NJ (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR, Inc., Madison, Wis.). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wis.); BLASTP, BLASTN, BLASTX (Altschul, et al., J. Mol. Biol. 215:403-410 (1990); DNASTAR (DNASTAR, Inc., Madison, Wis.); and the FASTA program incorporating the Smith-Waterman algorithm (Pearson, Comput. Methods Genome Res., [Proc. Int. Symp.] (1994), Meeting Date 1992, 111-20. Editor(s): Suhai, Sandor. Publisher: Plenum, New York, N.Y. Within the context of this disclosure it will be understood that where sequence analysis software is used for analysis, the results of the analysis are based on the “default values” of the program referenced. “Default values” will mean any set of values or parameters, which originally load with the software when first initialized.

(vii) Reference Levels Derived from Control Populations. Obtained values for parameters associated with in vivo gene therapy and/or HSPC mobilization described herein can be compared to a reference level derived from a control population, and this comparison can indicate whether an in vivo gene therapy described herein is effective for a subject in need thereof administered the gene therapy. Parameters associated with in vivo gene therapy and/or HSPC mobilization can include, for example: number of total white blood cells, neutrophils, monocytes, lymphocytes, and/or platelets; time required to reach normal lymphocyte counts; percent CD3+CD45RA+ T cells; number of TRECs per 10⁶ cells; percent of cells that are CD4+; percent of cells that are CD8+; the ratio of CD4/CD8; percent of TCRαβ+ cells in CD3+ T cells; diversity of TCR; frequency of gene corrected lymphocytes; diversity of clonal repertoire of gene corrected lymphocytes; number of unique retroviral integration site (RIS) clones; primary and secondary antibody responses to bacteriophage injection; rate of bacteriophage inactivation; percentage of cells that are gene corrected; level of immunoglobulins IgA, IgM, and/or IgG; resistance of bone marrow derived cells to mitomycin C; percent of living cells in methylcellulose and mitomycin C; functionality of γC-dependent signaling pathway; and percent phosphorylation of STAT3 with IL-21/mitogen stimulation of cells. Reference levels can be obtained from one or more relevant datasets from a control population. A “dataset” as used herein is a set of numerical values resulting from evaluation of a sample (or population of samples) under a desired condition. The values of the dataset can be obtained, for example, by experimentally obtaining measures from a sample and constructing a dataset from these measurements. As is understood by one of ordinary skill in the art, the reference level can be based on e.g., any mathematical or statistical formula useful and known in the art for arriving at a meaningful aggregate reference level from a collection of individual datapoints; e.g., mean, median, median of the mean, etc. Alternatively, a reference level or dataset to create a reference level can be obtained from a service provider such as a laboratory, or from a database or a server on which the dataset has been stored.

A reference level from a dataset can be derived from previous measures derived from a control population. A “control population” is any grouping of subjects or samples of like specified characteristics. The grouping could be according to, for example, clinical parameters, clinical assessments, therapeutic regimens, disease status, severity of condition, etc. In particular embodiments, the grouping is based on age range (e.g., 0-2 years) and non-immunocompromised status. In particular embodiments, a normal control population includes individuals that are age-matched to a test subject and non-immune compromised. In particular embodiments, age-matched includes, e.g., 0-6 months old; 0-1 year old; 0-2 years old; 0-3 years old; 10-15 years old, as is clinically relevant under the circumstances).

In particular embodiments, the relevant reference level for values of a particular parameter associated with in vivo gene therapy and/or HSPC mobilization described herein is obtained based on the value of a particular corresponding parameter associated with in vivo gene therapy and/or HSPC mobilization in a control population to determine whether an in vivo gene therapy disclosed herein has been therapeutically effective for a subject in need thereof administered the gene therapy.

In particular embodiments, a control population can include those that are healthy and do not have immune deficiencies. In particular embodiments, a control population can include those that have an immune deficiency and have not been administered a therapeutically effective amount of (i) a formulation including a foamy viral vector including a PGK promoter associated with a therapeutic gene; and (ii) mobilization factors. In particular embodiments, a control population can include those that have an immune deficiency and have been administered a therapeutically effective amount of a formulation including a foamy viral vector including a PGK promoter associated with a therapeutic gene and not including mobilization factors. As an example, the relevant reference level can be the value of the particular parameter associated with in vivo gene therapy and/or HSPC mobilization in the control subjects.

In particular embodiments, conclusions are drawn based on whether a sample value is statistically significantly different or not statistically significantly different from a reference level. A measure is not statistically significantly different if the difference is within a level that would be expected to occur based on chance alone. In contrast, a statistically significant difference or increase is one that is greater than what would be expected to occur by chance alone. Statistical significance or lack thereof can be determined by any of various methods well-known in the art. An example of a commonly used measure of statistical significance is the p-value. The p-value represents the probability of obtaining a given result equivalent to a particular datapoint, where the datapoint is the result of random chance alone. A result is often considered significant (not random chance) at a p-value less than or equal to 0.05. In particular embodiments, a sample value is “comparable to” a reference level derived from a normal control population if the sample value and the reference level are not statistically significantly different.

In particular embodiments, values obtained for parameters associated with in vivo gene therapy and/or HSPC mobilization described herein and/or other dataset components can be subjected to an analytic process with chosen parameters. The parameters of the analytic process may be those disclosed herein or those derived using the guidelines described herein. The analytic process used to generate a result may be any type of process capable of providing a result useful for classifying a sample, for example, comparison of the obtained value with a reference level, a linear algorithm, a quadratic algorithm, a decision tree algorithm, or a voting algorithm. The analytic process may set a threshold for determining the probability that a sample belongs to a given class. The probability preferably is at least 60%, at least 70%, at least 80%, at least 90%, at least 95% or higher.

Particular embodiments disclosed herein include obtaining a sample from a subject having an immune deficiency and administered a therapeutically effective amount of a formulation including a foamy viral vector including a PGK promoter associated with a therapeutic gene, but not including mobilization factors; assaying the sample to obtain one or more values of parameters associated with in vivo gene therapy described herein; comparing the one or more values of parameters associated with in vivo gene therapy described herein to a reference level; determining from the comparison whether an in vivo gene therapy disclosed herein was effective for the subject having an immune deficiency and administered the gene therapy.

Particular embodiments disclosed herein include obtaining a sample from a subject having an immune deficiency and administered a therapeutically effective amount of (i) a formulation including a foamy viral vector including a PGK promoter associated with a therapeutic gene; and (ii) mobilization factors; assaying the sample to obtain one or more values of parameters associated with in vivo gene therapy and/or cell mobilization described herein; comparing the one or more values of parameters associated with in vivo gene therapy and/or cell mobilization described herein to a reference level; determining from the comparison whether an in vivo gene therapy disclosed herein was effective for the subject having an immune deficiency and administered the gene therapy.

(viii) Kits. Combinations of formulations and mobilization factors disclosed herein that can be used to treat a subject in need thereof can also be provided as kits. Kits for treating a subject in need thereof can include: a formulation including a therapeutically effective amount of a foamy viral vector including a PGK promoter associated with a therapeutic gene; and a pharmaceutically acceptable carrier; and one or more mobilization factors. In particular embodiments, the foamy viral vector includes a SEQ ID NO. from FIG. 25. In particular embodiments, the foamy viral vector includes a sequence selected from SEQ ID NOs: 1-3, 6-10, 16-20, 26, 28-30, 32, and 33. In particular embodiments, the foamy viral vector includes a sequence encoding a sequence selected from SEQ ID NOs: 4, 5, 11-15, 21-25, and 27. In particular embodiments, the PGK promoter includes a human PGK promoter. In particular embodiments, the PGK promoter includes SEQ ID NO: 28. In particular embodiments, the therapeutic gene includes γC, JAK3, PNP, ADA, RAG1, and/or RAG2. In particular embodiments, the therapeutic gene includes γC. In particular embodiments, the therapeutic gene includes FancA, FancC, FancE, FancF, and/or FancG. In particular embodiments, the therapeutic gene includes FancA. In particular embodiments, the therapeutic gene includes a sequence selected from SEQ ID NOs: 1-3, 6-10, and 16-20. In particular embodiments, the therapeutic gene includes a sequence encoding a sequence selected from SEQ ID NOs: 4, 5, 11-15, and 21-25. In particular embodiments, the foamy viral vector further includes an in vivo selection marker. In particular embodiments, the in vivo selection marker is MGMT P140K. In particular embodiments, the one or more mobilization factors include G-CSF/Filgrastim (Amgen), GM-CSF, AMD3100 (Sigma), SCF, and/or a chemotherapeutic agent. In particular embodiments, the chemotherapeutic agent is selected from cyclophosphamide, etoposide, ifosfamide, cisplatin, and cytarabine. In particular embodiments, the one or more mobilization factors include G-CSF/Filgrastim (Amgen) and AMD3100 (Sigma). In particular embodiments, the one or more mobilization factors include SEQ ID NOs: 34-39.

Kits can also include a notice in the form prescribed by a governmental agency regulating the manufacture, use, or sale of pharmaceuticals or biological products, which notice reflects approval by the agency of manufacture, use, or sale for human administration. The notice may state that the provided active ingredients can be administered to a subject. The kits can include further instructions for using the kit, for example, instructions regarding administration of the formulation and/or mobilization factors. The instructions can be in the form of printed instructions provided within the kit or the instructions can be printed on a portion of the kit itself. Instructions may be in the form of a sheet, pamphlet, brochure, CD-Rom, or computer-readable device, or can provide directions to instructions at a remote location, such as a website. In particular embodiments, kits can also include some or all of the necessary medical supplies needed to use the kit effectively, such as syringes, ampules, tubing, facemask, an injection cap, sponges, sterile adhesive strips, Chloraprep, gloves, and the like. Variations in contents of any of the kits described herein can be made.

The Exemplary Embodiments and Examples below are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

### Exemplary Embodiments

1. A method of treating an immune deficiency in a subject in need thereof including administering a therapeutically effective amount of a formulation including a foamy viral vector including a PGK promoter associated with a therapeutic gene.
2. A method of embodiment 1 further including administering a therapeutically effective amount of one or more mobilization factors.
3. A method of embodiment 2, wherein the one or more mobilization factors include G-CSF/Filgrastim (Amgen), GM-CSF, AMD3100 (Sigma), SCF, and/or a chemotherapeutic agent.
4. A method of embodiment 3, wherein the chemotherapeutic agent is selected from cyclophosphamide, etoposide, ifosfamide, cisplatin, and cytarabine.
5. A method of any of embodiments 2-4, wherein the one or more mobilization factors include G-CSF/Filgrastim (Amgen) and AMD3100 (Sigma).
6. A method of any of embodiments 2-5, wherein the one or more mobilization factors include SEQ ID NOs: 34-39.
7. A method of any of embodiments 3-6, wherein G-CSF includes a sequence selected from SEQ ID NOs: 34-37.
8. A method of any of embodiments 1-7, wherein the formulation further includes an in vivo selection marker.
9. A method of embodiment 8, wherein the in vivo selection marker is MGMT P140K.
10. A method of any of embodiments 1-9, wherein the subject is in need thereof due to a primary or secondary immune deficiency.
11. A method of any of embodiments 1-10, wherein the subject is in need thereof due to severe combined immunodeficiency (SCID).
12. A method of embodiment 11, wherein SCID is SCID-X1.
13. A method of any of embodiments 1-12, wherein the therapeutic gene encodes common gamma chain (γC) protein.
14. A method of embodiment 13, wherein the γC protein restores functionality to a γC-dependent signaling pathway.
15. A method of embodiment 14, wherein the functionality of a γC-dependent signaling pathway is determined by measuring tyrosine phosphorylation of STAT3 and/or STAT5 in cells from the subject following in vitro stimulation with IL-21 and/or IL-2, respectively.
16. A method of embodiment 15, wherein the tyrosine phosphorylation of STAT3 and/or STAT5 is measured by intracellular antibody staining.
17. A method of embodiment 15 or 16, wherein the cells are peripheral blood mononuclear cells (PBMCs).
18. A method of any of embodiments 1-10, wherein the subject is in need thereof due to Fanconi anemia (FA).
19. A method of any of embodiments 1-10 and 18, wherein the therapeutic gene encodes FancA.
20. A method of embodiment 19, wherein the FancA protein increases resistance of bone marrow derived cells to mitomycin C (MMC).
21. A method of embodiment 20, wherein the resistance of bone marrow derived cells to MMC is measured by a cell survival assay in methylcellulose and MMC.
22. A method of any of embodiments 1-21, wherein the therapeutically effective amount of the foamy viral vector is 1×10⁸ to 10×10⁸ infection units (IU).
23. A method of any of embodiments 1-22, wherein the therapeutically effective amount of the foamy viral vector is 4×10⁸ IU.
24. A method of any of embodiments 3-23, wherein the therapeutically effective amount of G-CSF is 1 μg/kg to 10 μg/kg.
25. A method of any of embodiments 3-24, wherein the therapeutically effective amount of G-CSF is 5 μg/kg.
26. A method of any of embodiments 3-23, wherein the therapeutically effective amount of G-CSF is 1 μg to 10 μg.
27. A method of any of embodiments 3-23 and 26, wherein the therapeutically effective amount of G-CSF is 3 μg.
28. A method of any of embodiments 3-27, wherein the therapeutically effective amount of AMD3100 is 1 mg/kg to 10 mg/kg.
29. A method of any of embodiments 3-28, wherein the therapeutically effective amount of AMD3100 is 4 mg/kg.
30. A method of any of embodiments 3-28, wherein the therapeutically effective amount of AMD3100 is 5 mg/kg.
31. A method of any of embodiments 3-30, wherein the therapeutically effective amount of G-CSF is administered for 4 consecutive days prior to administration of the therapeutically effective amount of the formulation including the foamy viral vector.
32. A method of any of embodiments 3-31 including a 5 day treatment protocol, wherein G-CSF is administered on day 1, day 2, day 3, and day 4 and on day 5, G-CSF and AMD3100 are administered 6 to 8 hours prior to administration of the foamy viral vector.
33. A method of any of embodiments 3-31, wherein G-CSF is administered every 12 hours for 4 consecutive days, followed by administration of AMD3100 14 hours after the last dose of G-CSF and 1 hour prior to collection of peripheral blood.
34. A method of any of embodiments 3-33, wherein the therapeutically effective amount of AMD3100 is administered following the administration of the therapeutically effective amount of G-CSF.
35. A method of any of embodiments 3-32 and 34, wherein the therapeutically effective amount of AMD3100 is administered concurrently with the therapeutically effective amount of G-CSF.
36. A method of any of embodiments 3-35, wherein the therapeutically effective amount of AMD3100 is administered prior to the administration of the therapeutically effective amount of the formulation including the foamy viral vector.
37. A method of any of embodiments 2-36, wherein one or more mobilization factors is part of the formulation including the foamy viral vector.
38. A method of any of embodiments 1-37, wherein the foamy viral vector includes a sequence selected from SEQ ID NOs: 1-3, 6-10, 16-20, 26, 28-30, 32, and/or 33.
39. A method of any of embodiments 1-38, wherein the foamy viral vector includes a sequence encoding a sequence selected from SEQ ID NOs: 4, 5, 11-15, 21-25, and/or 27.
40. A method of any of embodiments 1-39, wherein the administration of the formulation and/or one or more mobilization factors are by subcutaneous delivery, intravenous delivery, and/or intra bone marrow delivery.
41. A method of any of embodiments 1-40, wherein the administration targets mesenchymal stem cells.
42. A method of any of embodiments 1-41, wherein the method restores T-cell mediated immune responses in the subject in need thereof.
43. A method of embodiment 42, wherein restoring T-cell mediated immune responses in the subject in need thereof includes increasing thymic output.
44. A method of embodiment 43, wherein increasing thymic output includes increasing the frequency of CD3+ T cells expressing CD45RA in peripheral blood to a level comparable to that of a reference level derived from a normal control population.
45. A method of embodiment 43, wherein increasing thymic output includes increasing the frequency of CD3+ T cells expressing CD45RA in peripheral blood to a level greater than that of a subject in need thereof not administered the therapeutically effective amount of the formulation.
46. A method of embodiment 43, wherein increasing thymic output includes increasing the frequency of CD3+ T cells expressing CD45RA in peripheral blood to a level greater than that of a subject in need thereof administered the therapeutically effective amount of the formulation and not including the one or more mobilization factors.
47. A method of embodiment 43 or 44, wherein increasing thymic output includes increasing the number of T cell receptor excision circles (TRECs) per 10⁶ maturing T cells to a level comparable to that of a reference level derived from a normal control population.
48. A method of embodiment 43 or 45, wherein increasing thymic output includes increasing the number of T cell receptor excision circles (TRECs) per 10⁶ maturing T cells to a level greater than that of a subject in need thereof not administered the therapeutically effective amount of the formulation.
49. A method of embodiment 43 or 46, wherein increasing thymic output includes increasing the number of T cell receptor excision circles (TRECs) per 10⁶ maturing T cells to a level greater than that of a subject in need thereof administered the therapeutically effective amount of the formulation and not including the one or more mobilization factors.
50. A method of any of embodiments 42-49, wherein restoring T-cell mediated immune responses in a subject in need thereof includes restoring normal T lymphocyte development.
51. A method of embodiment 50, wherein restoring normal T lymphocyte development includes restoring the ratio of CD4+ cells: CD8+ cells to 2.
52. A method of embodiment 50 or 51, wherein restoring normal T lymphocyte development includes detecting the presence of αβ TCR in circulating T-lymphocytes.
53. A method of embodiment 52, wherein detecting the presence of αβ TCR in circulating T-lymphocytes includes detecting the αβ TCR by flow cytometry.
54. A method of any of embodiments 50-53, wherein restoring normal T lymphocyte development includes detecting the presence of a diverse TCR repertoire comparable to that of a reference level derived from a normal control population.
55. A method of embodiment 54, wherein detecting the presence of a diverse TCR repertoire includes spectratyping TCRVβ.
56. A method of any of embodiments 50-55, wherein restoring normal T lymphocyte development includes restoring one or more T-cell specific signaling pathways.
57. A method of embodiment 56, wherein restoring one or more T-cell specific signaling pathways can be assessed by lymphocyte proliferation following exposure to T cell mitogen phytohemagglutinin (PHA).
58. A method of any of embodiments 50-57, wherein restoring normal T lymphocyte development includes increasing white blood cell count, neutrophil cell count, monocyte cell count, lymphocyte cell count, and/or platelet cell count to a level comparable to that of a reference level derived from a normal control population.
59. A method of any of embodiments 50-57, wherein restoring normal T lymphocyte development includes increasing white blood cell count, neutrophil cell count, monocyte cell count, lymphocyte cell count, and/or platelet cell count to a level greater than that of a subject in need thereof not administered the therapeutically effective amount of the formulation.
60. A method of any of embodiments 50-57, wherein restoring normal T lymphocyte development includes increasing white blood cell count, neutrophil cell count, monocyte cell count, lymphocyte cell count, and/or platelet cell count to a level greater than that of a subject in need thereof administered the therapeutically effective amount of the formulation and not including the one or more mobilization factors.
61. A method of any of embodiments 1-60, wherein the method improves the kinetics and clonal diversity of lymphocyte reconstitution in the subject in need thereof.
62. A method of embodiment 61, wherein improving the kinetics of lymphocyte reconstitution includes increasing the number of circulating T lymphocytes to within a range of a reference level derived from a normal control population.
63. A method of embodiment 61, wherein improving the kinetics of lymphocyte reconstitution includes increasing the number of circulating T lymphocytes as compared to that of a subject in need thereof not administered the therapeutically effective amount of the formulation.
64. A method of embodiment 61, wherein improving the kinetics of lymphocyte reconstitution includes increasing the number of circulating T lymphocytes as compared to that of a subject in need thereof administered the therapeutically effective amount of the formulation and not including the one or more mobilization factors.
65. A method of embodiment 61 or 63, wherein improving the kinetics of lymphocyte reconstitution includes reducing the time required to reach normal lymphocyte counts as compared to that of a subject in need thereof not administered the therapeutically effective amount of the formulation.
66. A method of embodiment 61 or 64, wherein improving the kinetics of lymphocyte reconstitution includes reducing the time required to reach normal lymphocyte counts as compared to that of a subject in need thereof administered the therapeutically effective amount of the formulation and not including the one or more mobilization factors.
67. A method of embodiment 61 or 62, wherein improving the kinetics of lymphocyte reconstitution includes increasing the absolute CD3+ lymphocyte count to within a range of a reference level derived from a normal control population.
68. A method of any of embodiments 61, 63, or 65, wherein improving the kinetics of lymphocyte reconstitution includes increasing the absolute CD3+ lymphocyte count as compared to that of a subject in need thereof not administered the therapeutically effective amount of the formulation.
69. A method of any of embodiments 61, 64, or 66, wherein improving the kinetics of lymphocyte reconstitution includes increasing the absolute CD3+ lymphocyte count as compared to that of a subject in need thereof administered the therapeutically effective amount of the formulation and not including the one or more mobilization factors.
70. A method of any of embodiments 61, 66, 65, or 68, wherein improving the kinetics of lymphocyte reconstitution includes increasing the frequency of gene corrected lymphocytes as compared to a subject in need thereof not administered the therapeutically effective amount of the formulation.
71. A method of any of embodiments 61, 64, 66, or 69, wherein improving the kinetics of lymphocyte reconstitution includes increasing the frequency of gene corrected lymphocytes as compared to a subject in need thereof administered the therapeutically effective amount of the formulation and not including the one or more mobilization factors.
72. A method of any of embodiments 61, 63, 65, 68, or 70, wherein improving the kinetics of lymphocyte reconstitution includes increasing diversity of clonal repertoire of gene corrected lymphocytes in the subject as compared to a subject in need thereof not administered the therapeutically effective amount of the formulation.
73. A method of any of embodiments 61, 64, 66, 69, or 71, wherein improving the kinetics of lymphocyte reconstitution includes increasing diversity of clonal repertoire of gene corrected lymphocytes in the subject as compared to a subject in need thereof administered the therapeutically effective amount of the formulation and not including the one or more mobilization factors.
74. A method of embodiment 72 or 73, wherein increasing diversity of clonal repertoire of gene corrected lymphocytes includes increasing the number of unique retroviral integration site (RIS) clones as measured by a RIS analysis.
75. A method of any of embodiments 1-74, wherein the method restores bone marrow function in the subject in need thereof.
76. A method of embodiment 75, wherein restoring bone marrow function includes improving bone marrow repopulation with gene corrected cells in the subject as compared to a subject in need thereof not administered the therapeutically effective amount of the formulation.
77. A method of embodiment 75, wherein restoring bone marrow function includes improving bone marrow repopulation with gene corrected cells in the subject as compared to a subject in need thereof administered the therapeutically effective amount of the formulation and not including the one or more mobilization factors.
78. A method of embodiment 76 or 77, wherein improving bone marrow repopulation with gene corrected cells includes increasing the percentage of cells that are gene corrected.
79. A method of any of embodiments 76-78, wherein the cells are selected from white blood cells and bone marrow derived cells.
80. A method of embodiment 78 or 79, wherein the percentage of cells that are gene corrected is measured using an assay selected from quantitative real time PCR and/or flow cytometry.
81. A method of any of embodiments 1-80, wherein the method normalizes primary and secondary antibody responses to immunization in the subject in need thereof.
82. A method of embodiment 81, wherein normalizing primary and secondary antibody responses to immunization in the subject in need thereof includes restoring B-cell and/or T-cell cytokine signaling programs functioning in class switching and memory response to an antigen.
83. A method of embodiment 82, wherein restoring B-cell and/or T-cell cytokine signaling programs is measured by a bacteriophage immunization assay.
84. A method of any of embodiments 81-83, wherein normalizing primary and secondary antibody responses to immunization in a subject in need thereof includes increasing the level of one or more immunoglobulins selected from IgA, IgM, and IgG in a subject in need thereof to a level comparable to that of corresponding immunoglobulins in a reference level derived from a normal control population.
85. A method of any of embodiments 81-83, wherein normalizing primary and secondary antibody responses to immunization in a subject in need thereof includes increasing the level of one or more immunoglobulins selected from IgA, IgM, and IgG in a subject in need thereof to a level greater than that of corresponding immunoglobulins in a subject in need thereof not administered the therapeutically effective amount of the formulation.
86. A method of any of embodiments 81-83, wherein normalizing primary and secondary antibody responses to immunization in a subject in need thereof includes increasing the level of one or more immunoglobulins selected from IgA, IgM, and IgG in a subject in need thereof to a level greater than that of corresponding immunoglobulins in a subject in need thereof administered the therapeutically effective amount of the formulation and not including the one or more mobilization factors.
87. A method of any of embodiments 84-86, wherein the increase in the level of one or more immunoglobulins selected from IgA, IgM, and IgG in a subject in need thereof is measured by an assay selected from serum protein electrophoresis, immunoelectrophoresis, radial immunodiffusion, nephelometry and/or turbidimetry.
88. A formulation including: a therapeutically effective amount of a foamy viral vector including a PGK promoter associated with a therapeutic gene; and a pharmaceutically acceptable carrier.
89. The formulation of embodiment 88, wherein the foamy viral vector includes a sequence selected from SEQ ID NOs: 1-3, 6-10, 16-20, 26, 28-30, 32, and/or 33.
90. The formulation of embodiment 88 or 89, wherein the foamy viral vector includes a sequence encoding a sequence selected from SEQ ID NOs: 4, 5, 11-15, 21-25, and/or 27.
91. The formulation of any of embodiments 88-90, wherein the PGK promoter includes a human PGK promoter.
92. The formulation of any of embodiments 88-91, wherein the PGK promoter includes SEQ ID NO: 28.
93. The formulation of any of embodiments 88-92, wherein the therapeutic gene includes γC.
94. The formulation of any of embodiments 88-93, wherein the therapeutic gene includes FancA.
95. The formulation of any of embodiments 88-94, wherein the therapeutic gene includes a sequence selected from SEQ ID NOs: 1-3, 6-10, and/or 16-20.
96. The formulation of any of embodiments 88-95, wherein the therapeutic gene includes a sequence encoding a sequence selected from SEQ ID NOs: 4, 5, 11-15, and/or 21-25.
97. The formulation of any of embodiments 88-96, wherein the foamy viral vector further includes an in vivo selection marker.
98. The formulation of embodiment 97, wherein the in vivo selection marker is MGMT P140K.
99. The formulation of any of embodiments 88-98 further including one or more mobilization factors.
100. The formulation of embodiment 99, wherein the one or more mobilization factors include G-CSF/Filgrastim (Amgen), GM-CSF, AMD3100 (Sigma), SCF, and/or a chemotherapeutic agent.
101. The formulation of embodiment 100, wherein the chemotherapeutic agent is selected from cyclophosphamide, etoposide, ifosfamide, cisplatin, and cytarabine.
102. The formulation of any of embodiments 99-101, wherein the one or more mobilization factors include G-CSF/Filgrastim (Amgen) and AMD3100 (Sigma).
103. The formulation of any of embodiments 99-102, wherein the one or more mobilization factors include SEQ ID NOs: 34-39.
104. A kit including:
   a formulation including a therapeutically effective amount of a foamy viral vector including a PGK promoter associated with a therapeutic gene; and a pharmaceutically acceptable carrier; and one or more mobilization factors.
105. The kit of embodiment 104, wherein the foamy viral vector includes a sequence selected from SEQ ID NOs: 1-3, 6-10, 16-20, 26, 28-30, 32, and/or 33.
106. The kit of embodiment 104 or 105, wherein the foamy viral vector includes a sequence encoding a sequence selected from SEQ ID NOs: 4, 5, 11-15, 21-25, and/or 27.
107. The kit of any of embodiments 104-106, wherein the PGK promoter includes a human PGK promoter.
108. The kit of any of embodiments 104-107, wherein the PGK promoter includes SEQ ID NO: 28.
109. The kit of any of embodiments 104-108, wherein the therapeutic gene includes γC.
110. The kit of any of embodiments 104-109, wherein the therapeutic gene includes FancA.
111. The kit of any of embodiments 104-110, wherein the therapeutic gene includes a sequence selected from SEQ ID NOs: 1-3, 6-10, and/or 16-20.
112. The kit of any of embodiments 104-111, wherein the therapeutic gene includes a sequence encoding a sequence selected from SEQ ID NOs: 4, 5, 11-15, and/or 21-25.
113. The kit of any of embodiments 104-112, wherein the foamy viral vector further includes an in vivo selection marker.
114. The kit of embodiment 113, wherein the in vivo selection marker is MGMT P140K.
115. The kit of any of embodiments 104-114, wherein the one or more mobilization factors include G-CSF/Filgrastim (Amgen), GM-CSF, AMD3100 (Sigma), SCF, and/or a chemotherapeutic agent.
116. The kit of embodiment 115, wherein the chemotherapeutic agent is selected from cyclophosphamide, etoposide, ifosfamide, cisplatin, and cytarabine.
117. The kit of any of embodiments 104-116, wherein the one or more mobilization factors include G-CSF/Filgrastim (Amgen) and AMD3100 (Sigma).
118. The kit of any of embodiments 104-117, wherein the one or more mobilization factors include SEQ ID NOs: 34-39.
119. The kit of any of embodiments 104-118 further including instructions on administering the formulation and/or the one or more mobilization factors.

### EXAMPLE 1

HSPC gene therapy is a promising treatment for X-linked severe combined immunodeficiency disease (SCID-X1), but currently requires recipient conditioning, extensive cell manipulation and sophisticated facilities. With these limitations in mind, a simpler therapeutic approach for SCID-X1 by direct intravenous administration of foamy virus (FV) vectors in the canine model was explored in this Example. FV vectors were used because they have a favorable integration site profile and are resistant to serum inactivation. Improved efficacy was shown for an in vivo gene therapy platform using mobilization with G-CSF and AMD3100 prior to injection of a FV vector incorporating the human phosphoglycerate kinase enhancer-less promoter (FV.PGK.γC). FV vector delivery into mobilized canines accelerated kinetics of CD3+ lymphocyte recovery, promoted thymopoiesis, and increased immune clonal diversity. Gene-corrected T-lymphocytes exhibited a normal CD4/CD8 ratio, a broad T-cell receptor repertoire, and showed restored γC-dependent signaling function. Treated animals showed normal primary and secondary antibody responses to bacteriophage immunization and evidence for immunoglobulin class switching. These results demonstrate safety and efficacy for an accessible, portable, and translatable platform with no conditioning regimen for the treatment of SCID-X1 and other genetic diseases.

Materials and Methods. Animal Care and breeding strategy. All experiments were performed in accordance with protocols approved by the University of Pennsylvania and Fred Hutchinson Cancer Research Center Institutional Animal Care and Use Committees (IACUC). The SCID-X1 dogs used in this study contain a 4 base-pair null mutation on exon 1 of the IL2RG gene that results in premature termination of the protein. Progenies were derived from the breeding of SCID-X1 affected male with hemizygous affected females. SCID-X1 pups were identified by flow cytometry with the absence of CD3+ cells in peripheral blood and by genotyping analysis. All SCID-X1 affected dogs were housed in a HEPA-filtered facility at the Fred Hutchinson Cancer Research Center. R2258 and R2260 were born to SCID-X1 dogs cured with bone marrow transplant or in vivo FV gene therapy. H864 and H867 were sired by mating in vivo FV gene therapy cured SCID-X1 affected R2260 male with heterozygous SCID-X1 carrier female.

Mobilization regimen. SCID-X1 affected neonatal pups at 1 kg (3 weeks old) were injected subcutaneously with 5 μg/kg cG-CSF BID SQ for 4 days and a last single dose of cG-CSF (5 μg/kg, SQ) with AMD3100 (4 mg/kg SQ) 6-8 hr prior on the day of FV vector injection for mobilization of HSPCs, the dose regimen is as previously published by Thakar M S et al. (2010) Blood 115: 916-917. 0.5 ml of peripheral blood was collected 6 hours after AMD3100 administration and immediately prior to FV vector injection to measure CD34+ cells frequency in peripheral blood by staining with anti-canine CD34 monoclonal antibody (clone 1H6, Serotec, Raleigh, N.C.).

Foamy virus vectors. FV vectors were produced by polyethylenimine transfection of four plasmids in HEK293T cells as previously described (Kiem H P et al. (2010) Gene Therapy 17: 37-49), with the exception that 37.7 μg of transfer plasmid and 10.8, 16.1 and 0.8 μg of FV helper plasmids pFVGagCO, pFVPolCO, pFVEnvCO and 198.6 μl of 1 μg/μl polyethylenimine were used per 15-cm plate. The FV helper plasmids were codon-optimized to improve expression and to eliminate the potential for recombination. Vector-containing supernatant was passed through a 0.45-μm filter, concentrated 100-fold by ultracentrifugation at 23° C., and frozen at −80 ° C. until use in Iscove's Modified Dulbecco's Medium (IMDM) media containing 5% DMSO. Vector preparations were titered on HT1080 cells and cells expressing the fluorescent reporters were quantified by flow cytometry at 3 days post-transduction.

Foamy virus vector construction. The FV vector constructs used in this Example have been previously described in Burtner C R et al. (2014) Blood 123: 3578-3584. Briefly, the transfer vector includes a U3-deleted long terminal repeat and a 2.3 Kb FV cis acting region containing the 3′ region of pol and 5′ region of env required for efficient gene transfer, with introduced stop codons in the foamy partial gag sequence. The vector was made self-inactivating by deletion of the Tas (Bel-1) transactivator, which is required for transcription from the LTR. The transgene is expressed from the intron-less human elongation factor 1α (EF1α) or from the human phosphoglycerate kinase (PGK) promoters. EGFP and a codon-optimized human common gamma chain receptor (γC) are separated by the Thosea asigna T2A peptide (2A). A safety-modified woodchuck post-transcriptional regulatory element (WPRE) contains the X protein promoter, with four mutated ATG sequences as previously described (Schambach A. et al. (2006) Gene Therapy 13: 641-645). To generate EF1α.mCherry.2A.γC.FV and PGK.mCherry.2A.γC.FV, the EF1α.GFP.2A.γC and PGK.GFP.2A.γC DNA inserts were first extracted by digestion with enzymes Bglll/Notl, and ligation into BamHl/Notl of plasmid bluescript SK+ to generate pSK.EF1α.GFP.2A.γC and pSK.PGK.GFP.2A.γC. To construct pSK.PGK.mCherry.2A.γC, the mCherry sequence was PCR amplified using High Fidelity Platinum Taq (Life Technologies) using forward primer 5′-GATCCACCGGTCGCCACCATG-3′ (SEQ ID NO: 40) and reverse primer 5′-GTCGACGCGGCCGCTTTACTTG-3′, (SEQ ID NO: 41) digested with Agel/BsrGl, and ligated into pSK.PGK.GFP.2A.γC cut with the same enzymes. pSK.EF1α.mCherry.2A.γC was constructed by amplification of the EF1α.mCherry sequence from a reference plasmid with forward primer 5′-ACTGCATGCCGATGGCTCCGGTGCCCGTC-3′ (SEQ ID NO: 42) and reverse primer 5′-GTCGACGCGGCCGCTTTACTTG-3′, (SEQ ID NO: 41) digestion with Sphl/BsrGl and ligation into pSK.EF1α.GFP.2A.γC cut with the same enzymes. PGK.mCherry.2A.γC.FV was constructed by ligation of the Agel/BamHl fragment from pSK.PGK.mCherry.2A.γC into PGK.GFP.2A.γC.FV. EF1α.mCherry.2A.γC.FV was constructed by ligation of the Sphl/Notl fragment from pSK.EF1α.mCherry.2A.γC.FV into PGK.GFP.2A.γC.FV. The PGK- and EF1α-FV constructs overall produced comparable vector titers.

Ex vivo transduction of canine and human CD34+ cells. Human CD34+ cells were collected from volunteers under institutional review board-approved protocol. Human and canine CD34+ cells were isolated, cultured, and transduced with FV vectors as described previously in Kiem HP et al. (2007) Blood 109: 65-70. Briefly, CD34+ cells were cultured overnight in IMDM containing 10% FBS and 100 ng/μL of the respective cytokines (FLT3, SCF, TPO for human; FLT3, TPO, cSCF, cG-CSF for canine), and transduced on CH296 fibronectin (Takara, New York, N.Y.) at 2 μg/mL using MOIs determined by HT1080 titers. Transduction efficiency was evaluated based on fluorophore expression measured by flow cytometry analysis.

Determination of in vivo gene marking and phenotypic analysis. Gene marking and phenotype analysis in peripheral blood leukocytes were determined by flow cytometry using antibodies described in Burtner C R et al. (2014) Blood 123: 3578-3584. Blood was collected in EDTA or heparin tubes, subjected to hemolysis, and washed in phosphate-buffered saline plus 2% fetal bovine serum. Flow cytometry analysis was performed on either a FACSCalibur or FACSCanto flow cytometer (Becton Dickinson, San Jose, Calif.) to measure fluorescent gene marking or fluorescent antibody cell surface receptor phenotyping.

Foamy Retroviral integration site (RIS) analysis. RIS analysis was performed as previously described (Adair J E et al. (2012) Science Translational Medicine 4(133): 133ra157; Adair J E et al. (2014) J Clin Invest 124(9): 4082-4092). Genomic DNA (gDNA) was extracted from leukocytes collected at various time points from either PB or BM, or from the tissues harvested at necropsy by Qiagen Blood DNA Mini Kit or Gentra Puregene Blood kit (both from QIAGEN), per manufacturer's instructions. FV vector LTR-genome junctions were amplified by modified genomic sequencing (MGS)-PCR as described in Burtner C R et al. (2014) Blood 123: 3578-3584. Resulting sequence libraries were subjected to paired end Illumina MiSeq platform sequencing. RISs were identified using a bioinformatics method as described in detail previously. Valid integration sites were scored after locating primer sequence, foamy virus LTR, absence of foamy virus vector sequence, and potential canine genomic DNA. Potential genomic sequences were mapped to the canine genome (canFam3) using a stand-alone version of BLAT available from the UCSC Genome Browser. Sequences corresponding to the same genomic locus were grouped together to determine the total number of unique RIS events (clones) identified in the sample. Contributions of each clone were normalized by dividing the number of integration site-associated sequence reads corresponding to that clone by the total number of integration site-associated sequence reads from the same sample. A custom R script was used to generate contribution graphs. Additional details on bioinformatic analysis of data is given below.

TCR Spectratyping and TREC analysis. For spectratyping analysis, peripheral blood was hemolysed and RNA was extracted from 5×10⁶ white blood cells using the RNeasy Mini Kit (Cat#74104; Qiagen, Valencia, Calif.). cDNA was generated from 100-400 ng RNA using 200 U of SuperScript 11 Reverse Transcriptase (Cat#18064-022; Invitrogen, Grand Island, N.Y.) and oligo dT, following the manufacturer's instructions. cDNA was amplified using 17 specific forward TCRVβ primers and a common 6-FAM-conjugated reverse primer, as previously published in Vernau W et al. (2007) Biology of Blood & Marrow Transplantation 13(9): 1005-1015. The products were analyzed on an Applied Biosystems ABI 3730xl DNA Analyzer, and GeneMapper Software v4.0 was used for the analysis of peak sizes (Life Technologies, Grand Island, N.Y.). For TREC analysis, peripheral blood was lysed and DNA was extracted from 5×10⁶ cells using the Qiagen QIAamp DNA Blood Mini Kit (cat # 51106). A real-time quantitative PCR method was used as previously described to detect signal joint TRECs (Kennedy D R et al. (2011) Vet Immunol Immunopathol 142: 36-48).

In vitro T-lymphocyte functional assay. In the mitogen-induced proliferation assay, peripheral blood mononuclear cells (PBMCs) were isolated by ficoll centrifugation and 1-2×10⁶ cells were stimulated with 5 μg/mL phytohemagglutinin (PHA) (Sigma, St. Louis, Mo.) for 48 hours in complete medium at 37° C. and 5% CO₂. Cell proliferation was assessed using flow cytometric CellTracker™ dye assay (Thermo Fisher Scientific, Waltham, Mass.) as per manufacturer's instruction. For pSTAT5 and pSTAT3 analysis, PBMCs were incubated for 4-6 hours at 37° C. and 5% CO₂ in complete medium (RPMI with 10% fetal calf serum, 1% L-glutamine and 0.5% Pen/Strep), after which they were stimulated with IL-2 or IL-21 for 20-25 minutes as described previously (Burtner C R et al. (2014) Blood 123: 3578-3584). pSTAT3 and pSTAT5 phosphorylation were subsequently monitored by intracellular staining with pSTAT3 antibody (BD Phosflow cat# 557815) and pSTAT5 y694 antibody (BD Phosflow cat# 612599), respectively, and analyzed by flow cytometry.

Bacteriophage immunization assay. Generation of specific antibody responses and immunoglobulin class-switching was assessed after immunization with the T cell dependent neoantigen bacteriophage φX174. This bacteriophage does not replicate or cause illness in human subjects and induced helper T cell-dependent antibody response when used as immunogen (Ochs H D et al. (1971) J. Clin Invest 50: 2559-2568). Mobilized (H864 and H867) and non-mobilized (R2258 and R2260) animals were injected with a first dose of bacteriophage φX174 at 8-12 months post FV vector treatment and with a second dose 6 weeks later, and immune response was assessed at 1, 2 and 4 weeks post injection. Total φX-174-specific antibody in each plasma sample was determined by using a standardized phage neutralization assay (Wedgwood R J et al. Immunodeficiency in man and animals. in The Recognition and Classification of Immunodeficiency Diseases with Bacteriophage ΦX174. March of Dimes Birth Defects Original Article Series X1 (ed. Bergsma, D.) 331-338 (Sinaur, Sunderland, Mass., 1975)) and was expressed as the rate of phage inactivation or K value (Kv) as derived from a standard formula. Specific antibody levels were plotted as log Kv against time. Total antibody production (pan IgG, IgA, IgM) was quantitatively measured (Phoenix Laboratories, Seattle, Test Code:SO633) from serum collected from FV treated animals.

Statistics. To assess differences in kinetics of gene marking and CD3+ lymphocyte reconstitution for animals treated with EF1α.γC.FV (n=5) or PGK.γC.FV (n=4 or 5) vectors, a line of best fit (estimated with a zero intercept) was estimated for each dog, including EF1α. γC.FV-treated dogs not shown in this Example, using data ranging from day 0 to day 60 post treatment. The mean slope was calculated for each experimental group and means were compared via a two-sample t-test, using Welch's approximation to estimate the degrees of freedom. P-values were two-sided and values less than 0.05 were considered to indicate statistical significance.

Transduction filter methods. Collisions, integration site (IS) appearing in distinct samples originating from unique transduction events, were present in the data. Theoretically, this should never be observed and is likely the result of contamination, barcode swapping or other errors in processing. In some cases, it was possible to determine which sample the IS originated from by comparing the number of genomically aligned sequence reads representing the IS in each sample. When examining collisions, the genomically aligned sequence counts were used instead of normalized frequencies to avoid biases introduced by low capture frequency in samples with few genomically aligned reads, because the log base ten-fold difference between the most (129,408) and fewest (108) genomically aligned reads across samples was large (3.08).

Using a custom python script, a list of all collisions was generated. Each transduction event was parsed for observations of ISs in the collision list. For each transduction event in which a collision IS was detected, the mean count of the IS for samples in which it was detected was recorded. For example, an IS at chr10:630,220 was observed in two transduction events. In the first transduction event, it was observed in two samples where it was represented by 100 and 197 genomically aligned sequence reads. It was observed in one sample from another transduction event where it was represented by 23 genomically aligned reads. The mean count of the IS in the first transduction event was 148.5 and 23 for the other transduction event.

The ratio of mean counts from each transduction event was compared to the maximum mean count from a single transduction event. If a transduction event had a mean count greater than or equal to one half of the maximum mean count for the IS, the IS was discarded from the dataset; otherwise, the IS was kept for the transduction event in which it had the highest count and removed from the other samples. In other words, if the ratio of the maximum mean count to the next highest mean count was greater than 1:2 (½ or 0.5), the IS was discarded. If the ratio was less than 1:2, the IS was retained in the transduction event where it had the highest count and removed from all others. Returning to the previous example, 23:148.5 is 0.154. In this case, the non-maximum genomically aligned read count fell below 0.5 and the IS was retained in the first transduction event dataset and removed from the other transduction event dataset. Overall, from an initial set of 12,624 unique IS, 965 collisions (7.6%) were detected and 60 (0.5%) were unresolvable (removed from all datasets).

Results. 1) Improved gene marking and lymphocyte reconstitution in vivo using vector PGK.γC.FV. The initial in vivo delivery study employed a FV vector construct containing the short elongation factor 1 alpha promoter (EF1α) driving expression of the human codon optimized common gamma chain (γC) gene (EF1α.γC.FV) (Burtner C R et al. (2014) Blood 123: 3578-3584). Animals treated by intravenous injection using this FV vector showed expansion of gene-marked lymphocytes, but the overall kinetics of T cell reconstitution was slower than that seen for human ex vivo gene therapy and the animals eventually developed chronic infections.

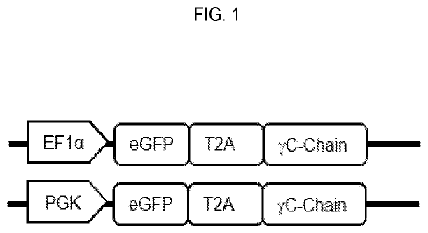

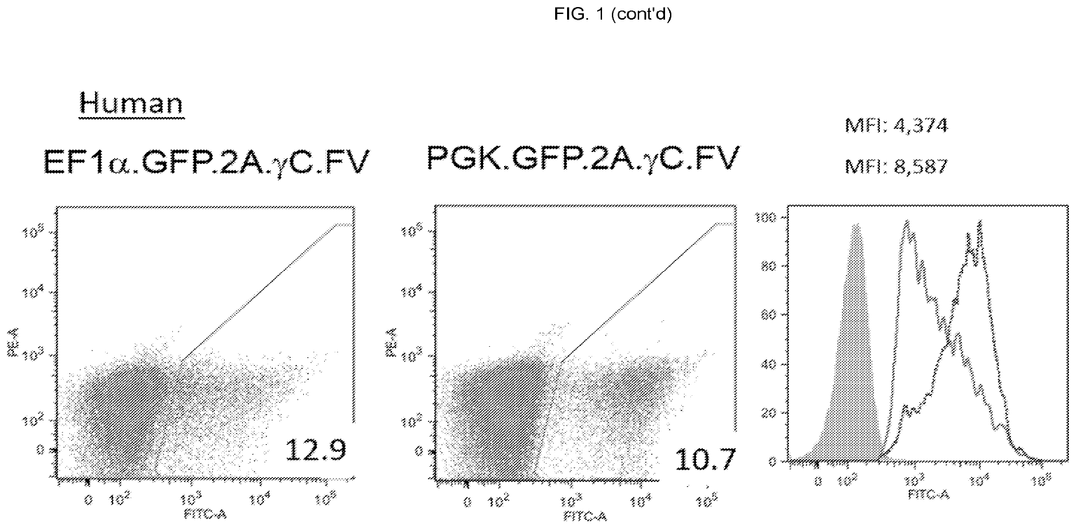

To improve the in vivo gene therapy approach, the FV vector design was modified by substituting the EF1α promoter for the human phosphoglycerate promoter (PGK). An FV vector utilizing the EF1α promoter (EF1α.γC.FV) and an identical vector containing the PGK promoter (PGK.γC.FV) were compared. In vitro transduction of human and canine CD34+ cells using matching doses of each vector showed increased expression by 2-fold in both cell types for PGK.γC.FV, determined by cis-linked fluorophore expression as surrogate marker (FIG. 1). EF1α.γC.FV and PGK.γC.FV were then directly compared in vivo using a competitive repopulation assay, in which two newborn SCID-X1 animals were injected intravenously with equal doses of each FV vector (FIGS. 3, R2258 and R2260). A distinct cis-linked fluorophore, GFP or mCherry (linked to the γC gene with a T2A peptide), was used to track immune reconstitution and the configuration of the fluorophores was permuted in the second animal to rule out any effect of the reporter gene. As shown in FIG. 2A and FIG. 3, R2258 was injected with a combination of EF1α.GFP.2A.γC.FV and PGK.mCherry.2A.γC.FV while R2260 was injected with EF1α.mCherry.2A.γC.FV and PGK.GFP.2A.γC.FV.

The absolute number of circulating lymphocytes steadily increased in both treated dogs during the course of 2½ years post treatment while remaining within normal range (FIG. 2B). Strikingly, the majority of gene marking (70-90%) in peripheral blood came from the PGK.γC.FV vector in both animals, while marking from the EF1α.γC.FV vector only included a small fraction (5-10%) (FIG. 2C). Interestingly, the early kinetics of gene marking in peripheral blood lymphocytes in these two animals were substantially improved as compared to animals treated with the EF1α.γC.FV vector in a previous study (Burtner C R et al. (2014) Blood 123: 3578-3584; R2202 and R2203, FIG. 3). As shown in FIG. 4C, the fraction of gene corrected peripheral lymphocytes reached 40% in both R2258 and R2260 at 6 weeks post injection, as compared to less than 5% for the EF1α.γC.FV-treated animals (Burtner et al. (2014) supra, compare diamond and circle lines). These results demonstrated superior therapeutic performance of the PGK.γC.FV vector as compared to EF1α.γC.FV, and will thus be the preferred vector platform for subsequent experiments using in vivo delivery.

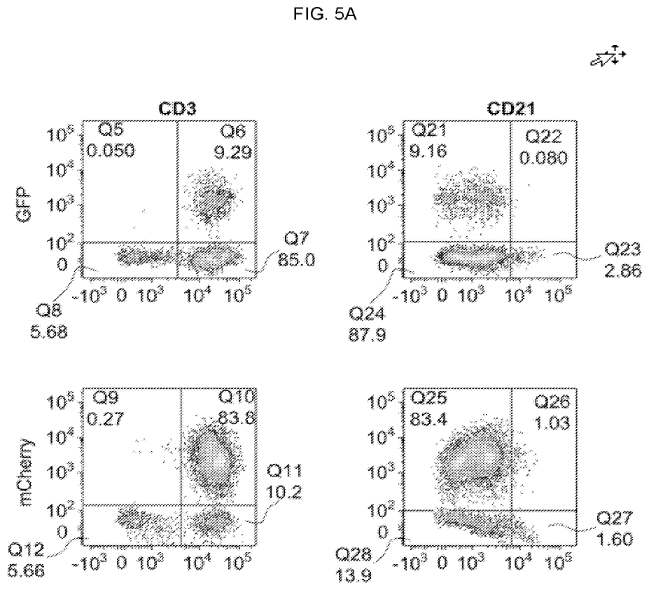

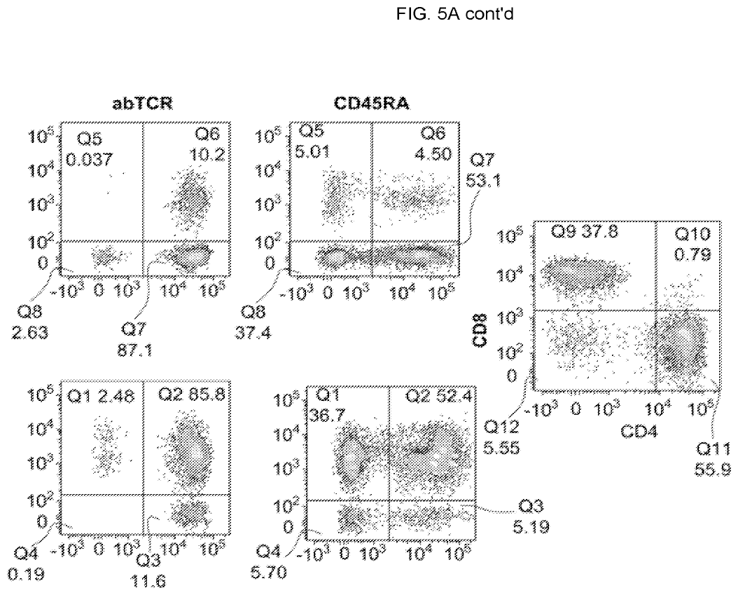

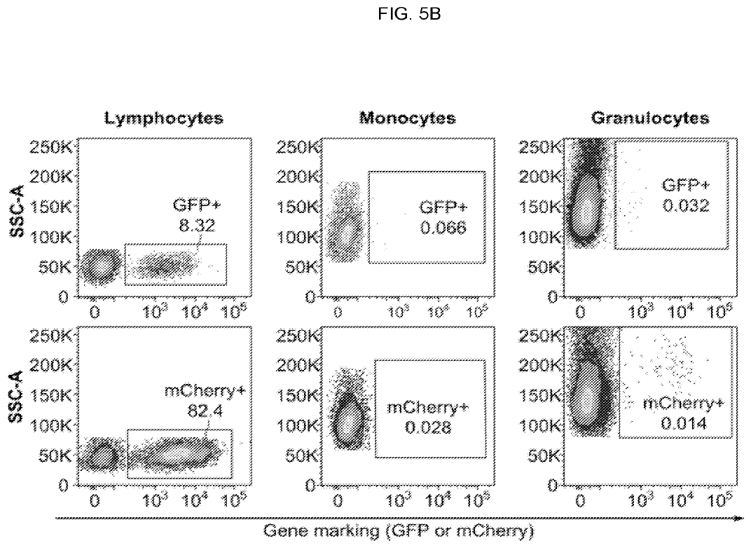

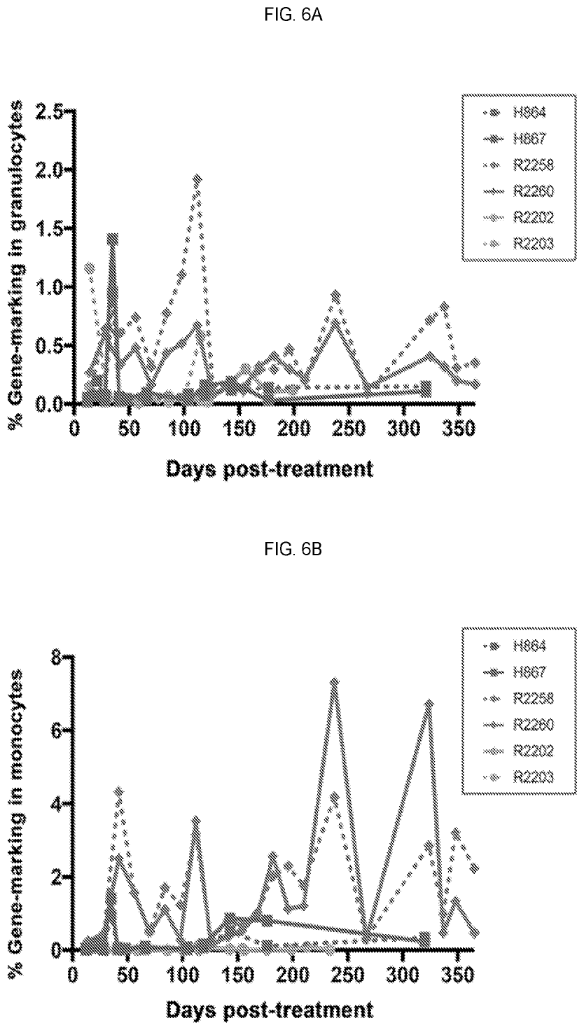

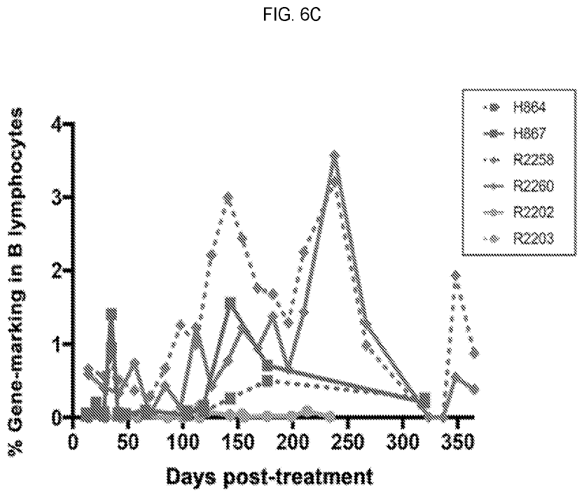

2) G-CSF/AMD3100 mobilization enhances kinetics of T-lymphocyte expansion and immune clonal diversity in FV treated animals. While the majority of circulating T lymphocytes expressed the γC transgene in treated SCID-X1 dogs, marking in cell lineages with no selective advantage such as B lymphocytes and myeloid cells was low, albeit above background (FIGS. 5A-5B, FIGS. 6A-6C), suggesting that the delivery approach did not effectively target the most primitive HSPCs. Previous studies have shown that a combination of granulocyte-colony stimulating factor (G-CSF) and AMD3100 efficiently mobilize multipotent HSPCs in peripheral blood in animal models and in patients (Broxmeyer H E et al. (2005) J Exp Med 201: 1307-1318; Larochelle A et al. (2006) Blood 107: 3772-3778; Dar A et al. (2011) Leukemia 25: 1286-1296; Richter M et al. (2016) Blood 128: 2206-2217). Based on these findings, two SCID-X1 canine pups were treated at 3 weeks of age with 5 μg/kg of G-CSF twice a day from day −4 to −1 prior to FV injection and a single dose of G-CSF with 4 mg/kg of AMD3100 on the morning of the injection (FIG. 4A). Treatment was well tolerated and resulted in a 6.4-fold to 7.2-fold increase in circulating CD34+ cells at 6 hours post AM D3100 administration (2.30% and 2.59% of total white blood cells in the two mobilized SCID-X1 animals as compared to 0.36% in a non-mobilized normal littermate control, consistent with values for steady state hematopoiesis (Gougeon M L et al. (1990) J Immunol 145: 2873-2879), FIG. 4B).

Injection of vector PGK.mCherry.2A.γC.FV at 6 hours post AMD3100 administration significantly increased kinetics of lymphocyte expansion and gene marking as compared to non-mobilized, FV-treated animals. The fraction of gene corrected lymphocytes in peripheral blood of mobilized animals reached 80% at 6 weeks post treatment while it took over 20 weeks in non-mobilized animals to reach similar levels (FIG. 4C). Accordingly, the time required to reach normal lymphocyte counts was markedly reduced in the mobilized animals (FIG. 4D); absolute CD3+ lymphocyte count reached 2,500 cells/μL in mobilized animals at 40 days post-FV vector injection as compared to 100 and 400 cells/μL in non-mobilized animals injected with EF1α.γC.FV, or competitively injected with PGK.γC.FV and EF1α.γC.FV, respectively (FIG. 4E). While low sample number precluded statistical analysis between mobilized and non-mobilized groups, statistical significance (p<0.01) in gene marking levels was found between animals treated with EF1α.γC.FV (n=5, including all animals from previous study (Burtner C R et al. (2014), supra)) versus animals treated with PGK.γC.FV (n=4) when comparing the mean linear regression slopes of each experimental group over the first 60 days (0.11 for EF1α, n=5 vs. 1.01 for PGK, n=4, Material and Methods). A difference in kinetics of CD3+ lymphocyte count was also noted between the same two groups over the same time frame but did not reach statistical significance due to low number of subjects and considerable variation within each group (mean slope =6.15 for EF1α, n=5, vs. 23.40 for PGK, n=4; p=0.17).

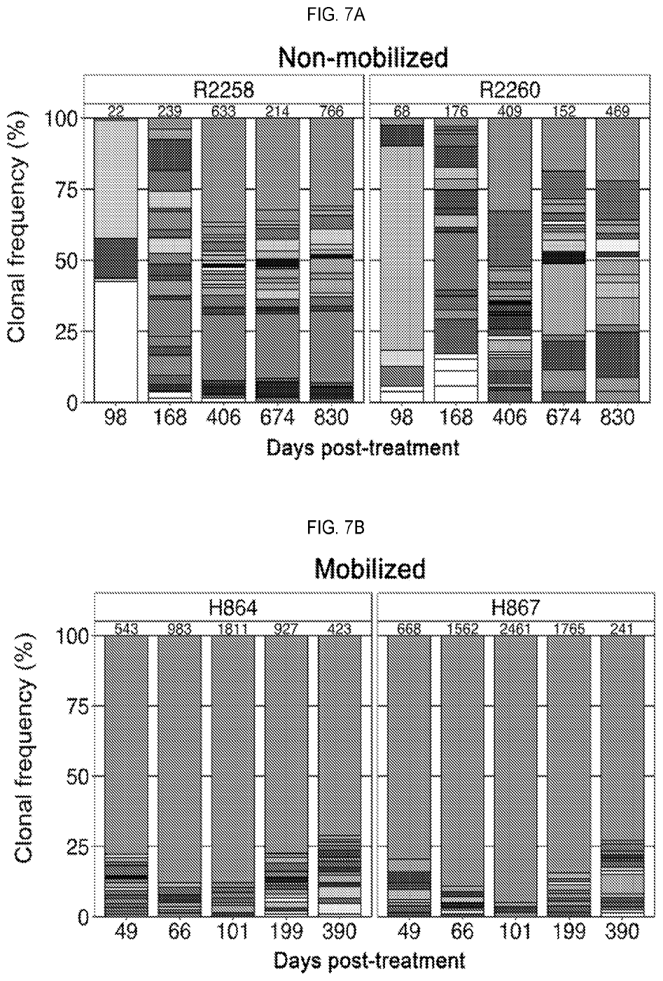

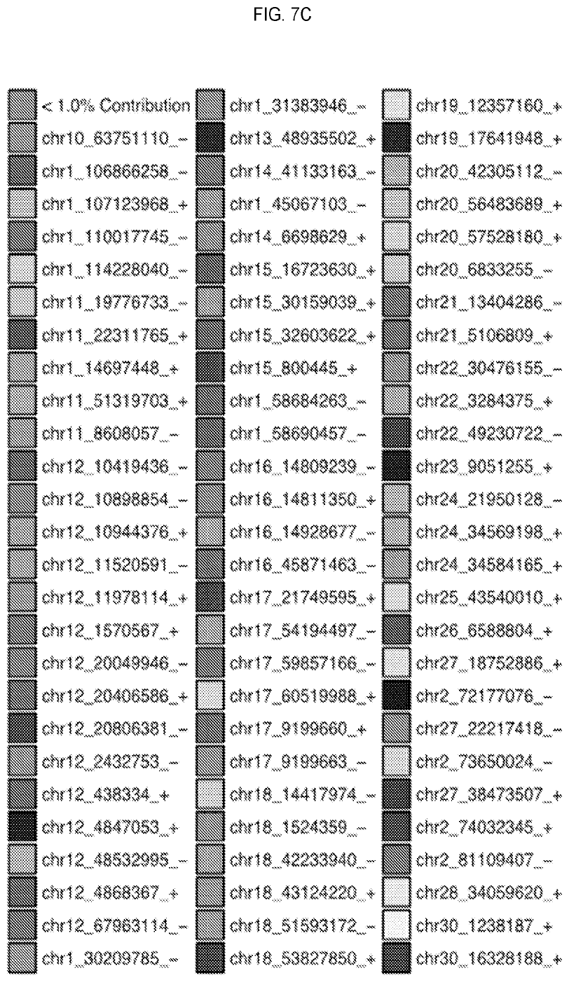

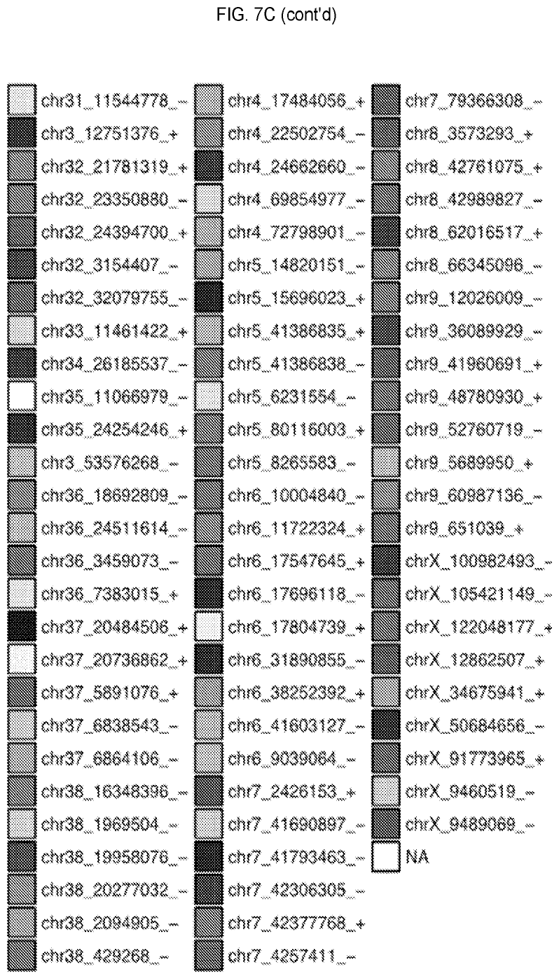

It was hypothesized that in addition to improving kinetics of lymphocyte reconstitution, mobilization may also enhance clonal diversity of gene-corrected cells. Retroviral integration site (RIS) analysis from peripheral white blood cells DNA showed a marked increase in integration events (i.e. clones) in mobilized dogs H864 and H867 as compared to non-mobilized dogs R2258 and R2260, despite use of an equal dose of PGK.γC.FV vector (FIG. 3). As shown in FIGS. 7A and 7B (legend in FIG. 7C), the fraction of clones that contributed to less than 1% of total gene marking (represented by gray area) was higher in the mobilized animals as compared to non-mobilized animals (<35% in R2258 and R2260, FIG. 7A, vs. 75-85% in H864 and H867, FIG. 7B). No clonal dominance was observed in any animal, but some persisting clones contributing to more than 10% of total gene marking were found in the non-mobilized animals, albeit with no indication of expansion (FIG. 7A). Taken together, these data suggest that G-CSF/AMD3100 treatment prior to intravenous FV vector delivery increases both the kinetics of lymphocyte recovery and the diversity of immune reconstitution in SCID-X1 canines.

Improved thymic output and broad TCR repertoire in mobilized FV treated animals. Lymphocytes originating from the thymus that have not yet been exposed to antigens express a naïve CD45RA+ phenotype, thus providing a measure of thymic output. The two non-mobilized, FV vector treated animals (R2258 and R2260) initially showed normal frequency (90%) of CD3+ CD45RA+ T-cells in peripheral blood but their frequency subsequently declined to 50% at one year post treatment (FIG. 8A). In comparison, levels of CD3+CD45RA+ cells remained stable in mobilized dogs H864 and H867 for at least 15 months post treatment and continue to be monitored (FIG. 8B). Thymic output was assessed independently by analysis of T cell receptor excision circles (TRECs) originating from TCR gene rearrangement during T-lymphocyte maturation. In the non-mobilized animals, TRECs were initially 10-fold lower in treated dogs as compared to a normal littermate control, then gradually declined over time (FIG. 8C). In contrast, TRECs in the mobilized animals reached normal levels as early as 3 months post treatment and remained comparable to the littermate control (FIG. 8D). Therefore, mobilization prior to FV vector injection of SCID-X1 canines increased thymic output to levels comparable to a healthy control.

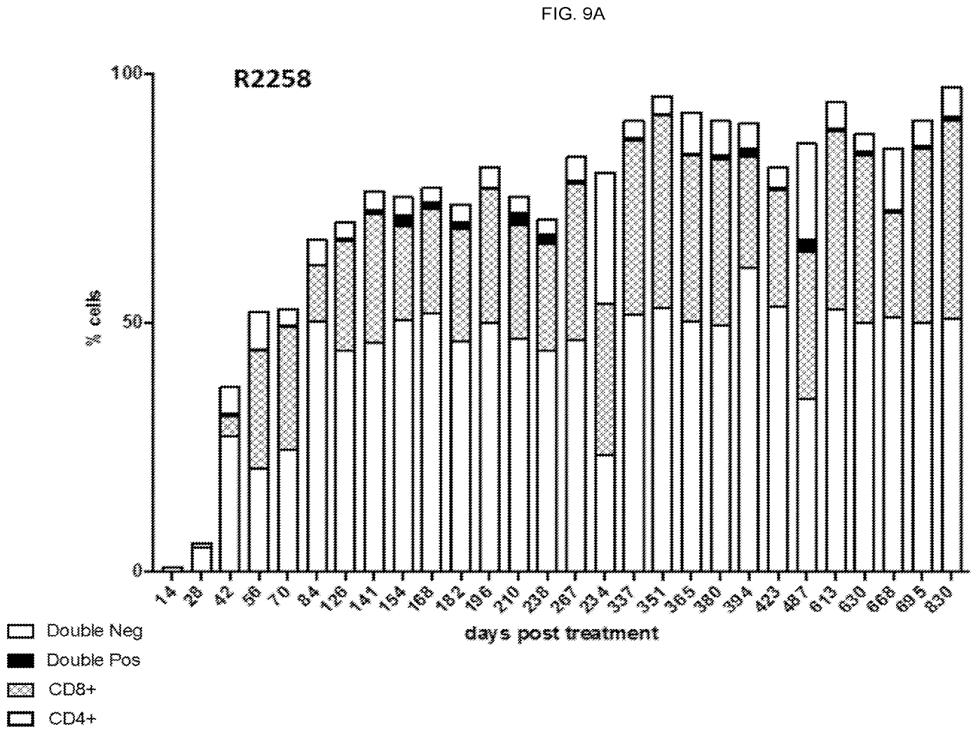

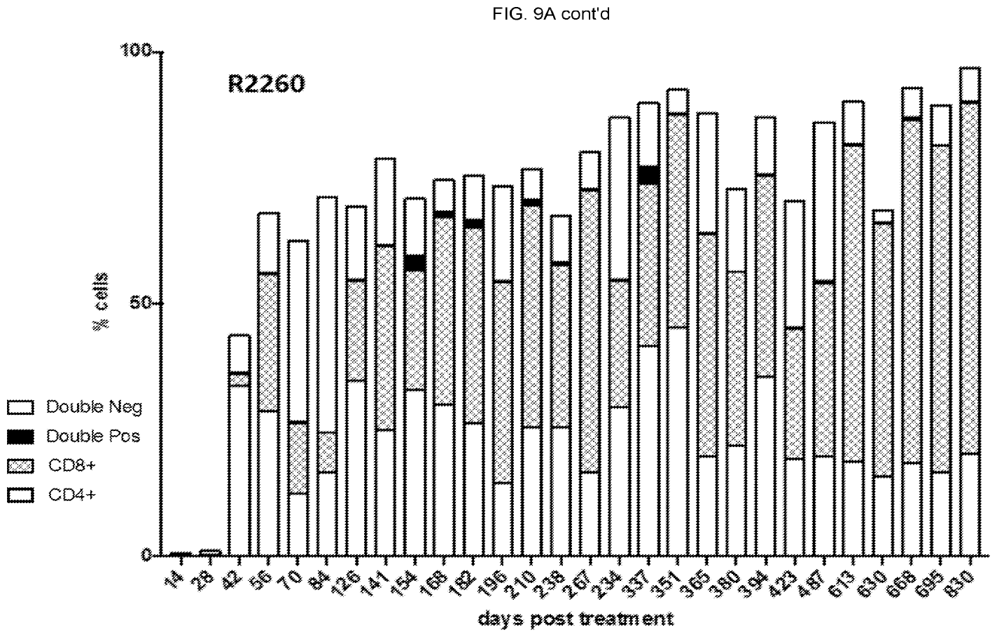

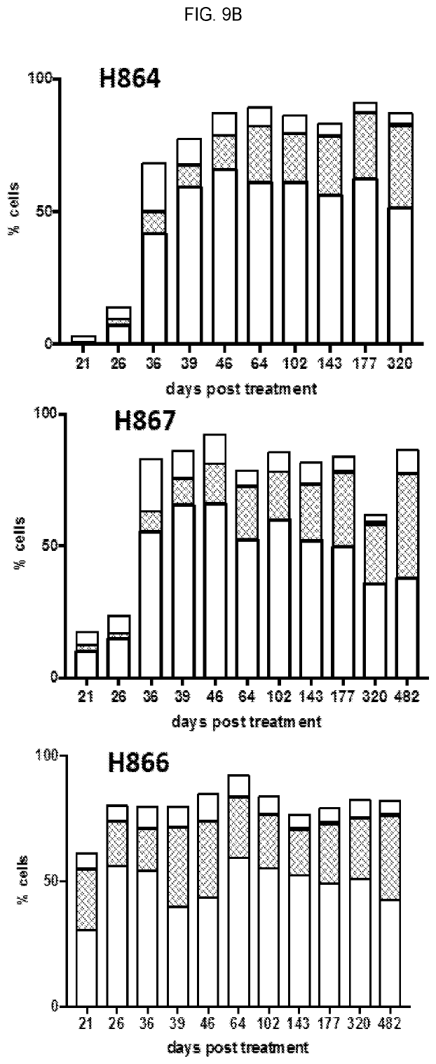

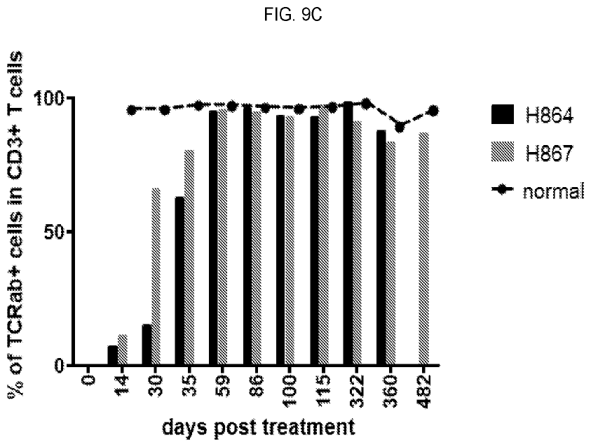

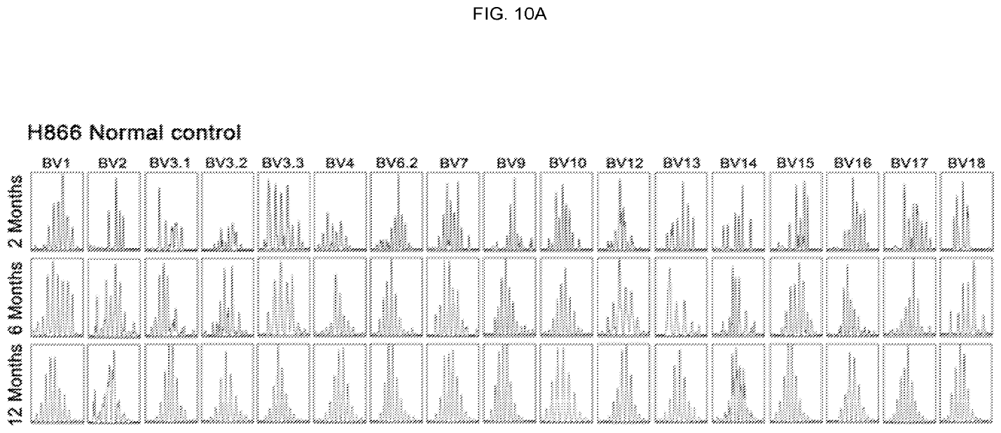

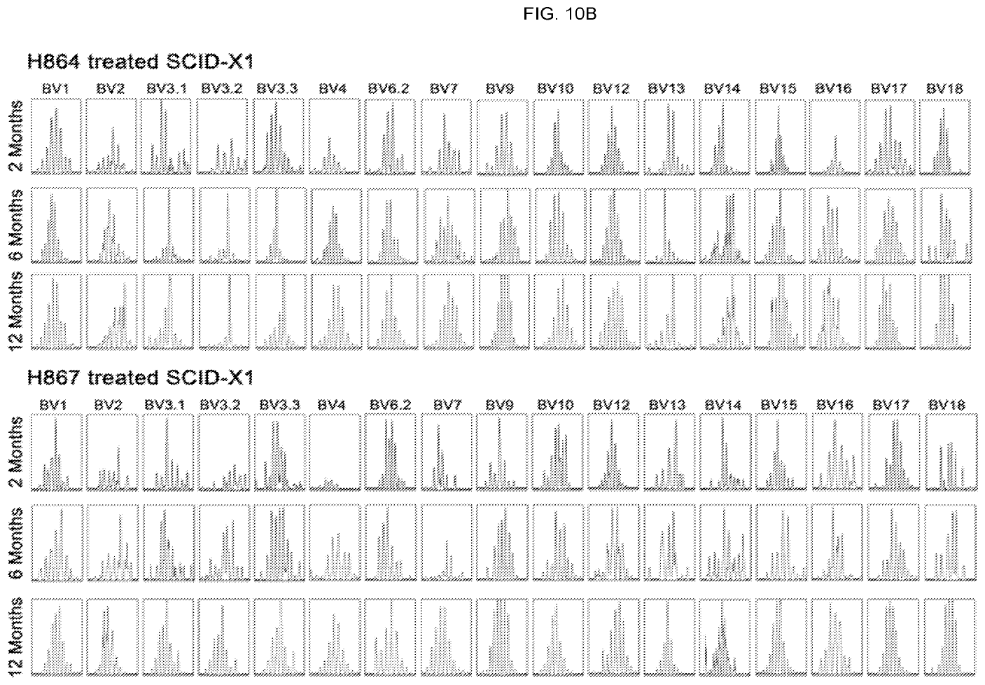

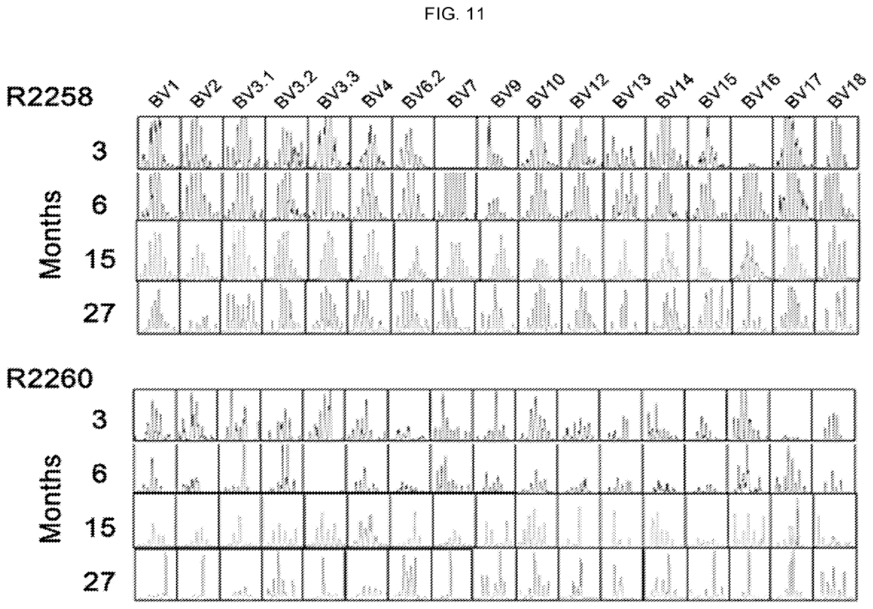

The majority of expanded CD3+ lymphocytes were mature, expressing the coreceptors CD4 or CD8, with a small fraction of cells being CD4/CD8 double positive or double negative (FIGS. 9A and 9B). Both mobilized animals H864 and H867, as well as non-mobilized animal R2258, showed normal CD4 to CD8 ratios averaging 2, whereas non-mobilized animal R2260 showed an inverted ratio. The reason for this skewed ratio is not well understood but may be caused by ongoing chronic infections as was observed in some animals from a previous study (Burtner C R et al. (2014), supra). Most circulating T-lymphocytes in H864 and H867 also stained positive for TCR αβ starting at 2 months post treatment, consistent with observations from healthy canines and humans (FIG. 9C). T cell receptor (TCR) diversity was assessed in each treated animal by TCRVbeta spectratyping, which analyzes genetic rearrangement of the variable region of the TCR beta gene. The two animals mobilized with G-CSF/AMD3100 showed robust spectratype profiles, characterized by Gaussian distribution of fragments sized across 17 families of TCRVbeta segments up to at least a year post treatment, similar to that of an aged-matched normal littermate (FIGS. 10A, 10B). In comparison, the spectratype profile of non-mobilized animal R2258 appeared normal throughout the experiment but R2260's profile was weaker, suggesting lower TCR diversity (FIG. 11). Therefore, mobilization prior to FV vector injection of SCID-X1 animals resulted in normal T cell maturation and TCR diversity that is representative of healthy animals.

4) Restoration of T- and B-lymphocyte function in PGK.γC.FV treated animals. Functionality of the γC-dependent signaling pathways in corrected lymphocytes obtained from all FV treated animals was verified by measuring tyrosine phosphorylation of the effector molecule STAT3 following in vitro stimulation with IL-21. As compared to cells obtained from a normal littermate control, equivalent levels of STAT3 phosphorylation were detected in CD3+ lymphocytes from non-mobilized SCID-X1 dogs R2258 and R2260 (FIG. 12A), or from mobilized FV treated animals H864 and H867 (FIG. 12B).The ability of T-lymphocytes isolated from treated animals to respond and proliferate when exposed to the T cell mitogen phytohemagglutinin (PHA) was further confirmed, a process that is highly dependent on functional IL-2 receptor signaling (FIG. 12C). Therefore, these results demonstrated restoration of T-cell specific signaling pathways in FV treated SCID-X1 dogs.

Primary and secondary antibody responses and immunoglobulin (Ig) class-switching after immunization with the T-cell dependent neoantigen bacteriophage ϕX174 were next evaluated. All treated animals exhibited primary and secondary antibody responses that were within the range of normal canine control (compare diamond and square lines with “x” line, FIG. 13A) with no noticeable differences between mobilized and non-mobilized animals. Polyclonal Ig class M, G and A concentrations were also measured from serum of mobilized dogs at 15 months post treatment and were mostly comparable to the littermate control and within normal range (FIG. 13B). Thus, the in vivo FV vector gene therapy restored both B- and T-cell cytokine signaling programs required for class switching and memory response despite low levels of peripheral B lymphocyte correction detected in these animals (ranging from 0 to 4%, FIGS. 5A and 6C).

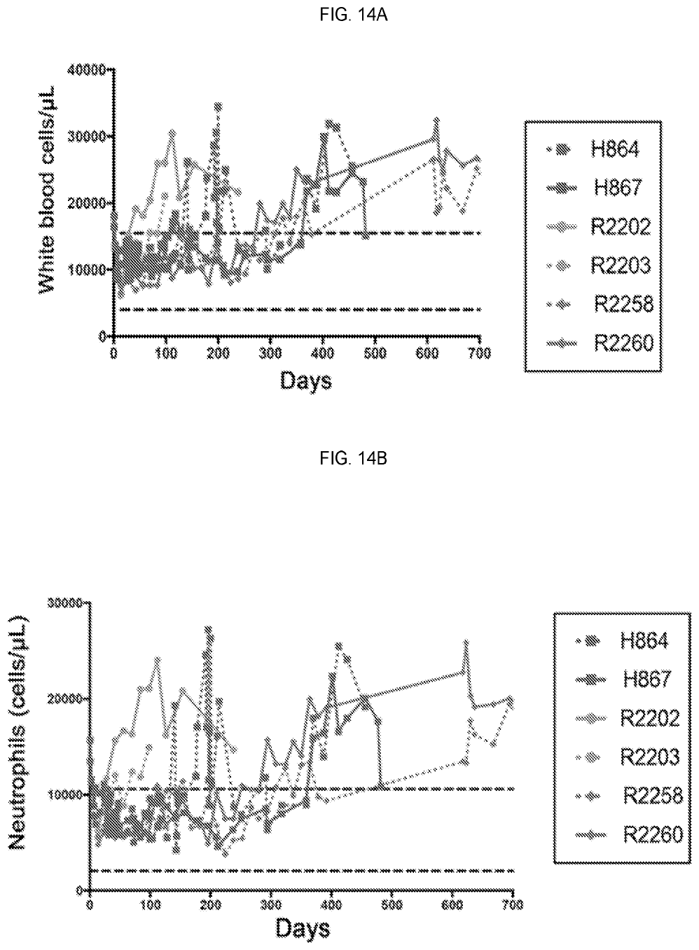

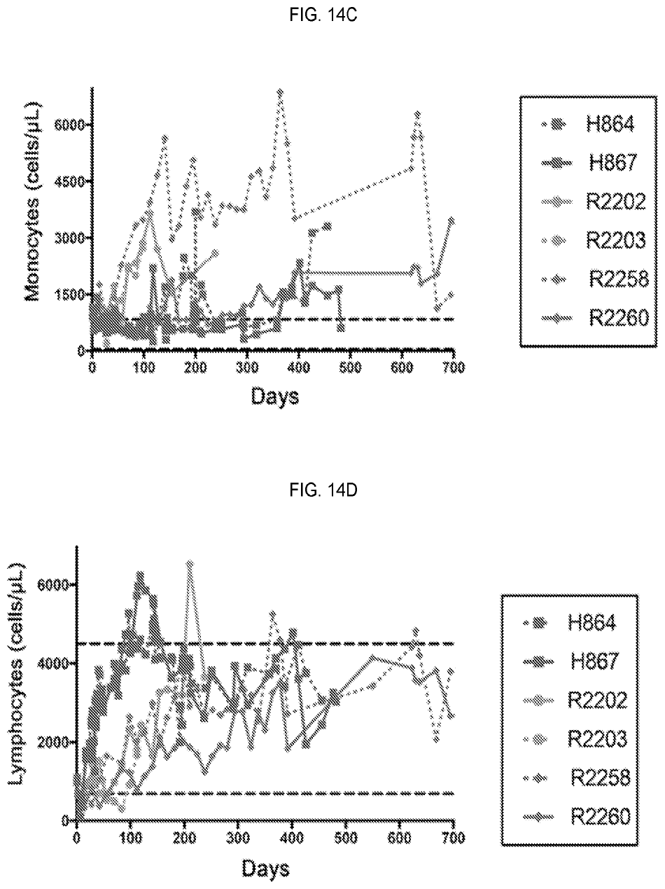

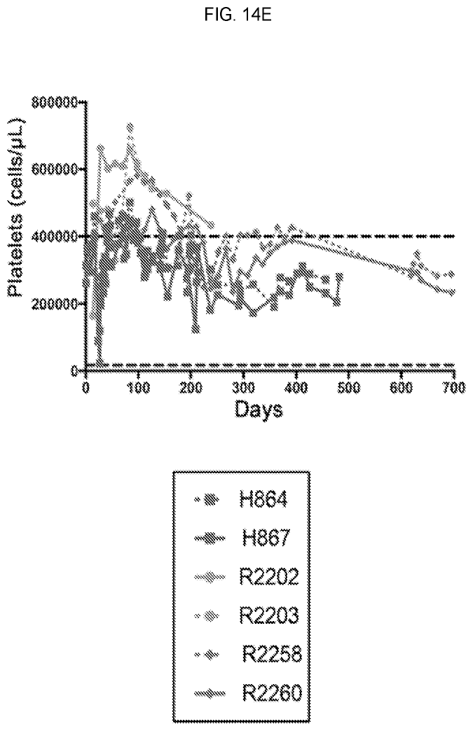

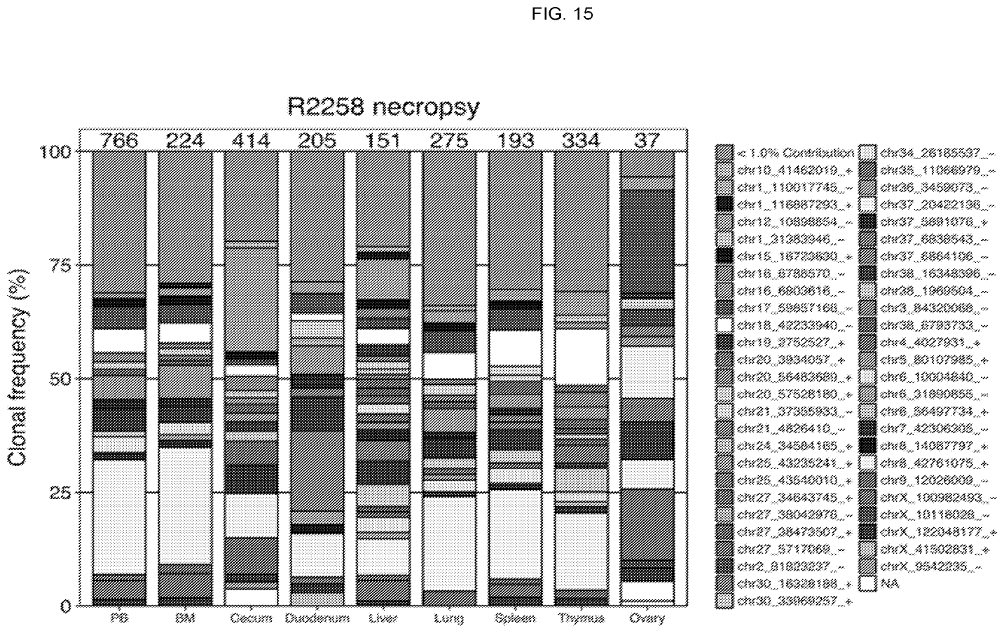

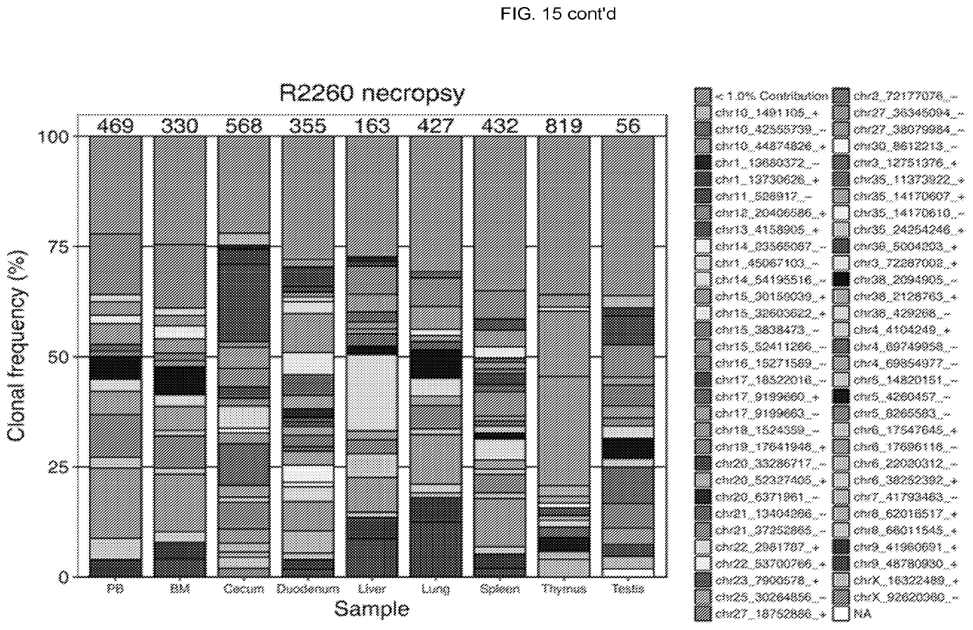

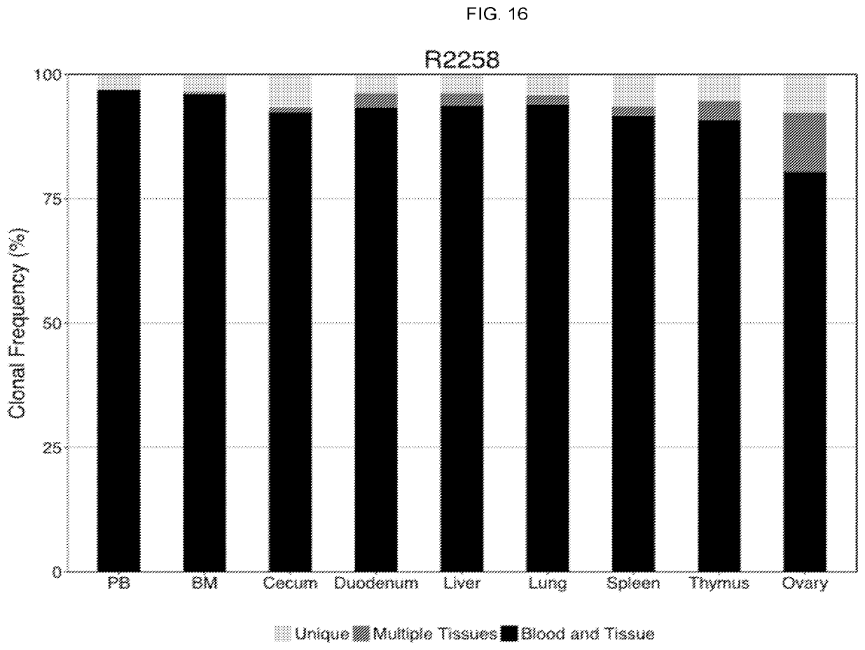

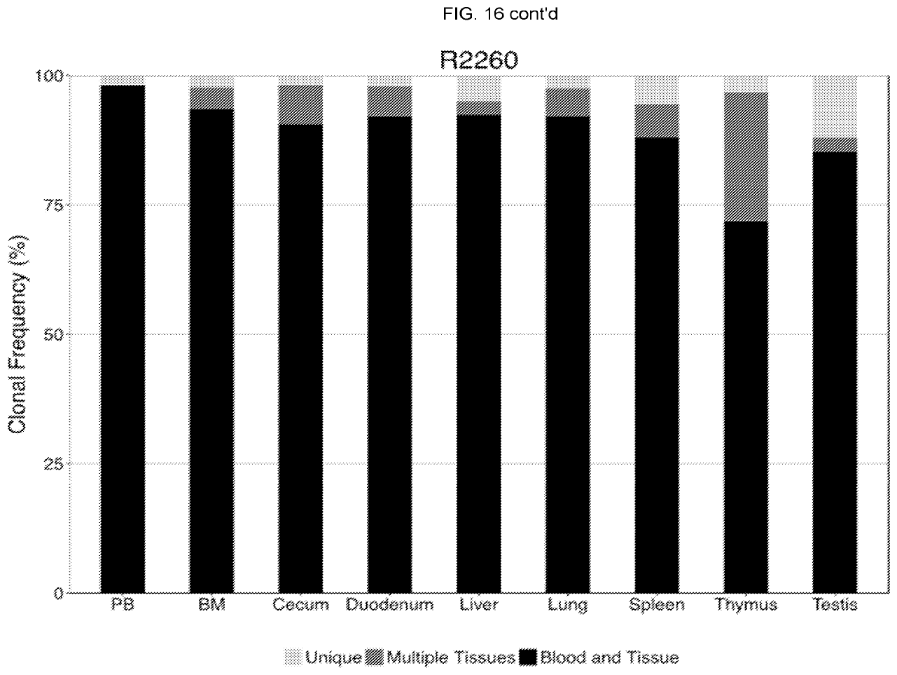

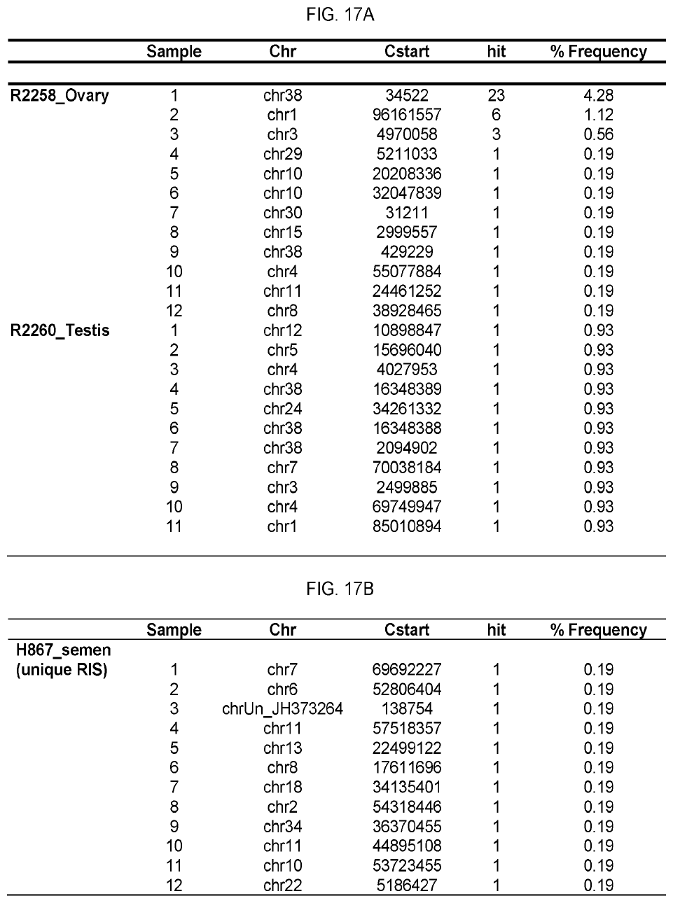

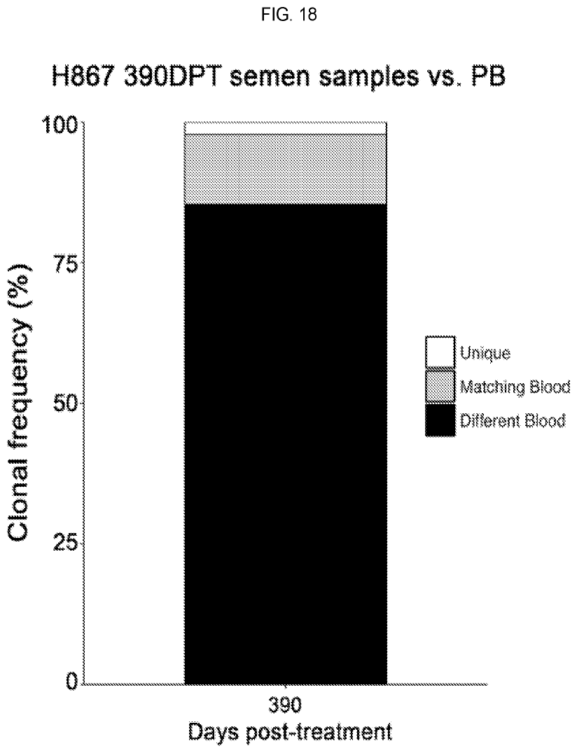

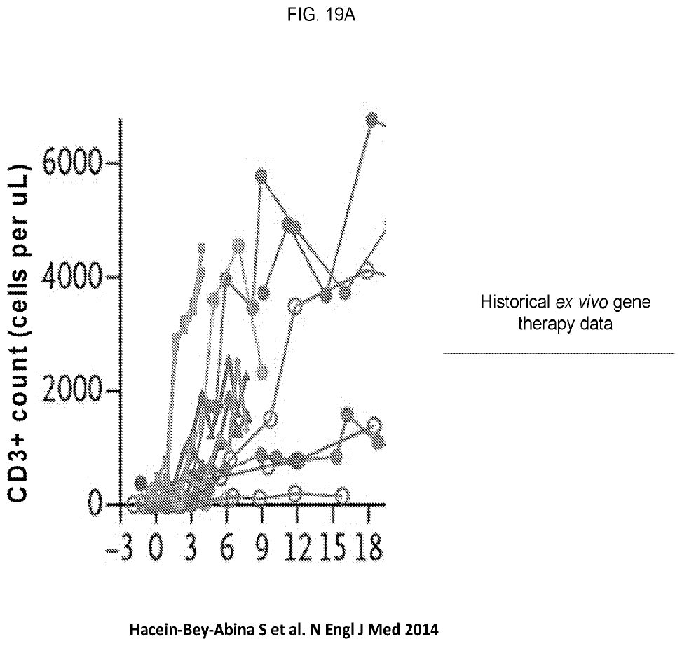

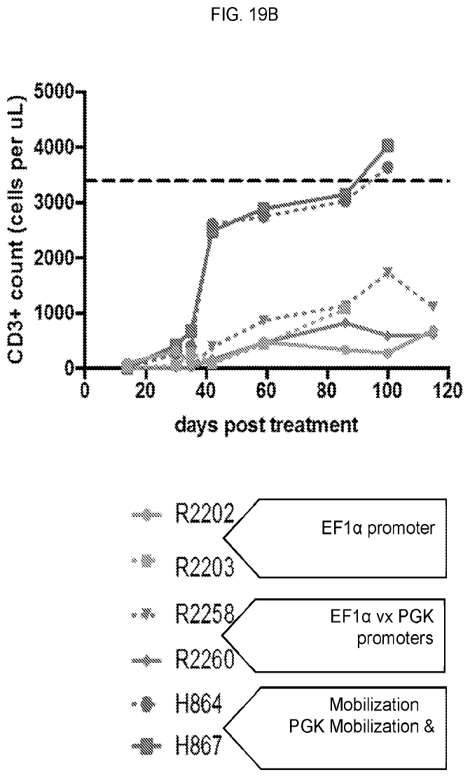

5) Safety of in vivo FV vector gene therapy and G-CSF/AMD3100 treatment. In vivo gene therapy proved beneficial for all SCID-X1 dogs: the non-mobilized animals (R2258 and R2260) lived for over 2½ years in a non-sterile environment, while the two mobilized animals (H864 and H867) are currently over 16 months of age. Complete blood cell count (CBC) analysis for all animals remained within normal range in the first year post treatment but neutrophil and monocyte counts gradually increased at later time points (FIGS. 14A-14E), probably reflecting inflammatory responses to parasitic or viral infection. R2258 and R2260 eventually developed papilloma virus infections, similar to observations from SCID-X1 canines (Goldschmidt M H et al. (2006) J Virol 80: 6621-6628) or patients treated with BMT or gene therapy (De Ravin S S et al. (2016) Sci Transl Med. 8(335): 335ra357; Laffort C et al. (2004) Lancet 363: 2051-2054), and had to be euthanized at 830 days post treatment. Tissues from these animals were collected and analyzed by RIS for biodistribution assessment of the foamy provirus. The vast majority of RIS (>90%) detected in tissues (FIG. 15) were also found in peripheral blood samples at the same time point, indicating that they originated from contaminating blood cells present in perfused tissues (FIG. 16). Ovaries and testis showed the smallest number of integration events (37 and 56, respectively, as compared to 766 and 469 in blood) and none of the RISs found exclusively in the gonad tissues (i.e. unique RIS) appeared at biologically relevant frequency except for one integration site in the ovaries (Chr38: 34,522, 4.28%, FIG. 17A). No unique RIS at biologically relevant frequency was detected in semen from male H867 (FIGS. 18 and 17B). Taken together, these results suggested that off-target transduction events by in vivo FV vector treatment is a very rare event with no compelling evidence of provirus integration in the germline, a finding also supported by study of progeny issued from FV treated male R2260 (as discussed below).

Discussion. Ex vivo HSPC gene therapy clinical trials involving SCID-X1 patients are demonstrating clear clinical benefits (reviewed in Cavazzana M et al. (2016) Hum Gene Ther 27: 108-116) but require elaborate protocols, sophisticated facilities, and genotoxic conditioning. Here a simpler, safer, and more versatile gene therapy approach for SCID-X1 that involves the direct intravenous injection of FV vectors with no prior conditioning regiment is disclosed. Important improvements in methods were made by mobilizing HSPCs with G-CSF and AMD3100 prior to injection with a modified FV vector containing a stronger enhancer-less PGK promoter. Kinetics of lymphocyte reconstitution was markedly increased presumably due to transduction of a greater pool of lymphocyte precursors in blood, which also enhanced thymopoiesis and clonal diversity. As compared to a previous study where all EF1α.γC.FV-treated animals succumbed to chronic infections by 330 days post treatment (Burtner C R et al. (2014), supra), survival was improved in all treated SCID-X1 dogs, which lived for as long as 2½ years.

A critical parameter in vector design is the choice of promoter-enhancer element with sufficient strength to drive efficient immune reconstitution and with minimal risk for inadvertent enhancer-mediated gene transactivation. All current SCID-X1 clinical trials use viral vectors containing the EF1α promoter to drive expression of the γC gene (Hacein-Bey-Abina S et al. (2014) NEJM 371(15): 1407-1417; De Ravin S S et al. (2016) Sci Transl Med. 8(335): 335ra357). This choice of promoter was validated in previous studies that compared genotoxic risks associated with the physiological cellular promoters from the human EF1α or PGK genes, or from the endogenous γC gene (Zychlinski D et al. (2008) Molecular Therapy 16: 718-725; Zhou S et al. (2010) Blood 116: 900-908). In addition, in a murine model of SCID-X1, HSPCs transduced with the EF1α-containing LV vector completely restored lymphoid development and immune function, whereas cells modified with PGK-containing vector resulted in poor immune reconstitution (Ginn S L et al. (2010) Molecular Therapy 18: 965-976). Nevertheless, the long form (1,200 bp) of EF1α promoter was used in this study, while the EF1α-γC-FV vector described in the present disclosure contains the shorter form (250 bp), similar to the clinically approved vector (De Ravin S S et al. (2016) Sci Transl Med. 8(335): 335ra357). The competitive injections of EF1α.γC.FV and PGK.γC.FV vectors of SCID-X1 pups clearly showed superiority of the PGK vector, which accounted for over 80% gene marking and no evidence for clonal dominance. Superior performance of the PGK promoter was similarly demonstrated in a radiation-sensitive SCID murine model with complete functional correction of the Artemis gene achieved with a PGK-LV vector, whereas CMV- or EF1α-LV lead to incomplete correction (Mostoslaysky G et al. (2006) PNAS 103: 16406-16411), and efficacy of a PGK.γC SIN LV vector was also validated for ex vivo SCID-X1 gene therapy using a murine model (Huston M W et al. (2011) Molecular Therapy 19: 1867-1877). Stronger γC expression correlated with increased therapeutic performance in the presently described model.

Most HSPCs reside in the bone marrow space and are thus not accessible to intravenously injected FV vectors. G-CSF and AMD3100 in combination have been used successfully to increase CD34+ cells in peripheral blood of mice, nonhuman primates and humans (Broxmeyer H E et al. (2005) J Exp Med 201: 1307-1318; Larochelle A et al. (2006) Blood 107: 3772-3778; Richter M et al. (2016) Blood 128: 2206-2217). Both G-CSF and AMD3100 have mobilizing properties by acting on distinct cellular pathways, and combinatory treatment resulted in additive effects (Liles W C et al. (2003) Blood 102: 2728-2730). G-CSF suppresses osteoblast lineage cells in the bone marrow niche, leading to reduced levels of signaling molecules (eg.: CXCL12, VLA-4, c-Kit), which are essential for HSPC retention (Winkler I G et al. (2012) Leukemia 26: 1594-1601). The bicyclam AMD3100 is a potent, selective, and reversible antagonist of the CXCR4 chemokine receptor and disrupts the binding of CXCR4 to SDF-1, thereby mobilizing HSPCs into the blood (Dar A et al. (2011) Leukemia 25: 1286-1296; Rosenkilde M M et al. (2004) J Biol Chem 279: 3033-3041). Kinetics of HSPCs mobilization by AMD3100 alone was previously assessed in adult dogs (Burroughs L et al. (2005) Blood 106: 4002-4008), in which the treatment was well tolerated and circulating CD34+ cells increased 3- to 10-fold with peak mobilization at 8-10 hours post treatment. Similarly, G-CSF/AMD3100 mobilization of immunodeficient humanized mice increased colony forming units (CFUs) isolated from peripheral blood by 2.3 and 8.2-fold, respectively (Richter M et al. (2016) Blood 128: 2206-2217). Mobilization of SCID-X1 pups with G-CSF/AMD3100 in this Example showed a comparable 7-fold increase in circulating CD34+ cells at 6h post treatment.

Beyond mobilizing HSPCs in peripheral blood, G-CSF/AMD3100 treatment also affects other cell lineages. Circulating lymphocyte and monocyte counts were increased by medians of 1.5- and 4-fold, respectively, in mobilized adult canines as compared to untreated controls (Burroughs L et al. (2005) Blood 106: 4002-4008). In rhesus macaques, AMD3100 increased numbers of B and T lymphocytes, which included CD4+ and CD8+ T cells, central and effector memory T cells, as well as NK cells in peripheral blood (Kean L S et al. (2011) Blood 118: 6580-6590). In addition, macaques transplanted with G-CSF/AMD3100-mobilized CD34+ cells manifested faster lymphocyte recovery as compared to non-mobilized animals, likely due to increased blood count of lymphoid precursors (Uchida N et al. (2011) Exp Hematol 39: 795-805). The demonstration of faster blood T-lymphocyte recovery, increased thymopoiesis and clonal diversity in G-CSF/AMD3100 mobilized SCID-X1 pups in this Example imply that a larger pool of HSPCs was transduced following intravenously delivered FV vectors. These corrected HSPCs may directly home to the thymus, as suggested by previous findings (Weerkamp F et al. (2006) Blood 107: 3131-3137), and subsequently differentiate into mature CD4+ and CD8+ T-lymphocytes. The decline in both TRECs and CD45RA+ naïve T cells at 8-9 months post treatment in the two non-mobilized canines can either be explained by the inability of the hypoplastic SCID thymus to sustain thymopoiesis, or to the poor engraftment of gene corrected HSPCs capable of self-renewal and continuous production of functional T cells. G-CSF/AMD3100 mobilization substantially increased TREC levels in treated SCID-X1 animals and may help prolong thymic output.

The two non-mobilized FV treated animals developed cutaneous warts due to severe papillomavirus (PV) disease at 28 months post treatment after arrival in the new canine colony. Both SCID-X1 canines and human patients are known to be susceptible to PV infections (Laffort C et al. (2004) Lancet 363: 2051-2054; Goldschmidt M H et al. (2006) J Virol 80: 6621-6628). A retrospective study of 41 SCID patients who survived over 10 years following bone marrow transplant reported that 50% of patients developed chronic severe PV infection (Laffort C et al. (2004) Lancet 363: 2051-2054). No correlation could be drawn between PV disease and NK counts or function despite their capacity to directly eliminate PV infected cells. Deficiency in keratinocyte function, the target cells for PV infection, may alternatively provide an explanation for PV susceptibility, since they express γC-dependent cytokine receptors such as interleukin 4, which activates the release of proinflammatory cytokines under normal conditions.

Upon sexual maturity, FV vector treated male R2260 sired three litters via artificial insemination, overall resulting in 2 SCID-X1 pups, 4 SCID-X1 carriers and 4 wild-type pups. This normal pedigree argues against a possible transduction of the germline by FV vectors, thus corroborating the RIS data showing absence of relevant integration site in semen obtained from H867. While one unique integration site was documented in ovaries from R2258, it may originate from transduction of accessory cells and not from germ cells.

BMT has traditionally been the preferred treatment for SCID-X1 patients, but recent findings demonstrated faster T cell reconstitution in SCID-X1 patients treated by ex vivo gene therapy as compared to haplo-identical HSPC transplantation (Touzot F et al. (2015) Blood 125: 3563-3569), suggesting that gene therapy can become the front line therapeutic modality. Host conditioning seems to be required for efficient engraftment and multi-lineage gene marking, such as NK and B-cells (reviewed in Cavazzana M et al. (2016) Hum Gene Ther 27: 108-116). Unfortunately, conditioning regimens also carry a high risk of treatment related morbidity in older patients and patients with potential and/or existing organ damage. Here, a simpler, safer, and more versatile gene therapy approach for SCID-X1 that involves the direct intravenous injection of FV vectors with no prior conditioning is disclosed. Improved conditions using G-CSF/AMD3100 mobilization prior to FV vector injection resulted in comparable or higher CD3+ cell reconstitution in treated SCID-X1 pups as compared to SCID-X1 children treated with ex vivo SIN-RV gene therapy (Hacein-Bey-Abina S et al. (2014) NEJM 371(15): 1407-1417). Overall, this Example demonstrates safety, feasibility and efficacy of FV vectors for in vivo gene therapy, which can provide prompt treatment of newborn SCID-X1 patients following routine genetic screening without complicated ex vivo manipulation of HSPCs and genotoxic conditioning, and could therefore be adopted at many institutions worldwide including those in developing countries.

### EXAMPLE 2

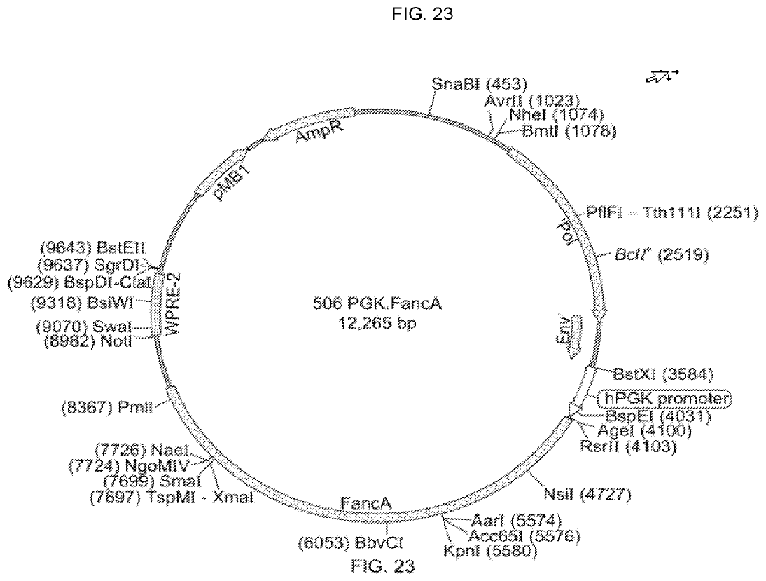

Construction and in vitro validation of FancA FV vectors. An FV-FancA construct containing a codon optimized human FancA gene under the control of the human phosphoglycerate kinase (PGK) promoter was generated using a previously published pFV SIN plasmid backbone (Burtner, 2014, supra; FIG. 24A). Briefly, FancA gene was obtained from lentiviral plasmid #18 (FIG. 22 and SEQ ID NO: 31; Adair J E et al. (2012) J Mol Med (Berl) 90(11): 1283-1294) by digestion with Agel/Notl/Ahdl. The largest band resulting from the digestion contained FancA up to the start of WPRE (4.8 kbp). FancA was then cloned into foamy vector #506 backbone (FIG. 21 and SEQ ID NO: 30) digested with Agel/Notl to remove the transgene (2 kbp). Diagnostic digestion using Agel/Notl showed 2 potential clones; clone #1 was validated by sequencing. An FV vector including a PGK promoter associated with FancA gene is shown in FIG. 23 (SEQ ID NO: 33).

The FV-FancA construct was then derived into FV-FancA-GFP, which additionally contains the GFP fluorophore under the control of the human Ef1α promoter to facilitate tracking of transduced cells (FIG. 24A). Briefly, EF1α-GFP (1 kbp) was PCR amplified with EF1α-F-Notl forward primer (5′-ATTAGCGGCCGCAGGCTCCGGTGCCCGTCAGT-3′, SEQ ID NO: 43) and GFP-R-Notl reverse primer (5′-ATTAGCGGCCGCTTACTTGTACAGCTCGTCCATG-3′, SEQ ID NO: 44) from an AAV construct and cloned into the Notl site of construct FV-FancA (described above). Three clones were obtained and validated in forward orientation by Notl and then Kpnl digestion.

Results. High titer FV vectors were prepared using each construct, achieving 1×10⁸ IU/mL for FV-FancA (100×concentrated titer). A 6-fold drop in titer was observed with FV-FancA-GFP as compared to FV-FancA, probably due to the large size of the transgenic cassette (over 6 kbp). FancA function in these FV vectors was validated first using a mitomycin C (MMC) sensitivity assay. The FancA−/− human fibroblast cell line GM06914 was transduced with two different multiplicities of infection (MOI of 1 and 5) of FV-FancA and showed a significant increase in survival of these cells as compared to untransduced cells when exposed to increasing concentrations of MMC (FIG. 24B). Interestingly, FV-FancA transduction at a MOI of 1 achieved comparable MMC resistance to transduction with a previously validated LV-FancA (Becker P S et al. (2010) Gene Therapy 17: 1244-1252) at the higher MOI of 5. In a different assay, the functionality of FV-FancA-GFP was validated by showing enrichment of FancA−/− cells transduced with FV-FancA/GFP when grown under increasing concentrations of MMC (FIG. 24C), indicating resistance to MMC for the FV-FancA/GFP-transduced cells. In contrast, the proportion of GFP+ cells remained unchanged under these conditions when FV-GFP was used for transduction (FIG. 24C).

### EXAMPLE 3

(Prophetic). In vivo delivery of FV-FANCA combined with HSPC mobilization to treat FA. Material and Methods. Animals. All animal procedures conform to protocols approved by the Fred Hutchinson Cancer Research Center Institutional Animal Care and Use Committee (IACUC). 129/SvJ-derived Fanconi Complementation Group A knockout (fanca−/−) mice (Rio P et al. (2002) Blood 100: 2032-2039) are obtained, and a colony is maintained. Genotyping is performed to identify the homozygous and heterozygous affected offspring mice.

Mobilization regimen. The mobilization regimen can be as described in Pulliam A C et al. (2008) Exp Hematol. 36(9): 1084-1090, as follows. Fanca−/− mice are injected subcutaneously (s.c.) with 3 μg G-CSF in 0.1 ml phospho-buffered saline/0.1% bovine serum albumin (PBS/0.1% BSA) every 12 hours for four consecutive days. Control animals receive a similar volume of PBS/0.1% BSA for four consecutive days. AMD3100 is administered at a dose of 5 mg/kg s.c. 14 hours following the last dose of G-CSF and one hour prior to FV delivery.

Gene marking and phenotypic analysis can be as described in Adair J E et al. (2012) J Mol Med (Berl) 90(11): 1283-1294. Mice are bled via the retro-orbital sinus at regular intervals to monitor kinetics of hematopoietic recovery and to collect samples for gene marking and RIS studies. At day +35, survival bone marrow aspirates are performed to assess gene marking in white blood cells (WBCs) and colony forming cells (CFCs). At days +85 through +92, mice are euthanized by CO₂, necropsies are performed, and blood, bone marrow and spleens are collected for additional gene marking analyses. Transduction efficiency and percent transduced cells are determined by: flow cytometry for GFP; quantitative real-time PCR of DNA isolated from transduced cells; and/or methylcellulose colonies. It will be observed that cells obtained from mice administered FV-FancA or FV-FancA-GFP are transduced with these vectors.

Methylcellulose and MMC resistance assays can be as described in Adair J E et al. (2012) J Mol Med (Berl) 90(11): 1283-1294. Hemolyzed BM cells are plated at a concentration of thirty thousand cells per 35 mm dish containing 1.2 ml methylcellulose (Stem Cell Technologies) and mitomycin C (MMC; Ben Venue Laboratories, Inc., Bedford, Ohio) at 0, 10 nM, or 20 nM in triplicate. Plates are incubated at 37° C. inside humidified (85%) and hypoxic (5% O₂) chambers. Colony numbers are counted after 14 days in culture by light microscopy and scored for transduction efficiency (GFP expression) by fluorescence microscopy. An average of 24 to 36 colonies from each treatment condition are picked up and the colony DNA is subjected to PCR using FV-specific primers to determine the presence or absence of the transduced FV vector and also genotyping primers to determine the total number of engrafted heterozygous cells. Alternatively, cells are also assayed for MMC resistance by suspension cell culture. Hemolyzed BM and spleen cells are cultured in IMDM medium containing 10% FBS in the presence of growth factors including G-CSF, SCF, Flt3 ligand, and TPO at 100 ng/ml each, on treated tissue culture vessels in the absence or presence of MMC. The surviving cell fraction is determined at 48 hours and 96 hours by the CellTiter Glo™ (Promega, Fitchburg, Wis.) luminescent cell viability assay. It will be observed that BM and spleen cells transduced with FV-FancA vectors are resistant to MMC.

Cytogenetic analysis can be as described in Adair J E et al. (2012) J Mol Med (Berl) 90(11): 1283-1294. Two million mouse bone marrow cells are used to inoculate 5 ml of RPMI media supplemented with 16% FBS and 10% PEN/Strep or MarrowMax media. The cultures are incubated at 37° C. with 5% CO₂ for 15 to 30 hours before addition of colcemid (final concentration 0.04 μg/mL) to arrest the cells in metaphase. Cells are harvested with a hypotonic solution (0.075 M KCl) pre-warmed to 37° C. and fixed in methanol and glacial acetic (2.5:1 ratio). Fixed cells are dropped on glass slides, treated with 0.025% trypsin in 0.9% NaCl, and stained with 1:4 diluted Wright's stain (pH 6.8) for Giemsa-Trypsin-Wright banding. Metaphases are analyzed under a microscope at a magnification of 1250× for chromosome count and structural integrity. A minimum of twenty metaphases are analyzed for each sample. Karyotypes are written according to the ISCN2009 guideline using the mouse chromosome band designation specified in ideogram found from the following website: www.informatics.jax.org/silver/images/figure5-2.gif. It will be observed that FV-FancA vector transduced cells exhibit normal karyotypes.

Results. A fanca−/− mouse model developed by Noll et al. (Noll M et al. (2002) Experimental Hematology 30: 679-688) is used to assess efficacy of FV-FANCA combined with HSPC mobilization to treat FA (Prophetic). It will be observed that fanca −/− mice administered FV vector including a PGK promoter associated with a FANCA gene or fanca −/− mice administered FV vector including a PGK promoter associated with a FANCA gene along with mobilization factors G-CSF and AMD3100 have one or more of the following: increased thymic output; restored T lymphocyte development; diverse TCR repertoire; restored T-cell specific signaling pathways; increased white blood cell count, neutrophil cell count, monocyte cell count, lymphocyte cell count, and/or platelet cell count; increased number of circulating T lymphocytes; increased absolute CD3+ lymphocyte count; increased frequency of gene corrected lymphocytes or bone marrow derived cells; increased diversity of clonal repertoire of gene corrected lymphocytes or bone marrow derived cells; restored bone marrow function; improved bone marrow repopulation with gene corrected cells; normalized primary and secondary antibody responses to immunization; restored B-cell and/or T-cell cytokine signaling programs functioning in class switching and memory response to an antigen; increased level of one or more immunoglobulins selected from IgA, IgM, and IgG; and/or increased resistance of bone marrow derived cells to mitomycin C (MMC).

### EXAMPLE 4

(Prophetic). Intra bone marrow delivery of FV-FANCA to treat FA. Material and Methods. Animal procedures, gene marking, phenotypic analysis, methylcellulose and MMC resistance assays, and cytogenetic analyses are performed as described in Example 3.

Intra bone marrow delivery of FV-FANCA can be performed using a delivery method as described in Kushida T et al. (2001) Blood 97:3292-3299. The region from the inguen to the knee joint is shaved of hair with a razor and a 5-mm incision is made on the thigh. The knee is flexed to 90° and the proximal side of the tibia is drawn to the anterior. A 26-gauge needle is inserted into the joint surface of the tibia through the patellar tendon and then inserted into the bone marrow cavity. A microsyringe can be used to inject FV-FANCA into the bone marrow. Intra bone marrow delivery can target delivery of FV-FANCA to mesenchymal stem cells.

Results. A fanca−/− mouse model developed by Noll et al. (Noll M et al. (2002) Experimental Hematology 30: 679-688) is used to assess efficacy of intra bone marrow delivery of FV-FANCA to treat FA (Prophetic). It will be observed that fanca −/− mice administered FV vector including a PGK promoter associated with a FANCA gene have one or more of the following: increased thymic output; restored T lymphocyte development; diverse TCR repertoire; restored T-cell specific signaling pathways; increased white blood cell count, neutrophil cell count, monocyte cell count, lymphocyte cell count, and/or platelet cell count; increased number of circulating T lymphocytes; increased absolute CD3+ lymphocyte count; increased frequency of gene corrected lymphocytes or bone marrow derived cells; increased diversity of clonal repertoire of gene corrected lymphocytes or bone marrow derived cells; restored bone marrow function; improved bone marrow repopulation with gene corrected cells; normalized primary and secondary antibody responses to immunization; restored B-cell and/or T-cell cytokine signaling programs functioning in class switching and memory response to an antigen; increased level of one or more immunoglobulins selected from IgA, IgM, and IgG; and/or increased resistance of bone marrow derived cells to mitomycin C (MMC).

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means includes, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect would cause a statistically-significant decrease in primary and secondary antibody responses to immunization in a SCID-X1 or FA subject administered a FV vector including a PGK promoter associated with γC (for SCID-X1) or a FV vector including a PGK promoter associated with FANCA for FA.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary, 3^{rd} Edition or a dictionary known to those of ordinary skill in the art, such as the Oxford Dictionary of Biochemistry and Molecular Biology (Ed. Anthony Smith, Oxford University Press, Oxford, 2004).

What is claimed is:

1. A method of treating X-linked severe combined immunodeficiency (SCID-X1) in a subject in need thereof comprising administering a therapeutically effective amount of (i) a formulation comprising a foamy viral vector comprising a PGK promoter associated with a sequence encoding a γC protein; (ii) G-CSF; and (iii) AMD3100, thereby treating SCID-X1 in the subject in need thereof.

2. The method of claim 1, wherein the foamy viral vector comprises a sequence selected from SEQ ID NOs: 1-3, 26, and 28-30.

3. The method of claim 1, wherein the foamy viral vector comprises a sequence encoding a sequence selected from SEQ ID NOs: 4, 5, and 27.

4. The method of claim 1, wherein the γC protein restores functionality to a γC-dependent signaling pathway.

5. The method of claim 4, wherein the functionality of a γC-dependent signaling pathway is determined by measuring tyrosine phosphorylation of STAT3 and/or STAT5 in cells from the subject following in vitro stimulation with IL-21 and IL-2, respectively.

6. The method of claim 5, wherein the tyrosine phosphorylation of STAT3 and/or STAT5 is measured by intracellular antibody staining.

7. The method of claim 5, wherein the cells are peripheral blood mononuclear cells (PBMCs).

8. The method of claim 1, wherein the therapeutically effective amount of the foamy viral vector is 1×10⁸ to 10×10⁸ infection units (IU).

9. The method of claim 1, wherein the therapeutically effective amount of G-CSF is 1 μg/kg to 10 μg/kg.

10. The method of claim 1, wherein the therapeutically effective amount of AMD3100 is 1 mg/kg to 10 mg/kg.

11. The method of claim 1, wherein the therapeutically effective amount of G-CSF is administered prior to administration of the therapeutically effective amount of the formulation comprising the foamy viral vector.

12. The method of claim 1 comprising a 5 day treatment protocol, wherein G-CSF is administered on day 1, day 2, day 3, and day 4 and on day 5, G-CSF and AMD3100 are administered 6 to 8 hours prior to administration of the foamy viral vector.

13. A method of treating Fanconi anemia (FA) in a subject in need thereof comprising administering a therapeutically effective amount of a formulation comprising a foamy viral vector comprising a PGK promoter associated with a sequence encoding a FancA protein, thereby treating FA in the subject in need thereof.

14. The method of claim 13 further comprising administering a therapeutically effective amount of G-CSF and AMD3100.

15. The method of claim 13, wherein the foamy viral vector comprises a sequence selected from SEQ ID NOs: 16, 26, 28, 32, and 33.

16. The method of claim 13, wherein the foamy viral vector comprises a sequence encoding a sequence selected from SEQ ID NOs: 21 and 27.

17. The method of claim 13, wherein the FancA protein increases resistance of bone marrow derived cells to mitomycin C (MMC).

18. The method of claim 17, wherein the resistance of bone marrow derived cells to MMC is measured by a cell survival assay in methylcellulose and MMC.

19. The method of claim 17, wherein the therapeutically effective amount of the foamy viral vector is 1×10⁸ to 10×10⁸ infection units (IU).

20. The method of claim 14, wherein the therapeutically effective amount of G-CSF is 1 μg to 10 μg.

21. The method of claim 14, wherein the therapeutically effective amount of AMD3100 is 1 mg/kg to 10 mg/kg.

22. The method of claim 14, wherein the therapeutically effective amount of G-CSF is administered for at least 4 consecutive days prior to administration of the therapeutically effective amount of the formulation comprising the foamy viral vector.

23. The method of claim 14, wherein G-CSF is administered every 12 hours for at least 4 consecutive days, followed by administration of AMD3100 14 hours after the last dose of G-CSF and 1 hour prior to administration of the foamy viral vector.

24. The method of claim 1 or 14, wherein G-CSF comprises a sequence selected from SEQ ID NOs: 34-37.

25. The method of claim 1 or 13, wherein the administration of (i), (ii), and (iii) are selected from subcutaneous delivery, intravenous delivery, and intra bone marrow delivery.

26. The method of claim 1 or 13, wherein the administration targets mesenchymal stem cells.

27. The method of claim 1 or 14, wherein the therapeutically effective amount of AMD3100 is administered following the administration of the therapeutically effective amount of G-CSF.

28. The method of claim 1 or 14, wherein the therapeutically effective amount of AMD3100 is administered concurrently with the therapeutically effective amount of G-CSF.

29. The method of claim 1 or 14, wherein the therapeutically effective amount of AMD3100 is administered prior to the administration of the therapeutically effective amount of the formulation comprising the foamy viral vector.

30. The method of claim 1 or 14, wherein G-CSF is part of the formulation comprising the foamy viral vector.

31. The method of claim 1 or 14, wherein AMD3100 is part of the formulation comprising the foamy viral vector.

32. The method of claim 1 or 14, wherein G-CSF and AMD3100 are part of the formulation comprising the foamy viral vector.

33. The method of claim 1 or 13, wherein the foamy viral vector further comprises an in vivo selection marker.

34. The method of claim 33, wherein the in vivo selection marker is MGMT P140K.

35. A method of restoring T-cell mediated immune responses in a subject in need thereof comprising administering a therapeutically effective amount of (i) a formulation comprising a foamy viral vector comprising (a) a PGK promoter and (b) a sequence encoding a therapeutic protein; and (ii) mobilization factors, thereby restoring T-cell mediated immune responses in the subject in need thereof.

36. The method of claim 35, wherein restoring T-cell mediated immune responses in a subject in need thereof comprises increasing thymic output.

37. The method of claim 36, wherein increasing thymic output comprises increasing the frequency of CD3+ T cells expressing CD45RA in peripheral blood to a level comparable to that of a reference level derived from a normal control population.

38. The method of claim 36, wherein increasing thymic output comprises increasing the frequency of CD3+ T cells expressing CD45RA in peripheral blood to a level greater than that of a subject in need thereof not administered the therapeutically effective amount of the formulation.

39. The method of claim 36, wherein increasing thymic output comprises increasing the frequency of CD3+ T cells expressing CD45RA in peripheral blood to a level greater than that of a subject in need thereof administered the therapeutically effective amount of the formulation not comprising mobilization factors.

40. The method of claim 36, wherein increasing thymic output comprises increasing the number of T cell receptor excision circles (TRECs) per 10⁶ maturing T cells to a level comparable to that of a reference level derived from a normal control population.

41. The method of claim 36, wherein increasing thymic output comprises increasing the number of T cell receptor excision circles (TRECs) per 10⁶ maturing T cells to a level greater than that of a subject in need thereof not administered the therapeutically effective amount of the formulation.

42. The method of claim 36, wherein increasing thymic output comprises increasing the number of T cell receptor excision circles (TRECs) per 10⁶ maturing T cells to a level greater than that of a subject in need thereof administered the therapeutically effective amount of the formulation not comprising mobilization factors.

43. The method of claim 35, wherein restoring T-cell mediated immune responses in a subject in need thereof comprises restoring normal T lymphocyte development.

44. The method of claim 43, wherein restoring normal T lymphocyte development comprises restoring the ratio of CD4+ cells: CD8+ cells to 2.

45. The method of claim 43, wherein restoring normal T lymphocyte development comprises detecting the presence of αβ TCR in circulating T-lymphocytes.

46. The method of claim 43, wherein detecting the presence of αβ TCR in circulating T-lymphocytes comprises detecting the αβ TCR by flow cytometry.

47. The method of claim 43, wherein restoring normal T lymphocyte development comprises detecting the presence of a diverse TCR repertoire comparable to that of a reference level derived from a normal control population.

48. The method of claim 47, wherein detecting the presence of a diverse TCR repertoire comprises spectratyping TCRVβ.

49. The method of claim 48, wherein restoring normal T lymphocyte development comprises restoring one or more T-cell specific signaling pathways.

50. The method of claim 49, wherein restoring one or more T-cell specific signaling pathways can be assessed by lymphocyte proliferation following exposure to T cell mitogen phytohemagglutinin (PHA).

51. The method of claim 43, wherein restoring normal T lymphocyte development comprises increasing white blood cell count, neutrophil cell count, monocyte cell count, lymphocyte cell count, and/or platelet cell count to a level comparable to that of a reference level derived from a normal control population.

52. The method of claim 43, wherein restoring normal T lymphocyte development comprises increasing white blood cell count, neutrophil cell count, monocyte cell count, lymphocyte cell count, and/or platelet cell count to a level greater than that of a subject in need thereof not administered the therapeutically effective amount of the formulation.

53. The method of claim 43, wherein restoring normal T lymphocyte development comprises increasing white blood cell count, neutrophil cell count, monocyte cell count, lymphocyte cell count, and/or platelet cell count to a level greater than that of a subject in need thereof administered the therapeutically effective amount of the formulation not comprising mobilization factors.

54. A method of improving the kinetics and clonal diversity of lymphocyte reconstitution in a subject in need thereof comprising administering a therapeutically effective amount of (i) a formulation comprising a foamy viral vector comprising a PGK promoter associated with a sequence encoding a therapeutic protein; and (ii) mobilization factors, thereby improving the kinetics and clonal diversity of lymphocyte reconstitution in the subject in need thereof.

55. The method of claim 54, wherein improving the kinetics of lymphocyte reconstitution comprises increasing the number of circulating T lymphocytes to within a range of a reference level derived from a normal control population.

56. The method of claim 54, wherein improving the kinetics of lymphocyte reconstitution comprises increasing the number of circulating T lymphocytes as compared to that of a subject in need thereof not administered the therapeutically effective amount of the formulation.

57. The method of claim 54, wherein improving the kinetics of lymphocyte reconstitution comprises increasing the number of circulating T lymphocytes as compared to that of a subject in need thereof administered the therapeutically effective amount of the formulation not comprising mobilization factors.

58. The method of claim 54, wherein improving the kinetics of lymphocyte reconstitution comprises reducing the time required to reach normal lymphocyte counts as compared to that of a subject in need thereof not administered the therapeutically effective amount of the formulation.

59. The method of claim 54, wherein improving the kinetics of lymphocyte reconstitution comprises reducing the time required to reach normal lymphocyte counts as compared to that of a subject in need thereof administered the therapeutically effective amount of the formulation not comprising mobilization factors.

60. The method of claim 54, wherein improving the kinetics of lymphocyte reconstitution comprises increasing the absolute CD3+ lymphocyte count to within a range of a reference level derived from a normal control population.

61. The method of claim 54, wherein improving the kinetics of lymphocyte reconstitution comprises increasing the absolute CD3+ lymphocyte count as compared to that of a subject in need thereof not administered the therapeutically effective amount of the formulation.

62. The method of claim 54, wherein improving the kinetics of lymphocyte reconstitution comprises increasing the absolute CD3+ lymphocyte count as compared to that of a subject in need thereof administered the therapeutically effective amount of the formulation not comprising mobilization factors.

63. The method of claim 54, wherein improving the kinetics of lymphocyte reconstitution comprises increasing the frequency of gene corrected lymphocytes as compared to a subject in need thereof not administered the therapeutically effective amount of the formulation.

64. The method of claim 54, wherein improving the kinetics of lymphocyte reconstitution comprises increasing the frequency of gene corrected lymphocytes as compared to a subject in need thereof administered the therapeutically effective amount of the formulation not comprising mobilization factors.

65. The method of claim 54, wherein improving the kinetics of lymphocyte reconstitution comprises increasing diversity of clonal repertoire of gene corrected lymphocytes in the subject as compared to a subject in need thereof not administered the therapeutically effective amount of the formulation.

66. The method of claim 54, wherein improving the kinetics of lymphocyte reconstitution comprises increasing diversity of clonal repertoire of gene corrected lymphocytes in the subject as compared to a subject in need thereof administered the therapeutically effective amount of the formulation not comprising mobilization factors.

67. The method of claim 65 or 66, wherein increasing diversity of clonal repertoire of gene corrected lymphocytes comprises increasing the number of unique retroviral integration site (RIS) clones as measured by a RIS analysis.

68. A method of restoring bone marrow function in a subject in need thereof comprising administering a therapeutically effective amount of (i) a formulation comprising a foamy viral vector comprising a PGK promoter associated with a sequence encoding a therapeutic protein; and (ii) mobilization factors, thereby restoring bone marrow function in the subject in need thereof.

69. The method of claim 68, wherein restoring bone marrow function comprises improving bone marrow repopulation with gene corrected cells in the subject as compared to a subject in need thereof not administered the therapeutically effective amount of the formulation.

70. The method of claim 68, wherein restoring bone marrow function comprises improving bone marrow repopulation with gene corrected cells in the subject as compared to a subject in need thereof administered the therapeutically effective amount of the formulation not comprising mobilization factors.

71. The method of claim 69 or 70, wherein improving bone marrow repopulation with gene corrected cells comprises increasing the percentage of cells that are gene corrected.

72. The method of claim 69 or 70, wherein the cells are selected from white blood cells and bone marrow derived cells.

73. The method of claim 71, wherein the percentage of cells that are gene corrected is measured using an assay selected from quantitative real time PCR and flow cytometry.

74. A method of normalizing primary and secondary antibody responses to immunization in a subject in need thereof comprising administering a therapeutically effective amount of (i) a formulation comprising a foamy viral vector comprising a PGK promoter associated with a sequence encoding a therapeutic protein; and (ii) mobilization factors, thereby normalizing primary and secondary antibody responses to immunization in the subject in need thereof.

75. The method of claim 74, wherein normalizing primary and secondary antibody responses to immunization in a subject in need thereof comprises restoring B-cell and/or T-cell cytokine signaling programs functioning in class switching and memory response to an antigen.

76. The method of claim 75, wherein restoring B-cell and/or T-cell cytokine signaling programs is measured by a bacteriophage immunization assay.

77. The method of claim 74, wherein normalizing primary and secondary antibody responses to immunization in a subject in need thereof comprises increasing the level of one or more immunoglobulins selected from IgA, IgM, and IgG in a subject in need thereof to a level comparable to that of corresponding immunoglobulins in a reference level derived from a normal control population.

78. The method of claim 77, wherein normalizing primary and secondary antibody responses to immunization in a subject in need thereof comprises increasing the level of one or more immunoglobulins selected from IgA, IgM, and IgG in a subject in need thereof to a level greater than that of corresponding immunoglobulins in a subject in need thereof not administered the therapeutically effective amount of the formulation.

79. The method of claim 77, wherein normalizing primary and secondary antibody responses to immunization in a subject in need thereof comprises increasing the level of one or more immunoglobulins selected from IgA, IgM, and IgG in a subject in need thereof to a level greater than that of corresponding immunoglobulins in a subject in need thereof administered the therapeutically effective amount of the formulation not comprising mobilization factors.

80. The method of any one of claims 77-79, wherein the increase in the level of one or more immunoglobulins selected from IgA, IgM, and IgG in a subject in need thereof is measured by an assay selected from serum protein electrophoresis, immunoelectrophoresis, radial immunodiffusion, nephelometry and turbidimetry.

81. The method of any of claim 35-66, 68-70 or 74-79, wherein the subject is in need thereof due to a primary or secondary immune deficiency.

82. The method of claim 81, wherein the subject is in need thereof due to SCID.

83. The method of claim 82, wherein SCID is SCID-X1.

84. The method of claim 81, wherein the subject is in need thereof due to FA.

85. The method of any of claim 35-66, 68-70 or 74-79, wherein the therapeutic protein comprises the common gamma chain gene (γC) and/or FancA.

86. The method of any of claim 35-66, 68-70 or 74-79, wherein the foamy viral vector comprises a sequence selected from SEQ ID NOs: 1-3, 6-10, 16-20, 26, 28-30, 32, and 33.

87. The method of any of claim 35-66, 68-70 or 74-79, wherein the foamy viral vector comprises a sequence encoding a sequence selected from SEQ ID NOs: 4, 5, 11-15, 21-25, and 27.

88. The method of any one of claim 35-66, 68-70 or 74-79, wherein the mobilization factors comprise G-CSF/Filgrastim (Amgen), GM-CSF, AMD3100 (Sigma), SCF, and/or a chemotherapeutic agent.

89. The method of claim 88, wherein the chemotherapeutic agent is selected from cyclophosphamide, etoposide, ifosfamide, cisplatin, and cytarabine.

90. The method of claim 88, wherein the mobilization factors comprise G-CSF/Filgrastim (Amgen) and AMD3100 (Sigma).

91. The method of claim 88, wherein the mobilization factors comprise a sequence selected from SEQ ID NOs: 34-39.

92. The method of any of claim 35-66, 68-70 or 74-79, wherein the foamy viral vector further comprises an in vivo selection marker.

93. The method of claim 92, wherein the in vivo selection marker is MGMT P140K.

94. A formulation comprising: a therapeutically effective amount of a foamy viral vector comprising a PGK promoter associated with a therapeutic gene; and a pharmaceutically acceptable carrier.

95. The formulation of claim 94, wherein the foamy viral vector comprises a sequence selected from SEQ ID NOs: 1-3, 6-10, 16-20, 26, 28-30, 32, and 33.

96. The formulation of claim 94, wherein the foamy viral vector comprises a sequence encoding a sequence selected from SEQ ID NOs: 4, 5, 11-15, 21-25, and 27.

97. The formulation of claim 94, wherein the PGK promoter comprises a human PGK promoter.

98. The formulation of claim 94, wherein the PGK promoter comprises SEQ ID NO: 28.

99. The formulation of claim 94, wherein the therapeutic gene comprises γC.

100. The formulation of claim 94, wherein the therapeutic gene comprises FancA.

101. The formulation of claim 94, wherein the therapeutic gene comprises a sequence selected from SEQ ID NOs: 1-3, 6-10, and 16-20.

102. The formulation of claim 94, wherein the therapeutic gene comprises a sequence encoding a sequence selected from SEQ ID NOs: 4, 5, 11-15, and 21-25.

103. The formulation of claim 94, wherein the foamy viral vector further comprises an in vivo selection marker.

104. The formulation of claim 103, wherein the in vivo selection marker is MGMT P140K.

105. The formulation of claim 94 further comprising one or more mobilization factors.

106. The formulation of claim 105, wherein the one or more mobilization factors comprise G-CSF/Filgrastim (Amgen), GM-CSF, AMD3100 (Sigma), SCF, and/or a chemotherapeutic agent.

107. The formulation of claim 106, wherein the chemotherapeutic agent is selected from cyclophosphamide, etoposide, ifosfamide, cisplatin, and cytarabine.

108. The formulation of claim 106, wherein the one or more mobilization factors comprise G-CSF/Filgrastim (Amgen) and AMD3100 (Sigma).

109. The formulation of claim 105, wherein the one or more mobilization factors comprise SEQ ID NOs: 34-39.

110. A kit comprising:
a formulation comprising a therapeutically effective amount of a foamy viral vector comprising a PGK promoter associated with a therapeutic gene; and a pharmaceutically acceptable carrier; and
one or more mobilization factors.

111. The kit of claim 110, wherein the foamy viral vector comprises a sequence selected from SEQ ID NOs: 1-3, 6-10, 16-20, 26, 28-30, 32, and 33.

112. The kit of claim 110, wherein the foamy viral vector comprises a sequence encoding a sequence selected from SEQ ID NOs: 4, 5, 11-15, 21-25, and 27.

113. The kit of claim 110, wherein the PGK promoter comprises a human PGK promoter.

114. The kit of claim 110, wherein the PGK promoter comprises SEQ ID NO: 28.

115. The kit of claim 110, wherein the therapeutic gene comprises γC.

116. The kit of claim 110, wherein the therapeutic gene comprises FancA.

117. The kit of claim 110, wherein the therapeutic gene comprises a sequence selected from SEQ ID NOs: 1-3, 6-10, and 16-20.

118. The kit of claim 110, wherein the therapeutic gene comprises a sequence encoding a sequence selected from SEQ ID NOs: 4, 5, 11-15, and 21-25.

119. The kit of claim 110, wherein the foamy viral vector further comprises an in vivo selection marker.

120. The kit of claim 119, wherein the in vivo selection marker is MGMT P140K.

121. The kit of claim 110, wherein the one or more mobilization factors comprise G-CSF/Filgrastim (Amgen), GM-CSF, AMD3100 (Sigma), SCF, and/or a chemotherapeutic agent.

122. The kit of claim 121, wherein the chemotherapeutic agent is selected from cyclophosphamide, etoposide, ifosfamide, cisplatin, and cytarabine.

123. The kit of claim 110, wherein the one or more mobilization factors comprise G-CSF/Filgrastim (Amgen) and AMD3100 (Sigma).

124. The kit of claim 110, wherein the one or more mobilization factors comprise SEQ ID NOs: 34-39.

125. The kit of claim 110 further comprising instructions on administering the formulation and the one or more mobilization factors.


SEQUENCE LISTING (44 sequences)

SEQ ID NO: 1 - DNA, 1110 bp, artificial
  codon optimized human gammaC
atgctgaaaccaagcctgccctttacaagcctgctgttcctgcagctgccactgctgggggtcggactgaatactacaatcctgacaccaaacggaaatgaggacaccacagccgatttctttctgactaccatgcccactgacagtctgtcagtgagcaccctgccactgcccgaggtccagtgcttcgtgtttaacgtcgaatatatgaactgtacctggaatagctcctctgaacctcagccaacaaatctgactctgcactactggtataagaactctgacaatgataaggtgcagaaatgctcacattatctgttcagcgaggaaatcacctccggctgtcagctgcagaagaaagagattcacctgtaccagacatttgtggtccagctgcaggatccccgggaacctcggagacaggccactcagatgctgaagctgcagaacctggtcatcccatgggctcccgagaatctgaccctgcataaactgtccgagtctcagctggaactgaactggaacaataggttcctgaatcactgcctggagcatctggtgcagtaccgcacagactgggatcactcttggactgaacagagtgtggactatcgacataagtttagtctgccttcagtggatgggcagaaaaggtacacattcagggtccgctctcggttcaacccactgtgcggaagcgcccagcactggagcgagtggtcccaccccatccattgggggtctaacaccagcaaggagaatcctttcctgtttgccctggaagctgtggtcatttcagtgggaagcatgggcctgatcattagcctgctgtgcgtgtacttctggctggagcggaccatgcctagaatcccaacactgaagaacctggaggacctggtgacagaatatcacggcaatttttccgcttggtctggggtcagtaaaggactggcagagagcctgcagcccgattactccgagcggctgtgcctggtgtccgaaattccccctaaaggcggggcactgggagaaggccctggggcctccccctgcaaccagcactcaccctattgggcaccaccctgttacaccctgaaacccgaaacttaa

SEQ ID NO: 2 - DNA, 1110 bp, Homo sapiens
atgttgaagccatcattaccattcacatccctcttattcctgcagctgcccctgctgggagtggggctgaacacgacaattctgacgcccaatgggaatgaagacaccacagctgatttcttcctgaccactatgcccactgactccctcagcgtttccactctgcccctcccagaggttcagtgttttgtgttcaatgtcgagtacatgaattgcacttggaacagcagctctgagccccagcctaccaacctcactctgcattattggtacaagaactcggataatgataaagtccagaagtgcagccactatctattctctgaagaaatcacttctggctgtcagttgcaaaaaaaggagatccacctctaccaaacatttgttgttcagctccaggacccacgggaacccaggagacaggccacacagatgctaaaactgcagaatctggtgatcccctgggctccagagaacctaacacttcacaaactgagtgaatcccagctagaactgaactggaacaacagattcttgaaccactgtttggagcacttggtgcagtaccggactgactgggaccacagctggactgaacaatcagtggattatagacataagttctccttgcctagtgtggatgggcagaaacgctacacgtttcgtgttcggagccgctttaacccactctgtggaagtgctcagcattggagtgaatggagccacccaatccactgggggagcaatacttcaaaagagaatcctttcctgtttgcattggaagccgtggttatctctgttggctccatgggattgattatcagccttctctgtgtgtatttctggctggaacggacgatgccccgaattcccaccctgaagaacctagaggatcttgttactgaataccacgggaacttttcggcctggagtggtgtgtctaagggactggctgagagtctgcagccagactacagtgaacgactctgcctcgtcagtgagattcccccaaaaggaggggcccttggggaggggcctggggcctccccatgcaaccagcatagcccctactgggcccccccatgttacaccctaaagcctgaaacctga

SEQ ID NO: 3 - DNA, 1114 bp, Canis lupus familiaris
atgttgaagccaccattgccactcagatccctcttattcctgcagctgtctctgctgggggtggggctgaactccacggtccccatgcccaatgggaatgaagacatcacacctgatttcttcctgaccgctacaccctccgagaccctcagtgtttcctccctgcccctcccagaggtccagtgttttgtgttcaatgttgagtacatgaattgcacttggaacagcagctctgagccccggcccaccaacctgaccctgcactactggtataagaactccaatgatgataaagtccaggagtgtggccactacctattctctagagaggtcactgctggctgttggttgcagaaggaggagatccatctctacgaaacatttgttgtccagctccgggacccacgggaacccaggaggcagtccacacagaagctaaaactgcaaaatctggtgatcccctgggctccggagaacctaacccttcacaacctgagcgaatcccagctagaactgagctggagcaacagacacttggaccactgtttggagcatgttgtgcagtaccggagtgactgggaccgcagctggactgaacagtcagtggaccaccgaaatagcttctctctgcctagcgtggatgggcagaagttctacacgttccgtgtccgaagccgctataacccactctgtggaagcgctcagcgttggagtgaatggagccaccctatccactgggggagcaatacctccaaggagaatcctttgtttgcatcggaagctgtgcttatcccccttggctccatgggattgattattagccttatctgtgtgtactactggctggaacggtcgatcccccgaattcctaccctcaagaacctggaggatctggttactgaatatcacgggaatttttcggcctggagtggagtgtctaagggactggcggagagtctgcagccagactacagtgaatggctctgccacgtcagtgagattcccccaaaaggaggggctccaggggagggtcctgggggctccccctgcagccagcatagcccctactgggctcccccatgttataccctgaaacctgaaactggagccctga

SEQ ID NO: 4 - protein, 369 aa, Homo sapiens
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 5 - protein, 373 aa, Canis lupus familiaris
MLKPPLPLRSLLFLQLSLLGVGLNSTVPMPNGNEDITPDFFLTATPSETLSVSSLPLPEVQCFVFNVEYMNCTWNSSSEPRPTNLTLHYWYKNSNDDKVQECGHYLFSREVTAGCWLQKEEIHLYETFVVQLRDPREPRRQSTQKLKLQNLVIPWAPENLTLHNLSESQLELSWSNRHLDHCLEHVVQYRSDWDRSWTEQSVDHRNSFSLPSVDGQKFYTFRVRSRYNPLCGSAQRWSEWSHPIHWGSNTSKENPLFASEAVLIPLGSMGLIISLICVYYWLERSIPRIPTLKNLEDLVTEYHGNFSAWSGVSKGLAESLQPDYSEWLCHVSEIPPKGGAPGEGPGGSPCSQHSPYWAPPCYTLKPETGALIP

SEQ ID NO: 6 - DNA, 3375 bp, Homo sapiens
atggcacctccaagtgaagagacgcccctgatccctcagcgttcatgcagcctcttgtccacggaggctggtgccctgcatgtgctgctgcccgctcggggccccgggcccccccagcgcctatctttctcctttggggaccacttggctgaggacctgtgcgtgcaggctgccaaggccagcggcatcctgcctgtgtaccactccctctttgctctggccacggaggacctgtcctgctggttccccccgagccacatcttctccgtggaggatgccagcacccaagtcctgctgtacaggattcgcttttacttccccaattggtttgggctggagaagtgccaccgcttcgggctacgcaaggatttggccagtgctatccttgacctgccagtcctggagcacctctttgcccagcaccgcagtgacctggtgagtgggcgcctccccgtgggcctcagtctcaaggagcagggtgagtgtctcagcctggccgtgttggacctggcccggatggcgcgagagcaggcccagcggccgggagagctgctgaagactgtcagctacaaggcctgcctacccccaagcctgcgcgacctgatccagggcctgagcttcgtgacgcggaggcgtattcggaggacggtgcgcagagccctgcgccgcgtggccgcctgccaggcagaccggcactcgctcatggccaagtacatcatggacctggagcggctggatccagccggggccgccgagaccttccacgtgggcctccctggggcccttggtggccacgacgggctggggctgctccgcgtggctggtgacggcggcatcgcctggacccagggagaacaggaggtcctccagcccttctgcgactttccagaaatcgtagacattagcatcaagcaggccccgcgcgttggcccggccggagagcaccgcctggtcactgttaccaggacagacaaccagattttagaggccgagttcccagggctgcccgaggctctgtcgttcgtggcgctcgtggacggctacttccggctgaccacggactcccagcacttcttctgcaaggaggtggcaccgccgaggctgctggaggaagtggccgagcagtgccacggccccatcactctggactttgccatcaacaagctcaagactgggggctcacgtcctggctcctatgttctccgccgcagcccccaggactttgacagcttcctcctcactgtctgtgtccagaacccccttggtcctgattataagggctgcctcatccggcgcagccccacaggaaccttccttctggttggcctcagccgaccccacagcagtcttcgagagctcctggcaacctgctgggatggggggctgcacgtagatggggtggcagtgaccctcacttcctgctgtatccccagacccaaagaaaagtccaacctgatcgtggtccagagaggtcacagcccacccacatcatccttggttcagccccaatcccaataccagctgagtcagatgacatttcacaagatccctgctgacagcctggagtggcatgagaacctgggccatgggtccttcaccaagatttaccggggctgtcgccatgaggtggtggatggggaggcccgaaagacagaggtgctgctgaaggtcatggatgccaagcacaagaactgcatggagtcattcctggaagcagcgagcttgatgagccaagtgtcgtaccggcatctcgtgctgctccacggcgtgtgcatggctggagacagcaccatggtgcaggaatttgtacacctgggggccatagacatgtatctgcgaaaacgtggccacctggtgccagccagctggaagctgcaggtggtcaaacagctggcctacgccctcaactatctggaggacaaaggcctgccccatggcaatgtctctgcccggaaggtgctcctggctcgggagggggctgatgggagcccgcccttcatcaagctgagtgaccctggggtcagccccgctgtgttaagcctggagatgctcaccgacaggatcccctgggtggcccccgagtgtctccgggaggcgcagacacttagcttggaagctgacaagtggggcttcggcgccacggtctgggaagtgtttagtggcgtcaccatgcccatcagtgccctggatcctgctaagaaactccaattttatgaggaccggcagcagctgccggcccccaagtggacagagctggccctgctgattcaacagtgcatggcctatgagccggtccagaggccctccttccgagccgtcattcgtgacctcaatagcctcatctcttcagactatgagctcctctcagaccccacacctggtgccctggcacctcgtgatgggctgtggaatggtgcccagctctatgcctgccaagaccccacgatcttcgaggagagacacctcaagtacatctcacagctgggcaagggcaactttggcagcgtggagctgtgccgctatgacccgctaggcgacaatacaggtgccctggtggccgtgaaacagctgcagcacagcgggccagaccagcagagggactttcagcgggagattcagatcctcaaagcactgcacagtgatttcattgtcaagtatcgtggtgtcagctatggcccgggccgccagagcctgcggctggtcatggagtacctgcccagcggctgcttgcgcgacttcctgcagcggcaccgcgcgcgcctcgatgccagccgcctccttctctattcctcgcagatctgcaagggcatggagtacctgggctcccgccgctgcgtgcaccgcgacctggccgcccgaaacatcctcgtggagagcgaggcacacgtcaagatcgctgacttcggcctagctaagctgctgccgcttgacaaagactactacgtggtccgcgagccaggccagagccccattttctggtatgcccccgaatccctctcggacaacatcttctctcgccagtcagacgtctggagcttcggggtcgtcctgtacgagctcttcacctactgcgacaaaagctgcagcccctcggccgagttcctgcggatgatgggatgtgagcgggatgtccccgccctctgccgcctcttggaactgctggaggagggccagaggctgccggcgcctcctgcctgccctgctgaggttcacgagctcatgaagctgtgctgggcccctagcccacaggaccggccatcattcagcgccctgggcccccagctggacatgctgtggagcggaagccgggggtgtgagactcatgccttcactgctcacccagagggcaaacaccactccctgtccttttcatag

SEQ ID NO: 7 - DNA, 870 bp, Homo sapiens
atggagaacggatacacctatgaagattataagaacactgcagaatggcttctgtctcacactaagcaccgacctcaagttgcaataatctgtggttctggattaggaggtctgactgataaattaactcaggcccagatctttgactacggtgaaatccccaactttccccgaagtacagtgccaggtcatgctggccgactggtgtttgggttcctgaatggcagggcctgtgtgatgatgcagggcaggttccacatgtatgaagggtacccactctggaaggtgacattcccagtgagggttttccaccttctgggtgtggacaccctggtagtcaccaatgcagcaggagggctgaaccccaagtttgaggttggagatatcatgctgatccgtgaccatatcaacctacctggtttcagtggtcagaaccctctcagagggcccaatgatgaaaggtttggagatcgtttccctgccatgtctgatgcctacgaccggactatgaggcagagggctctcagtacctggaaacaaatgggggagcaacgtgagctacaggaaggcacctatgtgatggtggcaggccccagctttgagactgtggcagaatgtcgtgtgctgcagaagctgggagcagacgctgttggcatgagtacagtaccagaagttatcgttgcacggcactgtggacttcgagtctttggcttctcactcatcactaacaaggtcatcatggattatgaaagcctggagaaggccaaccatgaagaagtcttagcagctggcaaacaagctgcacagaaattggaacagtttgtctccattcttatggccagcattccactccctgacaaagccagttga

SEQ ID NO: 8 - DNA, 1092 bp, Homo sapiens
atggcccagacgcccgccttcgacaagcccaaagtagaactgcatgtccacctagacggatccatcaagcctgaaaccatcttatactatggcaggaggagagggatcgccctcccagctaacacagcagaggggctgctgaacgtcattggcatggacaagccgctcacccttccagacttcctggccaagtttgactactacatgcctgctatcgcgggctgccgggaggctatcaaaaggatcgcctatgagtttgtagagatgaaggccaaagagggcgtggtgtatgtggaggtgcggtacagtccgcacctgctggccaactccaaagtggagccaatcccctggaaccaggctgaaggggacctcaccccagacgaggtggtggccctagtgggccagggcctgcaggagggggagcgagacttcggggtcaaggcccggtccatcctgtgctgcatgcgccaccagcccaactggtcccccaaggtggtggagctgtgtaagaagtaccagcagcagaccgtggtagccattgacctggctggagatgagaccatcccaggaagcagcctcttgcctggacatgtccaggcctaccaggaggctgtgaagagcggcattcaccgtactgtccacgccggggaggtgggctcggccgaagtagtaaaagaggctgtggacatactcaagacagagcggctgggacacggctaccacaccctggaagaccaggccctttataacaggctgcggcaggaaaacatgcacttcgagatctgcccctggtccagctacctcactggtgcctggaagccggacacggagcatgcagtcattcggctcaaaaatgaccaggctaactactcgctcaacacagatgacccgctcatcttcaagtccaccctggacactgattaccagatgaccaaacgggacatgggctttactgaagaggagtttaaaaggctgaacatcaatgcggccaaatctagtttcctcccagaagatgaaaagagggagcttctcgacctgctctataaagcctatgggatgccaccttcagcctctgcagggcagaacctctga

SEQ ID NO: 9 - DNA, 3132 bp, Homo sapiens
atggcagcctctttcccacccaccttgggactcagttctgccccagatgaaattcagcacccacatattaaattttcagaatggaaatttaagctgttccgggtgagatcctttgaaaagacacctgaagaagctcaaaaggaaaagaaggattcctttgaggggaaaccctctctggagcaatctccagcagtcctggacaaggctgatggtcagaagccagtcccaactcagccattgttaaaagcccaccctaagttttcaaagaaatttcacgacaacgagaaagcaagaggcaaagcgatccatcaagccaaccttcgacatctctgccgcatctgtgggaattcttttagagctgatgagcacaacaggagatatccagtccatggtcctgtggatggtaaaaccctaggccttttacgaaagaaggaaaagagagctacttcctggccggacctcattgccaaggttttccggatcgatgtgaaggcagatgttgactcgatccaccccactgagttctgccataactgctggagcatcatgcacaggaagtttagcagtgccccatgtgaggtttacttcccgaggaacgtgaccatggagtggcacccccacacaccatcctgtgacatctgcaacactgcccgtcggggactcaagaggaagagtcttcagccaaacttgcagctcagcaaaaaactcaaaactgtgcttgaccaagcaagacaagcccgtcagcgcaagagaagagctcaggcaaggatcagcagcaaggatgtcatgaagaagatcgccaactgcagtaagatacatcttagtaccaagctccttgcagtggacttcccagagcactttgtgaaatccatctcctgccagatctgtgaacacattctggctgaccctgtggagaccaactgtaagcatgtcttttgccgggtctgcattctcagatgcctcaaagtcatgggcagctattgtccctcttgccgatatccatgcttccctactgacctggagagtccagtgaagtcctttctgagcgtcttgaattccctgatggtgaaatgtccagcaaaagagtgcaatgaggaggtcagtttggaaaaatataatcaccacatctcaagtcacaaggaatcaaaagagatttttgtgcacattaataaagggggccggccccgccaacatcttctgtcgctgactcggagagctcagaagcaccggctgagggagctcaagctgcaagtcaaagcctttgctgacaaagaagaaggtggagatgtgaagtccgtgtgcatgaccttgttcctgctggctctgagggcgaggaatgagcacaggcaagctgatgagctggaggccatcatgcagggaaagggctctggcctgcagccagctgtttgcttggccatccgtgtcaacaccttcctcagctgcagtcagtaccacaagatgtacaggactgtgaaagccatcacagggagacagatttttcagcctttgcatgcccttcggaatgctgagaaggtacttctgccaggctaccaccactttgagtggcagccacctctgaagaatgtgtcttccagcactgatgttggcattattgatgggctgtctggactatcatcctctgtggatgattacccagtggacaccattgcaaagaggttccgctatgattcagctttggtgtctgctttgatggacatggaagaagacatcttggaaggcatgagatcccaagaccttgatgattacctgaatggccccttcactgtggtggtgaaggagtcttgtgatggaatgggagacgtgagtgagaagcatgggagtgggcctgtagttccagaaaaggcagtccgtttttcattcacaatcatgaaaattactattgcccacagctctcagaatgtgaaagtatttgaagaagccaaacctaactctgaactgtgttgcaagccattgtgccttatgctggcagatgagtctgaccacgagacgctgactgccatcctgagtcctctcattgctgagagggaggccatgaagagcagtgaattaatgcttgagctgggaggcattctccggactttcaagttcatcttcaggggcaccggctatgatgaaaaacttgtgcgggaagtggaaggcctcgaggcttctggctcagtctacatttgtactctttgtgatgccacccgtctggaagcctctcaaaatcttgtcttccactctataaccagaagccatgctgagaacctggaacgttatgaggtctggcgttccaacccttaccatgagtctgtggaagaactgcgggatcgggtgaaaggggtctcagctaaacctttcattgagacagtcccttccatagatgcactccactgtgacattggcaatgcagctgagttctacaagatcttccagctagagataggggaagtgtataagaatcccaatgcttccaaagaggaaaggaaaaggtggcaggccacactggacaagcatctccggaagaagatgaacctcaaaccaatcatgaggatgaatggcaactttgccaggaagctcatgaccaaagagactgtggatgcagtttgtgagttaattccttccgaggagaggcacgaggctctgagggagctgatggatctttacctgaagatgaaaccagtatggcgatcatcatgccctgctaaagagtgcccagaatccctctgccagtacagtttcaattcacagcgttttgctgagctcctttctacgaagttcaagtataggtatgagggaaaaatcaccaattattttcacaaaaccctggcccatgttcctgaaattattgagagggatggctccattggggcatgggcaagtgagggaaatgagtctggtaacaaactgtttaggcgcttccggaaaatgaatgccaggcagtccaaatgctatgagatggaagatgtcctgaaacaccactggttgtacacctccaaatacctccagaagtttatgaatgctcataatgcattaaaaacctctgggtttaccatgaaccctcaggcaagcttaggggacccattaggcatagaggactctctggaaagccaagattcaatggaattttaa

SEQ ID NO: 10 - DNA, 1584 bp, Homo sapiens
atgtctctgcagatggtaacagtcagtaataacatagccttaattcagccaggcttctcactgatgaattttgatggacaagttttcttctttggacaaaaaggctggcccaaaagatcctgccccactggagttttccatctggatgtaaagcataaccatgtcaaactgaagcctacaattttctctaaggattcctgctacctccctcctcttcgctacccagccacttgcacattcaaaggcagcttggagtctgaaaagcatcaatacatcatccatggagggaaaacaccaaacaatgaggtttcagataagatttatgtcatgtctattgtttgcaagaacaacaaaaaggttacttttcgctgcacagagaaagacttggtaggagatgttcctgaagccagatatggtcattccattaatgtggtgtacagccgagggaaaagtatgggtgttctctttggaggacgctcatacatgccttctacccacagaaccacagaaaaatggaatagtgtagctgactgcctgccctgtgttttcctggtggattttgaatttgggtgtgctacatcatacattcttccagaacttcaggatgggctatcttttcatgtctctattgccaaaaatgacaccatctatattttaggaggacattcacttgccaataatatccggcctgccaacctgtacagaataagggttgatcttcccctgggtagcccagctgtgaattgcacagtcttgccaggaggaatctctgtctccagtgcaatcctgactcaaactaacaatgatgaatttgttattgttggtggctatcagcttgaaaatcaaaaaagaatgatctgcaacatcatctctttagaggacaacaagatagaaattcgtgagatggagaccccagattggaccccagacattaagcacagcaagatatggtttggaagcaacatgggaaatggaactgtttttcttggcataccaggagacaataaacaagttgtttcagaaggattctatttctatatgttgaaatgtgctgaagatgatactaatgaagagcagacaacattcacaaacagtcaaacatcaacagaagatccaggggattccactccctttgaagactctgaagaattttgtttcagtgcagaagcaaatagttttgatggtgatgatgaatttgacacctataatgaagatgatgaagaagatgagtctgagacaggctactggattacatgctgccctacttgtgatgtggatatcaacacttgggtaccattctattcaactgagctcaacaaacccgccatgatctactgctctcatggggatgggcactgggtccatgctcagtgcatggatctggcagaacgcacactcatccatctgtcagcaggaagcaacaagtattactgcaatgagcatgtggagatagcaagagctctacacactccccaaagagtcctacccttaaaaaagcctccaatgaaatccctccgtaaaaaaggttctggaaaaatcttgactcctgccaagaaatcctttcttagaaggttgtttgattag

SEQ ID NO: 11 - protein, 1124 aa, Homo sapiens
MAPPSEETPLIPQRSCSLLSTEAGALHVLLPARGPGPPQRLSFSFGDHLAEDLCVQAAKASGILPVYHSLFALATEDLSCWFPPSHIFSVEDASTQVLLYRIRFYFPNWFGLEKCHRFGLRKDLASAILDLPVLEHLFAQHRSDLVSGRLPVGLSLKEQGECLSLAVLDLARMAREQAQRPGELLKTVSYKACLPPSLRDLIQGLSFVTRRRIRRTVRRALRRVAACQADRHSLMAKYIMDLERLDPAGAAETFHVGLPGALGGHDGLGLLRVAGDGGIAWTQGEQEVLQPFCDFPEIVDISIKQAPRVGPAGEHRLVTVTRTDNQILEAEFPGLPEALSFVALVDGYFRLTTDSQHFFCKEVAPPRLLEEVAEQCHGPITLDFAINKLKTGGSRPGSYVLRRSPQDFDSFLLTVCVQNPLGPDYKGCLIRRSPTGTFLLVGLSRPHSSLRELLATCWDGGLHVDGVAVTLTSCCIPRPKEKSNLIVVQRGHSPPTSSLVQPQSQYQLSQMTFHKIPADSLEWHENLGHGSFTKIYRGCRHEVVDGEARKTEVLLKVMDAKHKNCMESFLEAASLMSQVSYRHLVLLHGVCMAGDSTMVQEFVHLGAIDMYLRKRGHLVPASWKLQVVKQLAYALNYLEDKGLPHGNVSARKVLLAREGADGSPPFIKLSDPGVSPAVLSLEMLTDRIPWVAPECLREAQTLSLEADKWGFGATVWEVFSGVTMPISALDPAKKLQFYEDRQQLPAPKWTELALLIQQCMAYEPVQRPSFRAVIRDLNSLISSDYELLSDPTPGALAPRDGLWNGAQLYACQDPTIFEERHLKYISQLGKGNFGSVELCRYDPLGDNTGALVAVKQLQHSGPDQQRDFQREIQILKALHSDFIVKYRGVSYGPGRQSLRLVMEYLPSGCLRDFLQRHRARLDASRLLLYSSQICKGMEYLGSRRCVHRDLAARNILVESEAHVKIADFGLAKLLPLDKDYYVVREPGQSPIFWYAPESLSDNIFSRQSDVWSFGVVLYELFTYCDKSCSPSAEFLRMMGCERDVPALCRLLELLEEGQRLPAPPACPAEVHELMKLCWAPSPQDRPSFSALGPQLDMLWSGSRGCETHAFTAHPEGKHHSLSFS

SEQ ID NO: 12 - protein, 289 aa, Homo sapiens
MENGYTYEDYKNTAEWLLSHTKHRPQVAIICGSGLGGLTDKLTQAQIFDYGEIPNFPRSTVPGHAGRLVFGFLNGRACVMMQGRFHMYEGYPLWKVTFPVRVFHLLGVDTLVVTNAAGGLNPKFEVGDIMLIRDHINLPGFSGQNPLRGPNDERFGDRFPAMSDAYDRTMRQRALSTWKQMGEQRELQEGTYVMVAGPSFETVAECRVLQKLGADAVGMSTVPEVIVARHCGLRVFGFSLITNKVIMDYESLEKANHEEVLAAGKQAAQKLEQFVSILMASIPLPDKAS

SEQ ID NO: 13 - protein, 363 aa, Homo sapiens
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNL

SEQ ID NO: 14 - protein, 1043 aa, Homo sapiens
MAASFPPTLGLSSAPDEIQHPHIKFSEWKFKLFRVRSFEKTPEEAQKEKKDSFEGKPSLEQSPAVLDKADGQKPVPTQPLLKAHPKFSKKFHDNEKARGKAIHQANLRHLCRICGNSFRADEHNRRYPVHGPVDGKTLGLLRKKEKRATSWPDLIAKVFRIDVKADVDSIHPTEFCHNCWSIMHRKFSSAPCEVYFPRNVTMEWHPHTPSCDICNTARRGLKRKSLQPNLQLSKKLKTVLDQARQARQHKRRAQARISSKDVMKKIANCSKIHLSTKLLAVDFPEHFVKSISCQICEHILADPVETNCKHVFCRVCILRCLKVMGSYCPSCRYPCFPTDLESPVKSFLSVLNSLMVKCPAKECNEEVSLEKYNHHISSHKESKEIFVHINKGGRPRQHLLSLTRRAQKHRLRELKLQVKAFADKEEGGDVKSVCMTLFLLALRARNEHRQADELEAIMQGKGSGLQPAVCLAIRVNTFLSCSQYHKMYRTVKAITGRQIFQPLHALRNAEKVLLPGYHHFEWQPPLKNVSSSTDVGIIDGLSGLSSSVDDYPVDTIAKRFRYDSALVSALMDMEEDILEGMRSQDLDDYLNGPFTVVVKESCDGMGDVSEKHGSGPVVPEKAVRFSFTIMKITIAHSSQNVKVFEEAKPNSELCCKPLCLMLADESDHETLTAILSPLIAEREAMKSSELMLELGGILRTFKFIFRGTGYDEKLVREVEGLEASGSVYICTLCDATRLEASQNLVFHSITRSHAENLERYEVWRSNPYHESVEELRDRVKGVSAKPFIETVPSIDALHCDIGNAAEFYKIFQLEIGEVYKNPNASKEERKRWQATLDKHLRKKMNLKPIMRMNGNFARKLMTKETVDAVCELIPSEERHEALRELMDLYLKMKPVWRSSCPAKECPESLCQYSFNSQRFAELLSTKFKYRYEGKITNYFHKTLAHVPEIIERDGSIGAWASEGNESGNKLFRRFRKMNARQSKCYEMEDVLKHHWLYTSKYLQKFMNAHNALKTSGFTMNPQASLGDPLGIEDSLESQDSMEF

SEQ ID NO: 15 - protein, 527 aa, Homo sapiens
MSLQMVTVSNNIALIQPGFSLMNFDGQVFFFGQKGWPKRSCPTGVFHLDVKHNHVKLKPTIFSKDSCYLPPLRYPATCTFKGSLESEKHQYIIHGGKTPNNEVSDKIYVMSIVCKNNKKVTFRCTEKDLVGDVPEARYGHSINVVYSRGKSMGVLFGGRSYMPSTHRTTEKWNSVADCLPCVFLVDFEFGCATSYILPELQDGLSFHVSIAKNDTIYILGGHSLANNIRPANLYRIRVDLPLGSPAVNCTVLPGGISVSSAILTQTNNDEFVIVGGYQLENQKRMICNIISLEDNKIEIREMETPDWTPDIKHSKIWFGSNMGNGTVFLGIPGDNKQVVSEGFYFYMLKCAEDDTNEEQTTFTNSQTSTEDPGDSTPFEDSEEFCFSAEANSFDGDDEFDTYNEDDEEDESETGYWITCCPTCDVDINTWVPFYSTELNKPAMIYCSHGDGHWVHAQCMDLAERTLIHLSAGSNKYYCNEHVEIARALHTPQRVLPLKKPPMKSLRKKGSGKILTPAKKSFLRRLFD

SEQ ID NO: 16 - DNA, 4368 bp, Homo sapiens
atgtccgactcgtgggtcccgaactccgcctcgggccaggacccagggggccgccggagggcctgggccgagctgctggcgggaagggtcaagagggaaaaatataatcctgaaagggcacagaaattaaaggaatcagctgtgcgcctcctgcgaagccatcaggacctgaatgcccttttgcttgaggtagaaggtccactgtgtaaaaaattgtctctcagcaaagtgattgactgtgacagttctgaggcctatgctaatcattctagttcatttataggctctgctttgcaggatcaagcctcaaggctgggggttcccgtgggtattctctcagccgggatggttgcctctagcgtgggacagatctgcacggctccagcggagaccagtcaccctgtgctgctgactgtggagcagagaaagaagctgtcttccctgttagagtttgctcagtatttattggcacacagtatgttctcccgtctttccttctgtcaagaattatggaaaatacagagttctttgttgcttgaagcggtgtggcatcttcacgtacaaggcattgtgagcctgcaagagctgctggaaagccatcccgacatgcatgctgtgggatcgtggctcttcaggaatctgtgctgcctttgtgaacagatggaagcatcctgccagcatgctgacgtcgccagggccatgctttctgattttgttcaaatgtttgttttgaggggatttcagaaaaactcagatctgagaagaactgtggagcctgaaaaaatgccgcaggtcacggttgatgtactgcagagaatgctgatttttgcacttgacgctttggctgctggagtacaggaggagtcctccactcacaagatcgtgaggtgctggttcggagtgttcagtggacacacgcttggcagtgtaatttccacagatcctctgaagaggttcttcagtcataccctgactcagatactcactcacagccctgtgctgaaagcatctgatgctgttcagatgcagagagagtggagctttgcgcggacacaccctctgctcacctcactgtaccgcaggctctttgtgatgctgagtgcagaggagttggttggccatttgcaagaagttctggaaacgcaggaggttcactggcagagagtgctctcctttgtgtctgccctggttgtctgctttccagaagcgcagcagctgcttgaagactgggtggcgcgtttgatggcccaggcattcgagagctgccagctggacagcatggtcactgcgttcctggttgtgcgccaggcagcactggagggcccctctgcgttcctgtcatatgcagactggttcaaggcctcctttgggagcacacgaggctaccatggctgcagcaagaaggccctggtcttcctgtttacgttcttgtcagaactcgtgccttttgagtctccccggtacctgcaggtgcacattctccacccacccctggttcccagcaagtaccgctccctcctcacagactacatctcattggccaagacacggctggccgacctcaaggtttctatagaaaacatgggactctacgaggatttgtcatcagctggggacattactgagccccacagccaagctcttcaggatgttgaaaaggccatcatggtgtttgagcatacggggaacatcccagtcaccgtcatggaggccagcatattcaggaggccttactacgtgtcccacttcctccccgccctgctcacacctcgagtgctccccaaagtccctgactcccgtgtggcgtttatagagtctctgaagagagcagataaaatccccccatctctgtactccacctactgccaggcctgctctgctgctgaagagaagccagaagatgcagccctgggagtgagggcagaacccaactctgctgaggagcccctgggacagctcacagctgcactgggagagctgagagcctccatgacagaccccagccagcgtgatgttatatcggcacaggtggcagtgatttctgaaagactgagggctgtcctgggccacaatgaggatgacagcagcgttgagatatcaaagattcagctcagcatcaacacgccgagactggagccacgggaacacattgctgtggacctcctgctgacgtctttctgtcagaacctgatggctgcctccagtgtcgctcccccggagaggcagggtccctgggctgccctcttcgtgaggaccatgtgtggacgtgtgctccctgcagtgctcacccggctctgccagctgctccgtcaccagggcccgagcctgagtgccccacatgtgctggggttggctgccctggccgtgcacctgggtgagtccaggtctgcgctcccagaggtggatgtgggtcctcctgcacctggtgctggccttcctgtccctgcgctctttgacagcctcctgacctgtaggacgagggattccttgttcttctgcctgaaattttgtacagcagcaatttcttactctctctgcaagttttcttcccagtcacgagatactttgtgcagctgcttatctccaggccttattaaaaagtttcagttcctcatgttcagattgttctcagaggcccgacagcctctttctgaggaggacgtagccagcctttcctggagacccttgcaccttccttctgcagactggcagagagctgccctctctctctggacacacagaaccttccgagaggtgttgaaagaggaagatgttcacttaacttaccaagactggttacacctggagctggaaattcaacctgaagctgatgctctttcagatactgaacggcaggacttccaccagtgggcgatccatgagcactttctccctgagtcctcggcttcagggggctgtgacggagacctgcaggctgcgtgtaccattcttgtcaacgcactgatggatttccaccaaagctcaaggagttatgaccactcagaaaattctgatttggtctttggtggccgcacaggaaatgaggatattatttccagattgcaggagatggtagctgacctggagctgcagcaagacctcatagtgcctctcggccacaccccttcccaggagcacttcctctttgagattttccgcagacggctccaggctctgacaagcgggtggagcgtggctgccagccttcagagacagagggagctgctaatgtacaaacggatcctcctccgcctgccttcgtctgtcctctgcggcagcagcttccaggcagaacagcccatcactgccagatgcgagcagttcttccacttggtcaactctgagatgagaaacttctgctcccacggaggtgccctgacacaggacatcactgcccacttcttcaggggcctcctgaacgcctgtctgcggagcagagacccctccctgatggtcgacttcatactggccaagtgccagacgaaatgccccttaattttgacctctgctctggtgtggtggccgagcctggagcctgtgctgctctgccggtggaggagacactgccagagcccgctgccccgggaactgcagaagctacaagaaggccggcagtttgccagcgatttcctctcccctgaggctgcctccccagcacccaacccggactggctctcagctgctgcactgcactttgcgattcaacaagtcagggaagaaaacatcaggaagcagctaaagaagctggactgcgagagagaggagctattggttttccttttcttcttctccttgatgggcctgctgtcgtcacatctgacctcaaatagcaccacagacctgccaaaggctttccacgtttgtgcagcaatcctcgagtgtttagagaagaggaagatatcctggctggcactctttcagttgacagagagtgacctcaggctggggcggctcctcctccgtgtggccccggatcagcacaccaggctgctgcctttcgctttttacagtcttctctcctacttccatgaagacgcggccatcagggaagaggccttcctgcatgttgctgtggacatgtacttgaagctggtccagctcttcgtggctggggatacaagcacagtttcacctccagctggcaggagcctggagctcaagggtcagggcaaccccgtggaactgataacaaaagctcgtctttttctgctgcagttaatacctcggtgcccgaaaaagagcttctcacacgtggcagagctgctggctgatcgtggggactgcgacccagaggtgagcgccgccctccagagcagacagcaggctgcccctgacgctgacctgtcccaggagcctcatctcttctga

SEQ ID NO: 17 - DNA, 1677 bp, Homo sapiens
atggctcaagattcagtagatctttcttgtgattatcagttttggatgcagaagctttctgtatgggatcaggcttccactttggaaacccagcaagacacctgtcttcacgtggctcagttccaggagttcctaaggaagatgtatgaagccttgaaagagatggattctaatacagtcattgaaagattccccacaattggtcaactgttggcaaaagcttgttggaatccttttattttagcatatgatgaaagccaaaaaattctaatatggtgcttatgttgtctaattaacaaagaaccacagaattctggacaatcaaaacttaactcctggatacagggtgtattatctcatatactttcagcactcagatttgataaagaagttgctcttttcactcaaggtcttgggtatgcacctatagattactatcctggtttgcttaaaaatatggttttatcattagcgtctgaactcagagagaatcatcttaatggatttaacactcaaaggcgaatggctcccgagcgagtggcgtccctgtcacgagtttgtgtcccacttattaccctgacagatgttgaccccctggtggaggctctcctcatctgtcatggacgtgaacctcaggaaatcctccagccagagttctttgaggctgtaaacgaggccattttgctgaagaagatttctctccccatgtcagctgtagtctgcctctggcttcggcaccttcccagccttgaaaaagcaatgctgcatctttttgaaaagctaatctccagtgagagaaattgtctgagaaggatcgaatgctttataaaagattcatcgctgcctcaagcagcctgccaccctgccatattccgggttgttgatgagatgttcaggtgtgcactcctggaaaccgatggggccctggaaatcatagccactattcaggtgtttacgcagtgctttgtagaagctctggagaaagcaagcaagcagctgcggtttgcactcaagacctactttccttacacttctccatctcttgccatggtgctgctgcaagaccctcaagatatccctcggggacactggctccagacactgaagcatatttctgaactgctcagagaagcagttgaagaccagactcatgggtcctgcggaggtccctttgagagctggttcctgttcattcacttcggaggatgggctgagatggtggcagagcaattactgatgtcggcagccgaaccccccacggccctgctgtggctcttggccttctactacggcccccgtgatgggaggcagcagagagcacagactatggtccaggtgaaggccgtgctgggccacctcctggcaatgtccagaagcagcagcctctcagcccaggacctgcagacggtagcaggacagggcacagacacagacctcagagctcctgcacaacagctgatcaggcaccttctcctcaacttcctgctctgggctcctggaggccacacgatcgcctgggatgtcatcaccctgatggctcacactgctgagataactcacgagatcattggctttcttgaccagaccttgtacagatggaatcgtcttggcattgaaagccctagatcagaaaaactggcccgagagctccttaaagagctgcgaactcaagtctag

SEQ ID NO: 18 - DNA, 1611 bp, Homo sapiens
atggcgacaccggacgcggggctccctggggctgagggcgtggagccggcgccctgggcgcagctggaggcccccgcccgcctcctgctgcaggcgctgcaggcggggcctgagggggcgcggcgcggcctgggggtgctccgggcgctgggcagccgcggctgggagcccttcgactggggtcgcttgctcgaggccctgtgccgggaggagccggtcgtgcaggggcctgacggccgtctggagctgaaaccactgttgctgcgattgccccggatatgccagaggaacctgatgtccctgctgatggccgttcggccatcgctgccggaaagtgggctcctctctgtgctgcagattgcccagcaggacctagcccctgacccagatgcctggctccgtgccctgggggaattgctgcgaagggatttgggggtggggacctccatggagggagcttctccactgtctgaaagatgccagagacagctccaaagtctatgtagggggctgggcctggggggcaggaggttgaaatccccccaggctccagaccctgaagaagaggagaacagggactcccagcagcctgggaaacgcagaaaggactcagaggaagaggctgccagtcctgaggggaagagggtccccaaaagattacggtgttgggaagaggaagaagatcatgagaaggagagacccgaacataagtcactggaatccctggcagatggaggaagtgcatctcctattaaggaccagcctgtcatggcagttaagactggcgaggacggttcgaatctggatgatgctaaaggtctggctgagagtttggagttgcccaaagctatccaggaccagcttcccaggctgcagcagctgctgaagaccttggaggaggggttagagggattggaggatgcccccccagttgagctacagcttcttcacgaatgtagtcccagccagatggacttgctgtgtgcccagctgcagctccctcagctctcagacctcggtctcctgcggctctgcacctggctgctggccctttcacctgatctcagcctcagcaatgctactgtgctgaccagaagcctctttcttggacggatcctctccttgacttcctcagcctcccgcctgcttacaactgccctgacctccttctgtgccaaatatacataccctgtctgcagcgccctccttgaccctgtgctccaggccccaggcacaggtcctgctcaaacagagttactgtgttgccttgtgaagatggagtccctggagccagatgcacaggttctaatgctgggacagatcttggagctgccctggaaggaggaaactttcttggtgttgcagtcactcctagagcggcaggtggagatgacccctgagaagttcagtgtcttaatggagaagctctgtaaaaaggggctggcagccaccacctccatggcctatgccaagctcatgctgacagtgatgaccaagtatcaggctaacatcactgagacccagaggctgggcctggctatggccctagaacctaacaccaccttcctgaggaagtccctgaaggccgccttgaaacatttgggcccctga

SEQ ID NO: 19 - DNA, 1125 bp, Homo sapiens
atggaatcccttctgcagcacctggatcgcttttccgagcttctggcggtctcaagcactacctacgtcagcacctgggaccccgccaccgtgcgccgggccttgcagtgggcgcgctacctgcgccacatccatcggcgctttggtcggcatggccccattcgcacggctctggagcggcggctgcacaaccagtggaggcaagagggcggctttgggcggggtccagttccgggattagcgaacttccaggccctcggtcactgtgacgtcctgctctctctgcgcctgctggagaaccgggccctcggggatgcagctcgttaccacctggtgcagcaactctttcccggcccgggcgtccgggacgccgatgaggagacactccaagagagcctggcccgccttgcccgccggcggtctgcggtgcacatgctgcgcttcaatggctatagagagaacccaaatctccaggaggactctctgatgaagacccaggcggagctgctgctggagcgtctgcaggaggtggggaaggccgaagcggagcgtcccgccaggtttctcagcagcctgtgggagcgcttgcctcagaacaacttcctgaaggtgatagcggtggcgctgttgcagccgcctttgtctcgtcggccccaagaagagttggaacccggcatccacaaatcacctggagaggggagccaagtgctagtccactggcttctggggaattcggaagtctttgctgccttttgtcgcgccctcccagccgggcttttgactttagtgactagccgccacccagcgctgtctcctgtctatctgggtctgctaacagactggggtcaacgtttgcactatgaccttcagaaaggcatttgggttggaactgagtcccaagatgtgccctgggaggagttgcacaataggtttcaaagcctctgtcaggcccctccacctctgaaagataaagttctaactgccctggagacctgtaaagcgcaggatggagattttgaagtacctggtcttagcatctggacagacctcttattagctcttcgtagtggtgcatttaggaaaagacaagttttgggtctcagcgcaggcctcagttctgtatag

SEQ ID NO: 20 - DNA, 1869 bp, Homo sapiens
atgtcccgccagaccacctctgtgggctccagctgcctggacctgtggagggaaaagaatgaccggctcgttcgacaggccaaggtggctcagaactccggtctgactctgaggcgacagcagttggctcaggatgcactggaagggctcagagggctcctccatagtctgcaagggctccctgcagctgttcctgttcttcccttggagctgactgtcacctgcaacttcattatcctgagggcaagcttggcccagggtttcacagaggatcaggcccaggatatccagcggagcctagagagagtgctggagacacaggagcagcaggggcccaggttggaacaggggctcagggagctgtgggactctgtccttcgtgcttcctgccttctgccggagctgctgtctgccctgcaccgcctggttggcctgcaggctgccctctggttgagtgctgaccgtcttggggacctggccttgttactagagaccctgaatggcagccagagtggagcctctaaggatctgctgttacttctgaaaacttggagtcccccagctgaggaattagatgctccattgaccctgcaggatgcccagggattgaaggatgtcctcctgacagcatttgcctaccgccaaggtctccaggagctgatcacagggaacccagacaaggcactaagcagccttcatgaagcggcctcaggcctgtgtccacggcctgtgttggtccaggtgtacacagcactggggtcctgtcaccgtaagatgggaaatccacagagagcactgttgtacttggttgcagccctgaaagagggatcagcctggggtcctccacttctggaggcctctaggctctatcagcaactgggggacacaacagcagagctggagagtctggagctgctagttgaggccttgaatgtcccatgcagttccaaagccccgcagtttctcattgaggtagaattactactgccaccacctgacctagcctcaccccttcattgtggcactcagagccagaccaagcacatactagcaagcaggtgcctacagacggggagggcaggagacgctgcagagcattacttggacctgctggccctgttgctggatagctcggagccaaggttctccccacccccctcccctccagggccctgtatgcctgaggtgtttttggaggcagcggtagcactgatccaggcaggcagagcccaagatgccttgactctatgtgaggagttgctcagccgcacatcatctctgctacccaagatgtcccggctgtgggaagatgccagaaaaggaaccaaggaactgccatactgcccactctgggtctctgccacccacctgcttcagggccaggcctgggttcaactgggtgcccaaaaagtggcaattagtgaatttagcaggtgcctcgagctgctcttccgggccacacctgaggaaaaagaacaaggggcagctttcaactgtgagcagggatgtaagtcagatgcggcactgcagcagcttcgggcagccgccctaattagtcgtggactggaatgggtagccagcggccaggataccaaagccttacaggacttcctcctcagtgtgcagatgtgcccaggtaatcgagacacttactttcacctgcttcagactctgaagaggctagatcggagggatgaggccactgcactctggtggaggctggaggcccaaactaaggggtcacatgaagatgctctgtggtctctccccctgtacctagaaagctatttgagctggatccgtccctctgatcgtgacgccttccttgaagaatttcggacatctctgccaaagtcttgtgacctgtag

SEQ ID NO: 21 - protein, 1455 aa, Homo sapiens
MSDSWVPNSASGQDPGGRRRAWAELLAGRVKREKYNPERAQKLKESAVRLLRSHQDLNALLLEVEGPLCKKLSLSKVIDCDSSEAYANHSSSFIGSALQDQASRLGVPVGILSAGMVASSVGQICTAPAETSHPVLLTVEQRKKLSSLLEFAQYLLAHSMFSRLSFCQELWKIQSSLLLEAVWHLHVQGIVSLQELLESHPDMHAVGSWLFRNLCCLCEQMEASCQHADVARAMLSDFVQMFVLRGFQKNSDLRRTVEPEKMPQVTVDVLQRMLIFALDALAAGVQEESSTHKIVRCWFGVFSGHTLGSVISTDPLKRFFSHTLTQILTHSPVLKASDAVQMQREWSFARTHPLLTSLYRRLFVMLSAEELVGHLQEVLETQEVHWQRVLSFVSALVVCFPEAQQLLEDWVARLMAQAFESCQLDSMVTAFLVVRQAALEGPSAFLSYADWFKASFGSTRGYHGCSKKALVFLFTFLSELVPFESPRYLQVHILHPPLVPSKYRSLLTDYISLAKTRLADLKVSIENMGLYEDLSSAGDITEPHSQALQDVEKAIMVFEHTGNIPVTVMEASIFRRPYYVSHFLPALLTPRVLPKVPDSRVAFIESLKRADKIPPSLYSTYCQACSAAEEKPEDAALGVRAEPNSAEEPLGQLTAALGELRASMTDPSQRDVISAQVAVISERLRAVLGHNEDDSSVEISKIQLSINTPRLEPREHIAVDLLLTSFCQNLMAASSVAPPERQGPWAALFVRTMCGRVLPAVLTRLCQLLRHQGPSLSAPHVLGLAALAVHLGESRSALPEVDVGPPAPGAGLPVPALFDSLLTCRTRDSLFFCLKFCTAAISYSLCKFSSQSRDTLCSCLSPGLIKKFQFLMFRLFSEARQPLSEEDVASLSWRPLHLPSADWQRAALSLWTHRTFREVLKEEDVHLTYQDWLHLELEIQPEADALSDTERQDFHQWAIHEHFLPESSASGGCDGDLQAACTILVNALMDFHQSSRSYDHSENSDLVFGGRTGNEDIISRLQEMVADLELQQDLIVPLGHTPSQEHFLFEIFRRRLQALTSGWSVAASLQRQRELLMYKRILLRLPSSVLCGSSFQAEQPITARCEQFFHLVNSEMRNFCSHGGALTQDITAHFFRGLLNACLRSRDPSLMVDFILAKCQTKCPLILTSALVWWPSLEPVLLCRWRRHCQSPLPRELQKLQEGRQFASDFLSPEAASPAPNPDWLSAAALHFAIQQVREENIRKQLKKLDCEREELLVFLFFFSLMGLLSSHLTSNSTTDLPKAFHVCAAILECLEKRKISWLALFQLTESDLRLGRLLLRVAPDQHTRLLPFAFYSLLSYFHEDAAIREEAFLHVAVDMYLKLVQLFVAGDTSTVSPPAGRSLELKGQGNPVELITKARLFLLQLIPRCPKKSFSHVAELLADRGDCDPEVSAALQSRQQAAPDADLSQEPHLF

SEQ ID NO: 22 - protein, 558 aa, Homo sapiens
MAQDSVDLSCDYQFWMQKLSVWDQASTLETQQDTCLHVAQFQEFLRKMYEALKEMDSNTVIERFPTIGQLLAKACWNPFILAYDESQKILIWCLCCLINKEPQNSGQSKLNSWIQGVLSHILSALRFDKEVALFTQGLGYAPIDYYPGLLKNMVLSLASELRENHLNGFNTQRRMAPERVASLSRVCVPLITLTDVDPLVEALLICHGREPQEILQPEFFEAVNEAILLKKISLPMSAVVCLWLRHLPSLEKAMLHLFEKLISSERNCLRRIECFIKDSSLPQAACHPAIFRVVDEMFRCALLETDGALEIIATIQVFTQCFVEALEKASKQLRFALKTYFPYTSPSLAMVLLQDPQDIPRGHWLQTLKHISELLREAVEDQTHGSCGGPFESWFLFIHFGGWAEMVAEQLLMSAAEPPTALLWLLAFYYGPRDGRQQRAQTMVQVKAVLGHLLAMSRSSSLSAQDLQTVAGQGTDTDLRAPAQQLIRHLLLNFLLWAPGGHTIAWDVITLMAHTAEITHEIIGFLDQTLYRWNRLGIESPRSEKLARELLKELRTQV

SEQ ID NO: 23 - protein, 536 aa, Homo sapiens
MATPDAGLPGAEGVEPAPWAQLEAPARLLLQALQAGPEGARRGLGVLRALGSRGWEPFDWGRLLEALCREEPVVQGPDGRLELKPLLLRLPRICQRNLMSLLMAVRPSLPESGLLSVLQIAQQDLAPDPDAWLRALGELLRRDLGVGTSMEGASPLSERCQRQLQSLCRGLGLGGRRLKSPQAPDPEEEENRDSQQPGKRRKDSEEEAASPEGKRVPKRLRCWEEEEDHEKERPEHKSLESLADGGSASPIKDQPVMAVKTGEDGSNLDDAKGLAESLELPKAIQDQLPRLQQLLKTLEEGLEGLEDAPPVELQLLHECSPSQMDLLCAQLQLPQLSDLGLLRLCTWLLALSPDLSLSNATVLTRSLFLGRILSLTSSASRLLTTALTSFCAKYTYPVCSALLDPVLQAPGTGPAQTELLCCLVKMESLEPDAQVLMLGQILELPWKEETFLVLQSLLERQVEMTPEKFSVLMEKLCKKGLAATTSMAYAKLMLTVMTKYQANITETQRLGLAMALEPNTTFLRKSLKAALKHLGP

SEQ ID NO: 24 - protein, 374 aa, Homo sapiens
MESLLQHLDRFSELLAVSSTTYVSTWDPATVRRALQWARYLRHIHRRFGRHGPIRTALERRLHNQWRQEGGFGRGPVPGLANFQALGHCDVLLSLRLLENRALGDAARYHLVQQLFPGPGVRDADEETLQESLARLARRRSAVHMLRFNGYRENPNLQEDSLMKTQAELLLERLQEVGKAEAERPARFLSSLWERLPQNNFLKVIAVALLQPPLSRRPQEELEPGIHKSPGEGSQVLVHWLLGNSEVFAAFCRALPAGLLTLVTSRHPALSPVYLGLLTDWGQRLHYDLQKGIWVGTESQDVPWEELHNRFQSLCQAPPPLKDKVLTALETCKAQDGDFEVPGLSIWTDLLLALRSGAFRKRQVLGLSAGLSSV

SEQ ID NO: 25 - protein, 622 aa, Homo sapiens
MSRQTTSVGSSCLDLWREKNDRLVRQAKVAQNSGLTLRRQQLAQDALEGLRGLLHSLQGLPAAVPVLPLELTVTCNFIILRASLAQGFTEDQAQDIQRSLERVLETQEQQGPRLEQGLRELWDSVLRASCLLPELLSALHRLVGLQAALWLSADRLGDLALLLETLNGSQSGASKDLLLLLKTWSPPAEELDAPLTLQDAQGLKDVLLTAFAYRQGLQELITGNPDKALSSLHEAASGLCPRPVLVQVYTALGSCHRKMGNPQRALLYLVAALKEGSAWGPPLLEASRLYQQLGDTTAELESLELLVEALNVPCSSKAPQFLIEVELLLPPPDLASPLHCGTQSQTKHILASRCLQTGRAGDAAEHYLDLLALLLDSSEPRFSPPPSPPGPCMPEVFLEAAVALIQAGRAQDALTLCEELLSRTSSLLPKMSRLWEDARKGTKELPYCPLWVSATHLLQGQAWVQLGAQKVAISEFSRCLELLFRATPEEKEQGAAFNCEQGCKSDAALQQLRAAALISRGLEWVASGQDTKALQDFLLSVQMCPGNRDTYFHLLQTLKRLDRRDEATALWWRLEAQTKGSHEDALWSLPLYLESYLSWIRPSDRDAFLEEFRTSLPKSCDL

SEQ ID NO: 26 - DNA, 3417 bp, artificial
  synthesized foamy virus Pol gene
atgaatcccctccaactgttgcagcctctgcccgcagagatcaaagggactaaactgctggctcattgggactctggagcaaccataacatgcataccagaaagcttccttgaggacgagcagcctatcaaaaaaacattgattaagacgatccacggggaaaagcagcagaacgtgtattacgttacctttaaggtgaagggccggaaagtcgaggccgaggtcattgcctctccatacgaatacattctgctctcacccaccgacgtgccatggttgacccagcagcctcttcagctgactatcctggtccctttgcaggagtaccaggaaaagattctgagcaagacggcgcttcccgaagatcagaaacagcagctgaagaccctcttcgtgaaatacgataatctctggcagcactgggaaaaccaggtgggccatcggaagattcgaccccacaatatcgccacgggcgactatccacctaggcctcagaagcagtatcccatcaacccaaaagcaaaaccaagcatccagatcgtcatcgatgatttgcttaagcaaggagtgctcaccccacaaaatagcactatgaacaccccagtgtaccccgtgcccaaaccggacggcagatggagaatggtattggactatcgcgaagttaacaaaaccatacctttgaccgcagcccagaatcaacacagcgccggcatcttggctacgatcgtgagacagaagtacaaaacaactctcgatctggccaacggcttttgggctcacccaatcactccagagagctactggcttaccgcctttacatggcaggggaaacaatactgttggacccggctgcctcaggggttcttgaattcacccgcactgtttacagctgacgtcgttgatctgctgaaagaaatccccaatgtgcaggtatacgtggacgacatctatctttcccacgacgatccaaaagagcatgttcagcagctcgaaaaagttttccagatcctgctgcaggctggttatgtcgtctcactcaagaagtctgagataggacaaaagactgtggagtttctgggatttaacatcaccaaggaaggacggggattgactgatacgttcaagactaagctgctcaacattactcctcccaaggatcttaagcagctgcagagtattcttggcttgctcaattttgcccggaattttatccctaacttcgctgagcttgttcagcccctgtataatctgatagcctccgccaagggtaagtacatcgaatggagcgaggagaatactaaacagttgaacatggtgattgaggcacttaacactgcctccaacttggaggaacgactgccagagcagcgacttgtgattaaagtgaacacctcaccaagtgcggggtacgtgcgctactacaacgagacaggcaaaaagcccataatgtacctgaactatgtcttctcaaaagctgagctcaagtttagcatgctcgagaagctgcttactaccatgcacaaggccctgataaaggccatggaccttgccatggggcaagaaatcctcgtgtacagccccatcgtttccatgacgaagatccagaaaacaccactgcccgaacgaaaggccttgcctatcagatggattacttggatgacctaccttgaggacccccgcatccagtttcattatgataagaccctgcctgaactgaaacacatcccagacgtgtacacctccagtcagtccccagtcaagcacccttctcaatatgaaggagtgttttataccgatgggagtgccatcaaatcccctgaccccacaaaaagtaacaacgccggtatgggtatcgtccacgcgacctataagcccgagtatcaggtactgaaccagtggtccatcccgctggggaatcataccgcccagatggcggaaattgccgcagtcgagtttgcctgcaaaaaggcattgaaaatcccagggcctgtcctggtcatcaccgactctttctacgtagccgagtcagccaataaggaactgccctattggaaaagtaatggcttcgtgaacaacaagaagaagccactgaaacatattagcaaatggaaatctattgccgagtgtctgtctatgaagcccgacatcactatccagcacgaaaagggccatcagcccaccaacactagtatccatacggagggaaacgctctggccgataagctagccactcaagggagttacgtcgtgaactgcaacaccaagaaacctaaccttgacgccgaattggaccaattgctgcagggacattacataaagggctaccccaagcagtatacctattttctggaagacggcaaggtaaaagtgtcccggccagagggcgtcaagatcatcccgccacaaagcgacagacagaaaatcgttctgcaggcccacaacctcgctcatactgggcgcgaagctactctgctcaagattgccaatctgtattggtggccgaatatgagaaaagacgtcgtaaagcaactggggcgctgtcagcagtgtttgatcactaacgcaagtaacaaagcaagtgggccgattcttcgaccagaccgccctcagaaaccgttcgataagttttttatagattacattggacctctgcctcccagtcaaggctacctctacgtgctggtagtggtcgatggcatgacgggattcacatggctgtacccgaccaaggcgccgagtacttccgcgacggtcaagagccttaacgttctcacctccatagctatccccaaagttatccactccgaccagggcgcagctttcaccagctctaccttcgcggagtgggccaaagagagggggattcacttggaattctcaacgccttaccacccccaatctagcggaaaggtcgagagaaaaaattcagatatcaaaagactgttgaccaagctgcttgttggccgccctacaaagtggtatgacctcctgcctgtcgtccagctggcactgaacaacacctacagccccgtgctcaagtatacacctcatcagttgctgtttggtattgatagtaacactcctttcgcaaatcaggatacgttggatctcactcgcgaagaagagctcagtttgctgcaggagatacgcacgagtctgtaccacccttccactcctcccacttctagtaggtcttggtctccagttgtgggacagcttgttcaggaaagagtcgcccggcccgcatcactgcggccccggtggcacaaaccgtctactgtactgaaggtgctcaacccacggacggtggtaatccttgaccatctcggaaacaaccggacagtgtcaatcgataacctcaagccaacctcccaccaaaacggcacaaccaatgacacagccacaatggatcattag

SEQ ID NO: 27 - protein, 1138 aa, artificial
  synthesized foamy virus Pol protein
MNPLQLLQPLPAEIKGTKLLAHWDSGATITCIPESFLEDEQPIKKTLIKTIHGEKQQNVYYVTFKVKGRKVEAEVIASPYEYILLSPTDVPWLTQQPLQLTILVPLQEYQEKILSKTALPEDQKQQLKTLFVKYDNLWQHWENQVGHRKIRPHNIATGDYPPRPQKQYPINPKAKPSIQIVIDDLLKQGVLTPQNSTMNTPVYPVPKPDGRWRMVLDYREVNKTIPLTAAQNQHSAGILATIVRQKYKTTLDLANGFWAHPITPESYWLTAFTWQGKQYCWTRLPQGFLNSPALFTADVVDLLKEIPNVQVYVDDIYLSHDDPKEHVQQLEKVFQILLQAGYVVSLKKSEIGQKTVEFLGFNITKEGRGLTDTFKTKLLNITPPKDLKQLQSILGLLNFARNFIPNFAELVQPLYNLIASAKGKYIEWSEENTKQLNMVIEALNTASNLEERLPEQRLVIKVNTSPSAGYVRYYNETGKKPIMYLNYVFSKAELKFSMLEKLLTTMHKALIKAMDLAMGQEILVYSPIVSMTKIQKTPLPERKALPIRWITWMTYLEDPRIQFHYDKTLPELKHIPDVYTSSQSPVKHPSQYEGVFYTDGSAIKSPDPTKSNNAGMGIVHATYKPEYQVLNQWSIPLGNHTAQMAEIAAVEFACKKALKIPGPVLVITDSFYVAESANKELPYWKSNGFVNNKKKPLKHISKWKSIAECLSMKPDITIQHEKGHQPTNTSIHTEGNALADKLATQGSYVVNCNTKKPNLDAELDQLLQGHYIKGYPKQYTYFLEDGKVKVSRPEGVKIIPPQSDRQKIVLQAHNLAHTGREATLLKIANLYWWPNMRKDVVKQLGRCQQCLITNASNKASGPILRPDRPQKPFDKFFIDYIGPLPPSQGYLYVLVVVDGMTGFTWLYPTKAPSTSATVKSLNVLTSIAIPKVIHSDQGAAFTSSTFAEWAKERGIHLEFSTPYHPQSSGKVERKNSDIKRLLTKLLVGRPTKWYDLLPVVQLALNNTYSPVLKYTPHQLLFGIDSNTPFANQDTLDLTREEELSLLQEIRTSLYHPSTPPTSSRSWSPVVGQLVQERVARPASLRPRWHKPSTVLKVLNPRTVVILDHLGNNRTVSIDNLKPTSHQNGTTNDTATMDH

SEQ ID NO: 28 - DNA, 511 bp, Homo sapiens
ggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcggggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccag

SEQ ID NO: 29 - DNA, 8509 bp, artificial
  synthetic foamy virus vector containing PGK promoter associated with gammaC gene
tcgcgcgttctcgaggagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacgctaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgcaaatgggcggtaggcgtgtacggtgggaggtctatataagcagagcttctagattgtacgggagctctctcttcactactcgctgcgtcgagagtgtacgagactctccaggtttggtaagaaatattttatattgttataatgttactatgatccattaacactctgcttatagattgtaagggtgattgcaatgctttctgcataaaactttggttttcttgttaatcaataaaccgacttgattcgagaacctactcatatattattgtctcttttatactttattaagtaaaaggatttgtatattagccttgctaagggagacatctagtgatataagtgtgaactacacttatcttaaatgatgtaactccttaggataatcaatatacaaaattccatgacaattggcgcccaacgtggggctcgaatataagtcgggtttatttgtaaattatccctagggacctccgagcatagcgggaggcatataaaagccaatagacaatggctagcaggaagtaatgttgaagaatatgaacttgatgttgaagctctggttgtaattttaagagatagaaatataccaagaaatcctttacatggagaagttataggtcttcgccttactgaaggatggtggggacaaattgagagatttcagatggtacgttgattcgaattaaggctatggatttggccatgggacaagaaatattagtttatagtcccattgtatctatgactaaaatacaaaaaactccactaccagaaagaaaagctttacccattagatggataacatggatgacttatttagaagatccaagaatccaatttcattatgataaaaccttaccagaacttaagcatattccagatgtatatacatctagtcagtctcctgttaaacatccttctcaatatgaaggagtgttttatactgatggctcggccatcaaaagtcctgatcctacaaaaagcaataatgctggcatgggaatagtacatgccacatacaaacctgaatatcaagttttgaatcaatggtcaataccactaggtaatcatactgctcagatggctgaaatagctgcagttgaatttgcctgtaaaaaagctttaaaaatacctggtcctgtattagttataactgatagtttctatgtagcagaaagtgctaataaagaattaccatactggaaatctaatgggtttgttaataataagaaaaagcctcttaaacatatctccaaatggaaatctattgctgagtgtttatctatgaaaccagacattactattcaacatgaaaaaggcatcagcctacaaataccagtattcatactgaaaggcaatgccctagcagataagcttgccacccaaggaagttatgtggttaattgtaataccaaaaaaccaaacctggatgcagagttggatcaattattacagggtcattatataaaaggatatcccaaacaatatacatattttttagaagatggcaaagtaaaagtttccagacctgaaggggttaaaattattccccctcagtcagacagacaaaaaattgtgcttcaagcccacaatttggctcacaccggacgtgaagccactcttttaaaaattgccaacctttattggtggccaaatatgagaaaggatgtggttaaacaactaggacgctgtcaacagtgtttaatcacaaatgcttccaacaaagcctctggtcctattctaagaccagataggcctcaaaaaccttttgataaattctttattgactatattggacctttgccaccttcacagggatacctatatgtattagtagttgttgatggaatgacaggattcacttggttataccccactaaggctccttctactagcgcaactgttaaatctctcaatgtactcactagtattgcaattccaaaggtgattcactctgatcaaggtgcagcattcacttcttcaacctttgctgaatgggcaaaggaaagaggtatacatttggaattcagtactccttatcacccccaaagtggtagtaaggtggaaaggaaaaatagtgatataaaacgacttttaactaaactgctagtaggaagacccacaaagtggtatgacctattgcctgttgtacaacttgctttaaacaacacctatagccctgtattaaaatatactccacatcaactcttatttggtatagattcaaatactccatttgcaaatcaagatacacttgacttgaccagagaagaagaactttctcttttacaggaaattcgtacttctttataccatccatccacccctccagcctcctctcgttcctggtctcctgttgttggccaattggtccaggagagggtggctaggcctgcttctttgagacctcgttggcataaaccgtctactgtacttaaggtgttgaatccaaggactgttgttattttggaccatcttggcaacaacagaactgtaagtatagataatttaaaacctacttctcatcagaatggcaccaccaatgacactgcaacaatggatcatttggaaaaaaatgaataaagcgcatgaggcacttcaaaatacaacaactgtgactgaacagcagaaggaacaaattatactggacattcaaaatgaagaagtacaaccaactaggagagataaatttagatatctgctttatacttgttgtgctactagctcaagagtattggcctggatgtttttagtttgtatattgttaatcattgttttggtttcatgctttgtgactatatccagaatacaatggaataaggatattcaggtattaggacctgtaatagactggaatgttactcaaagagctgtttatcaacccttacagactagaaggattgcacgttcccttagaatgcagcatcctgttccaaaatatgtggaggtaaatatgactagtattccacaaggtgtatactatgaaccccatccggcgcgccagatctgcatgcccacggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcggggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagggggatccaccggtcgccgccaccatgctgaaaccaagcctgccctttacaagcctgctgttcctgcagctgccactgctgggggtcggactgaatactacaatcctgacaccaaacggaaatgaggacaccacagccgatttctttctgactaccatgcccactgacagtctgtcagtgagcaccctgccactgcccgaggtccagtgcttcgtgtttaacgtcgaatatatgaactgtacctggaatagctcctctgaacctcagccaacaaatctgactctgcactactggtataagaactctgacaatgataaggtgcagaaatgctcacattatctgttcagcgaggaaatcacctccggctgtcagctgcagaagaaagagattcacctgtaccagacatttgtggtccagctgcaggatccccgggaacctcggagacaggccactcagatgctgaagctgcagaacctggtcatcccatgggctcccgagaatctgaccctgcataaactgtccgagtctcagctggaactgaactggaacaataggttcctgaatcactgcctggagcatctggtgcagtaccgcacagactgggatcactcttggactgaacagagtgtggactatcgacataagtttagtctgccttcagtggatgggcagaaaaggtacacattcagggtccgctctcggttcaacccactgtgcggaagcgcccagcactggagcgagtggtcccaccccatccattgggggtctaacaccagcaaggagaatcctttcctgtttgccctggaagctgtggtcatttcagtgggaagcatgggcctgatcattagcctgctgtgcgtgtacttctggctggagcggaccatgcctagaatcccaacactgaagaacctggaggacctggtgacagaatatcacggcaatttttccgcttggtctggggtcagtaaaggactggcagagagcctgcagcccgattactccgagcggctgtgcctggtgtccgaaattccccctaaaggcggggcactgggagaaggccctggggcctccccctgcaaccagcactcaccctattgggcaccaccctgttacaccctgaaacccgaaacttaagcggccgtcgagggctgcaggaattcgagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttatagcctctgtatctagctattgcttcccgtacggctttcgttttctcctccttgtataaatcctggttgctgtctcttttagaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaagctgacgtcctttcgaattcgatatcaagcttatcgataccgtcgacggttaccaagcagctatggaagcttatggacctcagagaggaagtaacgaggagagggtgtggtggaatgccactagaaaccagggaaaacaaggaggagagtattacagggaaggaggtgaagaacctcattacccaaatactcctgctcctcatagacgtacctgggatgagagacacaaggttcttaaattgtcctcattcgctactccctctgacatccaacgctgggctactaactctagattgtacgggagctctcttcactactcgctgcgtcgagagtgtacgagactctccaggtttggtaagaaatattttatattgttataatgttactatgatccattaacactctgcttatagattgtaagggtgattgcaatgctttctgcataaaactttggttttcttgttaatcaataaaccgacttgattcgagaacctactcatatattattgtctcttttatactttattaagtaaaaggatttgtatattagccttgctaagggagacatctagtgatataagtgtgaactacacttatcttaaatgatgtaactccttaggataatcaatatacaaaattccatgacaattggcgatacccagctgcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 30 - DNA, 9284 bp, artificial
  synthetic foamy virus vector containing PGK promoter associated with GFP-2A-gammaC
tcgcgcgttctcgaggagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacgctaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgcaaatgggcggtaggcgtgtacggtgggaggtctatataagcagagcttctagattgtacgggagctctctcttcactactcgctgcgtcgagagtgtacgagactctccaggtttggtaagaaatattttatattgttataatgttactatgatccattaacactctgcttatagattgtaagggtgattgcaatgctttctgcataaaactttggttttcttgttaatcaataaaccgacttgattcgagaacctactcatatattattgtctcttttatactttattaagtaaaaggatttgtatattagccttgctaagggagacatctagtgatataagtgtgaactacacttatcttaaatgatgtaactccttaggataatcaatatacaaaattccatgacaattggcgcccaacgtggggctcgaatataagtcgggtttatttgtaaattatccctagggacctccgagcatagcgggaggcatataaaagccaatagacaatggctagcaggaagtaatgttgaagaatatgaacttgatgttgaagctctggttgtaattttaagagatagaaatataccaagaaatcctttacatggagaagttataggtcttcgccttactgaaggatggtggggacaaattgagagatttcagatggtacgttgattcgaattaaggctatggatttggccatgggacaagaaatattagtttatagtcccattgtatctatgactaaaatacaaaaaactccactaccagaaagaaaagctttacccattagatggataacatggatgacttatttagaagatccaagaatccaatttcattatgataaaaccttaccagaacttaagcatattccagatgtatatacatctagtcagtctcctgttaaacatccttctcaatatgaaggagtgttttatactgatggctcggccatcaaaagtcctgatcctacaaaaagcaataatgctggcatgggaatagtacatgccacatacaaacctgaatatcaagttttgaatcaatggtcaataccactaggtaatcatactgctcagatggctgaaatagctgcagttgaatttgcctgtaaaaaagctttaaaaatacctggtcctgtattagttataactgatagtttctatgtagcagaaagtgctaataaagaattaccatactggaaatctaatgggtttgttaataataagaaaaagcctcttaaacatatctccaaatggaaatctattgctgagtgtttatctatgaaaccagacattactattcaacatgaaaaaggcatcagcctacaaataccagtattcatactgaaaggcaatgccctagcagataagcttgccacccaaggaagttatgtggttaattgtaataccaaaaaaccaaacctggatgcagagttggatcaattattacagggtcattatataaaaggatatcccaaacaatatacatattttttagaagatggcaaagtaaaagtttccagacctgaaggggttaaaattattccccctcagtcagacagacaaaaaattgtgcttcaagcccacaatttggctcacaccggacgtgaagccactcttttaaaaattgccaacctttattggtggccaaatatgagaaaggatgtggttaaacaactaggacgctgtcaacagtgtttaatcacaaatgcttccaacaaagcctctggtcctattctaagaccagataggcctcaaaaaccttttgataaattctttattgactatattggacctttgccaccttcacagggatacctatatgtattagtagttgttgatggaatgacaggattcacttggttataccccactaaggctccttctactagcgcaactgttaaatctctcaatgtactcactagtattgcaattccaaaggtgattcactctgatcaaggtgcagcattcacttcttcaacctttgctgaatgggcaaaggaaagaggtatacatttggaattcagtactccttatcacccccaaagtggtagtaaggtggaaaggaaaaatagtgatataaaacgacttttaactaaactgctagtaggaagacccacaaagtggtatgacctattgcctgttgtacaacttgctttaaacaacacctatagccctgtattaaaatatactccacatcaactcttatttggtatagattcaaatactccatttgcaaatcaagatacacttgacttgaccagagaagaagaactttctcttttacaggaaattcgtacttctttataccatccatccacccctccagcctcctctcgttcctggtctcctgttgttggccaattggtccaggagagggtggctaggcctgcttctttgagacctcgttggcataaaccgtctactgtacttaaggtgttgaatccaaggactgttgttattttggaccatcttggcaacaacagaactgtaagtatagataatttaaaacctacttctcatcagaatggcaccaccaatgacactgcaacaatggatcatttggaaaaaaatgaataaagcgcatgaggcacttcaaaatacaacaactgtgactgaacagcagaaggaacaaattatactggacattcaaaatgaagaagtacaaccaactaggagagataaatttagatatctgctttatacttgttgtgctactagctcaagagtattggcctggatgtttttagtttgtatattgttaatcattgttttggtttcatgctttgtgactatatccagaatacaatggaataaggatattcaggtattaggacctgtaatagactggaatgttactcaaagagctgtttatcaacccttacagactagaaggattgcacgttcccttagaatgcagcatcctgttccaaaatatgtggaggtaaatatgactagtattccacaaggtgtatactatgaaccccatccggcgcgccagatctgcatgccacggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcggggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagggggatccaccggtcgccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggtgagggcagaggaagtcttctaacatgcggtgacgtggaggagaatccgggccctatgctgaaaccaagcctgccctttacaagcctgctgttcctgcagctgccactgctgggggtcggactgaatactacaatcctgacaccaaacggaaatgaggacaccacagccgatttctttctgactaccatgcccactgacagtctgtcagtgagcaccctgccactgcccgaggtccagtgcttcgtgtttaacgtcgaatatatgaactgtacctggaatagctcctctgaacctcagccaacaaatctgactctgcactactggtataagaactctgacaatgataaggtgcagaaatgctcacattatctgttcagcgaggaaatcacctccggctgtcagctgcagaagaaagagattcacctgtaccagacatttgtggtccagctgcaggatccccgggaacctcggagacaggccactcagatgctgaagctgcagaacctggtcatcccatgggctcccgagaatctgaccctgcataaactgtccgagtctcagctggaactgaactggaacaataggttcctgaatcactgcctggagcatctggtgcagtaccgcacagactgggatcactcttggactgaacagagtgtggactatcgacataagtttagtctgccttcagtggatgggcagaaaaggtacacattcagggtccgctctcggttcaacccactgtgcggaagcgcccagcactggagcgagtggtcccaccccatccattgggggtctaacaccagcaaggagaatcctttcctgtttgccctggaagctgtggtcatttcagtgggaagcatgggcctgatcattagcctgctgtgcgtgtacttctggctggagcggaccatgcctagaatcccaacactgaagaacctggaggacctggtgacagaatatcacggcaatttttccgcttggtctggggtcagtaaaggactggcagagagcctgcagcccgattactccgagcggctgtgcctggtgtccgaaattccccctaaaggcggggcactgggagaaggccctggggcctccccctgcaaccagcactcaccctattgggcaccaccctgttacaccctgaaacccgaaacttaagcggccgcgtcgagggctgcaggaattcgagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttatagcctctgtatctagctattgcttcccgtacggctttcgttttctcctccttgtataaatcctggttgctgtctcttttagaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaagctgacgtcctttcgaattcgatatcaagcttatcgataccgtcgacggttaccaagcagctatggaagcttatggacctcagagaggaagtaacgaggagagggtgtggtggaatgccactagaaaccagggaaaacaaggaggagagtattacagggaaggaggtgaagaacctcattacccaaatactcctgctcctcatagacgtacctgggatgagagacacaaggttcttaaattgtcctcattcgctactccctctgacatccaacgctgggctactaactctagattgtacgggagctctcttcactactcgctgcgtcgagagtgtacgagactctccaggtttggtaagaaatattttatattgttataatgttactatgatccattaacactctgcttatagattgtaagggtgattgcaatgctttctgcataaaactttggttttcttgttaatcaataaaccgacttgattcgagaacctactcatatattattgtctcttttatactttattaagtaaaaggatttgtatattagccttgctaagggagacatctagtgatataagtgtgaactacacttatcttaaatgatgtaactccttaggataatcaatatacaaaattccatgacaattggcgatacccagctgcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 31 - DNA, 11529 bp, artificial
  lentiviral vector containing PGK promoter associated with FancA gene
caggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagattggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttccgatcacgagactagcctcgagaagcttgatatcgaattcccacggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcggggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagggggatccaccggtccgccaaggccatgtccgactcgtgggtcccgaactccgcctcgggccaggacccagggggccgccggagggcctgggccgagctgctggcgggaagggtcaagagggaaaaatataatcctgaaagggcacagaaattaaaggaatcagctgtgcgcctcctgcgaagccatcaggacctgaatgcccttttgcttgaggtagaaggtccactgtgtaaaaaattgtctctcagcaaagtgattgactgtgacagttctgaggcctatgctaatcattctagttcatttataggctctgctttgcaggatcaagcctcaaggctgggggttcccgtgggtattctctcagccgggatggttgcctctagcgtgggacagatctgcacggctccagcggagaccagtcaccctgtgctgctgactgtggagcagagaaagaagctgtcttccctgttagagtttgctcagtatttattggcacacagtatgttctcccgtctttccttctgtcaagaattatggaaaatacagagttctttgttgcttgaagcggtgtggcatcttcacgtacaaggcattgtgagcctgcaagagctgctggaaagccatcccgacatgcatgctgtgggatcgtggctcttcaggaatctgtgctgcctttgtgaacagatggaagcatcctgccagcatgctgacgtcgccagggccatgctttctgattttgttcaaatgtttgttttgaggggatttcagaaaaactcagatctgagaagaactgtggagcctgaaaaaatgccgcaggtcacggttgatgtactgcagagaatgctgatttttgcacttgacgctttggctgctggagtacaggaggagtcctccactcacaagatcgtgaggtgctggttcggagtgttcagtggacacacgcttggcagtgtaatttccacagatcctctgaagaggttcttcagtcataccctgactcagatactcactcacagccctgtgctgaaagcatctgatgctgttcagatgcagagagagtggagctttgcgcggacacaccctctgctcacctcactgtaccgcaggctctttgtgatgctgagtgcagaggagttggttggccatttgcaagaagttctggaaacgcaggaggttcactggcagagagtgctctcctttgtgtctgccctggttgtctgctttccagaagcgcagcagctgcttgaagactgggtggcgcgtttgatggcccaggcattcgagagctgccagctggacagcatggtcactgcgttcctggttgtgcgccaggcagcactggagggcccctctgcgttcctgtcatatgcagactggttcaaggcctcctttgggagcacacgaggctaccatggctgcagcaagaaggccctggtcttcctgtttacgttcttgtcagaactcgtgccttttgagtctccccggtacctgcaggtgcacattctccacccacccctggttcccagcaagtaccgctccctcctcacagactacatctcattggccaagacacggctggccgacctcaaggtttctatagaaaacatgggactctacgaggatttgtcatcagctggggacattactgagccccacagccaagctcttcaggatgttgaaaaggccatcatggtgtttgagcatacggggaacatcccagtcaccgtcatggaggccagcatattcaggaggccttactacgtgtcccacttcctccccgccctgctcacacctcgagtgctccccaaagtccctgactcccgtgtggcgtttatagagtctctgaagagagcagataaaatccccccatctctgtactccacctactgccaggcctgctctgctgctgaagagaagccagaagatgcagccctgggagtgagggcagaacccaactctgctgaggagcccctgggacagctcacagctgcactgggagagctgagagcctccatgacagaccccagccagcgtgatgttatatcggcacaggtggcagtgatttctgaaagactgagggctgtcctgggccacaatgaggatgacagcagcgttgagatatcaaagattcagctcagcatcaacacgccgagactggagccacgggaacacattgctgtggacctcctgctgacgtctttctgtcagaacctgatggctgcctccagtgtcgctcccccggagaggcagggtccctgggctgccctcttcgtgaggaccatgtgtggacgtgtgctccctgcagtgctcacccggctctgccagctgctccgtcaccagggcccgagcctgagtgccccacatgtgctggggttggctgccctggccgtgcacctgggtgagtccaggtctgcgctcccagaggtggatgtgggtcctcctgcacctggtgctggccttcctgtccctgcgctctttgacagcctcctgacctgtaggacgagggattccttgttcttctgcctgaaattttgtacagcagcaatttcttactctctctgcaagttttcttcccagtcacgagatactttgtgcagctgcttatctccaggccttattaaaaagtttcagttcctcatgttcagattgttctcagaggcccgacagcctctttctgaggaggacgtagccagcctttcctggagacccttgcaccttccttctgcagactggcagagagctgccctctctctctggacacacagaaccttccgagaggtgttgaaagaggaagatgttcacttaacttaccaagactggttacacctggagctggaaattcaacctgaagctgatgctctttcagatactgaacggcaggacttccaccagtgggcgatccatgagcactttctccctgagtcctcggcttcagggggctgtgacggagacctgcaggctgcgtgtaccattcttgtcaacgcactgatggatttccaccaaagctcaaggagttatgaccactcagaaaattctgatttggtctttggtggccgcacaggaaatgaggatattatttccagattgcaggagatggtagctgacctggagctgcagcaagacctcatagtgcctctcggccacaccccttcccaggagcacttcctctttgagattttccgcagacggctccaggctctgacaagcgggtggagcgtggctgccagccttcagagacagagggagctgctaatgtacaaacggatcctcctccgcctgccttcgtctgtcctctgcggcagcagcttccaggcagaacagcccatcactgccagatgcgagcagttcttccacttggtcaactctgagatgagaaacttctgctcccacggaggtgccctgacacaggacatcactgcccacttcttcaggggcctcctgaacgcctgtctgcggagcagagacccctccctgatggtcgacttcatactggccaagtgccagacgaaatgccccttaattttgacctctgctctggtgtggtggccgagcctggagcctgtgctgctctgccggtggaggagacactgccagagcccgctgccccgggaactgcagaagctacaagaaggccggcagtttgccagcgatttcctctcccctgaggctgcctccccagcacccaacccggactggctctcagctgctgcactgcactttgcgattcaacaagtcagggaagaaaacatcaggaagcagctaaagaagctggactgcgagagagaggagctattggttttccttttcttcttctccttgatgggcctgctgtcgtcacatctgacctcaaatagcaccacagacctgccaaaggctttccacgtttgtgcagcaatcctcgagtgtttagagaagaggaagatatcctggctggcactctttcagttgacagagagtgacctcaggctggggcggctcctcctccgtgtggccccggatcagcacaccaggctgctgcctttcgctttttacagtcttctctcctacttccatgaagacgcggccatcagggaagaggccttcctgcatgttgctgtggacatgtacttgaagctggtccagctcttcgtggctggggatacaagcacagtttcacctccagctggcaggagcctggagctcaagggtcagggcaaccccgtggaactgataacaaaagctcgtctttttctgctgcagttaatacctcggtgcccgaaaaagagcttctcacacgtggcagagctgctggctgatcgtggggactgcgacccagaggtgagcgccgccctccagagcagacagcaggctgcccctgacgctgacctgtcccaggagcctcatctcttctgacgggacctgccactgcacaccagcccagctcccgtgtaaataatttattacaagcataacatggagctcttgttgcactaaaaagtggattacaaatctcctcgactgctttagtggggaaaggaatcaattatttatgaactgtccggccccgagtcactcagcgtttgcgggaaaataaaccactggtcccagagcagaggaaggctacttgagccggacaccaagcccgcctccagcaccaagggcgggcagcaccctccgaccctcccatgcgggtgcacacgaagggtgaggctgacacagccactgcggagtccaggctgctagaggtgctcatcctcactgccgtcctcaggtgggttcgggcttcaccgcctggccctctgtggtcacagaggggctcggtggcccaggtggtggttccgcctccaggggcagggccttgtcctgggtctgtgtcagcgggtgcaccatggacatgtgtacaagtaaagcggccgcgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctaggcttttgcgtcgagacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcc

SEQ ID NO: 32 - DNA, 4904 bp, artificial
  PGK promoter associated with FANCA gene
ggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcggggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagggggatccaccggtccgccaaggccatgtccgactcgtgggtcccgaactccgcctcgggccaggacccagggggccgccggagggcctgggccgagctgctggcgggaagggtcaagagggaaaaatataatcctgaaagggcacagaaattaaaggaatcagctgtgcgcctcctgcgaagccatcaggacctgaatgcccttttgcttgaggtagaaggtccactgtgtaaaaaattgtctctcagcaaagtgattgactgtgacagttctgaggcctatgctaatcattctagttcatttataggctctgctttgcaggatcaagcctcaaggctgggggttcccgtgggtattctctcagccgggatggttgcctctagcgtgggacagatctgcacggctccagcggagaccagtcaccctgtgctgctgactgtggagcagagaaagaagctgtcttccctgttagagtttgctcagtatttattggcacacagtatgttctcccgtctttccttctgtcaagaattatggaaaatacagagttctttgttgcttgaagcggtgtggcatcttcacgtacaaggcattgtgagcctgcaagagctgctggaaagccatcccgacatgcatgctgtgggatcgtggctcttcaggaatctgtgctgcctttgtgaacagatggaagcatcctgccagcatgctgacgtcgccagggccatgctttctgattttgttcaaatgtttgttttgaggggatttcagaaaaactcagatctgagaagaactgtggagcctgaaaaaatgccgcaggtcacggttgatgtactgcagagaatgctgatttttgcacttgacgctttggctgctggagtacaggaggagtcctccactcacaagatcgtgaggtgctggttcggagtgttcagtggacacacgcttggcagtgtaatttccacagatcctctgaagaggttcttcagtcataccctgactcagatactcactcacagccctgtgctgaaagcatctgatgctgttcagatgcagagagagtggagctttgcgcggacacaccctctgctcacctcactgtaccgcaggctctttgtgatgctgagtgcagaggagttggttggccatttgcaagaagttctggaaacgcaggaggttcactggcagagagtgctctcctttgtgtctgccctggttgtctgctttccagaagcgcagcagctgcttgaagactgggtggcgcgtttgatggcccaggcattcgagagctgccagctggacagcatggtcactgcgttcctggttgtgcgccaggcagcactggagggcccctctgcgttcctgtcatatgcagactggttcaaggcctcctttgggagcacacgaggctaccatggctgcagcaagaaggccctggtcttcctgtttacgttcttgtcagaactcgtgccttttgagtctccccggtacctgcaggtgcacattctccacccacccctggttcccagcaagtaccgctccctcctcacagactacatctcattggccaagacacggctggccgacctcaaggtttctatagaaaacatgggactctacgaggatttgtcatcagctggggacattactgagccccacagccaagctcttcaggatgttgaaaaggccatcatggtgtttgagcatacggggaacatcccagtcaccgtcatggaggccagcatattcaggaggccttactacgtgtcccacttcctccccgccctgctcacacctcgagtgctccccaaagtccctgactcccgtgtggcgtttatagagtctctgaagagagcagataaaatccccccatctctgtactccacctactgccaggcctgctctgctgctgaagagaagccagaagatgcagccctgggagtgagggcagaacccaactctgctgaggagcccctgggacagctcacagctgcactgggagagctgagagcctccatgacagaccccagccagcgtgatgttatatcggcacaggtggcagtgatttctgaaagactgagggctgtcctgggccacaatgaggatgacagcagcgttgagatatcaaagattcagctcagcatcaacacgccgagactggagccacgggaacacattgctgtggacctcctgctgacgtctttctgtcagaacctgatggctgcctccagtgtcgctcccccggagaggcagggtccctgggctgccctcttcgtgaggaccatgtgtggacgtgtgctccctgcagtgctcacccggctctgccagctgctccgtcaccagggcccgagcctgagtgccccacatgtgctggggttggctgccctggccgtgcacctgggtgagtccaggtctgcgctcccagaggtggatgtgggtcctcctgcacctggtgctggccttcctgtccctgcgctctttgacagcctcctgacctgtaggacgagggattccttgttcttctgcctgaaattttgtacagcagcaatttcttactctctctgcaagttttcttcccagtcacgagatactttgtgcagctgcttatctccaggccttattaaaaagtttcagttcctcatgttcagattgttctcagaggcccgacagcctctttctgaggaggacgtagccagcctttcctggagacccttgcaccttccttctgcagactggcagagagctgccctctctctctggacacacagaaccttccgagaggtgttgaaagaggaagatgttcacttaacttaccaagactggttacacctggagctggaaattcaacctgaagctgatgctctttcagatactgaacggcaggacttccaccagtgggcgatccatgagcactttctccctgagtcctcggcttcagggggctgtgacggagacctgcaggctgcgtgtaccattcttgtcaacgcactgatggatttccaccaaagctcaaggagttatgaccactcagaaaattctgatttggtctttggtggccgcacaggaaatgaggatattatttccagattgcaggagatggtagctgacctggagctgcagcaagacctcatagtgcctctcggccacaccccttcccaggagcacttcctctttgagattttccgcagacggctccaggctctgacaagcgggtggagcgtggctgccagccttcagagacagagggagctgctaatgtacaaacggatcctcctccgcctgccttcgtctgtcctctgcggcagcagcttccaggcagaacagcccatcactgccagatgcgagcagttcttccacttggtcaactctgagatgagaaacttctgctcccacggaggtgccctgacacaggacatcactgcccacttcttcaggggcctcctgaacgcctgtctgcggagcagagacccctccctgatggtcgacttcatactggccaagtgccagacgaaatgccccttaattttgacctctgctctggtgtggtggccgagcctggagcctgtgctgctctgccggtggaggagacactgccagagcccgctgccccgggaactgcagaagctacaagaaggccggcagtttgccagcgatttcctctcccctgaggctgcctccccagcacccaacccggactggctctcagctgctgcactgcactttgcgattcaacaagtcagggaagaaaacatcaggaagcagctaaagaagctggactgcgagagagaggagctattggttttccttttcttcttctccttgatgggcctgctgtcgtcacatctgacctcaaatagcaccacagacctgccaaaggctttccacgtttgtgcagcaatcctcgagtgtttagagaagaggaagatatcctggctggcactctttcagttgacagagagtgacctcaggctggggcggctcctcctccgtgtggccccggatcagcacaccaggctgctgcctttcgctttttacagtcttctctcctacttccatgaagacgcggccatcagggaagaggccttcctgcatgttgctgtggacatgtacttgaagctggtccagctcttcgtggctggggatacaagcacagtttcacctccagctggcaggagcctggagctcaagggtcagggcaaccccgtggaactgataacaaaagctcgtctttttctgctgcagttaatacctcggtgcccgaaaaagagcttctcacacgtggcagagctgctggctgatcgtggggactgcgacccagaggtgagcgccgccctccagagcagacagcaggctgcccctgacgctgacctgtcccaggagcctcatctcttctga

SEQ ID NO: 33 - DNA, 12265 bp, artificial
  synthetic foamy virus vector containing PGK promoter associated with FancA gene
tcgcgcgttctcgaggagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacgctaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgcaaatgggcggtaggcgtgtacggtgggaggtctatataagcagagcttctagattgtacgggagctctctcttcactactcgctgcgtcgagagtgtacgagactctccaggtttggtaagaaatattttatattgttataatgttactatgatccattaacactctgcttatagattgtaagggtgattgcaatgctttctgcataaaactttggttttcttgttaatcaataaaccgacttgattcgagaacctactcatatattattgtctcttttatactttattaagtaaaaggatttgtatattagccttgctaagggagacatctagtgatataagtgtgaactacacttatcttaaatgatgtaactccttaggataatcaatatacaaaattccatgacaattggcgcccaacgtggggctcgaatataagtcgggtttatttgtaaattatccctagggacctccgagcatagcgggaggcatataaaagccaatagacaatggctagcaggaagtaatgttgaagaatatgaacttgatgttgaagctctggttgtaattttaagagatagaaatataccaagaaatcctttacatggagaagttataggtcttcgccttactgaaggatggtggggacaaattgagagatttcagatggtacgttgattcgaattaaggctatggatttggccatgggacaagaaatattagtttatagtcccattgtatctatgactaaaatacaaaaaactccactaccagaaagaaaagctttacccattagatggataacatggatgacttatttagaagatccaagaatccaatttcattatgataaaaccttaccagaacttaagcatattccagatgtatatacatctagtcagtctcctgttaaacatccttctcaatatgaaggagtgttttatactgatggctcggccatcaaaagtcctgatcctacaaaaagcaataatgctggcatgggaatagtacatgccacatacaaacctgaatatcaagttttgaatcaatggtcaataccactaggtaatcatactgctcagatggctgaaatagctgcagttgaatttgcctgtaaaaaagctttaaaaatacctggtcctgtattagttataactgatagtttctatgtagcagaaagtgctaataaagaattaccatactggaaatctaatgggtttgttaataataagaaaaagcctcttaaacatatctccaaatggaaatctattgctgagtgtttatctatgaaaccagacattactattcaacatgaaaaaggcatcagcctacaaataccagtattcatactgaaaggcaatgccctagcagataagcttgccacccaaggaagttatgtggttaattgtaataccaaaaaaccaaacctggatgcagagttggatcaattattacagggtcattatataaaaggatatcccaaacaatatacatattttttagaagatggcaaagtaaaagtttccagacctgaaggggttaaaattattccccctcagtcagacagacaaaaaattgtgcttcaagcccacaatttggctcacaccggacgtgaagccactcttttaaaaattgccaacctttattggtggccaaatatgagaaaggatgtggttaaacaactaggacgctgtcaacagtgtttaatcacaaatgcttccaacaaagcctctggtcctattctaagaccagataggcctcaaaaaccttttgataaattctttattgactatattggacctttgccaccttcacagggatacctatatgtattagtagttgttgatggaatgacaggattcacttggttataccccactaaggctccttctactagcgcaactgttaaatctctcaatgtactcactagtattgcaattccaaaggtgattcactctgatcaaggtgcagcattcacttcttcaacctttgctgaatgggcaaaggaaagaggtatacatttggaattcagtactccttatcacccccaaagtggtagtaaggtggaaaggaaaaatagtgatataaaacgacttttaactaaactgctagtaggaagacccacaaagtggtatgacctattgcctgttgtacaacttgctttaaacaacacctatagccctgtattaaaatatactccacatcaactcttatttggtatagattcaaatactccatttgcaaatcaagatacacttgacttgaccagagaagaagaactttctcttttacaggaaattcgtacttctttataccatccatccacccctccagcctcctctcgttcctggtctcctgttgttggccaattggtccaggagagggtggctaggcctgcttctttgagacctcgttggcataaaccgtctactgtacttaaggtgttgaatccaaggactgttgttattttggaccatcttggcaacaacagaactgtaagtatagataatttaaaacctacttctcatcagaatggcaccaccaatgacactgcaacaatggatcatttggaaaaaaatgaataaagcgcatgaggcacttcaaaatacaacaactgtgactgaacagcagaaggaacaaattatactggacattcaaaatgaagaagtacaaccaactaggagagataaatttagatatctgctttatacttgttgtgctactagctcaagagtattggcctggatgtttttagtttgtatattgttaatcattgttttggtttcatgctttgtgactatatccagaatacaatggaataaggatattcaggtattaggacctgtaatagactggaatgttactcaaagagctgtttatcaacccttacagactagaaggattgcacgttcccttagaatgcagcatcctgttccaaaatatgtggaggtaaatatgactagtattccacaaggtgtatactatgaaccccatccggcgcgccagatctgcatgccacggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcggggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagggggatccaccggtccgccaaggccatgtccgactcgtgggtcccgaactccgcctcgggccaggacccagggggccgccggagggcctgggccgagctgctggcgggaagggtcaagagggaaaaatataatcctgaaagggcacagaaattaaaggaatcagctgtgcgcctcctgcgaagccatcaggacctgaatgcccttttgcttgaggtagaaggtccactgtgtaaaaaattgtctctcagcaaagtgattgactgtgacagttctgaggcctatgctaatcattctagttcatttataggctctgctttgcaggatcaagcctcaaggctgggggttcccgtgggtattctctcagccgggatggttgcctctagcgtgggacagatctgcacggctccagcggagaccagtcaccctgtgctgctgactgtggagcagagaaagaagctgtcttccctgttagagtttgctcagtatttattggcacacagtatgttctcccgtctttccttctgtcaagaattatggaaaatacagagttctttgttgcttgaagcggtgtggcatcttcacgtacaaggcattgtgagcctgcaagagctgctggaaagccatcccgacatgcatgctgtgggatcgtggctcttcaggaatctgtgctgcctttgtgaacagatggaagcatcctgccagcatgctgacgtcgccagggccatgctttctgattttgttcaaatgtttgttttgaggggatttcagaaaaactcagatctgagaagaactgtggagcctgaaaaaatgccgcaggtcacggttgatgtactgcagagaatgctgatttttgcacttgacgctttggctgctggagtacaggaggagtcctccactcacaagatcgtgaggtgctggttcggagtgttcagtggacacacgcttggcagtgtaatttccacagatcctctgaagaggttcttcagtcataccctgactcagatactcactcacagccctgtgctgaaagcatctgatgctgttcagatgcagagagagtggagctttgcgcggacacaccctctgctcacctcactgtaccgcaggctctttgtgatgctgagtgcagaggagttggttggccatttgcaagaagttctggaaacgcaggaggttcactggcagagagtgctctcctttgtgtctgccctggttgtctgctttccagaagcgcagcagctgcttgaagactgggtggcgcgtttgatggcccaggcattcgagagctgccagctggacagcatggtcactgcgttcctggttgtgcgccaggcagcactggagggcccctctgcgttcctgtcatatgcagactggttcaaggcctcctttgggagcacacgaggctaccatggctgcagcaagaaggccctggtcttcctgtttacgttcttgtcagaactcgtgccttttgagtctccccggtacctgcaggtgcacattctccacccacccctggttcccagcaagtaccgctccctcctcacagactacatctcattggccaagacacggctggccgacctcaaggtttctatagaaaacatgggactctacgaggatttgtcatcagctggggacattactgagccccacagccaagctcttcaggatgttgaaaaggccatcatggtgtttgagcatacggggaacatcccagtcaccgtcatggaggccagcatattcaggaggccttactacgtgtcccacttcctccccgccctgctcacacctcgagtgctccccaaagtccctgactcccgtgtggcgtttatagagtctctgaagagagcagataaaatccccccatctctgtactccacctactgccaggcctgctctgctgctgaagagaagccagaagatgcagccctgggagtgagggcagaacccaactctgctgaggagcccctgggacagctcacagctgcactgggagagctgagagcctccatgacagaccccagccagcgtgatgttatatcggcacaggtggcagtgatttctgaaagactgagggctgtcctgggccacaatgaggatgacagcagcgttgagatatcaaagattcagctcagcatcaacacgccgagactggagccacgggaacacattgctgtggacctcctgctgacgtctttctgtcagaacctgatggctgcctccagtgtcgctcccccggagaggcagggtccctgggctgccctcttcgtgaggaccatgtgtggacgtgtgctccctgcagtgctcacccggctctgccagctgctccgtcaccagggcccgagcctgagtgccccacatgtgctggggttggctgccctggccgtgcacctgggtgagtccaggtctgcgctcccagaggtggatgtgggtcctcctgcacctggtgctggccttcctgtccctgcgctctttgacagcctcctgacctgtaggacgagggattccttgttcttctgcctgaaattttgtacagcagcaatttcttactctctctgcaagttttcttcccagtcacgagatactttgtgcagctgcttatctccaggccttattaaaaagtttcagttcctcatgttcagattgttctcagaggcccgacagcctctttctgaggaggacgtagccagcctttcctggagacccttgcaccttccttctgcagactggcagagagctgccctctctctctggacacacagaaccttccgagaggtgttgaaagaggaagatgttcacttaacttaccaagactggttacacctggagctggaaattcaacctgaagctgatgctctttcagatactgaacggcaggacttccaccagtgggcgatccatgagcactttctccctgagtcctcggcttcagggggctgtgacggagacctgcaggctgcgtgtaccattcttgtcaacgcactgatggatttccaccaaagctcaaggagttatgaccactcagaaaattctgatttggtctttggtggccgcacaggaaatgaggatattatttccagattgcaggagatggtagctgacctggagctgcagcaagacctcatagtgcctctcggccacaccccttcccaggagcacttcctctttgagattttccgcagacggctccaggctctgacaagcgggtggagcgtggctgccagccttcagagacagagggagctgctaatgtacaaacggatcctcctccgcctgccttcgtctgtcctctgcggcagcagcttccaggcagaacagcccatcactgccagatgcgagcagttcttccacttggtcaactctgagatgagaaacttctgctcccacggaggtgccctgacacaggacatcactgcccacttcttcaggggcctcctgaacgcctgtctgcggagcagagacccctccctgatggtcgacttcatactggccaagtgccagacgaaatgccccttaattttgacctctgctctggtgtggtggccgagcctggagcctgtgctgctctgccggtggaggagacactgccagagcccgctgccccgggaactgcagaagctacaagaaggccggcagtttgccagcgatttcctctcccctgaggctgcctccccagcacccaacccggactggctctcagctgctgcactgcactttgcgattcaacaagtcagggaagaaaacatcaggaagcagctaaagaagctggactgcgagagagaggagctattggttttccttttcttcttctccttgatgggcctgctgtcgtcacatctgacctcaaatagcaccacagacctgccaaaggctttccacgtttgtgcagcaatcctcgagtgtttagagaagaggaagatatcctggctggcactctttcagttgacagagagtgacctcaggctggggcggctcctcctccgtgtggccccggatcagcacaccaggctgctgcctttcgctttttacagtcttctctcctacttccatgaagacgcggccatcagggaagaggccttcctgcatgttgctgtggacatgtacttgaagctggtccagctcttcgtggctggggatacaagcacagtttcacctccagctggcaggagcctggagctcaagggtcagggcaaccccgtggaactgataacaaaagctcgtctttttctgctgcagttaatacctcggtgcccgaaaaagagcttctcacacgtggcagagctgctggctgatcgtggggactgcgacccagaggtgagcgccgccctccagagcagacagcaggctgcccctgacgctgacctgtcccaggagcctcatctcttctgacgggacctgccactgcacaccagcccagctcccgtgtaaataatttattacaagcataacatggagctcttgttgcactaaaaagtggattacaaatctcctcgactgctttagtggggaaaggaatcaattatttatgaactgtccggccccgagtcactcagcgtttgcgggaaaataaaccactggtcccagagcagaggaaggctacttgagccggacaccaagcccgcctccagcaccaagggcgggcagcaccctccgaccctcccatgcgggtgcacacgaagggtgaggctgacacagccactgcggagtccaggctgctagaggtgctcatcctcactgccgtcctcaggtgggttcgggcttcaccgcctggccctctgtggtcacagaggggctcggtggcccaggtggtggttccgcctccaggggcagggccttgtcctgggtctgtgtcagcgggtgcaccatggacatgtgtacaagtaaagcggccgcgtcgagggctgcaggaattcgagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttatagcctctgtatctagctattgcttcccgtacggctttcgttttctcctccttgtataaatcctggttgctgtctcttttagaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaagctgacgtcctttcgaattcgatatcaagcttatcgataccgtcgacggttaccaagcagctatggaagcttatggacctcagagaggaagtaacgaggagagggtgtggtggaatgccactagaaaccagggaaaacaaggaggagagtattacagggaaggaggtgaagaacctcattacccaaatactcctgctcctcatagacgtacctgggatgagagacacaaggttcttaaattgtcctcattcgctactccctctgacatccaacgctgggctactaactctagattgtacgggagctctcttcactactcgctgcgtcgagagtgtacgagactctccaggtttggtaagaaatattttatattgttataatgttactatgatccattaacactctgcttatagattgtaagggtgattgcaatgctttctgcataaaactttggttttcttgttaatcaataaaccgacttgattcgagaacctactcatatattattgtctcttttatactttattaagtaaaaggatttgtatattagccttgctaagggagacatctagtgatataagtgtgaactacacttatcttaaatgatgtaactccttaggataatcaatatacaaaattccatgacaattggcgatacccagctgcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 34 - protein, 175 aa, artificial
  synthetic granulocyte colony stimulating factor (G-CSF) filgrastim
MTPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 35 - protein, 207 aa, Homo sapiens
MAGPATQSPMKLMALQLLLWHSALWTVQEATPLGPASSLPQSFLLKCLEQVRKIQGDGAALQEKLVSECATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 36 - protein, 204 aa, Homo sapiens
MAGPATQSPMKLMALQLLLWHSALWTVQEATPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 37 - protein, 171 aa, Homo sapiens
MAGPATQSPMKLMALQLLLWHSALWTVQEATPLGPASSLPQSFLLKCLEQVRKIQGDGAALQEKLVSEAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 38 - protein, 144 aa, Homo sapiens
MWLQSLLLLGTVACSISAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 39 - protein, 273 aa, Homo sapiens
MKKTQTWILTCIYLQLLLFNPLVKTEGICRNRVTNNVKDVTKLVANLPKDYMITLKYVPGMDVLPSHCWISEMVVQLSDSLTDLLDKFSNISEGLSNYSIIDKLVNIVDDLVECVKENSSKDLKKSFKSPEPRLFTPEEFFRIFNRSIDAFKDFVVASETSDCVVSSTLSPEKDSRVSVTKPFMLPPVAASSLRNDSSSSNRKAKNPPGDSSLHWAAMALPALFSLIIGFAFGALYWKKRQPSLTRAVENIQINEEDNEISMLQEKEREFQEV

SEQ ID NO: 40 - DNA, 21 bp, artificial
  forward primer for mCherry
gatccaccggtcgccaccatg

SEQ ID NO: 41 - DNA, 22 bp, artificial
  reverse primer for mCherry
gtcgacgcggccgctttacttg

SEQ ID NO: 42 - DNA, 29 bp, artificial
  forward primer for EF1a-mCherry
actgcatgccgatggctccggtgcccgtc

SEQ ID NO: 43 - DNA, 32 bp, artificial
  EF1a-F-NotI forward primer for EF1a-GFP
attagcggccgcaggctccggtgcccgtcagt

SEQ ID NO: 44 - DNA, 34 bp, artificial
  GFP-R-NotI reverse primer for EF1a-GFP
attagcggccgcttacttgtacagctcgtccatg